SEQUENCE LISTING (418 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 202 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 5 aa, synthetic construct
SYYIH

SEQ ID NO: 4 - protein, 17 aa, synthetic construct
CIYPGNVNTNYNEKFKD

SEQ ID NO: 5 - protein, 11 aa, synthetic construct
SHYGLDWNFDV

SEQ ID NO: 6 - protein, 11 aa, synthetic construct
HASQNIYVWLN

SEQ ID NO: 7 - protein, 7 aa, synthetic construct
KASNLHT

SEQ ID NO: 8 - protein, 9 aa, synthetic construct
QQGQTYPYT

SEQ ID NO: 9 - protein, 120 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 10 - protein, 107 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 11 - protein, 5 aa, synthetic construct
DYGVH

SEQ ID NO: 12 - protein, 16 aa, synthetic construct
VIWAGGGTNYNPSLKS

SEQ ID NO: 13 - protein, 12 aa, synthetic construct
DKGYSYYYSMDY

SEQ ID NO: 14 - protein, 15 aa, synthetic construct
RASESVEYYVTSLMQ

SEQ ID NO: 15 - protein, 7 aa, synthetic construct
AASNVES

SEQ ID NO: 16 - protein, 9 aa, synthetic construct
QQSRKVPYT

SEQ ID NO: 17 - protein, 120 aa, synthetic construct
QVQLQESGPGLVKPSQTLSLTCTVSGFSLSDYGVHWVRQPPGKCLEWLGVIWAGGGTNYNPSLKSRKTISKDTSKNQVSLKLSSVTAADTAVYYCARDKGYSYYYSMDYWGQGTTVTVSS

SEQ ID NO: 18 - protein, 120 aa, synthetic construct
QVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSS

SEQ ID NO: 19 - protein, 111 aa, synthetic construct
DIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGCGTKLEIK

SEQ ID NO: 20 - protein, 111 aa, synthetic construct
DIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGGGTKLEIK

SEQ ID NO: 21 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 22 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 23 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 143 aa, Homo sapiens
QSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 26 - protein, 160 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 27 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 28 - protein, 150 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 29 - protein, 5 aa, synthetic construct
RYTMH

SEQ ID NO: 30 - protein, 17 aa, synthetic construct
YINPSRGYTNYNQKFKD

SEQ ID NO: 31 - protein, 10 aa, synthetic construct
YYDDHYCLDY

SEQ ID NO: 32 - protein, 10 aa, synthetic construct
SASSSVSYMN

SEQ ID NO: 33 - protein, 7 aa, synthetic construct
DTSKLAS

SEQ ID NO: 34 - protein, 9 aa, synthetic construct
QQWSSNPFT

SEQ ID NO: 35 - protein, 119 aa, synthetic construct
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSS

SEQ ID NO: 36 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 37 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 38 - protein, 327 aa, Homo sapiens
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 39 - protein, 5 aa, synthetic construct
KYAMN

SEQ ID NO: 40 - protein, 19 aa, synthetic construct
RIRSKYNNYATYYADSVKD

SEQ ID NO: 41 - protein, 14 aa, synthetic construct
HGNFGNSYISYWAY

SEQ ID NO: 42 - protein, 14 aa, synthetic construct
GSSTGAVTSGYYPN

SEQ ID NO: 43 - protein, 7 aa, synthetic construct
GTKFLAP

SEQ ID NO: 44 - protein, 9 aa, synthetic construct
ALWYSNRWV

SEQ ID NO: 45 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 46 - protein, 125 aa, synthetic construct
EVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSS

SEQ ID NO: 47 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 48 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVL

SEQ ID NO: 49 - protein, 5 aa, synthetic construct
KAWMH

SEQ ID NO: 50 - protein, 19 aa, synthetic construct
QIKDKSNSYATYYADSVKG

SEQ ID NO: 51 - protein, 10 aa, synthetic construct
VYYALSPFDY

SEQ ID NO: 52 - protein, 16 aa, synthetic construct
KSSQSLVHNNANTYLS

SEQ ID NO: 53 - protein, 7 aa, synthetic construct
KVSNRFS

SEQ ID NO: 54 - protein, 9 aa, synthetic construct
GQGTQYPFT

SEQ ID NO: 55 - protein, 121 aa, synthetic construct
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 56 - protein, 112 aa, synthetic construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 57 - protein, 570 aa, Homo sapiens
LAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALS

SEQ ID NO: 58 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 59 - protein, 1233 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 60 - protein, 5 aa, synthetic construct
GYYMH

SEQ ID NO: 61 - protein, 5 aa, synthetic construct
DTYIH

SEQ ID NO: 62 - protein, 17 aa, synthetic construct
RIYPTNGYTRYADSVKG

SEQ ID NO: 63 - protein, 11 aa, synthetic construct
WGGDGFYAMDY

SEQ ID NO: 64 - protein, 11 aa, synthetic construct
RASQDVNTAVA

SEQ ID NO: 65 - protein, 7 aa, synthetic construct
SASFLYS

SEQ ID NO: 66 - protein, 9 aa, synthetic construct
QQHYTTPPT

SEQ ID NO: 67 - protein, 120 aa, synthetic construct [sequence omitted]

SEQ ID NO: 68 - protein, 107 aa, synthetic construct [sequence omitted]

SEQ ID NO: 69 - protein, 5 aa, synthetic construct
NYGVH

SEQ ID NO: 70 - protein, 16 aa, synthetic construct
VIWSGGNTDYNTPFTS

SEQ ID NO: 71 - protein, 11 aa, synthetic construct
ALTYYDYEFAY

SEQ ID NO: 72 - protein, 11 aa, synthetic construct
RASQSIGTNIH

SEQ ID NO: 73 - protein, 7 aa, synthetic construct
YASESIS

SEQ ID NO: 74 - protein, 9 aa, synthetic construct
QQNNNWPTT

SEQ ID NO: 75 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 76 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 77 - protein, 7 aa, synthetic construct
SGSYYWS

SEQ ID NO: 78 - protein, 16 aa, synthetic construct
YIYYSGSTNYNPSLKS

SEQ ID NO: 79 - protein, 9 aa, synthetic construct
EGKNGAFDI

SEQ ID NO: 80 - protein, 11 aa, synthetic construct
RASQSISSYLN

SEQ ID NO: 81 - protein, 7 aa, synthetic construct
AASSLQS

SEQ ID NO: 82 - protein, 9 aa, synthetic construct
QQSYSTPLT

SEQ ID NO: 83 - protein, 119 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 84 - protein, 107 aa, synthetic construct
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 85 - protein, 17 aa, synthetic construct
RINPNSGGTNYAQKFQG

SEQ ID NO: 86 - protein, 14 aa, synthetic construct
GRTEYIVVAEGFDY

SEQ ID NO: 87 - protein, 16 aa, synthetic construct
RSSQSLVYSDGNTHLN

SEQ ID NO: 88 - protein, 7 aa, synthetic construct
KVSNRDS

SEQ ID NO: 89 - protein, 9 aa, synthetic construct
MQGTHWPYT

SEQ ID NO: 90 - protein, 123 aa, synthetic construct
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARGRTEYIVVAEGFDYWGQGTLVTVSS

SEQ ID NO: 91 - protein, 112 aa, synthetic construct
DVVMTQSPLSLPVTLGQPASISCRSSQSLVYSDGNTHLNWFQQRPGQSPRRLIYKVSNRDSGVPDRFSGSGSGTDFTLKISRVEAEDVGVYYCMQGTHWPYTFGQGTKLEIK

SEQ ID NO: 92 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 93 - protein, 20 aa, Homo sapiens
MYRMQLLSCIALSLALVTNS

SEQ ID NO: 94 - protein, 25 aa, Homo sapiens
MGVLLTQRTLLSLVLALLFPSMASM

SEQ ID NO: 95 - protein, 18 aa, Homo sapiens
MASLWLLSCFSLVGAAFG

SEQ ID NO: 96 - protein, 15 aa, Homo sapiens
MNLLLILTFVAAAVA

SEQ ID NO: 97 - protein, 24 aa, Homo sapiens
MALWMRLLPLLALLALWGPDPAAA

SEQ ID NO: 98 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 99 - protein, 12 aa, synthetic construct
EPKSCDKTHTCP

SEQ ID NO: 100 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 101 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 106 aa, synthetic construct [sequence omitted]

SEQ ID NO: 103 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 330 aa, synthetic construct [sequence omitted]

SEQ ID NO: 110 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 112 - protein, 12 aa, synthetic construct
ESKYGPPCPSCP

SEQ ID NO: 113 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 114 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 106 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 116 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 216 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 118 - protein, 223 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 222 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 120 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 121 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 122 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 124 - protein, 107 aa, synthetic construct [sequence omitted]

SEQ ID NO: 125 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 126 - protein, 81 aa, synthetic construct
PTLFPLVSCENSNPSSTVAVGCLAQDFLPDSITFSWKYNQSQKISSTRGFPSVLRGGKYAATSQVLLPSKDVMQGTDEHVC

SEQ ID NO: 127 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 232 aa, synthetic construct; identical to SEQ ID NO: 139

SEQ ID NO: 142 - protein, 231 aa, synthetic construct; identical to SEQ ID NO: 140

SEQ ID NO: 143 - protein, 232 aa, synthetic construct; identical to SEQ ID NO: 139

SEQ ID NO: 144 - protein, 231 aa, synthetic construct; identical to SEQ ID NO: 140

SEQ ID NO: 145 - protein, 217 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 147 - protein, 223 aa, synthetic construct
PCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 149 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 150 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 151 - protein, 230 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 152 - protein, 229 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 153 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 157 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 167 - protein, 330 aa, synthetic construct; identical to SEQ ID NO: 159

SEQ ID NO: 168 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 170 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 171 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 172 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 173 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 174 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 175 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 176 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 177 - protein, 220 aa, synthetic construct; identical to SEQ ID NO: 169

SEQ ID NO: 178 - protein, 219 aa, synthetic construct; identical to SEQ ID NO: 170

SEQ ID NO: 179 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 180 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 181 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 182 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 183 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 184 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 185 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 191 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 193 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 194 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 195 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 196 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 197 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 198 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 199 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 200 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 201 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 205 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 207 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 209 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 210 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 211 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 212 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 213 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 214 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 215 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 216 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 217 - protein, 4 aa, synthetic construct
GGGS

SEQ ID NO: 218 - protein, 8 aa, synthetic construct
GGGSGGGS

SEQ ID NO: 219 - protein, 12 aa, synthetic construct
GGGSGGGSGGGS

SEQ ID NO: 220 - protein, 16 aa, synthetic construct
GGGSGGGSGGGSGGGS

SEQ ID NO: 221 - protein, 5 aa, synthetic construct
GGGGS

SEQ ID NO: 222 - protein, 10 aa, synthetic construct
GGGGSGGGGS

SEQ ID NO: 223 - protein, 15 aa, synthetic construct
GGGGSGGGGSGGGGS

SEQ ID NO: 224 - protein, 20 aa, synthetic construct
GGGGSGGGGSGGGGSGGGGS

SEQ ID NO: 225 - protein, 8 aa, synthetic construct
GGGGGGGS

SEQ ID NO: 226 - protein, 16 aa, synthetic construct
GGGGGGGSGGGGGGGS

SEQ ID NO: 227 - protein, 24 aa, synthetic construct
GGGGGGGSGGGGGGGSGGGGGGGS

SEQ ID NO: 228 - protein, 32 aa, synthetic construct
GGGGGGGSGGGGGGGSGGGGGGGSGGGGGGGS

SEQ ID NO: 229 - protein, 5 aa, synthetic construct
SGGGG

SEQ ID NO: 230 - protein, 10 aa, synthetic construct
SGGGGSGGGG

SEQ ID NO: 231 - protein, 15 aa, synthetic construct
SGGGGSGGGGSGGGG

SEQ ID NO: 232 - protein, 20 aa, synthetic construct
SGGGGSGGGGSGGGGSGGGG

SEQ ID NO: 233 - protein, 7 aa, synthetic construct
GGGGGGS

SEQ ID NO: 234 - protein, 14 aa, synthetic construct
GGGGGGSGGGGGGS

SEQ ID NO: 235 - protein, 21 aa, synthetic construct
GGGGGGSGGGGGGSGGGGGGS

SEQ ID NO: 236 - protein, 28 aa, synthetic construct
GGGGGGSGGGGGGSGGGGGGSGGGGGGS

SEQ ID NO: 237 - protein, 714 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 238 - protein, 707 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 239 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 705 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 698 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 242 - protein, 708 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 715 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 244 - protein, 707 aa, synthetic construct
QVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 457 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTELPPKVSVFVPPRDGFFGNPRKSELICEATGFSPRQIQVSWLREGKQVGSGVTTDQVQAEAKESGPTTYDVTSTLTIKESDWLGQSMFTCRVDHRGLTFQQSASSMCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 246 - protein, 216 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 217 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTAELPPKVSVFVPPRDGFFGNPRKSKLICKATGFSPRQIQVSWLREGKQVGSGVTTKQVQAEAKESGPTTYKVTSTLTIKESDWLGQSMFTCRVDHRGLTFQQSASSMC

SEQ ID NO: 248 - protein, 713 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSGGGGSGGGGSGGGGSDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 249 - protein, 711 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSSGGGGSGGGGSGGGGSDIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGQGTKLEIK

SEQ ID NO: 250 - protein, 704 aa, synthetic construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGGGSGGGGSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 702 aa, synthetic construct
DIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGQGTKLEIKGGGGSGGGGSGGGGSQVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 252 - protein, 712 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQESGPGLVKPSQTLSLTCTVSGFSLSDYGVHWVRQPPGKCLEWLGVIWAGGGTNYNPSLKSRKTISKDTSKNQVSLKLSSVTAADTAVYYCARDKGYSYYYSMDYWGQGTTVTVSSGGGGSGGGGSGGGGSDIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGCGTKLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 714 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVESGGGVVQPGRSLRLSCAASGFTFTKAWMHWVRQAPGKCLEWVAQIKDKSNSYATYYADSVKGRFTISRDDSKNTLYLQMNSLRAEDTAVYYCRGVYYALSPFDYWGQGTLVTVSSGGGGSGGGGSGGGGSDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGCGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 254 - protein, 713 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 255 - protein, 706 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 256 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 704 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 697 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 259 - protein, 707 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 714 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 261 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 450 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 214 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 264 - protein, 446 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 265 - protein, 214 aa, synthetic construct; identical to SEQ ID NO: 263

SEQ ID NO: 266 - protein, 455 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 216 aa, synthetic construct; identical to SEQ ID NO: 246

SEQ ID NO: 268 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 269 - protein, 447 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 270 - protein, 571 aa, synthetic construct
QVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSSSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSRTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLGK

SEQ ID NO: 271 - protein, 342 aa, synthetic construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGQPKAAPDIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGQGTKLEIKTKGPSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 7 aa, synthetic construct
SGDYYWT

SEQ ID NO: 273 - protein, 16 aa, synthetic construct
HIYYSGNTNYNPSLKS

SEQ ID NO: 274 - protein, 9 aa, synthetic construct
DRVTGAFDI

SEQ ID NO: 275 - protein, 11 aa, synthetic construct
QASQDISNYLN

SEQ ID NO: 276 - protein, 7 aa, synthetic construct
DASNLET

SEQ ID NO: 277 - protein, 9 aa, synthetic construct
QHFDHLPLA

SEQ ID NO: 278 - protein, 119 aa, synthetic construct [sequence omitted]

SEQ ID NO: 279 - protein, 107 aa, synthetic construct [sequence omitted]

SEQ ID NO: 280 - protein, 5 aa, synthetic construct
TYGMH

SEQ ID NO: 281 - protein, 17 aa, synthetic construct
VIWDDGSYKYYGDSVKG

SEQ ID NO: 282 - protein, 16 aa, synthetic construct
DGITMVRGVMKDYFDY

SEQ ID NO: 283 - protein, 11 aa, synthetic construct
RASQDISSALV

SEQ ID NO: 284 - protein, 7 aa, synthetic construct
DASSLES

SEQ ID NO: 285 - protein, 9 aa, synthetic construct
QQFNSYPLT

SEQ ID NO: 286 - protein, 125 aa, synthetic construct [sequence omitted]

SEQ ID NO: 287 - protein, 107 aa, synthetic construct [sequence omitted]

SEQ ID NO: 288 - protein, 5 aa, synthetic construct
GYTMN

SEQ ID NO: 289 - protein, 17 aa, synthetic construct
LITPYNGASSYNQKFRG

SEQ ID NO: 290 - protein, 10 aa, synthetic construct
GGYDGRGFDY

SEQ ID NO: 291 - protein, 10 aa, synthetic construct
SASSSVSYMH

SEQ ID NO: 292 - protein, 7 aa, synthetic construct
DTSKLAS

SEQ ID NO: 293 - protein, 9 aa, synthetic construct
QQWSKHPLT

SEQ ID NO: 294 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 295 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 296 - protein, 5 aa, synthetic construct
SNWIG

SEQ ID NO: 297 - protein, 17 aa, synthetic construct
IIYPGDSDTRYSPSFQG

SEQ ID NO: 298 - protein, 10 aa, synthetic construct
QTGFLWSSDL

SEQ ID NO: 299 - protein, 11 aa, synthetic construct
RASQDISSALA

SEQ ID NO: 300 - protein, 7 aa, synthetic construct
DASSLES

SEQ ID NO: 301 - protein, 9 aa, synthetic construct
QQFNSYPLT

SEQ ID NO: 302 - protein, 119 aa, synthetic construct
EVQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQTGFLWSSDLWGRGTLVTVSS

SEQ ID NO: 303 - protein, 107 aa, synthetic construct
AIQLTQSPSSLSASVGDRVTITCRASQDISSALAWYQQKPGKAPKLLIYDASSLESGVPSRFSGYGSGTDFTLTINSLQPEDFATYYCQQFNSYPLTFGGGTKVEIK

SEQ ID NO: 304 - protein, 713 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVL

SEQ ID NO: 305 - protein, 706 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIK

SEQ ID NO: 306 - protein, 214 aa, synthetic construct
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 307 - protein, 704 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSASTKGPQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 697 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKGGGGSGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSASTKGPQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 309 - protein, 707 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 714 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 311 - protein, 713 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVL

SEQ ID NO: 312 - protein, 706 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIK

SEQ ID NO: 313 - protein, 213 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - protein, 704 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSASTKGPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 697 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKGGGGSGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSASTKGPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 316 - protein, 707 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 714 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 318 - protein, 10 aa, synthetic construct
DKTHTCPPCP

SEQ ID NO: 319 - protein, 16 aa, synthetic construct
EPKSCDGGGGSGGGGS

SEQ ID NO: 320 - protein, 708 aa, synthetic construct
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVL

SEQ ID NO: 321 - protein, 701 aa, synthetic construct
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIK

SEQ ID NO: 322 - protein, 216 aa, synthetic construct
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 699 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSASTKGPEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 692 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKGGGGSGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSASTKGPEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 325 - protein, 702 aa, synthetic construct
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYTFTSYYIHWVRQAPGQCLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGCGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 709 aa, synthetic construct
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGCGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 327 - protein, 5 aa, synthetic construct
EYIIH

SEQ ID NO: 328 - protein, 17 aa, synthetic construct
WFYPGSNDIQYNAQFKG

SEQ ID NO: 329 - protein, 12 aa, synthetic construct
RDDFSGYDALPY

SEQ ID NO: 330 - protein, 11 aa, synthetic construct
KTNENIYSNLA

SEQ ID NO: 331 - protein, 7 aa, synthetic construct
AATHLVE

SEQ ID NO: 332 - protein, 9 aa, synthetic construct
QHFWGTPCT

SEQ ID NO: 333 - protein, 120 aa, synthetic construct
VQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 334 - protein, 108 aa, synthetic construct
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIKR

SEQ ID NO: 335 - protein, 108 aa, synthetic construct
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPATFGGGTKLEIKR

SEQ ID NO: 336 - protein, 108 aa, synthetic construct
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPNTFGGGTKLEIKR

SEQ ID NO: 337 - protein, 5 aa, synthetic construct
EYYMY

SEQ ID NO: 338 - protein, 17 aa, synthetic construct
RIDPEDGSIDYVEKFKK

SEQ ID NO: 339 - protein, 9 aa, synthetic construct
GKFNYRFAY

SEQ ID NO: 340 - protein, 16 aa, synthetic construct
RSSQSLLHSSGNTYLN

SEQ ID NO: 341 - protein, 7 aa, synthetic construct
LVSKLES

SEQ ID NO: 342 - protein, 9 aa, synthetic construct
MQFTHYPYT

SEQ ID NO: 343 - protein, 118 aa, synthetic construct
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 344 - protein, 112 aa, synthetic construct
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 345 - protein, 118 aa, synthetic construct
EVQLQQSGPELQRPGASVKLSCKASGYIFTEYYMYWVKQRPKQQLELVGRIDPEDGSIDYVEKFKKKATLTADTSSNTAYMQLSSLTSEDTATYFCARGKFNYRFAYWGQGTLVTVSS

SEQ ID NO: 346 - protein, 111 aa, synthetic construct
DVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELK

SEQ ID NO: 347 - protein, 118 aa, synthetic construct
QVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 348 - protein, 112 aa, synthetic construct; identical to SEQ ID NO: 344

SEQ ID NO: 349 - protein, 117 aa, synthetic construct
EVQLVESGGGLVQPGGSLRLSCAASGFTFSDYWMSWVRQAPGKRLEWIGHIKYDGSYTNYAPSLKNRFTISRDNAKNSLYLQMNSLRSEDTAVYYCAREAPGAASYWGQGTLVTVSS

SEQ ID NO: 350 - protein, 112 aa, synthetic construct
DVVLTQTPLSLSVTPGQPASISCRSSQSLVHSNGNTYLEWFLQKPGQSPQLLIYKVSNRFSGVPDRFSGSGSGSDFTLKISRVEAEDVGVYYCFQGTHDPYTFGQGTKVEIK

SEQ ID NO: 351 - protein, 5 aa, synthetic construct
TYAMN

SEQ ID NO: 352 - protein, 19 aa, synthetic construct
RIRSKYNNYATYYADSVKD

SEQ ID NO: 353 - protein, 14 aa, synthetic construct
HGNFGNSYVSWFAY

SEQ ID NO: 354 - protein, 14 aa, synthetic construct
RSSTGAVTTSNYAN

SEQ ID NO: 355 - protein, 7 aa, synthetic construct
GTNKRAP

SEQ ID NO: 356 - protein, 9 aa, synthetic construct
ALWYSNLWV

SEQ ID NO: 357 - protein, 125 aa, synthetic construct
EVQLVESGGGLVQPGGSLRLSCAASGFTFSTYAMNWVRQAPGKGLEWVARIRSKYNNYATYYADSVKDRFTISRDDSKNSLYLQMNSLRAEDTAVYYCARHGNFGNSYVSWFAYWGQGTLVTVSS

SEQ ID NO: 358 - protein, 110 aa, synthetic construct
EIVLTQSPATLSLSPGERATLSCRSSTGAVTTSNYANWVQQKPGQAPRGLIGGTNKRAPGVPARFSGSLSGTDATLTISSLQPEDFAVYYCALWYSNLWVFGGGTKVEIK

SEQ ID NO: 359 - protein, 7 aa, synthetic construct
NNNAAWS

SEQ ID NO: 360 - protein, 18 aa, synthetic construct
RTYYRSKWLYDYAVSVKS

SEQ ID NO: 361 - protein, 8 aa, synthetic construct
GYSSSFDY

SEQ ID NO: 362 - protein, 14 aa, synthetic construct
TGTSSNIGTYKFVS

SEQ ID NO: 363 - protein, 7 aa, synthetic construct
EVSKRPS

SEQ ID NO: 364 - protein, 10 aa, synthetic construct
VSYAGSGTLL

SEQ ID NO: 365 - protein, 120 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 366 - protein, 110 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 367 - protein, 8 aa, synthetic construct
GFTFTDYT

SEQ ID NO: 368 - protein, 15 aa, synthetic construct
VNPNSGGSIYNQRFK

SEQ ID NO: 369 - protein, 9 aa, synthetic construct
LGPSFYFDY

SEQ ID NO: 370 - protein, 11 aa, synthetic construct
KASQDVSIGVA

SEQ ID NO: 371 - protein, 7 aa, synthetic construct
ASYRYTG

SEQ ID NO: 372 - protein, 10 aa, synthetic construct
QQYYIYPYTF

SEQ ID NO: 373 - protein, 119 aa, synthetic construct [sequence omitted]

SEQ ID NO: 374 - protein, 108 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 375 - protein, 5 aa, synthetic construct
DYYMY

SEQ ID NO: 376 - protein, 17 aa, synthetic construct
YINSGGGSTYYPDTVKG

SEQ ID NO: 377 - protein, 12 aa, synthetic construct
EALYDYDYAMDY

SEQ ID NO: 378 - protein, 16 aa, synthetic construct
KSSQSLLYSNGKTYLN

SEQ ID NO: 379 - protein, 7 aa, synthetic construct
LVSKLDS

SEQ ID NO: 380 - protein, 9 aa, synthetic construct
VQGTHFPLT

SEQ ID NO: 381 - protein, 121 aa, synthetic construct
EVQLVESGGGLVQPGGSLRLSCAASGFTFSDYYMYWVRQAPGKGLEWVAYINSGGGSTYYPDTVKGRFTISRDNAKNSLYLQMNSLRAEDTAVYYCAREALYDYDYAMDYWGQGTTVTVSS

SEQ ID NO: 382 - protein, 112 aa, synthetic construct
DIVMTQSPLSLSVTPGQPASISCKSSQSLLYSNGKTYLNWLLQKPGQSPQRLIYLVSKLDSGVPDRFSGSGSGTDFTLKISRVEAEDVGVYYCVQGTHFPLTFGGGTKVEIK

SEQ ID NO: 383 - protein, 5 aa, synthetic construct
GYYMH

SEQ ID NO: 384 - protein, 17 aa, synthetic construct
RINPYTGVPSYKHNFKD

SEQ ID NO: 385 - protein, 5 aa, synthetic construct
ELGGY

SEQ ID NO: 386 - protein, 14 aa, synthetic construct
RSSTGAVTTGNYPN

SEQ ID NO: 387 - protein, 7 aa, synthetic construct
GTNNRAP

SEQ ID NO: 388 - protein, 9 aa, synthetic construct
ALWFSSHWV

SEQ ID NO: 389 - protein, 114 aa, synthetic construct
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSS

SEQ ID NO: 390 - protein, 109 aa, synthetic construct
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVL

SEQ ID NO: 391 - protein, 17 aa, synthetic construct
RINPYTGVPSYKHKFQG

SEQ ID NO: 392 - protein, 14 aa, synthetic construct
ASSTGAVTTGNYPN

SEQ ID NO: 393 - protein, 7 aa, synthetic construct
GTNNKAS

SEQ ID NO: 394 - protein, 114 aa, synthetic construct
EVQLVQSGAEVKKPGASVKVSCKASGYSFTGYYMHWVRQAPGQGLEWIGRINPYTGVPSYKHKFQGRVTLTVDKSTSTAYMELSSLRSEDTAVYYCARELGGYWGQGTTVTVSS

SEQ ID NO: 395 - protein, 109 aa, synthetic construct
QAVVTQEPSLTVSPGGTVTLTCASSTGAVTTGNYPNWFQEKPGQAFRGLIAGTNNRAPWVPARFSGSLIGDKAALTLSGVQPEDEAEYFCALWFSSHWVFGGGTKLTVL

SEQ ID NO: 396 - protein, 114 aa, synthetic construct
QVQLVQSGAEVKKPGASVKVSCKASGYSFTGYYMHWVRQAPGQGLEWMGRINPYTGVPSYKHKFQGRVTMTVDKSTSTAYMELSSLRSEDTAVYYCARELGGYWGQGTTVTVSS

SEQ ID NO: 397 - protein, 109 aa, synthetic construct
QAVVTQEPSLTVSPGGTVTLTCASSTGAVTTGNYPNWFQQKPGQAFRGLIAGTNNKASWTPARFSGSLLGDKAALTLSGVQPEDEAEYYCALWFSSHWVFGGGTKLTVL

SEQ ID NO: 398 - protein, 9 aa, synthetic construct
QQWSGYPLT

SEQ ID NO: 399 - protein, 119 aa, synthetic construct
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 400 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 401 - protein, 6 aa, synthetic construct
FYFYAC

SEQ ID NO: 402 - protein, 18 aa, synthetic construct
CIYTAGSGSTYYASWAKG

SEQ ID NO: 403 - protein, 13 aa, synthetic construct
STANTRSTYYLNL

SEQ ID NO: 404 - protein, 11 aa, synthetic construct
QASQRISSYLS

SEQ ID NO: 405 - protein, 7 aa, synthetic construct
GASTLAS

SEQ ID NO: 406 - protein, 13 aa, synthetic construct
QSYAYFDSNNWHA

SEQ ID NO: 407 - protein, 123 aa, synthetic construct
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSS

SEQ ID NO: 408 - protein, 110 aa, synthetic construct
DVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 409 - protein, 5 aa, synthetic construct
SYWMS

SEQ ID NO: 410 - protein, 17 aa, synthetic construct
NIKQDGSEKYYVDSVKG

SEQ ID NO: 411 - protein, 15 aa, synthetic construct
VWDYYYDSSGDAFDI

SEQ ID NO: 412 - protein, 11 aa, synthetic construct
RASQGISSWLA

SEQ ID NO: 413 - protein, 7 aa, synthetic construct
AASNLQS

SEQ ID NO: 414 - protein, 9 aa, synthetic construct
QQANSFPLT

SEQ ID NO: 415 - protein, 124 aa, synthetic construct
QVQLVQSGGGLVQPGGSLRLSCAASGFTFSSYWMSWVRQAPGKGLEWVANIKQDGSEKYYVDSVKGRFTISRDNAKNSLYLQMNSLRAEDTAVYYCARVWDYYYDSSGDAFDIWGQGTMVTVSS

SEQ ID NO: 416 - protein, 107 aa, synthetic construct
VIWMTQSPSSVSASVGDRVTITCRASQGISSWLAWYQQKPGKAPKLLIYAASNLQSGVPSRFSGSGSGTDFTLTISSLQPEDFATYYCQQANSFPLTFGGGTKVDIK

SEQ ID NO: 417 - protein, 449 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 418 - protein, 213 aa, synthetic construct; identical to SEQ ID NO: 313

(19) United States
(12) Patent Application Publication
(10) Pub. No.: US 2024/0228620 A1
(43) Pub. Date: Jul. 11, 2024

(54) MULTISPECIFIC PROTEINS AND RELATED METHODS

(71) Applicant: Bicara Therapeutics Inc., Cambridge, MA (US)

(72) Inventors: Arvind Vittal GOSWAMI, Bangalore (IN); Jaya BHATNAGAR, Pune (IN); Reshmi NAIR, Jigani Hobli (IN); Veena SOMASUNDARAM, Bengaluru (IN); Seng-Lai TAN, Sudbury, MA (US); Avanish Kumar VARSHNEY, Westford, MA (US)

(21) Appl. No.: 18/481,511

(22) Filed: Oct. 5, 2023

Related U.S. Application Data
(60) Provisional application No. 63/413,765, filed on Oct. 6, 2022.

(51) Int. Cl. C07K 16/28; A61K 39/00; A61P 35/00; A61P 37/04; C07K 16/30

(52) U.S. Cl. CPC C07K 16/2818; A61K 39/4633; A61P 35/00; A61P 37/04; C07K 16/2806; C07K 16/30; C07K 2317/31; C07K 2317/622

(57) ABSTRACT

Provided herein are multispecific proteins and compositions (e.g., pharmaceutical compositions) comprising the same; as well as methods of making the multispecific proteins and compositions. The multispecific proteins provided herein are useful in pharmaceutical compositions and methods, including, e.g., methods of treating diseases (e.g., cancer), methods of activating T-cells, and methods of inducing or enhancing an immune response.

### RELATED APPLICATIONS

This application claims priority to U.S. Ser. No. 63/413,765, filed Oct. 6, 2022, the entire contents of which is incorporated herein by reference.

### SEQUENCE LISTING

The instant application contains a Sequence Listing which has been submitted electronically in XML format and is hereby incorporated by reference in its entirety. Said XML copy, created on Mar. 27, 2024, is named 62804_33US01_SL.xml and is 434,867 bytes in size.

### 1. FIELD

This disclosure relates to multispecific proteins (and polypeptides thereof) and polynucleotides encoding the same. The disclosure further relates to methods of making and utilizing the same.

### 2. BACKGROUND

T-cell engager immunotherapies function by activating T-cells (or a subset thereof, (e.g., CD8+ T cells)) to mediate e.g., tumor cell lysis. A subset of T-cell engagers are multispecific, aiming to target activated T-cells to the tumor cells by simultaneously binding a T-cell antigen (e.g., CD3) and an antigen expressed on the surface of tumor cells (e.g., a tumor associated antigen). Exemplary T-cell engagers include, bispecific T cell engagers (BiTE), bifunctional checkpoint-inhibitory T cell engagers (CiTEs), simultaneous multiple interaction T cell engagers (SMITEs), trispecific killer engagers (TriKEs), and BiTE-expressing chimeric antigen receptor (CAR) T cells (CART.BiTE cells).

### 3. SUMMARY

Provided herein are, inter alia, multispecific proteins (and polypeptides thereof) and polynucleotides encoding the same; methods of manufacturing; pharmaceutical compositions; and methods of use including e.g., methods of treating diseases (e.g., cancer) and methods of inducing an immune response.

In one aspect, provided herein are multispecific proteins comprising: (a) a full-length antibody comprising: (i) a first light chain comprising from N- to C-terminus a light chain variable region (VL) region and a light chain constant region (CL) region; (ii) a first heavy chain comprising from N- to C-terminus a heavy chain variable region (VH) region, a CH1 region, a hinge region, a CH2 region, and a CH3 region; (iii) a second heavy chain comprising from N- to C-terminus a VH region, a CH1 region, a hinge region, a CH2 region, and a CH3 region; (iv) a second light chain comprising from N- to C-terminus a VL region and a CL region; wherein the first light chain and the first heavy chain associate to form a first antigen binding domain; wherein the second light chain and the second heavy chain associate to form a second antigen binding domain; and wherein the first heavy chain and the second heavy chain associate to form a dimer; (b) a first single chain variable fragment (scFv) operably connected to the C-terminus of the CH3 region of the first heavy chain of the full-length antibody, wherein the first scFv comprises from N- to C-terminus (i) a VH region, a peptide linker, and a VL region, or (ii) a VL region, a peptide linker, and a VH region; and (c) a second scFv operably connected to the C-terminus of the CH3 region of the second heavy chain of the full-length antibody, wherein the second scFv comprises from N- to C-terminus (i) a VH region, a peptide linker, and a VL region, or (ii) a VL region, a peptide linker, and a VH region; wherein the first antigen binding domain of the full-length antibody specifically binds to a first human tumor associated antigen (hTAA); wherein the second antigen binding domain of the full-length antibody specifically binds to a second hTAA; wherein the first scFv specifically binds to a human T-cell co-stimulatory antigen (hTCSA) (e.g., hCD28, hCD2); wherein the second scFv specifically binds to human CD3 (hCD3); and wherein the CH3 region of the first heavy chain of the full-length antibody comprises one or more amino acid modification (e.g., substitution) relative to the amino acid sequence of a reference CH3 region that does not contain the one or more amino acid modification (e.g., a reference CH3 region, e.g., a wild-type CH3 region, e.g., SEQ ID NO: 101), wherein the CH3 region of the second heavy chain of the of the full-length antibody comprises one or more amino acid modification (e.g., substitution) relative to the amino acid sequence of a reference CH3 region that does not contain the one or more amino acid modification (e.g., a wild-type CH3 region, e.g., SEQ ID NO: 101); and wherein the one or more amino acid modification in the CH3 region of the first heavy chain of the full-length antibody is different from the one or more amino acid modification in the CH3 region of the second heavy chain of the full-length antibody; wherein the one or more amino acid modification in the CH3 region of the first heavy chain of the full-length antibody and the one or more amino acid modification in the CH3 region of the second heavy chain of the full-length antibody promote heterodimerization of the first and second heavy chain of the full-length antibody; wherein the CH2 region of the first heavy chain of the full-length antibody comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the CH2 region of the second heavy chain of the full-length antibody comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the one or more amino acid modification in the CH2 region of the first heavy chain of the full-length antibody and the one or more amino acid modification in the CH2 region of the second heavy chain of the full-length antibody reduce or abolish one or more of the following heavy chain effector functions relative to a reference heavy chain that does not contain the one or more amino acid modification (e.g., a heavy chain comprising a wild type CH2 region, e.g., SEQ ID NO: 100): antibody dependent cellular cytotoxicity (ADCC), antibody dependent cellular phagocytosis (ADCP), complement dependent cytotoxicity (CDC), and/or binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the first scFv is operably connected to the C-terminus of the CH3 region of the first heavy chain of the full-length antibody directly through a peptide bond.

In some embodiments, the first scFv is operably connected to the C-terminus of the CH3 region of the first heavy chain of the full-length antibody through a first peptide linker. In some embodiments, the amino acid sequence of the first peptide linker comprises or consists of glycine or glycine and serine amino acid residues. In some embodiments, the amino acid sequence of the first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

In some embodiments, the second scFv is operably connected to the C-terminus of the CH3 region of the second heavy chain of the full-length antibody directly through a peptide bond.

In some embodiments, the second scFv is operably connected to the C-terminus of the CH3 region of the second heavy chain of the full-length antibody through a second peptide linker. In some embodiments, the amino acid sequence of the second peptide linker comprises or consists of glycine or glycine and serine amino acid residues. In some embodiments, the amino acid of the second peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

In some embodiments, the first scFv comprises from N- to C-terminus: a VL region, a peptide linker, and a VH region; and wherein the N-terminus of the VL region of the scFv is operably connected to the C-terminus of the CH3 region of the first heavy chain of the full-length antibody. In some embodiments, the first scFv comprises from N- to C-terminus: a VH region, a peptide linker, and a VL region; and wherein the N-terminus of the VH region of the scFv is operably connected to the C-terminus of the CH3 region of the first heavy chain of the full-length antibody. In some embodiments, the second scFv comprises from N- to C-terminus: a VL region, a peptide linker, and a VH region; and wherein the N-terminus of the VL region of the scFv is operably connected to the C-terminus of the CH3 region of the second heavy chain of the full-length antibody. In some embodiments, the second scFv comprises from N- to C-terminus: a VH region, a peptide linker, and a VL region; and wherein the N-terminus of the VH region of the scFv is operably connected to the C-terminus of the CH3 region of the second heavy chain of the full-length antibody.

In some embodiments, the first hTAA and the second hTAA are expressed by (e.g., on the surface of) the same tumor cell. In some embodiments, the first hTAA and the second hTAA are the same. In some embodiments, the first scFv and the second scFv specifically bind the same epitope of the same hTAA. In some embodiments, the first scFv and the second scFv specifically bind different epitopes of the same hTAA. In some embodiments, the first tumor associated antigen is hEGFR, hMSLN, hPSMA, or hHER2; and the second tumor associated antigen is hEGFR, hMSLN, hPSMA, or hHER2.

In some embodiments, the amino acid sequence of the VH region of the first scFv and the amino acid sequence of the VL region of the first scFv each comprises a cysteine amino acid residue, wherein the cysteine amino acid residues are capable of forming a disulfide bond. In some embodiments, the amino acid sequence of the VH region of the first scFv comprises a cysteine at amino acid position 97, 98, 99, 100, 101, 102, 103, 104, 105, or 106, amino acid numbering according to Kabat; and the amino acid sequence of the VL region of the first scFv comprises a cysteine at amino acid position 40, 41, 42, 43, 44, 45, 46, or 47, amino acid numbering according to Kabat. In some embodiments, the amino acid sequence of the VH region of the second scFv comprises a cysteine at amino acid position 97, 98, 99, 100, 101, 102, 103, 104, 105, or 106, amino acid numbering according to Kabat; and the amino acid sequence of the VL region of the second scFv comprises a cysteine at amino acid position 40, 41, 42, 43, 44, 45, 46, or 47, amino acid numbering according to Kabat.

In some embodiments, the full-length antibody is a human IgG (hIgG) antibody. In some embodiments, the full-length antibody is a hIgG1 or hIgG4 antibody.

In some embodiments, the full-length antibody is an IgG1 antibody, and wherein the amino acid sequence of the first heavy chain of the full-length antibody comprises an amino acid substitution at amino acid positions T366, L368, and Y407, numbering according to the EU index of Kabat. In some embodiments, the full-length antibody is an IgG1 antibody, and wherein the amino acid sequence of the first heavy chain of the full-length antibody comprises a serine at amino acid position T366, an alanine at amino acid position L368, and a valine at amino acid position Y407, numbering according to the EU index of Kabat. In some embodiments, the full-length antibody is an IgG1 antibody, and wherein the amino acid sequence of the first heavy chain of the full-length antibody comprises an amino acid substitution at amino acid position Y349, numbering according to the EU index of Kabat. In some embodiments, the full-length antibody is an IgG1 antibody, and wherein the amino acid sequence of the first heavy chain of the full-length antibody comprises a cysteine at amino acid position Y349, numbering according to the EU index of Kabat. In some embodiments, the full-length antibody is an IgG1 antibody, and wherein the amino acid sequence of the second heavy chain of the full-length antibody comprises an amino acid substitution at amino acid position T366, numbering according to the EU index of Kabat. In some embodiments, the full-length antibody is an IgG1 antibody, and wherein the amino acid sequence of the second heavy chain of the full-length antibody comprises a tryptophan at amino acid position T366, numbering according to the EU index of Kabat. In some embodiments, the full-length antibody is an IgG1 antibody, and wherein the amino acid sequence of the second heavy chain of the full-length antibody comprises an amino acid substitution at amino acid position S354, numbering according to the EU index of Kabat. In some embodiments, the full-length antibody is an IgG1 antibody, and wherein the amino acid sequence of the second heavy chain of the full-length antibody comprises a cysteine at amino acid position S354, numbering according to the EU index of Kabat.

In some embodiments, the full-length antibody is an IgG1 antibody, and wherein the amino acid sequence of the second heavy chain of the full-length antibody comprises an amino acid substitution at amino acid positions T366, L368, and Y407, numbering according to the EU index of Kabat. In some embodiments, the full-length antibody is an IgG1 antibody, and wherein the amino acid sequence of the second heavy chain of the full-length antibody comprises a serine at amino acid position T366, an alanine at amino acid position L368, and a valine at amino acid position Y407, numbering according to the EU index of Kabat. In some embodiments, the full-length antibody is an IgG1 antibody, and wherein the amino acid sequence of the second heavy chain of the full-length antibody comprises an amino acid substitution at amino acid position Y349, numbering according to the EU index of Kabat. In some embodiments, the full-length antibody is an IgG1 antibody, and wherein the amino acid sequence of the second heavy chain of the full-length antibody comprises a cysteine at amino acid position Y349, numbering according to the EU index of Kabat. In some embodiments, the full-length antibody is an IgG1 antibody, and wherein the amino acid sequence of the first heavy chain of the full-length antibody comprises an amino acid substitution at amino acid position T366, numbering according to the EU index of Kabat. In some embodiments, the full-length antibody is an IgG1 antibody, and wherein the amino acid sequence of the first heavy chain of the full-length antibody comprises a tryptophan at amino acid position T366, numbering according to the EU index of Kabat. In some embodiments, the full-length antibody is an IgG1 antibody, and wherein the amino acid sequence of the first heavy chain of the full-length antibody comprises an amino acid substitution at amino acid position S354, numbering according to the EU index of Kabat. In some embodiments, the full-length antibody is an IgG1 antibody, and wherein the amino acid sequence of the first heavy chain of the full-length antibody comprises a cysteine at amino acid position S354, numbering according to the EU index of Kabat.

In some embodiments, the multispecific protein does not substantially mediate ADCC, does not substantially mediate ADCP, does not substantially mediate CDC, and/or does not bind to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the full-length antibody is an IgG1 antibody, and wherein the amino acid sequence of the first heavy chain and the second heavy chain each comprises an amino acid substitution at amino acid position L234, and/or an amino acid substitution at amino acid position L235, numbering according to the EU index of Kabat. In some embodiments, the full-length antibody is an IgG4 antibody, and wherein the amino acid sequence of the first heavy chain and the second heavy chain each comprises an alanine at amino acid position L234 and/or an alanine at amino acid position L235, numbering according to the EU index of Kabat. In some embodiments, the full-length antibody is an IgG1 antibody, and wherein the amino acid sequence of the first heavy chain and the second heavy chain each comprises an amino acid substitution at amino acid position L234, an amino acid substitution at amino acid position L235, and/or an amino acid substitution at amino acid position P329, numbering according to the EU index of Kabat.

In some embodiments, the full-length antibody is an IgG1 antibody, and wherein the amino acid sequence of the first heavy chain and the second heavy chain each comprises an alanine at amino acid position L234, an alanine at amino acid position L235, and/or an alanine at amino acid position P329, numbering according to the EU index of Kabat. In some embodiments, the full-length antibody is an IgG1 antibody, and wherein the amino acid sequence of the first heavy chain and the second heavy chain each comprises an alanine at amino acid position L234, an alanine at amino acid position L235, and/or a glycine at amino acid position P329, numbering according to the EU index of Kabat. In some embodiments, the full-length antibody is an IgG4 antibody, and wherein the amino acid sequence of the first heavy chain and the second heavy chain each comprises an amino acid substitution at amino acid position S228, an amino acid substitution at amino acid position F234, and/or an amino acid substitution at amino acid position E235, numbering according to the EU index of Kabat. In some embodiments, the full-length antibody is an IgG4 antibody, and wherein the amino acid sequence of the first heavy chain and the second heavy chain each comprises a proline at amino acid position S228, an alanine at amino acid position F234, and/or an alanine at amino acid position E235, numbering according to the EU index of Kabat.

In some embodiments, the hTCSA is hCD28, hCD2, hCD137, hCD27, hCD278, hCD134, or hCD40. In some embodiments, the hTCSA is hCD28. In some embodiments, the hTCSA is hCD2.

In one aspect, provided herein are multispecific proteins comprising: (a) a full-length antibody comprising: (i) a first light chain comprising from N- to C-terminus a VL region and a CL region; (ii) a first heavy chain comprising from N- to C-terminus a VH region, a CH1 region, a hinge region, a CH2 region, and a CH3 region; (iii) a second heavy chain comprising from N- to C-terminus a VH region, a CH1 region, a hinge region, a CH2 region, and a CH3 region; (iv) a second light chain comprising from N- to C-terminus a VL region and a CL region; wherein the first light chain and first heavy chain associate to form a first antigen binding domain; wherein the second light chain and the second heavy chain associate to form a second antigen binding domain; and wherein the first heavy chain and second heavy chain associate to form a dimer; (b) a first scFv operably connected to the N-terminus of the first heavy chain of the full-length antibody, wherein the first scFv comprises from N- to C-terminus (i) a VH region, a peptide linker, and a VL region, or (ii) a VL region, a peptide linker, and a VH region; and (c) a second scFv operably connected to the N-terminus of the second heavy chain of the full-length antibody, wherein the second scFv comprises from N- to C-terminus (i) a VH region, a peptide linker, and a VL region, or (ii) a VL region, a peptide linker, and a VH region; wherein the first antigen binding domain of the full-length antibody specifically binds to a first hTAA; wherein the second antigen binding domain of the full-length antibody specifically binds to a second hTAA; wherein the first scFv specifically binds to a hTCSA (e.g., hCD28, hCD2); wherein the second scFv specifically binds to hCD3; and wherein the CH3 region of the first heavy chain of the full-length antibody comprises one or more amino acid modification (e.g., substitution) relative to the amino acid sequence of a reference CH3 region that does not contain the one or more amino acid modification (e.g., a wild-type CH3 region, e.g., SEQ ID NO: 101), wherein the CH3 region of the second heavy chain of the of the full-length antibody comprises one or more amino acid modification (e.g., substitution) relative to the amino acid sequence of a reference CH3 region that does not contain the one or more amino acid modification (e.g., a wild-type CH3 region, e.g., SEQ ID NO: 101); and wherein the one or more amino acid modification in the CH3 region of the first heavy chain of the full-length antibody is different from the one or more amino acid modification in the CH3 region of the second heavy chain of the full-length antibody; wherein the one or more amino acid modification in the CH3 region of the first heavy chain of the full-length antibody and the one or more amino acid modification in the CH3 region of the second heavy chain of the full-length antibody promote heterodimerization of the first and second heavy chain of the full-length antibody; wherein the CH2 region of the first heavy chain of the full-length antibody comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the CH2 region of the second heavy chain of the full-length antibody comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the one or more amino acid modification in the CH2 region of the first heavy chain of the full-length antibody and the one or more amino acid modification in the CH2 region of the second heavy chain of the full-length antibody reduce or abolish one or more of the following heavy chain effector functions relative to a reference heavy chain that does not contain the one or more amino acid modification (e.g., a heavy chain comprising a wild type CH2 region, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the first scFv is operably connected to the N-terminus of the VH region of the first heavy chain of the full-length antibody directly through a peptide bond.

In some embodiments, the first scFv is operably connected to the N-terminus of the VH region of the first heavy chain of the full-length antibody through a first peptide linker. In some embodiments, the amino acid sequence of the first peptide linker comprises or consists of glycine or glycine and serine amino acid residues. In some embodiments, the amino acid sequence of the first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

In some embodiments, the second scFv is operably connected to the N-terminus of the VH region of the second heavy chain of the full-length antibody directly through a peptide bond.

In some embodiments, the second scFv is operably connected to the N-terminus of the VH region of the second heavy chain of the full-length antibody through a second peptide linker. In some embodiments, the amino acid sequence of the second peptide linker comprises or consists of glycine or glycine and serine amino acid residues. In some embodiments, the amino acid of the second peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

In some embodiments, the first scFv comprises from N- to C-terminus: a VL region, a peptide linker, and a VH region; and wherein the C-terminus of the VH region of the scFv is operably connected to the N-terminus of the VH region of the first heavy chain of the full-length antibody. In some embodiments, the first scFv comprises from N- to C-terminus: a VH region, a peptide linker, and a VL region; and wherein the C-terminus of the VL region of the scFv is operably connected to the N-terminus of the VH region of the first heavy chain of the full-length antibody. In some embodiments, the second scFv comprises from N- to C-terminus: a VL region, a peptide linker, and a VH region; and wherein the C-terminus of the VH region of the scFv is operably connected to the N-terminus of the VH region of the second heavy chain of the full-length antibody. In some embodiments, the second scFv comprises from N- to C-terminus: a VH region, a peptide linker, and a VL region; and wherein the C-terminus of the VL region of the scFv is operably connected to the N-terminus of the VH region of the second heavy chain of the full-length antibody.

In some embodiments, the first hTAA and the second hTAA are expressed by (e.g., on the surface of) the same tumor cell. In some embodiments, the first hTAA and the second hTAA are the same. In some embodiments, the first scFv and the second scFv specifically bind the same epitope of the same hTAA. In some embodiments, the first scFv and the second scFv specifically bind different epitopes of the same hTAA. In some embodiments, the first tumor associated antigen is hEGFR, hMSLN, hPSMA, or hHER2; and the second tumor associated antigen is hEGFR, hMSLN, hPSMA, or hHER2.

In some embodiments, the amino acid sequence of the VH region of the first scFv and the amino acid sequence of the VL region of the first scFv each comprises a cysteine amino acid residue, wherein the cysteine amino acid residues are capable of forming a disulfide bond. In some embodiments, the amino acid sequence of the VH region of the first scFv comprises a cysteine at amino acid position 97, 98, 99, 100, 101, 102, 103, 104, 105, or 106, amino acid numbering according to Kabat; and the amino acid sequence of the VL region of the first scFv comprises a cysteine at amino acid position 40, 41, 42, 43, 44, 45, 46, or 47, amino acid numbering according to Kabat. In some embodiments, the amino acid sequence of the VH region of the second scFv comprises a cysteine at amino acid position 97, 98, 99, 100, 101, 102, 103, 104, 105, or 106, amino acid numbering according to Kabat; and the amino acid sequence of the VL region of the second scFv comprises a cysteine at amino acid position 40, 41, 42, 43, 44, 45, 46, or 47, amino acid numbering according to Kabat.

In some embodiments, the full-length antibody is a human IgG (hIgG) antibody. In some embodiments, the full-length antibody is a hIgG1 or hIgG4 antibody.

In some embodiments, the full-length antibody is an IgG1 antibody, and wherein the amino acid sequence of the first heavy chain of the full-length antibody comprises an amino acid substitution at amino acid positions T366, L368, and Y407, numbering according to the EU index of Kabat. In some embodiments, the full-length antibody is an IgG1 antibody, and wherein the amino acid sequence of the first heavy chain of the full-length antibody comprises a serine at amino acid position T366, an alanine at amino acid position L368, and a valine at amino acid position Y407, numbering according to the EU index of Kabat. In some embodiments, the full-length antibody is an IgG1 antibody, and wherein the amino acid sequence of the first heavy chain of the full-length antibody comprises an amino acid substitution at amino acid position Y349, numbering according to the EU index of Kabat. In some embodiments, the full-length antibody is an IgG1 antibody, and wherein the amino acid sequence of the first heavy chain of the full-length antibody comprises a cysteine at amino acid position Y349, numbering according to the EU index of Kabat. In some embodiments, the full-length antibody is an IgG1 antibody, and wherein the amino acid sequence of the second heavy chain of the full-length antibody comprises an amino acid substitution at amino acid position T366, numbering according to the EU index of Kabat. In some embodiments, the full-length antibody is an IgG1 antibody, and wherein the amino acid sequence of the second heavy chain of the full-length antibody comprises a tryptophan at amino acid position T366, numbering according to the EU index of Kabat. In some embodiments, the full-length antibody is an IgG1 antibody, and wherein the amino acid sequence of the second heavy chain of the full-length antibody comprises an amino acid substitution at amino acid position S354, numbering according to the EU index of Kabat. In some embodiments, the full-length antibody is an IgG1 antibody, and wherein the amino acid sequence of the second heavy chain of the full-length antibody comprises a cysteine at amino acid position S354, numbering according to the EU index of Kabat.

In some embodiments, the full-length antibody is an IgG1 antibody, and wherein the amino acid sequence of the second heavy chain of the full-length antibody comprises an amino acid substitution at amino acid positions T366, L368, and Y407, numbering according to the EU index of Kabat. In some embodiments, the full-length antibody is an IgG1 antibody, and wherein the amino acid sequence of the second heavy chain of the full-length antibody comprises a serine at amino acid position T366, an alanine at amino acid position L368, and a valine at amino acid position Y407, numbering according to the EU index of Kabat. In some embodiments, the full-length antibody is an IgG1 antibody, and wherein the amino acid sequence of the second heavy chain of the full-length antibody comprises an amino acid substitution at amino acid position Y349, numbering according to the EU index of Kabat. In some embodiments, the full-length antibody is an IgG1 antibody, and wherein the amino acid sequence of the second heavy chain of the full-length antibody comprises a cysteine at amino acid position Y349, numbering according to the EU index of Kabat. In some embodiments, the full-length antibody is an IgG1 antibody, and wherein the amino acid sequence of the first heavy chain of the full-length antibody comprises an amino acid substitution at amino acid position T366, numbering according to the EU index of Kabat. In some embodiments, the full-length antibody is an IgG1 antibody, and wherein the amino acid sequence of the first heavy chain of the full-length antibody comprises a tryptophan at amino acid position T366, numbering according to the EU index of Kabat. In some embodiments, the full-length antibody is an IgG1 antibody, and wherein the amino acid sequence of the first heavy chain of the full-length antibody comprises an amino acid substitution at amino acid position S354, numbering according to the EU index of Kabat. In some embodiments, the full-length antibody is an IgG1 antibody, and wherein the amino acid sequence of the first heavy chain of the full-length antibody comprises a cysteine at amino acid position S354, numbering according to the EU index of Kabat.

In some embodiments, the multispecific protein does not substantially mediate ADCC, does not substantially mediate ADCP, does not substantially mediate CDC, and/or does not bind to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the full-length antibody is an IgG1 antibody, and wherein the amino acid sequence of the first heavy chain and the second heavy chain each comprises an amino acid substitution at amino acid position L234, and/or an amino acid substitution at amino acid position L235, numbering according to the EU index of Kabat. In some embodiments, the full-length antibody is an IgG4 antibody, and wherein the amino acid sequence of the first heavy chain and the second heavy chain each comprises an alanine at amino acid position L234 and/or an alanine at amino acid position L235, numbering according to the EU index of Kabat. In some embodiments, the full-length antibody is an IgG1 antibody, and wherein the amino acid sequence of the first heavy chain and the second heavy chain each comprises an amino acid substitution at amino acid position L234, an amino acid substitution at amino acid position L235, and/or an amino acid substitution at amino acid position P329, numbering according to the EU index of Kabat.

In some embodiments, the full-length antibody is an IgG1 antibody, and wherein the amino acid sequence of the first heavy chain and the second heavy chain each comprises an alanine at amino acid position L234, an alanine at amino acid position L235, and/or an alanine at amino acid position P329, numbering according to the EU index of Kabat. In some embodiments, the full-length antibody is an IgG1 antibody, and wherein the amino acid sequence of the first heavy chain and the second heavy chain each comprises an alanine at amino acid position L234, an alanine at amino acid position L235, and/or a glycine at amino acid position P329, numbering according to the EU index of Kabat. In some embodiments, the full-length antibody is an IgG4 antibody, and wherein the amino acid sequence of the first heavy chain and the second heavy chain each comprises an amino acid substitution at amino acid position S228, an amino acid substitution at amino acid position F234, and/or an amino acid substitution at amino acid position E235, numbering according to the EU index of Kabat. In some embodiments, the full-length antibody is an IgG4 antibody, and wherein the amino acid sequence of the first heavy chain and the second heavy chain each comprises a proline at amino acid position S228, an alanine at amino acid position F234, and/or an alanine at amino acid position E235, numbering according to the EU index of Kabat.

In one aspect, provided herein are multispecific proteins comprising: (a) first Fab comprising (i) a first Fab heavy chain comprising from N- to C-terminus a first VH region and a first CH1 region and (ii) a first light chain comprising from N- to C-terminus a first VL region and a first CL region; (b) a first scFv operably connected to the C-terminus of the first CH1 region of the first Fab, wherein the first scFv comprises from N- to C-terminus (i) a VH region, a peptide linker, and a VL region; or (ii) a VL region, a peptide linker, and a VH region; (c) a first Fc region operably connected to the C-terminus of the first scFv, wherein the first Fc region comprises from N- to C-terminus a CH2 region and a CH3 region; and (d) second Fab comprising (i) a second Fab heavy chain comprising from N- to C-terminus a second VH region and a second CH1 region and (ii) a second light chain comprising from N- to C-terminus a second VL region and a first CL region; (b) a second scFv operably connected to the C-terminus of the second CH1 of the second Fab, wherein the second scFv comprises from N- to C-terminus (i) a VH region, a peptide linker, and a VL region; or (ii) a VL region, a peptide linker, and a VH region; (c) a second Fc region operably connected to the C-terminus of the second scFv, wherein the second Fc region comprises from N- to C-terminus a CH2 region and a CH3 region; and wherein the first Fab specifically binds to a first human hTAA; wherein the second Fab specifically binds to a second hTAA; wherein the first scFv specifically binds to a hTCSA (e.g., hCD28, hCD2); wherein the second scFv specifically binds to hCD3; and wherein the CH3 region of the first Fc region comprises one or more amino acid modification (e.g., substitution) relative to the amino acid sequence of a reference CH3 region that does not contain the one or more amino acid modification (e.g., a wild-type CH3 region, e.g., SEQ ID NO: 101), wherein the CH3 region of the second Fc region comprises one or more amino acid modification (e.g., substitution) relative to the amino acid sequence of a reference CH3 region that does not contain the one or more amino acid modification (e.g., a wild-type CH3 region, e.g., SEQ ID NO: 101); and wherein the one or more amino acid modification in the CH3 region of the first Fc region is different from the one or more amino acid modification in the CH3 region of the Fc region; wherein the one or more amino acid modification in the CH3 region of the first Fc region and the one or more amino acid modification in the CH3 region of the second Fc region promote heterodimerization of the first and second Fc region; wherein the CH2 region of the first Fc region comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the CH2 region of the second Fc region comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the one or more amino acid modification in the CH2 region of the Fc region and the one or more amino acid modification in the CH2 region of the second Fc region reduce or abolish one or more of the following Fc region effector functions relative to a reference Fc region that does not contain the one or more amino acid modification (e.g., a heavy chain comprising a wild type CH2 domain, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the first Fab is operably connected to the first scFv directly through a peptide bond.

In some embodiments, the first Fab is operably connected the first scFv through a first peptide linker. In some embodiments, the amino acid sequence of the first peptide linker comprises or consists of glycine or glycine and serine amino acid residues. In some embodiments, the amino acid sequence of the first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

In some embodiments, the second Fab is operably connected to the second scFv directly through a peptide bond.

In some embodiments, the second Fab is operably connected to the second scFv through a second peptide linker. In some embodiments, the amino acid sequence of the second peptide linker comprises or consists of glycine or glycine and serine amino acid residues. In some embodiments, the amino acid of the second peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

In some embodiments, the first scFv comprises from N- to C-terminus: a VL region, a peptide linker, and a VH region; and wherein the N-terminus of the VL region of the scFv is operably connected to the C-terminus of the CH1 region of the first Fab. In some embodiments, the first scFv comprises from N- to C-terminus: a VH region, a peptide linker, and a VL region; and wherein the N-terminus of the VH region of the scFv is operably connected to the C-terminus of the CH1 region of the first Fab. In some embodiments, the second scFv comprises from N- to C-terminus: a VL region, a peptide linker, and a VH region; and wherein the C-terminus of the VH region of the scFv is operably connected to the N-terminus of the CH1 region of the second Fab. In some embodiments, the second scFv comprises from N- to C-terminus: a VH region, a peptide linker, and a VL region; and wherein the C-terminus of the VL region of the scFv is operably connected to the N-terminus of the CH1 region of the second Fab.

In some embodiments, the first hTAA and the second hTAA are expressed by (e.g., on the surface of) the same tumor cell. In some embodiments, the first hTAA and the second hTAA are the same. In some embodiments, the first scFv and the second scFv specifically bind the same epitope of the same hTAA. In some embodiments, the first scFv and the second scFv specifically bind different epitopes of the same hTAA. In some embodiments, the first tumor associated antigen is hEGFR, hMSLN, hPSMA, or hHER2; and the second tumor associated antigen is hEGFR, hMSLN, hPSMA, or hHER2.

In some embodiments, the amino acid sequence of the VH region of the first scFv and the amino acid sequence of the VL region of the first scFv each comprises a cysteine amino acid residue, wherein the cysteine amino acid residues are capable of forming a disulfide bond. In some embodiments, the amino acid sequence of the VH region of the first scFv comprises a cysteine at amino acid position 97, 98, 99, 100, 101, 102, 103, 104, 105, or 106, amino acid numbering according to Kabat; and the amino acid sequence of the VL region of the first scFv comprises a cysteine at amino acid position 40, 41, 42, 43, 44, 45, 46, or 47, amino acid numbering according to Kabat. In some embodiments, the amino acid sequence of the VH region of the second scFv comprises a cysteine at amino acid position 97, 98, 99, 100, 101, 102, 103, 104, 105, or 106, amino acid numbering according to Kabat; and the amino acid sequence of the VL region of the second scFv comprises a cysteine at amino acid position 40, 41, 42, 43, 44, 45, 46, or 47, amino acid numbering according to Kabat.

In some embodiments, the first Fc region and the second Fc region are a human IgG (hIgG) isotype. In some embodiments, the first Fc region and the second Fc region are hIgG1 or hIgG4 isotype.

In some embodiments, the first Fc region and the second Fc region are IgG1 isotype, and wherein the amino acid sequence of the first Fc region comprises an amino acid substitution at amino acid positions T366, L368, and Y407, numbering according to the EU index of Kabat. In some embodiments, the first Fc region and the second Fc region are IgG1 isotype, and wherein the amino acid sequence of the first Fc region comprises a serine at amino acid position T366, an alanine at amino acid position L368, and a valine at amino acid position Y407, numbering according to the EU index of Kabat. In some embodiments, the first Fc region and the second Fc region are IgG1 isotype, and wherein the amino acid sequence of the first Fc region comprises an amino acid substitution at amino acid position Y349, numbering according to the EU index of Kabat. In some embodiments, the first Fc region and the second Fc region are IgG1 isotype, and wherein the amino acid sequence of the first Fc region comprises a cysteine at amino acid position Y349, numbering according to the EU index of Kabat. In some embodiments, the first Fc region and the second Fc region are IgG1 isotype, and wherein the amino acid sequence of the second Fc region comprises an amino acid substitution at amino acid position T366, numbering according to the EU index of Kabat. In some embodiments, the first Fc region and the second Fc region are IgG1 isotype, and wherein the amino acid sequence of the second Fc region comprises a tryptophan at amino acid position T366, numbering according to the EU index of Kabat. In some embodiments, the first Fc region and the second Fc region are IgG1 isotype, and wherein the amino acid sequence of the second Fc region comprises an amino acid substitution at amino acid position S354, numbering according to the EU index of Kabat. In some embodiments, the first Fc region and the second Fc region are IgG1 isotype, and wherein the amino acid sequence of the second Fc region comprises a cysteine at amino acid position S354, numbering according to the EU index of Kabat.

In some embodiments, the first Fc region and the second Fc region are IgG1 isotype, and wherein the amino acid sequence of the second Fc region comprises an amino acid substitution at amino acid positions T366, L368, and Y407, numbering according to the EU index of Kabat. In some embodiments, the first Fc region and the second Fc region are IgG1 isotype, and wherein the amino acid sequence of the second Fc region comprises a serine at amino acid position T366, an alanine at amino acid position L368, and a valine at amino acid position Y407, numbering according to the EU index of Kabat. In some embodiments, the first Fc region and the second Fc region are IgG1 isotype, and wherein the amino acid sequence of the second Fc region comprises an amino acid substitution at amino acid position Y349, numbering according to the EU index of Kabat. In some embodiments, the first Fc region and the second Fc region are IgG1 isotype, and wherein the amino acid sequence of the second Fc region comprises a cysteine at amino acid position Y349, numbering according to the EU index of Kabat. In some embodiments, the first Fc region and the second Fc region are IgG1 isotype, and wherein the amino acid sequence of the first Fc region an amino acid substitution at amino acid position T366, numbering according to the EU index of Kabat. In some embodiments, the first Fc region and the second Fc region are IgG1 isotype, and wherein the amino acid sequence of the first Fc region comprises a tryptophan at amino acid position T366, numbering according to the EU index of Kabat. In some embodiments, the first Fc region and the second Fc region are IgG1 isotype, and wherein the amino acid sequence of the first Fc region comprises an amino acid substitution at amino acid position S354, numbering according to the EU index of Kabat. In some embodiments, the first Fc region and the second Fc region are IgG1 isotype, and wherein the amino acid sequence of the first Fc region comprises a cysteine at amino acid position S354, numbering according to the EU index of Kabat.

In some embodiments, the multispecific protein does not substantially mediate ADCC, does not substantially mediate ADCP, does not substantially mediate CDC, and/or does not bind to one or more Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

In some embodiments, the first Fc region and the second Fc region are IgG1 isotype, and wherein the amino acid sequence of the first Fc region and the second Fc region each comprises an amino acid substitution at amino acid position L234, and/or an amino acid substitution at amino acid position L235, numbering according to the EU index of Kabat. In some embodiments, the first Fc region and the second Fc region are IgG1 isotype, and wherein the amino acid sequence of the first Fc region and the second Fc region each comprises an alanine at amino acid position L234 and/or an alanine at amino acid position L235, numbering according to the EU index of Kabat. In some embodiments, the first Fc region and the second Fc region are IgG1 isotype, and wherein the amino acid sequence of the first Fc region and the second Fc region each comprises an amino acid substitution at amino acid position L234, an amino acid substitution at amino acid position L235, and/or an amino acid substitution at amino acid position P329, numbering according to the EU index of Kabat.

In some embodiments, the first Fc region and the second Fc region are IgG1 isotype, and wherein the amino acid sequence of the first Fc region and the second Fc region each comprises an alanine at amino acid position L234, an alanine at amino acid position L235, and/or an alanine at amino acid position P329, numbering according to the EU index of Kabat. In some embodiments, the first Fc region and the second Fc region are IgG1 isotype, and wherein the amino acid sequence of the first Fc region and the second Fc region each comprises an alanine at amino acid position L234, an alanine at amino acid position L235, and/or a glycine at amino acid position P329, numbering according to the EU index of Kabat. In some embodiments, the first Fc region and the second Fc region are IgG4 isotype, and wherein the amino acid sequence of the first Fc region and the second Fc region each comprises an amino acid substitution at amino acid position S228, an amino acid substitution at amino acid position F234, and/or an amino acid substitution at amino acid position E235, numbering according to the EU index of Kabat. In some embodiments, the first Fc region and the second Fc region are IgG4 isotype, and wherein the amino acid sequence of the first Fc region and the second Fc region each comprises a proline at amino acid position S228, an alanine at amino acid position F234, and/or an alanine at amino acid position E235, numbering according to the EU index of Kabat.

In some embodiments, the hTCSA is hCD28, hCD2, hCD137, hCD27, hCD278, hCD134, or hCD40. In some embodiments, the hTCSA is hCD28. In some embodiments, the hTCSA is hCD2.

In one aspect, provided herein are multispecific proteins comprising: (a) a scFv comprising from N- to C-terminus (i) a VH region, a peptide linker, and a VL region, or (ii) a VL region, a peptide linker, and a VH region; (b) a Fab comprising (i) a Fab heavy chain comprising from N- to C-terminus a VH region and a CH1 region and (ii) a light chain comprising from N- to C-terminus a VL region and a CL region; (c) a first Fc region comprising from N- to C-terminus a CH2 region and a CH3 region; (d) a second Fc region comprising from N- to C-terminus a CH2 region and a CH3 region; (e) an IgM CH2 mFab comprising (i) an IgM CH2 mFab heavy chain comprising from N- to C-terminus a VH region and an IgM CH2 region and (ii) an IgM CH2 mFab light chain comprising from N- to C-terminus a VL region and an IgM CH2 region; wherein the C-terminus of the scFv is operably connected to the N-terminus of the Fab heavy chain of the first Fab; wherein the C-terminus of the Fab heavy chain is operably connected to the N-terminus of the first Fc region; wherein the C-terminus of the IgM CH2 mFab heavy chain is operably connected to the N-terminus of the second Fc region; wherein the scFv specifically binds a hTCSA (e.g., hCD28, hCD2); wherein the Fab specifically binds hCD3; wherein the IgM CH2 mFab specifically binds a human tumor associated antigen (hTAA); wherein the CH3 region of the first Fc region comprises one or more amino acid modification (e.g., substitution) relative to the amino acid sequence of a reference CH3 region that does not contain the one or more amino acid modification (e.g., a wild-type CH3 region, e.g., SEQ ID NO: 101), wherein the CH3 region of the second Fc region comprises one or more amino acid modification (e.g., substitution) relative to the amino acid sequence of a reference CH3 region that does not contain the one or more amino acid modification (e.g., a wild-type CH3 region, e.g., SEQ ID NO: 101); and wherein the one or more amino acid modification in the CH3 region of the first Fc region is different from the one or more amino acid modification in the CH3 region of the Fc region; wherein the one or more amino acid modification in the CH3 region of the first Fc region and the one or more amino acid modification in the CH3 region of the second Fc region promote heterodimerization of the first and second heavy chain; wherein the CH2 region of the first Fc region comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the CH2 region of the second Fc region comprises one or more amino acid modification (e.g., substitution) relative to the amino acid sequence of a reference CH2 region that does not contain the one or more amino acid modification (e.g., a wild-type CH2 region, e.g., SEQ ID NO: 100); wherein the one or more amino acid modification in the CH2 region of the Fc region and the one or more amino acid modification in the CH2 region of the second Fc region reduce or abolish one or more of the following Fc region effector functions relative to a reference Fc region that does not contain the one or more amino acid modification (e.g., a heavy chain comprising a wild type CH2 domain, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the scFv is operably connected to the N-terminus of the Fab heavy chain of the first Fab directly through a peptide bond.

In some embodiments, the scFv is operably connected to the N-terminus of the Fab heavy chain of the first Fab through a first peptide linker. In some embodiments, the amino acid sequence of the first peptide linker comprises or consists of glycine or glycine and serine amino acid residues. In some embodiments, the amino acid sequence of the first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

In some embodiments, the scFv comprises from N- to C-terminus: a VL region, a peptide linker, and a VH region; and wherein the C-terminus of the VH region of the scFv is operably connected to the N-terminus of the Fab heavy chain of the first Fab.

In some embodiments, the scFv comprises from N- to C-terminus: a VH region, a peptide linker, and a VL region; and wherein the C-terminus of the VL region of the scFv is operably connected to the N-terminus of the Fab heavy chain of the first Fab.

In some embodiments, the tumor associated antigen is hEGFR, hMSLN, hPSMA, or hHER2; and the second tumor associated antigen is hEGFR, hMSLN, hPSMA, or hHER2.

In some embodiments, the amino acid sequence of the VH region of the first scFv and the amino acid sequence of the VL region of the first scFv each comprises a cysteine amino acid residue, wherein the cysteine amino acid residues are capable of forming a disulfide bond. In some embodiments, the amino acid sequence of the VH region of the first scFv comprises a cysteine at amino acid position 97, 98, 99, 100, 101, 102, 103, 104, 105, or 106, amino acid numbering according to Kabat; and the amino acid sequence of the VL region of the first scFv comprises a cysteine at amino acid position 40, 41, 42, 43, 44, 45, 46, or 47, amino acid numbering according to Kabat. In some embodiments, the amino acid sequence of the VH region of the second scFv comprises a cysteine at amino acid position 97, 98, 99, 100, 101, 102, 103, 104, 105, or 106, amino acid numbering according to Kabat; and the amino acid sequence of the VL region of the second scFv comprises a cysteine at amino acid position 40, 41, 42, 43, 44, 45, 46, or 47, amino acid numbering according to Kabat.

In some embodiments, the first Fc region and the second Fc region are a human IgG (hIgG) isotype. In some embodiments, the first Fc region and the second Fc region are hIgG1 or hIgG4 isotype.

In some embodiments, the first Fc region and the second Fc region are IgG1 isotype, and wherein the amino acid sequence of the first Fc region comprises an amino acid substitution at amino acid positions T366, L368, and Y407, numbering according to the EU index of Kabat. In some embodiments, the first Fc region and the second Fc region are IgG1 isotype, and wherein the amino acid sequence of the first Fc region comprises a serine at amino acid position T366, an alanine at amino acid position L368, and a valine at amino acid position Y407, numbering according to the EU index of Kabat. In some embodiments, the first Fc region and the second Fc region are IgG1 isotype, and wherein the amino acid sequence of the first Fc region comprises an amino acid substitution at amino acid position Y349, numbering according to the EU index of Kabat. In some embodiments, the first Fc region and the second Fc region are IgG1 isotype, and wherein the amino acid sequence of the first Fc region comprises a cysteine at amino acid position Y349, numbering according to the EU index of Kabat. In some embodiments, the first Fc region and the second Fc region are IgG1 isotype, and wherein the amino acid sequence of the second Fc region comprises an amino acid substitution at amino acid position T366, numbering according to the EU index of Kabat. In some embodiments, the first Fc region and the second Fc region are IgG1 isotype, and wherein the amino acid sequence of the second Fc region comprises a tryptophan at amino acid position T366, numbering according to the EU index of Kabat. In some embodiments, the first Fc region and the second Fc region are IgG1 isotype, and wherein the amino acid sequence of the second Fc region comprises an amino acid substitution at amino acid position S354, numbering according to the EU index of Kabat. In some embodiments, the first Fc region and the second Fc region are IgG1 isotype, and wherein the amino acid sequence of the second Fc region comprises a cysteine at amino acid position S354, numbering according to the EU index of Kabat.

In some embodiments, the first Fc region and the second Fc region are IgG1 isotype, and wherein the amino acid sequence of the second Fc region comprises an amino acid substitution at amino acid positions T366, L368, and Y407, numbering according to the EU index of Kabat. In some embodiments, the first Fc region and the second Fc region are IgG1 isotype, and wherein the amino acid sequence of the second Fc region comprises a serine at amino acid position T366, an alanine at amino acid position L368, and a valine at amino acid position Y407, numbering according to the EU index of Kabat. In some embodiments, the first Fc region and the second Fc region are IgG1 isotype, and wherein the amino acid sequence of the second Fc region comprises an amino acid substitution at amino acid position Y349, numbering according to the EU index of Kabat. In some embodiments, the first Fc region and the second Fc region are IgG1 isotype, and wherein the amino acid sequence of the second Fc region comprises a cysteine at amino acid position Y349, numbering according to the EU index of Kabat. In some embodiments, the first Fc region and the second Fc region are IgG1 isotype, and wherein the amino acid sequence of the first Fc region an amino acid substitution at amino acid position T366, numbering according to the EU index of Kabat. In some embodiments, the first Fc region and the second Fc region are IgG1 isotype, and wherein the amino acid sequence of the first Fc region comprises a tryptophan at amino acid position T366, numbering according to the EU index of Kabat. In some embodiments, the first Fc region and the second Fc region are IgG1 isotype, and wherein the amino acid sequence of the first Fc region comprises an amino acid substitution at amino acid position S354, numbering according to the EU index of Kabat. In some embodiments, the first Fc region and the second Fc region are IgG1 isotype, and wherein the amino acid sequence of the first Fc region comprises a cysteine at amino acid position S354, numbering according to the EU index of Kabat.

In some embodiments, the multispecific protein does not substantially mediate ADCC, does not substantially mediate ADCP, does not substantially mediate CDC, and/or does not bind to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the first Fc region and the second Fc region are IgG1 isotype, and wherein the amino acid sequence of the first Fc region and the second Fc region each comprises an amino acid substitution at amino acid position L234, and/or an amino acid substitution at amino acid position L235, numbering according to the EU index of Kabat. In some embodiments, the first Fc region and the second Fc region are IgG1 isotype, and wherein the amino acid sequence of the first Fc region and the second Fc region each comprises an alanine at amino acid position L234 and/or an alanine at amino acid position L235, numbering according to the EU index of Kabat. In some embodiments, the first Fc region and the second Fc region are IgG1 isotype, and wherein the amino acid sequence of the first Fc region and the second Fc region each comprises an amino acid substitution at amino acid position L234, an amino acid substitution at amino acid position L235, and/or an amino acid substitution at amino acid position P329, numbering according to the EU index of Kabat.

In some embodiments, the first Fc region and the second Fc region are IgG1 isotype, and wherein the amino acid sequence of the first Fc region and the second Fc region each comprises an alanine at amino acid position L234, an alanine at amino acid position L235, and/or an alanine at amino acid position P329, numbering according to the EU index of Kabat. In some embodiments, the first Fc region and the second Fc region are IgG1 isotype, and wherein the amino acid sequence of the first Fc region and the second Fc region each comprises an alanine at amino acid position L234, an alanine at amino acid position L235, and/or a glycine at amino acid position P329, numbering according to the EU index of Kabat. In some embodiments, the first Fc region and the second Fc region are IgG4 isotype, and wherein the amino acid sequence of the first Fc region and the second Fc region each comprises an amino acid substitution at amino acid position S228, an amino acid substitution at amino acid position F234, and/or an amino acid substitution at amino acid position E235, numbering according to the EU index of Kabat. In some embodiments, the first Fc region and the second Fc region are IgG4 isotype, and wherein the amino acid sequence of the first Fc region and the second Fc region each comprises a proline at amino acid position S228, an alanine at amino acid position F234, and/or an alanine at amino acid position E235, numbering according to the EU index of Kabat.

In some embodiments, the tumor associated antigen is hEGFR, hMSLN, hPSMA, or hHER2; and the second tumor associated antigen hEGFR, hMSLN, hPSMA, or hHER2.

In some embodiments, the hTCSA is hCD28, hCD2, hCD137, hCD27, hCD278, hCD134, or hCD40. In some embodiments, the hTCSA is hCD28. In some embodiments, the hTCSA is hCD2.

In one aspect, provided herein are polynucleotides encoding a multispecific protein described herein or one or more polypeptide thereof. In some embodiments, the polynucleotide is RNA (e.g., mRNA) or DNA. In some embodiments, the polynucleotide is codon optimized.

In one aspect, provided herein are expression vectors comprising a polynucleotide described herein. In some embodiments, the expression vector is a viral vector or a plasmid.

In one aspect, provided herein are host cells comprising a multispecific protein described herein, a polynucleotide described herein, or an expression vector described herein.

In one aspect, provided here are carriers comprising a multispecific protein described herein, a polynucleotide described herein, or an expression vector described herein.

In some embodiments, the carrier is a lipid nanoparticle, liposome, lipoplex, or nanoliposome.

In one aspect, provided herein are pharmaceutical compositions comprising a multispecific protein described herein, a polynucleotide described herein, an expression vector described herein, a host cell described herein, or a carrier described herein, and a pharmaceutically acceptable excipient.

In one aspect, provided herein are kits comprising a multispecific protein described herein, a polynucleotide described herein, an expression vector described herein, a host cell described herein, a carrier described herein, or a pharmaceutical composition described herein.

In one aspect, provided herein are methods of making a multispecific protein described herein, comprising: introducing into a population of in vitro or ex vivo cells a polynucleotide described herein or a vector described herein, culturing the population of cells under conditions sufficient for the population of cells to express the multispecific protein; and optionally isolating and/or purifying the multispecific protein.

In one aspect, provided herein are methods of delivering a multispecific protein, polynucleotide, expression vector, host cell, carrier, or pharmaceutical composition to a subject, the method comprising administering a multispecific protein described herein, a polynucleotide described herein, an expression vector described herein, a host cell described herein, a carrier described herein, or a pharmaceutical composition described herein, to thereby deliver the multispecific protein, polynucleotide, expression vector, host cell, carrier, or pharmaceutical composition to the subject.

In one aspect, provided herein are methods of inducing an immune response in a subject, the method comprising administering a multispecific protein described herein, a polynucleotide described herein, an expression vector described herein, a host cell described herein, a carrier described herein, or a pharmaceutical composition described herein to the subject, to thereby induce an immune response in the subject.

In one aspect, provided herein are methods of activating a T cell or population of T cells in a subject, the method comprising administering a multispecific protein described herein, a polynucleotide described herein, an expression vector described herein, a host cell described herein, a carrier described herein, or a pharmaceutical composition described herein to the subject, to thereby activate a T cell or population of T cells in the subject.

In one aspect, provided herein are methods of preventing or treating a cancer in a subject, the method comprising administering a multispecific protein described herein, a polynucleotide described herein, an expression vector described herein, a host cell described herein, a carrier described herein, or a pharmaceutical composition described herein to the subject in need thereof, to thereby prevent or treat the cancer in the subject. In some embodiments, the cancer is a solid tumor. In some embodiments, the cancer is breast cancer, ovarian cancer, endometrial cancer, uterine cancer, cervical cancer, anal cancer, prostate cancer, rectal cancer, kidney cancer, bladder cancer, colon cancer, liver cancer, pancreatic cancer, thyroid cancer, thymus cancer, lung cancer, bronchus cancer, skin cancer, brain cancer, spinal cord cancer, head cancer, neck cancer, lip cancer, or oral cavity cancer.

### 4. BRIEF DESCRIPTION OF THE FIGURES

FIG. 1 is a graphical depiction of a representative format of the BCA405 Format of a multispecific protein described herein. In the specific embodiment depicted, the multispecific protein comprises (i) a full-length antibody that specifically binds a first human tumor associated antigen (hTAA) and a second hTAA; (ii) a first scFv operably connected to the C-terminus of the first heavy chain of the full-length antibody, wherein the first scFv specifically binds human CD3 (hCD3); and a second scFv operably connected to the C-terminus of the second heavy chain of the full-length antibody, wherein the second scFv specifically binds a human T-cell co-stimulatory antigen (hTCSA) (e.g., human CD28 (hCD28), human CD2 (hCD2)). In some embodiments, the first and second hTAAs are the same. In some embodiments, the first scFv is operably connected to the C-terminus of the first heavy chain of the full-length antibody via a peptide linker; and the second scFv is operably connected to the C-terminus of the second heavy chain of the full-length antibody via a peptide linker.

FIG. 2 is a graphical depiction of a representative format of the BCA406 Format of a multispecific protein described herein. In the specific embodiment depicted, the multispecific protein comprises (i) a full-length antibody that specifically binds a first human hTAA and a second hTAA; (ii) a first scFv operably connected to the N-terminus of the first heavy chain of the full-length antibody, wherein the first scFv specifically binds hCD3; and a second scFv operably connected to the N-terminus of the second heavy chain of the full-length antibody, wherein the second scFv specifically binds a hTCSA (e.g., hCD28, hCD2). In some embodiments, the first and second hTAAs are the same. In some embodiments, the first scFv is operably connected to the N-terminus of the first heavy chain of the full-length antibody via a peptide linker; and the second scFv is operably connected to the N-terminus of the second heavy chain of the full-length antibody via a peptide linker.

FIG. 3 is a graphical depiction of a representative format of the BCA424 Format of a multispecific protein described herein. In the specific embodiment depicted, the multispecific protein comprises a first Fab operably connected to a first scFv operably connected to a first Fc region; and a second Fab operably connected to a second scFv operably connected to a second Fc region; wherein the first Fab specifically binds a first hTAA, the second Fab specifically binds a second hTAA, the first scFv specifically binds a hTCSA (e.g., hCD28, hCD2), and the second scFv specifically binds hCD3. In some embodiments, the first and second hTAAs are the same. In some embodiments, the first Fab is operably connected to the first scFv via a peptide linker; and the second Fab is operably connected to the second scFv via a peptide linker.

FIG. 4 is a graphical depiction of a representative format of the BCA418 Format of a multispecific protein described herein. In the specific embodiment depicted, the multispecific protein comprises a first scFv operably connected to the VH region of a first Fab operably, which is operably connected to a first Fc region; and a first IgM CH2 mFab operably connected to a second Fc region; wherein the first scFv specifically binds a hTCSA (e.g., hCD28, hCD2), the first Fab specifically binds hCD3, and the first IgM CH2 mFab specifically binds a hTAA. In some embodiments, the first Fab is operably connected to the VL region of the scFv. In some embodiments, the first Fab is operably connected to the VH region of the scFv. In some embodiments, the first Fab is operably connected to the first scFv via a peptide linker.

FIG. 5A is a line graph showing ELISA based HER2 binding of the indicated multispecific protein or control at the indicated concentrations. Graphs show representative data from at least two independent experiments. The table below the graph shows the EC50 values based on n=2 for BCA401 and n=3 for BCA405, BCA406 and BCA424. FIG. 5B is a line graph showing ELISA based HER2 binding of the indicated multispecific protein or control at the indicated concentrations. Graphs show representative data from at least two independent experiments. The table below the graph shows the EC50 values based on n=2 for BCA418 and BCA401 and n=3 for BCA418.

FIG. 6 is a line graph showing EGFR binding on A431 cells by the indicated multispecific protein (BCA405.EG, BCA406.EG, BCA424.EG, which are the same formats as shown in FIGS. 1, 2 and 3 with anti-EGFR as the TAA targeting arm) or control (Cetuximab, positive control for EGFR binding) at the indicated concentrations. The table below the graph shows the EC50 (nM) values.

FIG. 7A is a line graph showing ELISA based CD3 binding of the indicated multispecific protein or control at the indicated concentration. The graph shows representative data from at least two independent experiments. The table below the graph shows the EC50 value based on n=3 for BCA405, BCA406 and BCA424. FIG. 7B is a line graph showing ELISA based CD3 binding of the indicated multispecific protein or control at the indicated concentrations. The graph shows representative data from at least two independent experiments. The table below the graph shows the EC50 value based on n=2 for BCA418.

FIG. 8 is a line graph showing CD3 binding on CD28 knockout Jurkat cells by the indicated multispecific protein or control at the indicated concentrations. The graph shows representative data from two independent experiments. The table below the graph shows qualitative ranking based on shifts in curves relative to BCA403 (anti-CD3 mAb). Qualitative ranking done based on shifts in curves where +, ++, +++ represents low, mid and high binding compared to BCA403.

FIG. 9A is a line graph showing ELISA based CD28 binding of the indicated multispecific protein or control at the indicated concentrations. The graph shows representative data from at least two independent experiments. The table below the graph shows the EC50 value based on n=3 for BCA405, BCA406 and BCA424. FIG. 9B is a line graph showing CD28 binding of the indicated multispecific protein or control at the indicated concentrations. The graph shows representative data from at least two independent experiments. The table below the graph shows the EC50 value based on n=2 for BCA418.

FIG. 10 is a line graph showing CD28 binding on CD3 knockout Jurkat cells by the indicated multispecific protein or control at the indicated concentrations. The graph shows representative data from two independent experiments. The table below the graph qualitative ranking based on shifts in curves relative to BCA402 (anti-CD28 mAb). Qualitative ranking done based on shifts in curves where +, ++, +++ represents low, mid and high binding compared to BCA402.

FIG. 11 is a line graph showing the percent cytotoxicity mediated by the indicated multispecific protein or control at the indicated concentrations in the SKBR3-PBMC co-culture assay. Target cells (Tumor cells, T) were co-cultured with Human PBMCs (E) at T:E ratio of 1:10 for 48 h. Cytotoxicity was evaluated using a luminescence readout (BioGlo). Formula used: % Cytotoxicity=[100−(RLU^{N}/RLU^{Untreated SKBR3-Luc})×100], where RLU^{N}=RLU_{All groups}−RLU^{Unstimulated PBMC}. RLU=Relative Luminescence Units. Each point represents mean±SD from duplicate values in an experiment. The graph shows representative data from at least two independent experiments using two PBMC donors. Human IgG isotype control antibody (hIgG) was used as the negative control. The table below the graph shows the EC50 (nM) values.

FIG. 12 a line graph showing the percent cytotoxicity mediated by the indicated multispecific protein or control at the indicated concentrations in the BxPC3-PBMC co-culture assay. Target cells were co-cultured with Human PBMCs at T:E ratio of 1:10 for 48 h. Cytotoxicity was evaluated using a luminescence readout (BioGlo). Formula used: % Cytotoxicity=[100−(RLU^{N}/RLU^{Untreated SKBR3-Luc})×100], where RLU^{N}=RLU^{All groups}−RLU^{Unstimulated PBMC}. RLU=Relative Luminescence Units. The percent cytotoxicity is shown normalized to a hIgG control. Each point represents mean±SD from duplicate values in an experiment and are representative of two independent experiments.

FIG. 13A is a bar graph showing the level of IL-2 and IFNγ released from SKBR3luc-PBMC co-cultures treated with the indicated multispecific protein or control at the indicated concentrations. The data is plotted as mean±SD of duplicate wells from an experiment. FIG. 13B is a bar graph showing the level of granzyme B released from SKBR3luc-PBMC co-cultures treated with the indicated multispecific protein or control at the indicated concentrations. The data is plotted as mean±SD of duplicate wells from an experiment. FIG. 13C is a bar graph showing the level of IL-6 and TNFα released from SKBR3luc-PBMC co-cultures treated with the indicated multispecific protein or control at the indicated concentrations. The data is plotted as mean±SD of duplicate wells from an experiment. FIG. 13D is a bar graph showing the level of IL-2 and IFN7 released from PBMC cultures treated with the indicated multispecific protein or control at the indicated concentrations. FIG. 13E is a bar graph showing the level of Granzyme B released from PBMC cultures treated with the indicated multispecific protein or control at the indicated concentrations. The data is plotted as mean±SD of duplicate wells from an experiment. FIG. 13F is a bar graph showing the level of IL-6 and TNFα released from PBMC cultures treated with the indicated multispecific protein or control at the indicated concentrations.

FIG. 14A is a bar graph showing the frequency of activated T-cells (CD3+CD25+ cells) in a PBMC culture treated with the indicated multispecific protein or control at the indicated concentrations. The graph is representative from n=3 experiments for BCA405, BCA406, BCA424 and BCA410 and n=2 for BCA418 across two PBMC donors. FIG. 14B is a bar graph showing the frequency of Bcl-xL expressing T-cells (CD3+Bcl-xL+ cells) in a PBMC culture treated with the indicated multispecific protein or control at the indicated concentrations. The graph is representative from n=3 experiments for BCA405, BCA406, BCA424 and BCA410 and n=2 for BCA418 across two PBMC donors. FIG. 14C is a bar graph showing the frequency of activated T-cells (CD3+CD25+ cells) in a SKBR3-PBMC co-culture treated with the indicated multispecific protein or control at the indicated concentrations. The graph is representative from n=3 for BCA405, BCA406, BCA424 and BCA410 and n=2 for BCA418 across two PBMC donors. FIG. 14D is a bar graph showing the frequency of Bcl-xL expressing T-cells (CD3+Bcl-xL+ cells) in a SKBR3-PBMC co-culture treated with the indicated multispecific protein or control at the indicated concentrations. The graph is representative from n=3 for BCA405, BCA406, BCA424 and BCA410 and n=2 for BCA418 across two PBMC donors.

FIG. 15A is a bar graph showing the frequency of Bcl-xL expressing T-cells (CD3+Bcl-xL+ cells) in a PBMC culture treated with the indicated multispecific protein or control at the indicated concentration. FIG. 15B is a bar graph showing the frequency of Bcl-xL expressing T-cells (CD3+Bcl-xL+ cells) in a FaDu-PBMC co-culture treated with the indicated multispecific protein or control at the indicated concentrations.

FIG. 16A is a bar graph showing the frequency of Bcl-xL expressing T-cells (CD3+Bcl-xL+ cells) in a PBMC culture treated with the indicated multispecific protein or control at the indicated concentrations. FIG. 16B is a bar graph showing the frequency of Bcl-xL expressing T-cells (CD3+Bcl-xL+ cells) in a SKBR3-PBMC co-culture treated with the indicated multispecific protein or control at the indicated concentrations.

FIG. 17A is a line graph showing the percent cytotoxicity mediated by the indicated multispecific protein or control at the indicated concentrations in the SKBR3-PBMC co-culture assay. Target cells were co-cultured with Human PBMCs at T:E ratio of 1:10 for 48 h. Cytotoxicity was evaluated using a luminescence readout (BioGlo). Formula used: % Cytotoxicity=[100−(RLU^{N}/RLU^{Untreated SKBR3-Luc})×100], where RLU^{N}=RLU^{All groups}−RLU^{Unstimulated PBMC}. RLU=Relative Luminescence Units. Each point represents Mean±SD from duplicate values in an experiment. FIG. 17B a line graph showing the percent cytotoxicity mediated by the indicated multispecific protein or control at the indicated concentrations in the SKBR3-PBMC co-culture assay. Target cells were co-cultured with Human PBMCs at T:E ratio of 1:10 for 48 h. Cytotoxicity was evaluated using a luminescence readout (BioGlo). Formula used: % Cytotoxicity=[100−(RLU^{N}/RLU^{Untreated SKBR3-Luc})×100], where RLU^{N}=RLU^{All groups}−RLU^{Unstimulated PBMC}. RLU=Relative Luminescence Units. Each point represents Mean±SD from duplicate values in an experiment. FIG. 17C a line graph showing the percent cytotoxicity mediated by the indicated multispecific protein or control at the indicated concentrations in the SKBR3-PBMC co-culture assay. Target cells were co-cultured with Human PBMCs at T:E ratio of 1:10 for 48 h. Cytotoxicity was evaluated using a luminescence readout (BioGlo). Formula used: % Cytotoxicity=[100−(RLU^{N}/RLU^{Untreated SKBR3-Luc})×100], where RLU^{N}=RLU^{All groups}−RLU^{Unstimulated PBMC}. RLU=Relative Luminescence Units. Each point represents Mean±SD from duplicate values in an experiment. FIG. 17D a line graph showing the percent cytotoxicity mediated by the indicated multispecific protein or control at the indicated concentrations in the SKBR3-PBMC co-culture assay. Target cells were co-cultured with Human PBMCs at T:E ratio of 1:10 for 48 h. Cytotoxicity was evaluated using a luminescence readout (BioGlo). Formula used: % Cytotoxicity=[100−(RLU^{N}/RLU^{Untreated SKBR3-Luc})×100], where RLU^{N}=RLU^{All groups}−RLU^{Unstimulated PBMC}. RLU=Relative Luminescence Units. Each point represents Mean±SD from duplicate values in an experiment.

FIG. 18A is a bar graph showing the percent of activated CD4+ T-cells (CD4+CD25+ cells) in a FaDu-PBMC co-culture treated with the indicated multispecific protein or control at the indicated concentrations. FIG. 18B is a bar graph showing the percent of activated CD8+ T-cells (CD8+CD25+ cells) in a FaDu-PBMC co-culture treated with the indicated multispecific protein or control at the indicated concentrations. FIG. 18C is a bar graph showing the percent of activated CD4+ T-cells (CD4+CD25+ cells) in a PBMC culture treated with the indicated multispecific protein or control at the indicated concentrations. FIG. 18D is a bar graph showing the percent of activated CD8+ T-cells (CD8+CD25+ cells) in a PBMC culture treated with the indicated multispecific protein or control at the indicated concentrations.

FIG. 19A is a line graph showing the percent of activated CD4+CD25+ memory T-cells (CD45RO and CCR7 (Naïve CD45RO−CCR7+), (TEM CD45RO+CCR7−), (TCM CD45RO+CCR7+), (DN CD45RO−CCR7−) in a FaDu-PBMC co-culture treated with the indicated control at the indicated concentrations. FIG. 19B is a line graph showing the percent of activated CD8+CD25+ memory T-cells (CD45RO and CCR7 (Naïve CD45RO−CCR7+), (TEM CD45RO+CCR7−), (TCM CD45RO+CCR7+), (DN CD45RO−CCR7−) in a FaDu-PBMC co-culture treated with the indicated control at the indicated concentrations. FIG. 19C is a line graph showing the percent of activated CD4+CD25+ memory T-cells (CD45RO and CCR7 (Naïve CD45RO−CCR7+), (TEM CD45RO+CCR7−), (TCM CD45RO+CCR7+), (DN CD45RO−CCR7−)) in a FaDu-PBMC co-culture treated with the indicated multispecific protein at the indicated concentrations. FIG. 19D is a line graph showing the percent of activated CD8+CD25+ memory T-cells (CD45RO and CCR7 (Naïve CD45RO−CCR7+), (TEM CD45RO+CCR7−), (TCM CD45RO+CCR7+), (DN CD45RO−CCR7−)) in a FaDu-PBMC co-culture treated with the indicated multispecific protein at the indicated concentrations.

FIG. 20A is a line graph showing the level of IFN7 released from FaDu-PBMC co-cultures treated with the indicated multispecific protein or control at the indicated concentrations. FIG. 20B is a line graph showing the level of IL-2 released from FaDu-PBMC co-cultures treated with the indicated multispecific protein or control at the indicated concentrations. FIG. 20C is a line graph showing the level of TNF released from FaDu-PBMC co-cultures treated with the indicated multispecific protein or control at the indicated concentrations. FIG. 20D is a line graph showing the level of IL-6 released from FaDu-PBMC co-cultures treated with the indicated multispecific protein or control at the indicated concentrations. FIG. 20E is a line graph showing the level of IL-4 released from FaDu-PBMC co-cultures treated with the indicated multispecific protein or control at the indicated concentrations. FIG. 20F is a line graph showing the level of IL-10 released from FaDu-PBMC co-cultures treated with the indicated multispecific protein or control at the indicated concentrations. FIG. 20G is a line graph showing the level of IL-17A released from FaDu-PBMC co-cultures treated with the indicated multispecific protein or control at the indicated concentrations.

FIG. 21A is a graphical depiction of the structure of multispecific antibody BCA605. The structure of the multispecific is akin that of BCA405 (described in FIG. 1). The anti-TAA Fabs of BCA605 specifically bind MSLN. In some embodiments, the anti-CD3 and anti-hTCSA (e.g., CD28, CD2) scFv arms contain engineered disulfide bonds (as indicated graphically by the lines). FIG. 21B is a graphical depiction of the structure of multispecific antibody BCA606. The structure of the multispecific is akin that of BCA406 (described in FIG. 2). The anti-TAA Fabs of BCA605 specifically bind MSLN. In some embodiments, the anti-CD3 and anti-hTCSA (e.g., CD28, CD2) scFv arms contain engineered disulfide bonds (as indicated graphically by the lines). FIG. 21C is a graphical depiction of the structure of multispecific antibody BCA624. The structure of the multispecific is akin that of BCA424 (described in FIG. 3). The anti-TAA Fabs of BCA605 specifically bind MSLN. In some embodiments, the anti-CD3 and anti-hTCSA (e.g., CD28, CD2) scFv arms contain engineered disulfide bonds (as indicated graphically by the lines).

FIG. 22A is a line graph showing binding of the indicated multispecific protein (BCA605 (MOR), BCA606 (MOR), BCA624 (MOR)) or control (BCA429 (MOR), hIgG) at the indicated concentrations to MSLN expressed on the surface of OVACAR-3/CMV Luc cells. FIG. 22B is a line graph showing binding of the indicated multispecific protein (BCA605 (FB6), BCA606 (FB6), BCA624 (FB6)) or control (hIgG) at the indicated concentrations to MSLN expressed on the surface of OVACAR-3/CMV Luc cells.

FIG. 23A is a line graph showing binding of the indicated multispecific protein (BCA605 (MOR), BCA606 (MOR), BCA624 (MOR)) or control (BCA402, BCA403, hIgG) at the indicated concentrations to CD28 expressed on the surface of CD3eKO Jurkat cells as measured by flow cytometry. FIG. 23B is a line graph showing binding of the indicated multispecific protein (BCA605 (FB6), BCA606 (FB6), BCA624 (FB6)) or control (BCA402, BCA403, hIgG) at the indicated concentrations to CD28 expressed on the surface of CD3eKO Jurkat cells as measured by flow cytometry.

FIG. 24A is a line graph showing binding of the indicated multispecific protein (BCA605 (MOR), BCA606 (MOR), BCA624 (MOR)) or control (BCA402, BCA403, hIgG) at the indicated concentrations to CD3 expressed on the surface of CD28eKO Jurkat cells as measured by flow cytometry. FIG. 24B is a line graph showing binding of the indicated multispecific protein (BCA605 (FB6), BCA606 (FB6), BCA624 (FB6)) or control (BCA402, BCA403, hIgG) at the indicated concentrations to CD3 expressed on the surface of CD28eKO Jurkat cells as measured by flow cytometry. FIG. 24C is a line graph showing binding of the indicated multispecific protein (BCA605 (FB6), BCA606 (FB6), BCA624 (FB6)) or control (BCA403) at the indicated concentrations to CD3 as measured by ELISA.

FIG. 25A is a line graph showing the percent cytotoxicity mediated by the indicated multispecific protein (BCA605 (MOR), BCA606 (MOR), BCA624 (MOR), BCA605 (FB6), BCA606 (FB6), BCA624 (FB6)) at the indicated concentrations in the OVCAR3-PBMC (1:10) co-culture assay. FIG. 25B is a line graph showing the percent cytotoxicity mediated by the indicated multispecific protein (BCA605 (MOR), BCA606 (MOR), BCA624 (MOR), BCA605 (FB6), BCA606 (FB6), BCA624 (FB6)) at the indicated concentrations in the SCOV3-PBMC (1:10) co-culture assay.

FIG. 26A is a bar graph showing IL-2 release from tumor-PBMC co-cultures with the indicated multispecific protein (BCA605 (MOR), BCA606 (MOR), BCA624 (MOR)) or control (BCA429 (MOR), hIgG, unstimulated co-culture (target+effector (T+E))) at the indicated concentrations. FIG. 26B is a bar graph showing IL-6 release from tumor-PBMC co-cultures with the indicated multispecific protein (BCA605 (MOR), BCA606 (MOR), BCA624 (MOR)) or control (BCA429 (MOR), hIgG, unstimulated co-culture (target+effector (T+E))) at the indicated concentrations. FIG. 26C is a bar graph showing IFN7 release from tumor-PBMC co-cultures with the indicated multispecific protein (BCA605 (MOR), BCA606 (MOR), BCA624 (MOR)) or control (BCA429 (MOR), hIgG, unstimulated co-culture (target+effector (T+E))) at the indicated concentrations. FIG. 26D is a bar graph showing TNF release from tumor-PBMC co-cultures with the indicated multispecific protein (BCA605 (MOR), BCA606 (MOR), BCA624 (MOR)) or control (BCA429 (MOR), hIgG, unstimulated co-culture (target+effector (T+E))) at the indicated concentrations. FIG. 26E is a bar graph showing IL-10 release from tumor-PBMC co-cultures with the indicated multispecific protein (BCA605 (MOR), BCA606 (MOR), BCA624 (MOR)) or control (BCA429 (MOR), hIgG, unstimulated co-culture (target+effector (T+E))) at the indicated concentrations. FIG. 26F is a bar graph showing IL-17 release from tumor-PBMC co-cultures with the indicated multispecific protein (BCA605 (MOR), BCA606 (MOR), BCA624 (MOR)) or control (BCA429 (MOR), hIgG, unstimulated co-culture (target+effector (T+E))) at the indicated concentrations. FIG. 26G is a bar graph showing IL-4 release from tumor-PBMC co-cultures with the indicated multispecific protein (BCA605 (MOR), BCA606 (MOR), BCA624 (MOR)) or control (BCA429 (MOR), hIgG, unstimulated co-culture (target+effector (T+E))) at the indicated concentrations.

FIG. 27A is a bar graph showing IFNγ release from PBMCs treated with the indicated multispecific antibody (BCA605 (MOR), BCA606 (MOR), BCA624 (MOR)) or control (BCA403 (MOR), hIgG) either in soluble or tethered form. Data from two PBMC lots is shown. FIG. 27B is a bar graph showing IFNγ release from PBMCs treated with the indicated multispecific antibody (BCA605 (FB6), BCA606 (FB6), BCA624 (FB6)) or control (BCA403 (MOR), hIgG) either in soluble or tethered form. Data from two PBMC lots is shown.

FIG. 28 shows a graphical depiction of the format of the control multispecific protein referred to herein as BCA410.

FIG. 29 shows a graphical representation of the overall structure of a naturally occurring full length antibody. This structure is utilized in several control antibodies described herein, including e.g., BCA401 (anti-HER2 full length antibody), BCA402 (anti-CD28 full length antibody), BCA403 (anti-EGFR full length antibody), BCA403 (αCD3ε full length antibody), and cetuximab (anti-EGFR full length antibody).

### 5. DETAILED DESCRIPTION

T-cell engaging immunotherapies function by targeting T-cells (or a subset thereof, (e.g., CD8+ T cells)) to a tumor. For example, bispecific T-cell engaging single-chain antibodies (also called BiTEs) are single chain proteins that simultaneously bind an antigen on tumor cells and an antigen expressed on the surface of T-cells in order to activate the T-cells and induce tumor lysis. Despite the promising efficacy of T-cell engagers in clinical trials, they have also exhibited severe dose limiting adverse events, including cytokine release syndrome (CRS) and neurotoxicity. CRS is an uncontrolled systemic inflammatory response characterized by elevated levels of pro-inflammatory cytokines (e.g., IL-6) which are triggered by T-cell activation. CRS mediated by T-cell engagers results, inter alia, from peripheral toxicity—through the activation of peripheral T-cells outside of the tumor microenvironment. The inventors have, inter alia, discovered novel multispecific protein formats that specifically bind hCD3, a hTCSA (e.g., hCD28, hCD2), and a hTAA, that exhibit low to no peripheral toxicity. Accordingly, the novel multispecific proteins described herein are good candidates for the treatment of diseases (e.g., cancer). As such, the current disclosure provides, inter alia, novel multispecific proteins for use in pharmaceutical compositions for the treatment of diseases (e.g., cancer).

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” should be assumed to be within an acceptable error range for that particular value or composition.

Unless otherwise indicated or clear from the context, the use of the terms akin to “first and second,” or “(a) and (b)” or “(i) and (ii)” herein do not denote an order or orientation but are used to identity multiple components of a composition or method. It will be clear from the context to a person of ordinary skill in the art where these terms are intended to denote an order or orientation.

Where proteins and/or polypeptides are described herein, it is understood that polynucleotides (e.g., RNA (e.g., mRNA) or DNA polynucleotides) encoding the protein or polypeptide are also provided herein.

Where proteins, polypeptides, polynucleotides, cells, expression vectors, etc. are described herein, it is understood that isolated forms of the proteins, polypeptides, polynucleotides, cells, expression vectors, etc. are also provided herein.

Where proteins, polypeptides, polynucleotides, etc. are described herein, it is understood that recombinant forms of the proteins, polypeptides, polynucleotides, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

As used herein, the term “administering” refers to the physical introduction of an agent, e.g., a therapeutic agent (or a precursor of the therapeutic agent that is metabolized or altered within the body of the subject to produce the therapeutic agent in vitro) to a subject, using any of the various methods and delivery systems known to those skilled in the art. Administering can be performed, for example, once, a plurality of times, and/or over one or more extended periods.

As used herein the term “antibody dependent cell mediated cytotoxicity” or “ADCC” refers to an immune mechanism leading to the lysis of antibody (or an Fc region containing polypeptide or protein) (e.g., an Ig Fc containing fusion protein or polypeptide described herein)-coated target cells by immune effector cells (e.g., NK cells). As used herein, the term “reduced ADCC” and the like refers to either a reduction in the number of target cells that are lysed in a given time, at a given concentration of antibody (or an Ig Fc region containing polypeptide or protein) (e.g., an Fc region containing fusion protein or polypeptide described herein) in the medium surrounding the target cells, by the mechanism of ADCC defined above, and/or an increase in the concentration of antibody (or an Fc region containing polypeptide or protein) (e.g., an Fc containing fusion protein or polypeptide described herein) in the medium surrounding the target cells, required to achieve the lysis of a given number of target cells in a given time, by the mechanism of ADCC defined above. The reduction in ADCC is relative to the ADCC mediated by the same antibody (or an Fc region containing polypeptide or protein) (e.g., an Fc containing fusion protein or polypeptide described herein) produced by the same type of host cells, using the same standard production, purification, formulation and storage methods (which are known to those skilled in the art), but that has not been engineered (e.g., does not comprise one or more amino acid modification, e.g., amino acid substitution, that mediates a decrease in ADCC). For example the reduction in ADCC mediated by an antibody (or an Fc region containing polypeptide or protein) (e.g., an Fc containing fusion protein or polypeptide described herein) comprising in its Fc region an amino acid substitution that reduces ADCC, is relative to the ADCC mediated by the same antibody (or an Fc region containing polypeptide or protein) (e.g., an Fc containing fusion protein or polypeptide described herein) without said amino acid substitution in the Fc region.

As used herein, the term “affinity” refers to the strength of the binding of one protein (e.g., an Antibody) to another protein (e.g., an Antigen). The affinity of a protein is measured by the dissociation constant Kd, defined as [Antibody]×[Antigen]/[Antibody-Antigen] where [Antibody-Antigen] is the molar concentration of the Antibody-Antigen complex, [Antibody] is the molar concentration of the unbound Antibody and [Ligand] is the molar concentration of the unbound Antigen. The affinity constant Ka is defined by 1/Kd. Standard methods of measuring affinity are known to the person of ordinary skill in the art. Exemplary methods of measuring affinity are described herein, see for example, § 5.2.5.

As used herein, the term “antibody” or “antibodies” is used in the broadest sense and encompasses various immunoglobulin (Ig) (e.g., human Ig (hIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methods known in the art, e.g., recombinant production in host cells, see, e.g., § 5.3; or synthetic production.

As used herein, the term “antibody-like scaffold” refers to non-Ig based antigen binding domain. Various antibody-like scaffolds are known in the art. Various antibody-like scaffolds are known in the art. For example, 10th type III domain of fibronectin (e.g., AdNectins®) and designed ankyrin repeat proteins (e.g., DARPins®) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008), the full contents of each of which is incorporated by reference herein for all purposes. Exemplary antibody-like scaffolds include, but are not limited to, lipocalins (see, e.g., U.S. Pat. No. 7,250,297) (e.g., Anticalin®), protein A-derived molecules such as z-domains of protein a (see, e.g., U.S. Pat. No. 5,831,012) (e.g., Affibody®), A domains of membrane receptors stabilized by disulfide bonds and Ca2+(see, e.g., U.S. Pat. No. 7,803,907) (e.g., Avimer/Maxibody®), a serum transferrin (see, e.g., US2004023334) (e.g., Transbody®); a designed ankyrin repeat protein (see, e.g., U.S. Pat. No. 7,417,130) (e.g., DARPin®), a fibronectin (see, e.g., U.S. Pat. No. 6,818,418) (e.g., AdNectin®), a C-type lectin domain (see, e.g., US2004132094) (e.g., Tetranectin®); a human gamma-crystallin or ubiquitin (see, e.g., U.S. Pat. No. 7,838,629) (e.g., Affilin®); a kunitz type domain of human protease inhibitors (see, e.g., US2004209243), C-Type Lectins (see, e.g., US2004132094) (e.g., Tetranectins®), cysteine knots or knottins (see, e.g., U.S. Pat. No. 7,186,524) (e.g., Microbodies®), nucleic acid aptamers (see, e.g., U.S. Pat. No. 5,475,096), thioredoxin A scaffold (see, e.g., U.S. Pat. No. 6,004,746) (peptide aptamers), and 10th type III domain of fibronectin (see, e.g., U.S. Pat. No. 6,818,418) (e.g., AdNectins®), and cystine-dense peptides (see, e.g., WO2023023031). Additional exemplary antibody-like scaffolds are known in the art and for example described in Storz U. Intellectual property protection: strategies for antibody inventions. MAbs. 2011; 3(3):310-317. doi:10.4161/mabs.3.3.15530. The entire contents of each of the foregoing references is incorporated herein by reference for all purposes. Antibody like scaffolds include e.g., naturally occurring antigen binders, variant (e.g., functional variants) of naturally occurring antigen binders, fragments (e.g., functional fragments) of naturally occurring antigen binders, and synthetic antigen binders (i.e., not naturally occurring antigen binders).

The term “antigen binding domain” refers to a polypeptide or protein, or the portion of a polypeptide or protein, that is capable of specifically binding to an antigen. Exemplary antigen binding domains include, but are not limited to, single domain antibodies (e.g., VHH, (VHH)₂), single-chain Fvs (e.g., scFv; (scFv)₂), Fab fragments (e.g., Fab, single chain Fab (scFab), F(ab′)₂), and disulfide-linked Fvs (sdFv). The antigen binding domain can be part of a larger polypeptide or protein, e.g., a full-length antibody, an Fc fusion. In some embodiments, the antigen binding domain is part of a full-length antibody. In some embodiments, the antigen binding domain is operably connected to an Fc region. When an antigen binding domain is referred to using the target protein or polypeptide, the term “antigen” may be replaced with the name of the target protein or antigen. For example, an antigen binding domain that specifically binds hCD3 may also be referred to herein as a “hCD3 binding domain.”

The terms “cancer” and “tumor” are used interchangeably herein and refer to a broad group of various diseases characterized by the uncontrolled growth of abnormal cells in the body. Unregulated cell division and growth results in the formation of malignant tumors that can invade neighboring tissues and may also metastasize to distant parts of the body through, e.g., the lymphatic system or bloodstream.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991),the entire contents of each of which is incorporated herein by reference for all purposes. Unless otherwise specified, the term “CDR” is a CDR as defined by Kabat et al., J. Biol. Chem. 252, 6609-6616 (1977) and Kabat et al., Sequences of protein of immunological interest. (1991).

The terms “CH1” and “CH1 region” are used interchangeably herein and refer to the first constant region of an immunoglobulin heavy chain. The amino acid sequence of an exemplary reference hIgG1 CH1 region is set forth in SEQ ID NO: 98; and the amino acid sequence of an exemplary reference hIgG4 CH1 region is set forth in SEQ ID NO: 111.

The terms “CH2” and “CH2 region” are used interchangeably herein and refer to the second constant region of an immunoglobulin heavy chain. The amino acid sequence of an exemplary reference hIgG1 CH2 region is set forth in SEQ ID NO: 100; and the amino acid sequence of an exemplary reference hIgG4 CH2 region is set forth in SEQ ID NO: 113.

The terms “CH3” and “CH3 region” are used interchangeably herein and refer to the third constant region of an immunoglobulin heavy chain. The amino acid sequence of an exemplary reference hIgG1 CH3 region is set forth in SEQ ID NO: 101; and the amino acid sequence of an exemplary reference hIgG4 CH3 region is set forth in SEQ ID NO: 113.

The terms “constant region” and “constant domain” are used interchangeably herein and refer to a carboxyl terminal portion of a light and/or heavy chain of a full-length antibody which is not directly involved in binding of an antibody to antigen, but which can exhibit various effector functions, such as interaction with an Ig Fc receptor (e.g., Fc gamma receptor). The constant region of an Ig molecule generally has a more conserved amino acid sequence relative to an Ig variable domain.

As used herein, the term “derived from,” with reference to a polynucleotide refers to a polynucleotide that has at least 70% (e.g., at least 85%) sequence identity to a reference polynucleotide (e.g., a naturally occurring polynucleotide) or a fragment thereof. The term “derived from,” with reference to a polypeptide or protein refers to a polypeptide or protein that comprises an amino acid sequence that has at least 70% (e.g., at least 85%) sequence identity to the amino acid sequence of a reference polypeptide or protein (e.g., a naturally occurring polypeptide or protein). The term “derived from” as used herein does not denote any specific process or method for obtaining the polynucleotide, polypeptide, or protein. For example, the polynucleotide, polypeptide, or protein can be recombinant produced or chemically synthesized.

As used herein, the term “diagnosing” or “diagnosis” refers to a determination of the presence, absence, severity, or course of treatment of a disease (e.g., a cancer). The term “diagnosing” encompasses an initial determination as well as subsequent determinations (e.g., monitoring) after the initial determination.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “hinge” or “hinge region” are used interchangeably herein and refer to the hinge region of an immunoglobulin heavy chain. The amino acid sequence of an exemplary reference hIgG1 hinge region is set forth in SEQ ID NO: 99; and the amino acid sequence of an exemplary reference hIgG4 hinge region is set forth in SEQ ID NO: 112.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “effector function” when used in reference to an antibody refers to those biological activities attributable to the Fc region of an antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and C1q binding.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, the entire contents of each of which is incorporated herein by reference for all purposes.

As used herein, the term “Fab” refers to an antigen binding domain that comprises a Fab heavy chain that comprises from N- to C-terminus a VH region and a CH1 region; and a light chain comprising from N- to C-terminus a VL region and a CL region; and wherein the Fab heavy chain and the light chain associate to form an antigen binding domain.

The term “Fab-Fc” as used herein refers to an antibody that comprises a Fab operably linked to an Fc region. For example, a full-length antibody comprises a first Fab operably connected to a first Fc region and a second Fab operably connected to a second Fc region.

As used herein, the term “Fc region” refers to the C-terminal region of a hIg heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region (e.g., comprises one or more amino acid modification), see, e.g., § 5.2.7.1. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated herein by reference for all purposes). In some embodiments, the CH3 region comprises a deletion of one or more C-terminal amino acid residues relative to a wild type CH3 region (e.g., a C-terminal lysine; a C-terminal glycine-lysine).

As used herein, the term “Fc modified fusion protein or polypeptide” refers to a fusion polypeptide or protein comprising an Fc region, wherein the Fc region is modified (e.g., comprises one or more amino acid modification (e.g., one or more amino acid substitution, deletion, or addition) relative to a reference Fc region.

As used herein, the terms “first” and “second” with respect to Fc regions etc., are used for convenience of distinguishing when there is more than one of each type of moiety. Use of these terms is not intended to confer a specific order or orientation in the fusion protein unless explicitly so stated.

As used herein, the term “framework region” or “FR region” refers to the amino acid residues that are part of the variable region of an antibody, but are not part of the CDRs (e.g., using the Kabat definition of CDRs).

As used herein, the term “full-length antibody” refers to an antibody having a structure substantially similar to a native antibody structure. E.g., an antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the two heavy chains comprise a substantially identical amino acid sequence; and the two light chains comprise a substantially identical amino acid sequence. In some embodiments, the two heavy chains comprise a substantially identical amino acid sequence except for one or more amino acid modifications that promote heterodimerization of the correct heavy chains (e.g., as described herein); and the two light chains comprise a substantially identical amino acid sequence. Antibody chains may be substantially identical but not entirely identical if they differ due to post-translational modifications, such as C-terminal cleavage of lysine residues, alternative glycosylation patterns, etc.

The term “functional variant” as used herein in reference to a polypeptide or protein refers to a polypeptide or protein that comprises at least one but no more than 15%, not more than 12%, no more than 10%, no more than 8% amino acid variation (e.g., substitution, deletion, addition) compared to the amino acid sequence of a reference polypeptide or protein, wherein the polypeptide or protein retains at least one particular function of the reference polypeptide or protein. Not all functions of the reference polypeptide or protein (e.g., wild type) need be retained by the functional variant of the protein. In some instances, one or more functions are selectively reduced or eliminated. In some embodiments, the reference polypeptide or protein is a wild type protein.

The term “functional fragment” as used herein in reference to a polypeptide or protein refers to a fragment of a reference polypeptide or protein that retains at least one particular function. Not all functions of the reference polypeptide or protein need be retained by a functional fragment of the polypeptide or protein. In some instances, one or more functions are selectively reduced or eliminated. In some embodiments, the reference polypeptide or protein is a wild type protein.

As used herein, the term “fuse” and grammatical equivalents thereof refer to the operable connection of at least a first polypeptide to a second polypeptide, wherein the first and second polypeptides are not naturally found operably connected together. For example, the first and second polypeptides are derived from different proteins. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the first and second polypeptides are different and not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “heavy chain” refers to the portion of an immunoglobulin (e.g., a human Ig) that typically comprises from N- to C-terminus a heavy chain variable region (VH), a CH1 region, a hinge region, a CH2 region, and a CH3 region. The constant regions of the heavy chain (i.e., the CH1 region, the hinge region, the CH2 region, and the CH3 region) can be any distinct isotype, for example, human alpha (α), delta (δ), epsilon (ε), gamma (γ), and mu (μ), based on the amino acid sequence of the constant domain, which give rise to the hIgA, hIgD, IgE, hIgG, and hIgM classes of human antibodies, respectively, including subclasses of hIgG, e.g., hIgG₁, hIgG₂, hIgG₃, and hIgG₄. As used herein, the term “heavy chain” when used in reference to a human antibody can refer to any distinct type, e.g., alpha (α), delta (δ), epsilon (ε), gamma (γ), and mu (μ), based on the amino acid sequence of the constant domain, which give rise to human IgA, IgD, IgE, IgG, and IgM classes of antibodies, respectively, including subclasses of human IgG, e.g., IgG₁, IgG₂, IgG₃, and IgG₄.

As used herein, the term “half-life extension moiety” refers to a moiety (e.g., small molecule, polypeptide, polynucleotide, carbohydrate, lipid, synthetic polymer (e.g., polymers of PEG), etc.) that when conjugated or otherwise operably connected (e.g., fused) to a polypeptide or protein (the subject polypeptide or protein), increases the half-life of the subject polypeptide or protein in vivo when administered to a subject (e.g., a human subject). The pharmacokinetic properties of the polypeptide or protein can be evaluated utilizing in vitro and in vivo models known in the art.

As used herein, the term “half-life extension polypeptide” or “half-life extension protein” refers to a polypeptide that when operably connected to another polypeptide (the subject polypeptide or protein), increases the half-life of the subject polypeptide in vivo when administered to a subject (e.g., a human subject). The pharmacokinetic properties of the polypeptide or protein can be evaluated utilizing in vitro and in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, polynucleotide, carbohydrate, lipid, synthetic polymer (e.g., polymers of PEG), etc.) that is not joined to the polypeptide in nature. For example, a non-limiting example of a heterologous moiety is a heterologous polypeptide (as defined herein).

As used, herein the term “heterologous signal peptide” refers to a signal peptide that is not operably connected to a subject polypeptide or protein in nature. For example, in reference to a polypeptide comprising a signal peptide from human IL-2 (hIL-2) operably connected to hIL-12p40, the hIL-2 signal peptide would constitute a heterologous signal peptide.

As used herein, the term “homologous signal peptide” refers to a signal peptide that is operably connected to a subject polypeptide or protein in nature. For example, in reference to a polypeptide comprising a signal peptide from human IL-2 operably connected to hIL-2, the hIL-2 signal peptide would constitute a homologous signal peptide.

As used herein, the term “human T-cell co-stimulatory antigen” or “hTCSA” refers to a protein expressed on the surface of a human T-cell that is not part of the T-cell receptor complex, that enhances T-cell activation when bound by its cognate ligand. Exemplary hTCSAs are described herein and include, e.g., hCD28, hCD2, and h41BB.

As used herein, the term “human CD28” or “hCD28” refers to the human CD28 protein. hCD28 is a transmembrane protein expressed on T-cells that provides co-stimulatory signals required for T-cell activation and survival. The amino acid sequence of an exemplary reference mature hCD28 protein is set forth in SEQ ID NO: 2.

As used herein, the term “human CD2” or “hCD2” refers to the human CD2 protein. hCD2 is a transmembrane protein expressed on T-cells and NK cells that provides co-stimulatory signals. The amino acid sequence of an exemplary reference mature hCD2 protein is set forth in SEQ ID NO: 38.

As used herein, the term “human CD3” or “hCD3” refers to the human CD3 protein complex. The human CD3 protein complex includes 4 distinct proteins, namely, hCD3ε, hCD3ζ, hCD3γ, and hCD3δ. As such, as used herein the term “CD3” encompasses hCD3ε, hCD3ζ, hCD3γ, and hCD3δ. Where a specific hCD3 protein of the CD3 complex is intended the specific protein (hCD3ε, hCD3ζ, hCD3γ, or hCD3δ) will be specified. For example, reference to “an antigen binding domain that specifically binds hCD3” and the like, encompasses an antigen binding domain that specifically binds any one of hCD3ε, hCD3ζ, hCD3γ, or hCD3δ. Whereas, reference to “an antigen binding domain that specifically binds hCD3ε” and the like, refers to an antigen binding domain that specifically binds hCD3ε and does not specifically binds hCD3ζ, hCD3γ, or hCD3δ. The amino acid sequence of an exemplary reference mature hCD3ε protein is set forth in SEQ ID NO: 22. The amino acid sequence of an exemplary reference mature hCD3δ protein is set forth in SEQ ID NO: 28. The amino acid sequence of an exemplary reference mature hCD3γ protein is set forth in SEQ ID NO: 26. The amino acid sequence of an exemplary reference mature hCD3 protein is set forth in SEQ ID NO: 24.

As used herein, the term “human tumor associated antigen” or “hTAA” refers to a protein that is expressed on the surface of a human cancer cell that allows recruitment of a multispecific protein described herein to the human cancer cell. In some embodiments, the tumor associated antigen is expressed by both normal cells and cancer cells. In some embodiments, the tumor associated antigen is overexpressed by a cancer cell in comparison to a normal cell, for example, 1-fold over expression, 2-fold overexpression, 3-fold overexpression or more in comparison to a normal cell. In some embodiments, the tumor associated antigen is inappropriately synthesized by the cancer cell, for example, a protein that contains amino acid modifications (e.g., amino acid deletions, additions, and/or substitutions), in comparison to the protein expressed by a normal cell. In some embodiments, the tumor associated antigen is only expressed by the cancer cell and not expressed at detectable levels by normal cells. Methods to identify and verify tumor-associated proteins are known to a skilled person and described in the literature (see, e.g., Bornstein, AAPS J. (2015), vol. 17(3), p. 525-534; Hong et al., BMC Syst Biol. (2018), vol. 12 (Suppl 2), p. 17, the entire contents of each of which are incorporated by references herein for all purposes).

As used herein, the term “hIgM CH2 mFab” refers to a modified Fab that comprises a hIgM CH2 mFab heavy chain and a hIgM CH2 mFab light chain, wherein the hIgM CH2 mFab heavy chain and hIgM CH2 mFab light chain associate to form an antigen binding domain.

As used herein, the term, “hIgM CH2 mFab heavy chain” comprises from N- to C-terminus a VH region and an hIgM CH2 region. The amino acid sequence of an exemplary hIgM CH2 region is set forth in SEQ ID NO: 126.

As used herein, the term, “hIgM CH2 mFab light chain” comprises from N- to C-terminus a VL region and an hIgM CH2 region. The amino acid sequence of an exemplary hIgM CH2 region is set forth in SEQ ID NO: 126.

As used herein, the term “isolated” with reference to a polypeptide, protein, polynucleotide, vector (and the like) refers to a polypeptide, protein, polynucleotide, vector (or the like) that is substantially free of other cellular components with which it is associated in the natural state.

As used herein, the term “light chain” refers to the portion of an immunoglobulin (e.g., a human immunoglobulin) that comprises from N- to C-terminus a light chain variable region (VL) operably connected to a light chain constant region (CL). The CL can be any distinct type, e.g., kappa (x) or lambda (k) based on the amino acid sequence of the CL. In some embodiments, the multispecific proteins described herein comprise one or more light chain.

As used herein, the term translatable RNA refers to any RNA that encodes at least one peptide or protein and can be translated to produce the encoded peptide or protein in vitro, in vivo, in situ or ex vivo. In some embodiments, the translatable RNA is an mRNA. The translatable RNA can be linear or circular.

As used herein, the term “modification,” with reference to a polynucleotide, refers to a polynucleotide that comprises at least one substitution, alteration, inversion, addition, or deletion of nucleotide compared to a reference polynucleotide (e.g., one or more amino acid substitutions). Modifications can include the inclusion of non-naturally occurring nucleotide residues. As used herein, the term “modification,” with reference to an amino acid sequence refers to an amino acid sequence that comprises at least one substitution, alteration, inversion, addition, or deletion of an amino acid residue compared to a reference amino acid sequence. Modifications can include the inclusion of non-naturally occurring amino acid residues. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein.

A “modification that promotes heterodimerization of a first Fc region and a second Fc region” (or similar phrasing) is a manipulation of the peptide backbone or the post-translational modifications of an Fc region that reduces or prevents the association of a polypeptide comprising the Fc region with an identical polypeptide to form a homodimer. A modification promoting association as used herein particularly includes separate modifications made to each of the two Fc regions desired to associate (i.e., a first Fc region and a second Fc region), wherein the modifications are complementary to each other so as to promote association of the two Fc regions. For example, a modification promoting association may alter the structure or charge of one or both of the Fc regions so as to make their association sterically or electrostatically favorable, respectively. Thus, heterodimerization occurs between a polypeptide comprising the first Fc region and a polypeptide comprising the second Fc region, which might be non-identical in the sense that further components fused to each of the Fc regions (e.g., antigen binding domains) are not the same. In some embodiments the modification promoting association comprises an amino acid mutation in the Fc region, specifically an amino acid substitution. In a particular embodiment, the modification promoting association comprises a separate amino acid mutation, specifically one or more amino acid substitution, in each of the first Fc region and the second Fc region. See, e.g., § 5.2.7.2.

As used herein, the term “moiety” is used generically to describe any macro or micro molecule that can be operably connected to a polypeptide or protein described herein. Exemplary moieties include, but are not limited small molecules, polypeptides, proteins, polynucleotides (e.g., DNA, RNA), carbohydrates, lipids, synthetic polymers (e.g., polymers of PEG).

As used herein, the term “multispecific” with reference to a protein or polypeptide (e.g., a protein or polypeptide described herein), denotes that the protein or polypeptide comprises at least two antigen binding domains, wherein the at least two antigen binding domains bind different antigens. As such, the term multispecific includes, e.g., bispecific antibodies, trispecific antibodies, tetraspecific antibodies, etc. In some embodiments, the multispecific antibody is trispecific. In some embodiments, the multispecific antibody is trispecific and tetravalent. In some embodiments, the multispecific antibody is trispecific and trivalent.

As used herein, the term “operably connected” refers to the linkage of two moieties (e.g., two polypeptides or two polynucleotides) in a functional relationship. For example, a polypeptide is operably connected to another polypeptide when they are linked (either directly or indirectly via a peptide linker) in frame such that both polypeptides are functional (e.g., a fusion protein or polypeptide described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein or polypeptide).

The determination of “percent identity” between two sequences (e.g., peptide or protein (amino acid sequences) or polynucleotide (nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

The terms “polynucleotide” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” are used interchangeably herein.

As used herein, the term “protein” refers to a polypeptide or a set (i.e., at least two) polypeptides. In embodiments where the protein comprises a set of polypeptides, the set of polypeptides associate to form a functional unit (i.e., quaternary structure). In some embodiments, the polypeptide or set of polypeptides are folded into their three-dimensional structure (i.e., tertiary or quaternary structure). Where polypeptides or sets of polypeptides are contemplated herein, it should be understood that proteins comprising the polypeptides or sets of polypeptides folded into their three-dimensional structure (i.e., tertiary or quaternary structure) are also provided herein and vice versa.

A “prophylactic” treatment is a treatment administered to a subject who does not exhibit signs of a disease or exhibits only early signs for the purpose of decreasing the risk of developing pathology.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

The term “scFv” or “single chain variable fragment” refers to an antibody that comprises a VH region operably connected via a peptide linker to a VL region, wherein the VH and VL regions associate to specifically bind an antigen (e.g., form an antigen binding domain). In some embodiments, the scFv comprises from N- to C-terminus an VH region, a peptide linker, and an VL region. In some embodiments, the scFv comprises from N- to C-terminus an VL region, a peptide linker, and an VH region. In some embodiments, the scFv further comprises one or more engineered disulfide bonds that connect the VH region to the VL region.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide.

As used herein, the term “specifically binds” refers to the preferential interaction, i.e., significantly higher binding affinity, between a first moiety (e.g., protein (e.g., a ligand)) and a second moiety (e.g., protein (e.g., the ligand's cognate receptor)) relative to other moieties (e.g., amino acid sequences). For example, herein, when a first protein or polypeptide is said to “specifically bind” to a second protein or polypeptide, it is understood that the first protein or polypeptide specifically binds to an epitope of the second protein or polypeptide. The term “epitope” refers to the portion of the second protein or polypeptide that the first protein or polypeptide specifically recognizes. The term specifically binds includes molecules that are cross reactive with the same epitope of a different species. For example, an antibody that specifically binds human CD28 may be cross reactive with CD28 of another species (e.g., cynomolgus, murine, etc.), and still be considered herein to specifically bind human CD28. Moieties (e.g., proteins) can specifically bind more than one moiety (e.g., protein (e.g., epitope)).

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutically effective amount” of an agent (e.g., therapeutic agent) refers to any amount of the agent (e.g., therapeutic agent) that, when used alone or in combination with another agent (e.g., therapeutic agent), protects a subject against the onset of a disease or promotes disease regression evidenced by a decrease in severity of disease of infection symptoms, an increase in frequency and duration of disease or infection symptom-free periods, or a prevention of impairment or disability due to the disease or infection affliction. The ability of an agent (e.g., therapeutic agent) to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is prophylactic, i.e., the effect protects or prevents an occurrence or reoccurrence of a disease. To this end, the presently disclosed methods comprise administering a therapeutically effective amount of a compositions as described herein.

As used herein, the term “variable region” refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VH” and “VH region” are used interchangeably to refer to an immunoglobulin heavy chain variable region. A VH region can be incorporated into an antibody, e.g., a scFv, a Fab, a full-length antibody. For example, a scFv comprises a VH region operably connected via a peptide linker to a VL region.

The terms “VL” and “VL region” are used interchangeably to refer to an immunoglobulin light chain variable region. A VL region can be incorporated into an antibody, e.g., a scFv, a Fab, a full-length antibody. For example, a scFv comprises a VL region operably connected via a peptide linker to a VH region.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

### 5.2 Multispecific Proteins

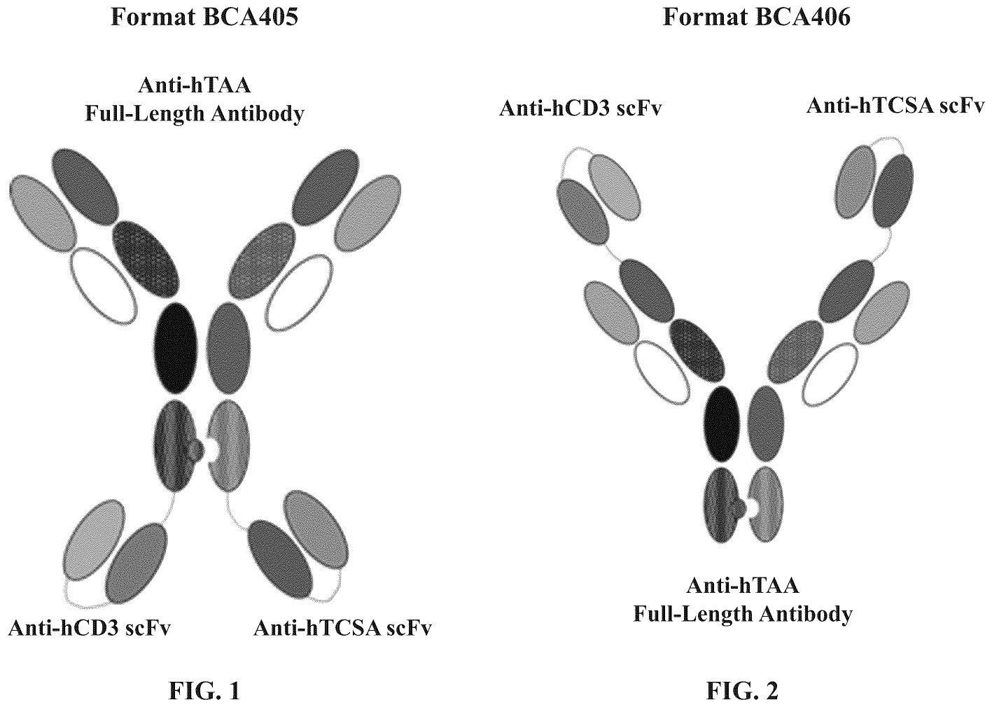

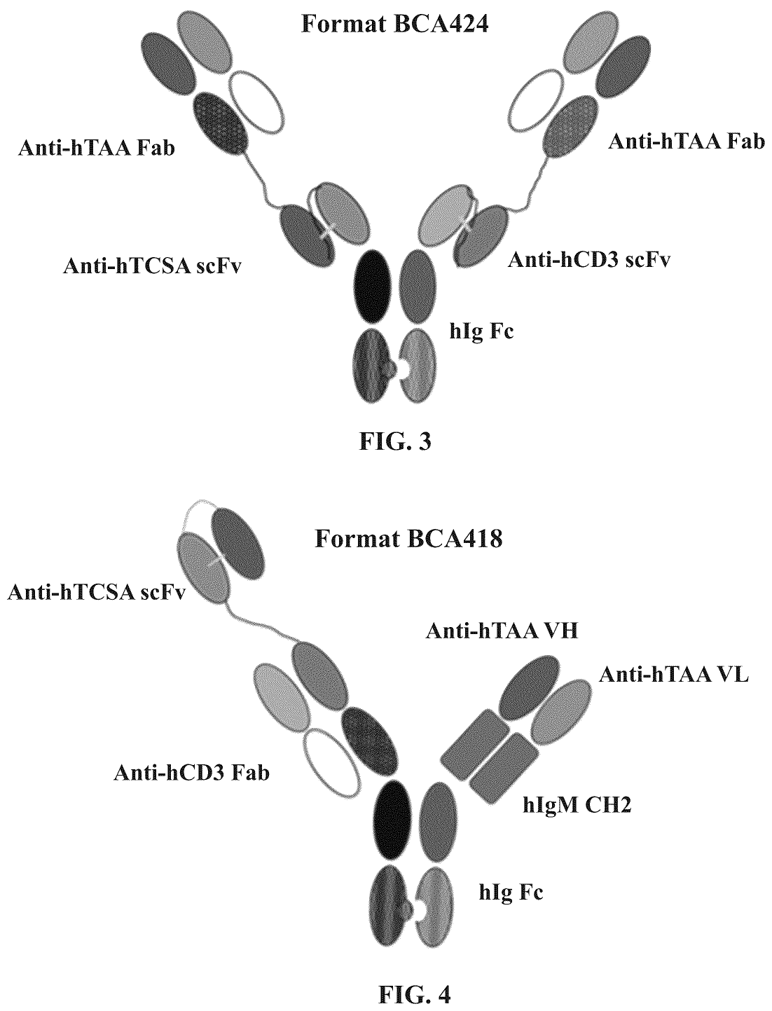

In one aspect, provided herein are multispecific proteins that specifically bind a TAA (e.g., a human tumor associated antigen (hTAA)), CD3 (e.g., human (hCD3)), and a TCSA (e.g., a human TCSA (hTCSA) (e.g., hCD28, hCD2)). The components of the multispecific proteins described herein can be arranged in one of the following formats: Format BCA405 (see, e.g., FIG. 1), Format BCA406 (see, e.g., FIG. 2), Format BCA424 (see, e.g., FIG. 3), or Format BCA418 (see, e.g., FIG. 4), as further described below.

### 5.2.1 Multispecific Protein Formats

### 5.2.1.1 Format BCA405

In one aspect, provided herein are multispecific proteins set forth in Format BCA405 (see, e.g., FIG. 1). Generally, Format BCA405 provides a multispecific protein comprising (i) a full-length antibody that specifically binds a first hTAA and a second hTAA; (ii) a first scFv operably connected to the C-terminus of the first heavy chain of the full-length antibody, wherein the first scFv specifically binds hCD3; and a second scFv operably connected to the C-terminus of the second heavy chain of the full-length antibody, wherein the second scFv specifically binds a hTCSA (e.g., hCD28, hCD2).

In some embodiments, the multispecific protein comprises (a) a full-length antibody comprising: (i) a first light chain comprising from N- to C-terminus a light chain variable region (VL) region and a light chain constant region (CL) region; (ii) a first heavy chain comprising from N- to C-terminus a heavy chain variable region (VH) region, a CH1 region, a hinge region, a CH2 region, and a CH3 region; (iii) a second heavy chain comprising from N- to C-terminus a VH region, a CH1 region, a hinge region, a CH2 region, and a CH3 region; (iv) a second light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer; (b) a first scFv operably connected to the C-terminus of said CH3 region of said first heavy chain of said full-length antibody, wherein said first scFv comprises from N- to C-terminus (i) a VH region, a peptide linker, and a VL region, or (ii) a VL region, a peptide linker, and a VH region; and (c) a second scFv operably connected to the C-terminus of said CH3 region of said second heavy chain of said full-length antibody, wherein said second scFv comprises from N- to C-terminus (i) a VH region, a peptide linker, and a VL region, or (ii) a VL region, a peptide linker, and a VH region; wherein said first antigen binding domain of said full-length antibody specifically binds to a first hTAA; wherein said second antigen binding domain of said full-length antibody specifically binds to a second hTAA; wherein said first scFv specifically binds to a hTCSA (e.g., hCD28, hCD2); and wherein said second scFv specifically binds to hCD3.

In some embodiments, the first scFv is operably connected to the C-terminus of the CH3 of the first heavy chain directly via a peptide bond. In some embodiments, the first scFv is operably connected to the C-terminus of the CH3 of the first heavy chain indirectly via a peptide linker (e.g., a linker described herein) (see, e.g., § 5.2.8). In some embodiments, the second scFv is operably connected to the C-terminus of the CH3 of the second heavy chain directly via a peptide bond. In some embodiments, the second scFv is operably connected to the C-terminus of the CH3 of the second heavy chain indirectly via a peptide linker (e.g., a linker described herein) (see, e.g., § 5.2.8). In some embodiments, the first scFv is operably connected to the C-terminus of the CH3 of the first heavy chain directly via a peptide bond; and the second scFv is operably connected to the C-terminus of the CH3 of the second heavy chain directly via a peptide bond. In some embodiments, the first scFv is operably connected to the C-terminus of the CH3 of the first heavy chain indirectly via a peptide linker (e.g., a linker described herein) (see, e.g., § 5.2.8); and the second scFv is operably connected to the C-terminus of the CH3 of the second heavy chain indirectly via a peptide linker (e.g., a linker described herein) (see, e.g., § 5.2.8). In some embodiments, the amino acid sequence of the peptide linker that operably connects the first scFv to the C-terminus of the CH3 region of the first heavy chain and the amino acid sequence of the peptide linker that operably connects the second scFv to the C-terminus of the CH3 region of the second heavy chain are at least 85%, 86%, 87%, 88%, 89%, 90%, 91%, 92%, 93%, 94%, 95%, 96%, 97%, 98%, 99%, or 100% identical. In some embodiments, the amino acid sequence of the peptide linker that operably connects the first scFv to the C-terminus of the CH3 region of the first heavy chain and the amino acid sequence of the peptide linker that operably connects the second scFv to the C-terminus of the CH3 region of the second heavy chain are 100% identical.

In some embodiments, the first scFv is operably connected to the C-terminus of the CH3 region of the first heavy chain (either directly or indirectly through a peptide linker) through the VH region of the first scFv. In some embodiments, the first scFv is operably connected to the C-terminus of the CH3 region of the first heavy chain (either directly or indirectly through a peptide linker) through the VL region of the first scFv. In some embodiments, the second scFv is operably connected to the C-terminus of the CH3 region of the second heavy chain (either directly or indirectly through a peptide linker) through the VH region of the second scFv. In some embodiments, the second scFv is operably connected to the C-terminus of the CH3 region of the second heavy chain (either directly or indirectly through a peptide linker) through the VL region of the second scFv. In some embodiments, the first scFv is operably connected to the C-terminus of the CH3 region of the first heavy chain (either directly or indirectly through a peptide linker) through the VH region of the first scFv; and the second scFv is operably connected to the C-terminus of the CH3 region of the second heavy chain (either directly or indirectly through a peptide linker) through the VH region of the second scFv. In some embodiments, the first scFv is operably connected to the C-terminus of the CH3 region of the first heavy chain (either directly or indirectly through a peptide linker) through the VL region of the first scFv; and the second scFv is operably connected to the C-terminus of the CH3 region of the second heavy chain (either directly or indirectly through a peptide linker) through the VL region of the second scFv.

In some embodiments the first hTAA and the second hTAA are the same. In some embodiments the first hTAA and the second hTAA are different. In some embodiments, the first hTAA and the second hTAA are different, but are expressed by the same cancer cell. In some embodiments, the first antigen binding domain of the full-length antibody specifically binds the same hTAA as the second antigen binding domain of the full-length antibody. In some embodiments, the first antigen binding domain of the full-length antibody specifically binds to same epitope as the second antigen binding domain of the full-length antibody. In some embodiments, the first antigen binding domain of the full-length antibody specifically binds the same hTAA but a different epitope as the second antigen binding domain of the full-length antibody. In some embodiments, the first antigen binding domain of the full-length antibody specifically binds the same hTAA but a different and non-overlapping epitope as the second antigen binding domain of the full-length antibody (e.g., the full-length antibody is biparatopic). In some embodiments, the first antigen binding domain of the full-length antibody specifically binds a different hTAA than the second antigen binding domain of the full-length antibody.

In some embodiments, the multispecific protein comprises (a) a first polypeptide comprising a first light chain comprising from N- to C-terminus a VL region and a CL region; (b) a second polypeptide comprising from N- to C-terminus: (i) a first heavy chain comprising from N- to C-terminus a VH region, a CH1 region, a hinge region, a CH2 region, and a CH3 region; (ii) an optional first peptide linker, and (iii) a first scFv comprising from N- to C-terminus (iii(a)) a VL region, a peptide linker, and a VH region, or (iii(b)) a VH region, a peptide linker, and a VL region; (c) third polypeptide comprising from N- to C-terminus: (i) a second heavy chain comprising from N- to C-terminus a VH region, a CH1 region, a hinge region, a CH2 region, and a CH3 region, (ii) an optional second peptide linker; and (iii) a second scFv comprising from N to C-terminus (iii(a)) a VL region, a peptide linker, and a VH region, or (iii(b)) a VH region, a peptide linker, and a VL region; and (d) a fourth polypeptide comprising a second light chain comprising from N- to C-terminus a VL region and a CL region; wherein said VL of said first light chain and said VH of said first heavy chain associate to form a first antigen binding domain that specifically binds a first hTAA; wherein said VH of said second heavy chain and said VL of second light chain associate to form a second antigen binding domain that specifically binds a second hTAA; wherein said first scFv specifically binds a hTCSA (e.g., hCD28, hCD2); and wherein said second scFv specifically binds hCD3.

In some embodiments, the second polypeptide comprises from N- to C-terminus: (i) a first heavy chain comprising from N- to C-terminus a VH region, a CH1 region, a hinge region, a CH2 region, and a CH3 region; (ii) a first peptide linker (e.g., a linker described herein) (see, e.g., § 5.2.8), and (iii) a first scFv comprising from N- to C-terminus (iii(a)) a VL region, a peptide linker, and a VH region, or (iii(b)) a VH region, a peptide linker, and a VL region. In some embodiments, the third polypeptide comprises from N- to C-terminus: (i) a second heavy chain comprising from N- to C-terminus a VH region, a CH1 region, a hinge region, a CH2 region, and a CH3 region, (ii) a second peptide linker (e.g., a linker described herein) (see, e.g., § 5.2.8); and (iii) a second scFv comprising from N- to C-terminus (iii(a)) a VL region, a peptide linker, and a VH region, or (iii(b)) a VH region, a peptide linker, and a VL region. In some embodiments, the second polypeptide comprises from N- to C-terminus: (i) a first heavy chain comprising from N- to C-terminus a VH region, a CH1 region, a hinge region, a CH2 region, and a CH3 region; (ii) a first peptide linker (e.g., a linker described herein) (see, e.g., § 5.2.8), and (iii) a first scFv comprising from N- to C-terminus (iii(a)) a VL region, a peptide linker, and a VH region, or (iii(b)) a VH region, a peptide linker, and a VL region; and the third polypeptide comprises from N- to C-terminus: (i) a second heavy chain comprising from N- to C-terminus a VH region, a CH1 region, a hinge region, a CH2 region, and a CH3 region, (ii) a second peptide linker (e.g., a linker described herein) (see, e.g., § 5.2.8); and (iii) a second scFv comprising from N- to C-terminus (iii(a)) a VL region, a peptide linker, and a VH region, or (iii(b)) a VH region, a peptide linker, and a VL region. In some embodiments, the amino acid sequence of the first peptide linker and the amino acid sequence of the second peptide linker are at least 85%, 86%, 87%, 88%, 89%, 90%, 91%, 92%, 93%, 94%, 95%, 96%, 97%, 98%, 99%, or 100% identical. In some embodiments, the amino acid sequence of the first peptide linker and the amino acid sequence of the second peptide linker are 100% identical.

In some embodiments, the first scFv comprises from N- to C-terminus a VL region, a peptide linker, and a VH region. In some embodiments, the first scFv comprises from N- to C-terminus a VH region, a peptide linker, and a VL region. In some embodiments, the second scFv comprises from N- to C-terminus a VL region, a peptide linker, and a VH region. In some embodiments, the second scFv comprises from N- to C-terminus a VH region, a peptide linker, and a VL region. In some embodiments, the first scFv comprises from N- to C-terminus a VL region, a peptide linker, and a VH region; and the second scFv comprises from N- to C-terminus a VL region, a peptide linker, and a VH region. In some embodiments, the first scFv comprises from N- to C-terminus a VH region, a peptide linker, and a VL region; and the second scFv comprises from N- to C-terminus a VH region, a peptide linker, and a VL region.

In some embodiments the first hTAA and the second hTAA are the same. In some embodiments the first hTAA and the second hTAA are different. In some embodiments, the first hTAA and the second hTAA are different, but are expressed by the same cancer cell. In some embodiments, the first antigen binding domain specifically binds the same hTAA as the second antigen binding domain. In some embodiments, the first antigen binding domain specifically binds to same epitope as the second antigen binding domain. In some embodiments, the first antigen binding domain specifically binds the same hTAA but a different epitope as the second antigen binding domain. In some embodiments, the first antigen binding domain specifically binds the same hTAA but a different and non-overlapping epitope as the second antigen binding domain. In some embodiments, the first antigen binding domain specifically binds a different hTAA than the second antigen binding domain.

Generally, Format BCA405 can also be described as a multispecific protein comprising: (a) a first polypeptide comprising a first light chain comprising from N- to C-terminus a VL region and a CL region; (b) a second polypeptide comprising from N- to C-terminus: (i) a first heavy chain comprising from N- to C-terminus a VH region, a CH1 region, a hinge region, a CH2 region, and a CH3 region; (ii) an optional first peptide linker (e.g., a linker described herein) (see, e.g., § 5.2.8), and (iii) a first scFv comprising from N- to C-terminus (iii(a)) a VL region, a peptide linker, and a VH region, or (iii(b)) a VH region, a peptide linker, and a VL region; (c) third polypeptide comprising from N- to C-terminus: (i) a second heavy chain comprising from N- to C-terminus a VH region, a CH1 region, a hinge region, a CH2 region, and a CH3 region, (ii) an optional second peptide linker (e.g., a linker described herein) (see, e.g., § 5.2.8); and (iii) a second scFv comprising from N- to C-terminus (iii(a)) a VL region, a peptide linker, and a VH region, or (iii(b)) a VH region, a peptide linker, and a VL region; and (d) a fourth polypeptide comprising a second light chain comprising from N- to C-terminus a VL region and a CL region; wherein said VL of said first light chain and said VH of said first heavy chain associate to form a first antigen binding domain that specifically binds a first hTAA; wherein said VH of said second heavy chain and said VL of second light chain associate to form a second antigen binding domain that specifically binds a second hTAA; wherein said first scFv specifically binds a hTCSA (e.g., hCD28, hCD2); wherein said second scFv specifically binds hCD3; and wherein said CH3 region of said first heavy chain comprises one or more amino acid modification (e.g., substitution) relative to the amino acid sequence of a reference CH3 region that does not contain said one or more amino acid modification (e.g., a wild-type CH3 region, e.g., SEQ ID NO: 101), wherein said CH3 region of said second heavy chain comprises one or more amino acid modification (e.g., substitution) relative to the amino acid sequence of a reference CH3 region that does not contain said one or more amino acid modification (e.g., a wild-type CH3 region, e.g., SEQ ID NO: 101); and wherein said one or more amino acid modification in said CH3 region of said first heavy chain of said full-length antibody is different from said one or more amino acid modification in said CH3 region of said second heavy chain of said full-length antibody; wherein said one or more amino acid modification in said CH3 region of said first heavy chain of said full-length antibody and said one or more amino acid modification in said CH3 region of said second heavy chain of said full-length antibody promote heterodimerization of said first and second heavy chain of said full-length antibody; wherein said CH2 region of said first heavy chain comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said CH2 region of said second heavy chain comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said one or more amino acid modification in said CH2 region of said first heavy chain and said one or more amino acid modification in said CH2 region of said second heavy chain reduce or abolish one or more of the following heavy chain effector functions relative to a reference heavy chain that does not contain said one or more amino acid modification (e.g., a heavy chain comprising a wild type CH2 domain, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In some embodiments the first hTAA and the second hTAA are the same. In some embodiments the first hTAA and the second hTAA are different. In some embodiments, the first hTAA and the second hTAA are different, but are expressed by the same cancer cell.

### 5.2.1.2 Format BCA406

In one aspect, provided herein are multispecific proteins set forth in Format BCA406 (see, e.g., FIG. 2). Generally, Format BCA406 provides a multispecific protein comprising (i) a full-length antibody that specifically binds a first hTAA and a second hTAA; (ii) a first scFv operably connected to the N-terminus of either the first heavy chain or the first light chain of the full-length antibody, wherein the first scFv specifically binds hCD3; and a second scFv operably connected to the N-terminus of either the second heavy chain or the second light chain of the full-length antibody, wherein the second scFv specifically binds a hTCSA (e.g., hCD28, hCD2).

In some embodiments, the multispecific protein comprises (a) a full-length antibody comprising: (i) a first light chain comprising from N- to C-terminus a VL region and a CL region; (ii) a first heavy chain comprising from N- to C-terminus a VH region, a CH1 region, a hinge region, a CH2 region, and a CH3 region; (iii) a second heavy chain comprising from N- to C-terminus a VH region, a CH1 region, a hinge region, a CH2 region, and a sCH3 region; (iv) a second light chain comprising from N- to C-terminus a VL region and a VH region; wherein the first light chain and first heavy chain associate to form a first antigen binding domain; wherein the second light chain and the second heavy chain associate to form a second antigen binding domain; and wherein the first heavy chain and second heavy chain associate to form a dimer; (b) a first scFv operably connected to the N-terminus of said first heavy chain of said full-length antibody, wherein said first scFv comprises from N- to C-terminus (i) a VH region, a peptide linker, and a VL region, or (ii) a VL region, a peptide linker, and a VH region; and (c) a second scFv operably connected to the N-terminus of said second heavy chain of said full-length antibody, wherein said second scFv comprises from N- to C-terminus (i) a VH region, a peptide linker, and a VL region, or (ii) a VL region, a peptide linker, and a VH region; wherein said first antigen binding domain of said full-length antibody specifically binds to a first hTAA; wherein said second antigen binding domain of said full-length antibody specifically binds to a second hTAA; wherein said first scFv specifically binds to a hTCSA (e.g., hCD28, hCD2); and wherein said second scFv specifically binds to hCD3.

In some embodiments, the first scFv is operably connected to the N-terminus of the first heavy chain of the full-length antibody directly via a peptide bond. In some embodiments, the first scFv is operably connected to the N-terminus of the first heavy chain of the full-length antibody indirectly via a peptide linker (e.g., a linker described herein) (see, e.g., § 5.2.8). In some embodiments, the second scFv is operably connected to the N-terminus of the second heavy chain of the full-length antibody directly via a peptide bond. In some embodiments, the second scFv is operably connected to the N-terminus of the second heavy chain of the full-length antibody indirectly via a peptide linker (e.g., a linker described herein) (see, e.g., § 5.2.8). In some embodiments, the first scFv is operably connected to the N-terminus of the first heavy chain of the full-length antibody directly via a peptide bond; and the second scFv is operably connected to the N-terminus of the second heavy chain of the full-length antibody directly via a peptide bond. In some embodiments, the first scFv is operably connected to the N-terminus of the first heavy chain of the full-length antibody indirectly via a peptide linker (e.g., a linker described herein) (see, e.g., § 5.2.8); and the second scFv is operably connected to the N-terminus of the second heavy chain of the full-length antibody indirectly via a peptide linker (e.g., a linker described herein) (see, e.g., § 5.2.8). In some embodiments, the amino acid sequence of the peptide linker that operably connects the first scFv to the N-terminus of the first heavy chain of the full-length antibody and the amino acid sequence of the peptide linker that operably connects the second scFv to the N-terminus of the second heavy chain of the full-length antibody are at least 85%, 86%, 87%, 88%, 89%, 90%, 91%, 92%, 93%, 94%, 95%, 96%, 97%, 98%, 99%, or 100% identical. In some embodiments, the amino acid sequence of the peptide linker that operably connects the first scFv to the N-terminus of the first heavy chain of the full-length antibody and the amino acid sequence of the peptide linker that operably connects the second scFv to the N-terminus of the second heavy chain of the full-length antibody chain are 100% identical.

In some embodiments, the first scFv is operably connected to the N-terminus of the first heavy chain of the full-length antibody (either directly or indirectly through a peptide linker) through the VH region of the first scFv. In some embodiments, the first scFv is operably connected to the N-terminus of the first heavy chain of the full-length antibody (either directly or indirectly through a peptide linker) through the VL region of the first scFv. In some embodiments, the second scFv is operably connected to the N-terminus of the second heavy chain of the full-length antibody (either directly or indirectly through a peptide linker) through the VH region of the second scFv. In some embodiments, the second scFv is operably connected to the N-terminus of the second heavy chain of the full-length antibody (either directly or indirectly through a peptide linker) through the VL region of the second scFv. In some embodiments, the first scFv is operably connected to the N-terminus of the first heavy chain of the full-length antibody (either directly or indirectly through a peptide linker) through the VH region of the first scFv; and the second scFv is operably connected to the N-terminus of the second heavy chain of the full-length antibody (either directly or indirectly through a peptide linker) through the VH region of the second scFv. In some embodiments, the first scFv is operably connected to the N-terminus of the first heavy chain of the full-length antibody (either directly or indirectly through a peptide linker) through the VL region of the first scFv; and the second scFv is operably connected to the N-terminus of the second heavy chain of the full-length antibody (either directly or indirectly through a peptide linker) through the VL region of the second scFv.

In some embodiments the first hTAA and the second hTAA are the same. In some embodiments the first hTAA and the second hTAA are different. In some embodiments, the first hTAA and the second hTAA are different, but are expressed by the same cancer cell. In some embodiments, the first antigen binding domain of the full-length antibody specifically binds the same hTAA as the second antigen binding domain of the full-length antibody. In some embodiments, the first antigen binding domain of the full-length antibody specifically binds to same epitope as the second antigen binding domain of the full-length antibody. In some embodiments, the first antigen binding domain of the full-length antibody specifically binds the same hTAA but a different epitope as the second antigen binding domain of the full-length antibody. In some embodiments, the first antigen binding domain of the full-length antibody specifically binds the same hTAA but a different and non-overlapping epitope as the second antigen binding domain of the full-length antibody (e.g., the full-length antibody is biparatopic). In some embodiments, the first antigen binding domain of the full-length antibody specifically binds a different hTAA than the second antigen binding domain of the full-length antibody.

In some embodiments, the multispecific protein comprises (a) a first polypeptide comprising a first light chain comprising from N- to C-terminus a VL region and a CL region; (b) a second polypeptide comprising from N- to C-terminus: (i) a first scFv comprising from N- to C-terminus (i(a)) a VL region, a peptide linker, and a VH region, or (ii(b)) a VH region, a peptide linker, and a VL region; (ii) an optional first peptide linker, and (iii) a first heavy chain comprising from N- to C-terminus a VH region, a CH1 region, a hinge region, a CH2 region, and a CH3 region; (c) a third polypeptide comprising from N- to C-terminus: (i) a second scFv comprising from N- to C-terminus (i(a)) a VL region, a peptide linker, and a VH region, or (ii(b)) a VH region, a peptide linker, and a VL region; (ii) an optional second peptide linker, and (iii) a second heavy chain comprising from N- to C-terminus a VH region, a CH1 region, a hinge region, a CH2 region, and a CH3 region; and (d) a fourth polypeptide comprising a second light chain comprising from N- to C-terminus a VL region and a CL region; wherein said VL region of said first light chain and said VH region of said first Ig heavy chain associate to form a first antigen binding domain that specifically binds a first hTAA; wherein said VH region of said second heavy chain and said VL region of second Ig light chain associate to form a second antigen binding domain that specifically binds a second hTAA; wherein said first scFv specifically binds a hTCSA (e.g., hCD28, hCD2); and wherein said second scFv specifically binds hCD3.

In some embodiments, the second polypeptide comprises from N- to C-terminus: (i) a first scFv comprising from N- to C-terminus (i(a)) a VL region, a peptide linker, and a VH region, or (ii(b)) a VH region, a peptide linker, and a VL region; (ii) a first peptide linker (e.g., a linker described herein) (see, e.g., § 5.2.8), and (iii) a first heavy chain comprising from N- to C-terminus a VH region, a CH1 region, a hinge region, a CH2 region, and a CH3 region. In some embodiments, the third polypeptide comprises from N- to C-terminus: (i) a second scFv comprising from N- to C-terminus (i(a)) a VL region, a peptide linker, and a VH region, or (ii(b)) a VH region, a peptide linker, and a VL region; (ii) a second peptide linker (e.g., a linker described herein) (see, e.g., § 5.2.8), and (iii) a second heavy chain comprising from N- to C-terminus a VH region, a CH1 region, a hinge region, a CH2 region, and a CH3 region. In some embodiments, the second polypeptide comprises from N- to C-terminus: (i) a first scFv comprising from N- to C-terminus (i(a)) a VL region, a peptide linker, and a VH region, or (ii(b)) a VH region, a peptide linker, and a VL region; (ii) a first peptide linker (e.g., a linker described herein) (see, e.g., § 5.2.8), and (iii) a first heavy chain comprising from N- to C-terminus a VH region, a CH1 region, a hinge region, a CH2 region, and a CH3 region; and the third polypeptide comprises from N- to C-terminus: (i) a second scFv comprising from N- to C-terminus (i(a)) a VL region, a peptide linker, and a VH region, or (ii(b)) a VH region, a peptide linker, and a VL region; (ii) a second peptide linker (e.g., a linker described herein) (see, e.g., § 5.2.8), and (iii) a second heavy chain comprising from N- to C-terminus a VH region, a CH1 region, a hinge region, a CH2 region, and a CH3 region. In some embodiments, the amino acid sequence of the first peptide linker and the amino acid sequence of the second peptide linker are at least 85%, 86%, 87%, 88%, 89%, 90%, 91%, 92%, 93%, 94%, 95%, 96%, 97%, 98%, 99%, or 100% identical. In some embodiments, the amino acid sequence of the first peptide linker and the amino acid sequence of the second peptide linker are 100% identical.

In some embodiments, the first scFv comprises from N- to C-terminus a VL region, a peptide linker, and a VH region. In some embodiments, the first scFv comprises from N- to C-terminus a VH region, a peptide linker, and a VL region. In some embodiments, the second scFv comprises from N- to C-terminus a VL region, a peptide linker, and a VH region. In some embodiments, the second scFv comprises from N- to C-terminus a VH region, a peptide linker, and a VL region. In some embodiments, the first scFv comprises from N- to C-terminus a VL region, a peptide linker, and a VH region; and the second scFv comprises from N- to C-terminus a VL region, a peptide linker, and a VH region. In some embodiments, the first scFv comprises from N- to C-terminus a VH region, a peptide linker, and a VL region; and the second scFv comprises from N- to C-terminus a VH region, a peptide linker, and a VL region.

In some embodiments the first hTAA and the second hTAA are the same. In some embodiments the first hTAA and the second hTAA are different. In some embodiments, the first hTAA and the second hTAA are different, but are expressed by the same cancer cell. In some embodiments, the first antigen binding domain specifically binds the same hTAA as the second antigen binding domain. In some embodiments, the first antigen binding domain specifically binds to same epitope as the second antigen binding domain. In some embodiments, the first antigen binding domain specifically binds the same hTAA but a different epitope as the second antigen binding domain. In some embodiments, the first antigen binding domain specifically binds the same hTAA but a different and non-overlapping epitope as the second antigen binding domain. In some embodiments, the first antigen binding domain specifically binds a different hTAA than the second antigen binding domain.

Generally, the BCA406 Format can also be described as a multispecific protein comprising: (a) a first polypeptide comprising a first light chain comprising from N- to C-terminus a VL region and a CL region; (b) a second polypeptide comprising from N- to C-terminus: (i) a first scFv comprising from N- to C-terminus (i(a)) a VL region, a peptide linker, and a VH region, or (ii(b)) a VH region, a peptide linker, and a VL region; (ii) an optional first peptide linker (e.g., a linker described herein) (see, e.g., § 5.2.8), and (iii) a first heavy chain comprising from N- to C-terminus a VH region, a CH1 region, a hinge region, a CH2 region, and a CH3 region; (c) a third polypeptide comprising from N- to C-terminus: (i) a second scFv comprising from N- to C-terminus (i(a)) a VL region, a peptide linker, and a VH region, or (ii(b)) a VH region, a peptide linker, and a VL region; (ii) an optional second peptide linker (e.g., a linker described herein) (see, e.g., § 5.2.8), and (iii) a second heavy chain comprising from N- to C-terminus a VH region, a CH1 region, a hinge region, a CH2 region, and a CH3 region; and (d) a fourth polypeptide comprising a second light chain comprising from N- to C-terminus a VL region and a CL region; wherein said VL region of said first light chain and said VH region of said first Ig heavy chain associate to form a first antigen binding domain that specifically binds a first hTAA; wherein said VH region of said second heavy chain and said VL region of second Ig light chain associate to form a second antigen binding domain that specifically binds a second hTAA; wherein said first scFv specifically binds a hTCSA (e.g., hCD28, hCD2); wherein said second scFv specifically binds hCD3; and wherein said CH3 region of said first heavy chain comprises one or more amino acid modification (e.g., substitution) relative to the amino acid sequence of a reference CH3 region that does not contain said one or more amino acid modification (e.g., a wild-type CH3 region, e.g., SEQ ID NO: 101), wherein said CH3 region of said second heavy chain comprises one or more amino acid modification (e.g., substitution) relative to the amino acid sequence of a reference CH3 region that does not contain said one or more amino acid modification (e.g., a wild-type CH3 region, e.g., SEQ ID NO: 101); and wherein said one or more amino acid modification in said CH3 region of said first heavy chain of said full-length antibody is different from said one or more amino acid modification in said CH3 region of said second heavy chain of said full-length antibody; wherein said one or more amino acid modification in said CH3 region of said first heavy chain of said full-length antibody and said one or more amino acid modification in said CH3 region of said second heavy chain of said full-length antibody promote heterodimerization of said first and second heavy chain of said full-length antibody; wherein said CH2 region of said first heavy chain comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said CH2 region of said second heavy chain comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said one or more amino acid modification in said CH2 region of said first heavy chain and said one or more amino acid modification in said CH2 region of said second heavy chain reduce or abolish one or more of the following heavy chain effector functions relative to a reference heavy chain that does not contain said one or more amino acid modification (e.g., a heavy chain comprising a wild type CH2 domain, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In some embodiments the first hTAA and the second hTAA are the same. In some embodiments the first hTAA and the second hTAA are different. In some embodiments, the first hTAA and the second hTAA are different, but are expressed by the same cancer cell.

### 5.2.1.3 Format BCA424

In one aspect, provided herein are multispecific proteins set forth in Format BCA424 (see, e.g., FIG. 3). Generally, Format BCA424 provides a multispecific protein comprising a first Fab operably connected (e.g., via a peptide linker) to a first scFv operably connected (e.g., via a peptide linker) to a first Fc region; and a second Fab operably connected (e.g., via a peptide linker) to a second scFv operably connected (e.g., via a peptide linker) to a second Fc region; wherein the first Fab specifically binds a first hTAA, the second Fab specifically binds a second hTAA, the first scFv specifically binds a hTCSA (e.g., hCD28, hCD2), and the second scFv specifically binds hCD3.

In some embodiments, the multispecific protein comprises (a) first Fab comprising (i) a first Fab heavy chain comprising from N- to C-terminus a first VH region and a first CH1 region and (ii) a first light chain comprising from N- to C-terminus a first VL region and a first CL region; (b) a first scFv operably connected to the C-terminus of said first CH1 region of said first Fab (e.g., via a peptide linker, e.g., any one of SEQ ID NOS: 217-236 or 318-319, e.g., SEQ ID NO: 319), wherein the first scFv comprises from N- to C-terminus (i) a VH region, a peptide linker, and a VL region; or (ii) a VL region, a peptide linker, and a VH region; (c) a first Fc region operably connected to the C-terminus of said first scFv, wherein said first Fc region comprises from N- to C-terminus a CH2 region and a CH3 region; and (d) second Fab comprising (i) a second Fab heavy chain comprising from N- to C-terminus a second VH region and a second CH1 region and (ii) a second light chain comprising from N- to C-terminus a second VL region and a first CL region; (b) a second scFv operably connected to the C-terminus of said second CH1 region of said second Fab (e.g., via a peptide linker, e.g., any one of SEQ ID NOS: 217-236 or 318-319, e.g., SEQ ID NO: 319), wherein said second scFv comprises from N- to C-terminus (i) a VH region, a peptide linker, and a VL region; or (ii) a VL region, a peptide linker, and a VH region; (c) a second Fc region operably connected to the C-terminus of said second scFv, wherein said second Fc region comprises from N- to C-terminus a CH2 region and a CH3 region; and wherein said first Fab specifically binds to a first hTAA; wherein said second Fab specifically binds to a second hTAA; wherein said first scFv specifically binds to a hTCSA (e.g., hCD28, hCD2); wherein said second scFv specifically binds to hCD3.

In some embodiments, the first scFv is operably connected to the C-terminus of said first CH1 region of said first Fab directly via a peptide bond. In some embodiments, the first scFv is operably connected to the C-terminus of said first CH1 region of said first Fab indirectly via a peptide linker (e.g., a linker described herein) (see, e.g., § 5.2.8, e.g., any one of SEQ ID NOS: 217-236 or 318-319, e.g., SEQ ID NO: 319). In some embodiments, the second scFv is operably connected to the C-terminus of said second CH1 region of said second Fab directly via a peptide bond. In some embodiments, the second scFv is operably connected to the C-terminus of said second CH1 region of said second Fab indirectly via a peptide linker (e.g., a linker described herein) (see, e.g., § 5.2.8, e.g., any one of SEQ ID NOS: 217-236 or 318-319, e.g., SEQ ID NO: 319). In some embodiments, the first scFv is operably connected to the C-terminus of said first CH1 region of said first Fab directly via a peptide bond; and the second scFv is operably connected to the C-terminus of said second CH1 region of said second Fab directly via a peptide bond. In some embodiments, the first scFv is operably connected to the C-terminus of said first CH1 region of said first Fab indirectly via a peptide linker (e.g., a linker described herein) (see, e.g., § 5.2.8, e.g., any one of SEQ ID NOS: 217-236 or 318-319, e.g., SEQ ID NO: 319); and the second scFv is operably connected to the C-terminus of said second CH1 region of said second Fab indirectly via a peptide linker (e.g., a linker described herein) (see, e.g., § 5.2.8, e.g., any one of SEQ ID NOS: 217-236 or 318-319, e.g., SEQ ID NO: 319). In some embodiments, the amino acid sequence of the peptide linker that operably connects the first scFv to the C-terminus of said first CH1 region of said first Fab and the amino acid sequence of the peptide linker that operably connects the second scFv to the C-terminus of said second CH1 region of said second Fab are at least 85%, 86%, 87%, 88%, 89%, 90%, 91%, 92%, 93%, 94%, 95%, 96%, 97%, 98%, 99%, or 100% identical. In some embodiments, the amino acid sequence of the peptide linker that operably connects the first scFv to the C-terminus of said first CH1 region of said first Fab and the amino acid sequence of the peptide linker that operably connects the second scFv to the C-terminus of said second CH1 region of said second Fab are 100% identical.

In some embodiments, the first Fc region is operably connected to the C-terminus of the first scFv directly via a peptide bond. In some embodiments, the first Fc region is operably connected to the C-terminus of the first scFv indirectly via a peptide linker (e.g., a linker described herein) (see, e.g., § 5.2.8). In some embodiments, the second Fc region is operably connected to the C-terminus of the second scFv directly via a peptide bond. In some embodiments, the second Fc region is operably connected to the C-terminus of the second scFv indirectly via a peptide linker (e.g., a linker described herein) (see, e.g., § 5.2.8). In some embodiments, the first Fc region is operably connected to the C-terminus of the first scFv directly via a peptide bond; and the second Fc region is operably connected to the C-terminus of the second scFv directly via a peptide bond. In some embodiments, the first Fc region is operably connected to the C-terminus of the first scFv indirectly via a peptide linker (e.g., a linker described herein) (see, e.g., § 5.2.8); and the second Fc region is operably connected to the C-terminus of the second scFv indirectly via a peptide linker (e.g., a linker described herein) (see, e.g., § 5.2.8). In some embodiments, the amino acid sequence of the peptide linker that operably connects the first Fc region is operably connected to the C-terminus of the first scFv and the amino acid sequence of the peptide linker that operably connects the second Fc region is operably connected to the C-terminus of the second scFv are at least 85%, 86%, 87%, 88%, 89%, 90%, 91%, 92%, 93%, 94%, 95%, 96%, 97%, 98%, 99%, or 100% identical. In some embodiments, the amino acid sequence of the peptide linker that operably connects the first Fc region is operably connected to the C-terminus of the first scFv and the amino acid sequence of the peptide linker that operably connects the first Fc region is operably connected to the C-terminus of the first scFv are 100% identical.

In some embodiments, the first Fc region comprises from N- to C-terminus a hinge region, a CH2 region and a CH3 region. In some embodiments, the second Fc region comprises from N- to C-terminus a hinge region, a CH2 region and a CH3 region. In some embodiments, the first Fc region comprises from N- to C-terminus a hinge region, a CH2 region and a CH3 region; and the second Fc region comprises from N- to C-terminus a hinge region, a CH2 region and a CH3 region. In some embodiments, the first Fc region comprises from N- to C-terminus a partial hinge region, a CH2 region and a CH3 region. In some embodiments, the second Fc region comprises from N- to C-terminus a partial hinge region, a CH2 region and a CH3 region. In some embodiments, the first Fc region comprises from N- to C-terminus a partial hinge region, a CH2 region and a CH3 region; and the second Fc region comprises from N- to C-terminus a partial hinge region, a CH2 region and a CH3 region.

In some embodiments, the first scFv operably connected to the C-terminus of said first CH1 region of said first Fab (either directly or indirectly through a peptide linker) through the VH region of the first scFv. In some embodiments, the first scFv operably connected to the C-terminus of said first CH1 region of said first Fab (either directly or indirectly through a peptide linker) through the VL region of the first scFv. In some embodiments, the second scFv operably connected to the C-terminus of said second CH1 region of said second Fab (either directly or indirectly through a peptide linker) through the VH region of the second scFv. In some embodiments, the second scFv operably connected to the C-terminus of said second CH1 region of said second Fab (either directly or indirectly through a peptide linker) through the VL region of the second scFv. In some embodiments, the first scFv operably connected to the C-terminus of said first CH1 region of said first Fab (either directly or indirectly through a peptide linker) through the VH region of the first scFv; and the second scFv operably connected to the C-terminus of said second CH1 region of said second Fab (either directly or indirectly through a peptide linker) through the VH region of the second scFv. In some embodiments, the first scFv operably connected to the C-terminus of said first CH1 region of said first Fab (either directly or indirectly through a peptide linker) through the VL region of the first scFv; and the second scFv operably connected to the C-terminus of second first CH1 region of said second Fab (either directly or indirectly through a peptide linker) through the VL region of the second scFv.

In some embodiments the first hTAA and the second hTAA are the same. In some embodiments the first hTAA and the second hTAA are different. In some embodiments, the first hTAA and the second hTAA are different, but are expressed by the same cancer cell. In some embodiments, the first Fab specifically binds the same hTAA as the second Fab. In some embodiments, the first Fab specifically binds to same epitope as the second Fab. In some embodiments, the first Fab specifically binds the same hTAA but a different epitope as the second Fab. In some embodiments, the first Fab specifically binds the same hTAA but a different and non-overlapping epitope as the second Fab. In some embodiments, the first Fab specifically binds a different hTAA than the second Fab.

In some embodiments, the multispecific protein comprises (a) a first polypeptide comprising from N- to C-terminus: (i) a first Fab heavy chain comprising from N- to C-terminus a VH region and a VL region; operably connected to (ii) a first scFv that comprises from N- to C-terminus (ii(a)) a VL region, a peptide linker, and a VH region, or (ii(b)) a VH region, a peptide linker, and a VL region; operably connected to a first Fc region that comprises from N- to C-terminus a CH2 region and a CH3 region; (b) a second polypeptide comprising a first light chain comprising from N- to C-terminus a VL region and a CL region; (c) a third polypeptide comprising from N- to C-terminus: (i) a second Fab heavy chain comprising from N- to C-terminus a VH region and a VL region; operably connected to (ii) a second scFv that comprises from N- to C-terminus (ii(a)) a VL region, a peptide linker, and a VH region, or (ii(b)) a VH region, a peptide linker, and a VL region; operably connected to a second Fc region that comprises from N- to C-terminus a CH2 region and a CH3 region; (d) a fourth polypeptide comprising a second light chain comprising from N- to C-terminus a VL region and a CL region; wherein said first Fab heavy chain and said first light chain associate to form a first antigen binding domain that specifically binds a first hTAA; wherein said second Fab heavy chain and said second light chain associate to form a second antigen binding domain that specifically binds a second hTAA; wherein said first scFv specifically binds a hTCSA (e.g., hCD28, hCD2); and wherein said second scFv specifically binds hCD3.

In some embodiments, the first polypeptide comprises from N- to C-terminus: (i) a first Fab heavy chain comprising from N- to C-terminus a VH region and a VL region; operably connected via a first peptide linker (e.g., a linker described herein) (see, e.g., § 5.2.8) to (ii) a first scFv that comprises from N- to C-terminus (ii(a)) a VL region, a peptide linker, and a VH region, or (ii(b)) a VH region, a peptide linker, and a VL region; operably connected to a first Fc region that comprises from N- to C-terminus a CH2 region and a CH3 region. In some embodiments, the third polypeptide comprises from N- to C-terminus: (i) a second Fab heavy chain comprising from N- to C-terminus a VH region and a VL region; operably connected via a second peptide linker (e.g., a linker described herein) (see, e.g., § 5.2.8, e.g., any one of SEQ ID NOS: 217-236 or 318-319, e.g., SEQ ID NO: 319) to (ii) a second scFv that comprises from N- to C-terminus (ii(a)) a VL region, a peptide linker, and a VH region, or (ii(b)) a VH region, a peptide linker, and a VL region; operably connected to a second Fc region that comprises from N- to C-terminus a CH2 region and a CH3 region. In some embodiments, the first polypeptide comprises from N- to C-terminus: (i) a first Fab heavy chain comprising from N- to C-terminus a VH region and a VL region; operably connected via a first peptide linker (e.g., a linker described herein) (see, e.g., § 5.2.8) to (ii) a first scFv that comprises from N- to C-terminus (ii(a)) a VL region, a peptide linker, and a VH region, or (ii(b)) a VH region, a peptide linker, and a VL region; operably connected to a first Fc region that comprises from N- to C-terminus a CH2 region and a CH3 region; and the third polypeptide comprises from N- to C-terminus: (i) a second Fab heavy chain comprising from N- to C-terminus a VH region and a VL region; operably connected via a second peptide linker (e.g., a linker described herein) (see, e.g., § 5.2.8, e.g., any one of SEQ ID NOS: 217-236 or 318-319, e.g., SEQ ID NO: 319) to (ii) a second scFv that comprises from N- to C-terminus (ii(a)) a VL region, a peptide linker, and a VH region, or (ii(b)) a VH region, a peptide linker, and a VL region; operably connected to a second Fc region that comprises from N- to C-terminus a CH2 region and a CH3 region. In some embodiments, the amino acid sequence of the first peptide linker and the amino acid sequence of the second peptide linker are at least 85%, 86%, 87%, 88%, 89%, 90%, 91%, 92%, 93%, 94%, 95%, 96%, 97%, 98%, 99%, or 100% identical. In some embodiments, the amino acid sequence of the first peptide linker and the amino acid sequence of the second peptide linker are 100% identical.

In some embodiments, the first polypeptide comprises from N- to C-terminus: (i) a first Fab heavy chain comprising from N- to C-terminus a VH region and a VL region; operably connected to (ii) a first scFv that comprises from N- to C-terminus (ii(a)) a VL region, a peptide linker, and a VH region, or (ii(b)) a VH region, a peptide linker, and a VL region; operably connected via a first peptide linker (e.g., a linker described herein) (see, e.g., § 5.2.8) to a first Fc region that comprises from N- to C-terminus a CH2 region and a CH3 region. In some embodiments, the third polypeptide comprises from N- to C-terminus: (i) a second Fab heavy chain comprising from N- to C-terminus a VH region and a VL region; operably connected to (ii) a second scFv that comprises from N- to C-terminus (ii(a)) a VL region, a peptide linker, and a VH region, or (ii(b)) a VH region, a peptide linker, and a VL region; operably connected via a second peptide linker (e.g., a linker described herein) (see, e.g., § 5.2.8) to a second Fc region that comprises from N- to C-terminus a CH2 region and a CH3 region. In some embodiments, the first polypeptide comprises from N- to C-terminus: (i) a first Fab heavy chain comprising from N- to C-terminus a VH region and a VL region; operably connected to (ii) a first scFv that comprises from N- to C-terminus (ii(a)) a VL region, a peptide linker, and a VH region, or (ii(b)) a VH region, a peptide linker, and a VL region; operably connected via a first peptide linker (e.g., a linker described herein) (see, e.g., § 5.2.8) to a first Fc region that comprises from N- to C-terminus a CH2 region and a CH3 region; and the third polypeptide comprises from N- to C-terminus: (i) a second Fab heavy chain comprising from N- to C-terminus a VH region and a VL region; operably connected to (ii) a second scFv that comprises from N- to C-terminus (ii(a)) a VL region, a peptide linker, and a VH region, or (ii(b)) a VH region, a peptide linker, and a VL region; operably connected via a second peptide linker (e.g., a linker described herein) (see, e.g., § 5.2.8) to a second Fc region that comprises from N- to C-terminus a CH2 region and a CH3 region. In some embodiments, the amino acid sequence of the first peptide linker and the amino acid sequence of the second peptide linker are at least 85%, 86%, 87%, 88%, 89%, 90%, 91%, 92%, 93%, 94%, 95%, 96%, 97%, 98%, 99%, or 100% identical. In some embodiments, the amino acid sequence of the first peptide linker and the amino acid sequence of the second peptide linker are 100% identical.

In some embodiments, the first polypeptide comprises from N- to C-terminus: (i) a first Fab heavy chain comprising from N- to C-terminus a VH region and a VL region; operably connected via a first peptide linker (e.g., a linker described herein) (see, e.g., § 5.2.8, e.g., any one of SEQ ID NOS: 217-236 or 318-319, e.g., SEQ ID NO: 319) to (ii) a first scFv that comprises from N- to C-terminus (ii(a)) a VL region, a peptide linker, and a VH region, or (ii(b)) a VH region, a peptide linker, and a VL region; operably connected via a third peptide linker (e.g., a linker described herein) (see, e.g., § 5.2.8) to a first Fc region that comprises from N- to C-terminus a CH2 region and a CH3 region. In some embodiments, the third polypeptide comprises from N- to C-terminus: (i) a second Fab heavy chain comprising from N- to C-terminus a VH region and a VL region; operably connected via a second peptide linker (e.g., a linker described herein) (see, e.g., § 5.2.8, e.g., any one of SEQ ID NOS: 217-236 or 318-319, e.g., SEQ ID NO: 319) to (ii) a second scFv that comprises from N- to C-terminus (ii(a)) a VL region, a peptide linker, and a VH region, or (ii(b)) a VH region, a peptide linker, and a VL region; operably connected via a fourth peptide linker (e.g., a linker described herein) (see, e.g., § 5.2.8) to a second Fc region that comprises from N- to C-terminus a CH2 region and a CH3 region. In some embodiments, the first polypeptide comprises from N- to C-terminus: (i) a first Fab heavy chain comprising from N- to C-terminus a VH region and a VL region; operably connected via a first peptide linker (e.g., a linker described herein) (see, e.g., § 5.2.8, e.g., any one of SEQ ID NOS: 217-236 or 318-319, e.g., SEQ ID NO: 319) to (ii) a first scFv that comprises from N- to C-terminus (ii(a)) a VL region, a peptide linker, and a VH region, or (ii(b)) a VH region, a peptide linker, and a VL region; operably connected via a third peptide linker (e.g., a linker described herein) (see, e.g., § 5.2.8) to a first Fc region that comprises from N- to C-terminus a CH2 region and a CH3 region; and the third polypeptide comprises from N- to C-terminus: (i) a second Fab heavy chain comprising from N- to C-terminus a VH region and a VL region; operably connected via a second peptide linker (e.g., a linker described herein) (see, e.g., § 5.2.8, e.g., any one of SEQ ID NOS: 217-236 or 318-319, e.g., SEQ ID NO: 319) to (ii) a second scFv that comprises from N- to C-terminus (ii(a)) a VL region, a peptide linker, and a VH region, or (ii(b)) a VH region, a peptide linker, and a VL region; operably connected via a fourth peptide linker (e.g., a linker described herein) (see, e.g., § 5.2.8) to a second Fc region that comprises from N- to C-terminus a CH2 region and a CH3 region. In some embodiments, the amino acid sequence of the third peptide linker and the amino acid sequence of the fourth peptide linker are at least 85%, 86%, 87%, 88%, 89%, 90%, 91%, 92%, 93%, 94%, 95%, 96%, 97%, 98%, 99%, or 100% identical. In some embodiments, the amino acid sequence of the third peptide linker and the amino acid sequence of the fourth peptide linker are 100% identical.

In some embodiments, the first Fc region comprises from N- to C-terminus a hinge region, a CH2 region and a CH3 region. In some embodiments, the second Fc region comprises from N- to C-terminus a hinge region, a CH2 region and a CH3 region. In some embodiments, the first Fc region comprises from N- to C-terminus a hinge region, a CH2 region and a CH3 region; and the second Fc region comprises from N- to C-terminus a hinge region, a CH2 region and a CH3 region. In some embodiments, the first Fc region comprises from N- to C-terminus a partial hinge region, a CH2 region and a CH3 region. In some embodiments, the second Fc region comprises from N- to C-terminus a partial hinge region, a CH2 region and a CH3 region. In some embodiments, the first Fc region comprises from N- to C-terminus a partial hinge region, a CH2 region and a CH3 region; and the second Fc region comprises from N- to C-terminus a partial hinge region, a CH2 region and a CH3 region.

In some embodiments, the first scFv comprises from N- to C-terminus a VL region, a peptide linker, and a VH region. In some embodiments, the first scFv comprises from N- to C-terminus a VH region, a peptide linker, and a VL region. In some embodiments, the second scFv comprises from N- to C-terminus a VL region, a peptide linker, and a VH region. In some embodiments, the second scFv comprises from N- to C-terminus a VH region, a peptide linker, and a VL region. In some embodiments, the first scFv comprises from N- to C-terminus a VL region, a peptide linker, and a VH region; and the second scFv comprises from N- to C-terminus a VL region, a peptide linker, and a VH region. In some embodiments, the first scFv comprises from N- to C-terminus a VH region, a peptide linker, and a VL region; and the second scFv comprises from N- to C-terminus a VH region, a peptide linker, and a VL region.

In some embodiments the first hTAA and the second hTAA are the same. In some embodiments the first hTAA and the second hTAA are different. In some embodiments, the first hTAA and the second hTAA are different, but are expressed by the same cancer cell. In some embodiments, the first antigen binding domain specifically binds the same hTAA as the second antigen binding domain. In some embodiments, the first antigen binding domain specifically binds to same epitope as the second antigen binding domain. In some embodiments, the first antigen binding domain specifically binds the same hTAA but a different epitope as the second antigen binding domain. In some embodiments, the first antigen binding domain specifically binds the same hTAA but a different and non-overlapping epitope as the second antigen binding domain. In some embodiments, the first antigen binding domain specifically binds a different hTAA than the second antigen binding domain.

Generally, the BCA424 Format can also be described as multispecific protein comprising: (a) a first polypeptide comprising from N- to C-terminus: (i) a first Fab heavy chain comprising from N- to C-terminus a VH region and a VL region; operably connected (via an optional first peptide linker (e.g., a linker described herein) (see, e.g., § 5.2.8, e.g., any one of SEQ ID NOS: 217-236 or 318-319, e.g., SEQ ID NO: 319)) to (ii) a first scFv that comprises from N- to C-terminus (ii(a)) a VL region, a peptide linker, and a VH region, or (ii(b)) a VH region, a peptide linker, and a VL region; operably connected to a first Fc region that comprises from N- to C-terminus a CH2 region and a CH3 region; (b) a second polypeptide comprising a first light chain comprising from N- to C-terminus a VL region and a CL region; (c) a third polypeptide comprising from N- to C-terminus: (i) a second Fab heavy chain comprising from N- to C-terminus a VH region and a VL region; operably connected (via an optional second peptide linker (e.g., a linker described herein) (see, e.g., § 5.2.8, e.g., any one of SEQ ID NOS: 217-236 or 318-319, e.g., SEQ ID NO: 319)) to (ii) a second scFv that comprises from N- to C-terminus (ii(a)) a VL region, a peptide linker, and a VH region, or (ii(b)) a VH region, a peptide linker, and a VL region; operably connected to a second Fc region that comprises from N- to C-terminus a CH2 region and a CH3 region; (d) a fourth polypeptide comprising a second light chain comprising from N- to C-terminus a VL region and a CL region; wherein said first Fab heavy chain and said first light chain associate to form a first antigen binding domain that specifically binds a first hTAA; wherein said second Fab heavy chain and said second light chain associate to form a second antigen binding domain that specifically binds a second hTAA; wherein said first scFv specifically binds a hTCSA (e.g., hCD28, hCD2); wherein said second scFv specifically binds hCD3; wherein said CH3 region of said first Fc region comprises one or more amino acid modification (e.g., substitution) relative to the amino acid sequence of a reference CH3 region that does not contain said one or more amino acid modification (e.g., a wild-type CH3 region, e.g., SEQ ID NO: 101), wherein said CH3 region of said second Fc region comprises one or more amino acid modification (e.g., substitution) relative to the amino acid sequence of a reference CH3 region that does not contain said one or more amino acid modification (e.g., a wild-type CH3 region, e.g., SEQ ID NO: 101); and wherein said one or more amino acid modification in said CH3 region of said first Fc region is different from said one or more amino acid modification in said CH3 region of said Fc region; wherein said one or more amino acid modification in said CH3 region of said first Fc region and said one or more amino acid modification in said CH3 region of said second Fc region promote heterodimerization of said first and second heavy chain of said full-length antibody; wherein said CH2 region of said first Fc region comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said CH2 region of said second Fc region comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said one or more amino acid modification in said CH2 region of said Fc region and said one or more amino acid modification in said CH2 region of said second Fc region reduce or abolish one or more of the following Fc region effector functions relative to a reference Fc region that does not contain said one or more amino acid modification (e.g., a heavy chain comprising a wild type CH2 domain, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In some embodiments the first hTAA and the second hTAA are the same. In some embodiments the first hTAA and the second hTAA are different. In some embodiments, the first hTAA and the second hTAA are different, but are expressed by the same cancer cell.

### 5.2.1.4 Format BCA418

In one aspect, provided herein are multispecific proteins set forth in Format BCA418 (see, e.g., FIG. 4). Generally, Format BCA418 provides a multispecific protein comprising a first scFv operably connected (e.g., via a peptide linker) to a first Fab operably connected (e.g., via a peptide linker) to a first Fc region; and a first IgM CH2 mFab operably connected (e.g., via a peptide linker) to a second Fc region; wherein the first scFv specifically binds a hTCSA (e.g., hCD28, hCD2), the first Fab specifically binds hCD3, and the first IgM CH2 mFab specifically binds a hTAA.

In some embodiments, the multispecific protein comprises (a) a scFv comprising from N- to C-terminus (i) a VH region, a peptide linker, and a VL region, or (ii) a VL region, a peptide linker, and a VH region; (b) a Fab comprising (i) a Fab heavy chain comprising from N- to C-terminus a VH region and a CH1 region and (ii) a light chain comprising from N- to C-terminus a VL region and a CL region; (c) a first Fc region comprising from N- to C-terminus a CH2 region and a CH3 region; (d) a second Fc region comprising from N- to C-terminus a CH2 region and a CH3 region; (e) an IgM CH2 mFab comprising (i) an IgM CH2 mFab heavy chain comprising from N- to C-terminus a VH region and an IgM CH2 region and (ii) an IgM CH2 mFab light chain comprising from N- to C-terminus a VL region and an IgM CH2 region; wherein the C-terminus of said scFv is operably connected to the N-terminus of said Fab heavy chain of said Fab; wherein the C-terminus of said Fab heavy chain is operably connected to the N-terminus of said first Fc region; wherein the C-terminus of said IgM CH2 mFab heavy chain is operably connected (e.g., via a peptide linker, e.g., any one of SEQ ID NOS: 217-236 or 318-319, e.g., SEQ ID NO: 318) to the N-terminus of said second Fc region; wherein said IgM CH2 mFab specifically binds a hTAA; wherein said scFv specifically binds a hTCSA (e.g., hCD28, hCD2); wherein said Fab specifically binds hCD3.

In some embodiments, the scFv is operably connected to the N-terminus of said Fab heavy chain directly via a peptide bond. In some embodiments, the scFv is operably connected to the N-terminus of said Fab heavy chain indirectly via a peptide linker (e.g., a linker described herein) (see, e.g., § 5.2.8).

In some embodiments, the scFv is operably connected to the N-terminus of said Fab heavy chain (either directly or indirectly through a peptide linker) through the VH region of the scFv. In some embodiments, the scFv is operably connected to the N-terminus of said Fab heavy chain (either directly or indirectly through a peptide linker) through the VL region of the scFv.

In some embodiments, the C-terminus of said Fab heavy chain is operably connected to the N-terminus of said first Fc region; wherein the C-terminus of said IgM CH2 mFab heavy chain is operably connected to the N-terminus of said second Fc region; wherein said IgM CH2 mFab specifically binds a hTAA via a peptide linker, e.g., any one of SEQ ID NOS: 217-236 or 318-319, e.g., SEQ ID NO: 318.

In some embodiments, the first Fc region comprises from N- to C-terminus a hinge region, a CH2 region and a CH3 region. In some embodiments, the second Fc region comprises from N- to C-terminus a hinge region, a CH2 region and a CH3 region. In some embodiments, the first Fc region comprises from N- to C-terminus a hinge region, a CH2 region and a CH3 region; and the second Fc region comprises from N- to C-terminus a hinge region, a CH2 region and a CH3 region. In some embodiments, the first Fc region comprises from N- to C-terminus a partial hinge region, a CH2 region and a CH3 region. In some embodiments, the second Fc region comprises from N- to C-terminus a partial hinge region, a CH2 region and a CH3 region. In some embodiments, the first Fc region comprises from N- to C-terminus a partial hinge region, a CH2 region and a CH3 region; and the second Fc region comprises from N- to C-terminus a partial hinge region, a CH2 region and a CH3 region.

In some embodiments, the multispecific protein comprises (a) a first polypeptide comprising a first light chain that comprises from N- to C-terminus a VL region and a CL region; (b) second polypeptide comprising from N- to C-terminus (i) a first scFv comprising from N- to C-terminus (i(a)) a VH region, a peptide linker, and a VL region, or (i(b)) a VL region, a peptide linker, and a VH region; (ii) a first heavy chain comprising from N- to C-terminus a VH region, an IgG CH1 region, an IgG hinge region, an IgG CH2 region, and an IgG CH3 region; (c) a third polypeptide comprising from N- to C-terminus a VH region, a first IgM CH2 region, and an Fc region comprising from N- to C-terminus an IgG hinge region, an IgG CH2 region, and an IgG CH3 region; and (d) a fourth polypeptide comprising from N- to C-terminus a VL region and a second IgM CH2 region; wherein said first scFv specifically binds a hTCSA (e.g., hCD28, hCD2); wherein said first light chain and said first heavy chain associate to form an antigen binding domain that specifically binds human CD3 (hCD3); wherein said third and fourth polypeptide associate to form an antigen binding domain that specifically binds a hTAA.

In some embodiments, the second polypeptide comprises from N- to C-terminus (i) a first scFv comprising from N- to C-terminus (i(a)) a VH region, a peptide linker, and a VL region, or (i(b)) a VL region, a peptide linker, and a VH region; a peptide linker (e.g., a linker described herein) (see, e.g., § 5.2.8); (ii) a first heavy chain comprising from N- to C-terminus a VH region, an IgG CH1 region, an IgG hinge region, an IgG CH2 region, and an IgG CH3 region

In some embodiments, the first scFv comprises from N- to C-terminus a VL region, a peptide linker, and a VH region. In some embodiments, the first scFv comprises from N- to C-terminus a VH region, a peptide linker, and a VL region. In some embodiments, the second scFv comprises from N- to C-terminus a VL region, a peptide linker, and a VH region. In some embodiments, the second scFv comprises from N- to C-terminus a VH region, a peptide linker, and a VL region. In some embodiments, the first scFv comprises from N- to C-terminus a VL region, a peptide linker, and a VH region; and the second scFv comprises from N- to C-terminus a VL region, a peptide linker, and a VH region. In some embodiments, the first scFv comprises from N- to C-terminus a VH region, a peptide linker, and a VL region; and the second scFv comprises from N- to C-terminus a VH region, a peptide linker, and a VL region.

Generally, the BCA418 Format can also be described as a multispecific protein comprising: (a) a first polypeptide comprising a first light chain that comprises from N- to C-terminus a VL region and a CL region; (b) second polypeptide comprising from N- to C-terminus (i) a first scFv comprising from N- to C-terminus (i(a)) a VH region, a peptide linker, and a VL region, or (i(b)) a VL region, a peptide linker, and a VH region; an optional first peptide linker (e.g., a linker described herein) (see, e.g., § 5.2.8); (iii) a first heavy chain comprising from N- to C-terminus a VH region, an IgG CH1 region, an IgG hinge region, an IgG CH2 region, and an IgG CH3 region; (c) a third polypeptide comprising from N- to C-terminus a VH region, a first IgM CH2 region, and an Fc region comprising from N- to C-terminus an IgG hinge region, an IgG CH2 region, and an IgG CH3 region; and (d) a fourth polypeptide comprising from N- to C-terminus a VL region and a second IgM CH2 region; wherein said first scFv specifically a hTCSA (e.g., hCD28, hCD2); wherein said first light chain and said first heavy chain associate to form an antigen binding domain that specifically binds human CD3 (hCD3); wherein said third and fourth polypeptide associate to form an antigen binding domain that specifically binds a human hTAA; and wherein said CH3 region of said first heavy chain comprises one or more amino acid modification (e.g., substitution) relative to the amino acid sequence of a reference CH3 region that does not contain said one or more amino acid modification (e.g., a wild-type CH3 region, e.g., SEQ ID NO: 101), wherein said CH3 region of said Fc region comprises one or more amino acid modification (e.g., substitution) relative to the amino acid sequence of a reference CH3 region that does not contain said one or more amino acid modification (e.g., a wild-type CH3 region, e.g., SEQ ID NO: 101); and wherein said one or more amino acid modification in said CH3 region of said first heavy chain is different from said one or more amino acid modification in said CH3 region of said Fc region; wherein said one or more amino acid modification in said CH3 region of said first heavy chain and said one or more amino acid modification in said CH3 region of said Fc region promote heterodimerization of said first heavy chain and said Fc region; wherein said CH2 region of said first heavy chain comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said CH2 region of said Fc region comprises one or more amino acid modification (e.g., substitution) relative to the amino acid sequence of a reference CH2 region that does not contain said one or more amino acid modification (e.g., a wild-type CH2 region, e.g., SEQ ID NO: 100); wherein said one or more amino acid modification in said CH2 region of said first heavy chain and said one or more amino acid modification in said CH2 region of said Fc region reduce or abolish one or more of the following Fc region effector functions relative to a reference Fc region that does not contain said one or more amino acid modification (e.g., a heavy chain comprising a wild-type Fc region, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). 5.2.1.5 Stabilized scFvs

The scFv components of the any of the multispecific proteins described herein include modified scFvs that promote stabilization of the scFv structure. Methods of stabilizing scFvs are known in the art, for example, the introduction of a cysteine residue in the VH region of the scFv and a cysteine residue in the VL region of the scFv, wherein the cysteines are able to form a disulfide bond (see, e.g., U.S. Pat. No. 6,147,203; Zhao, Jian-Xin et al. “Stabilization of the single-chain fragment variable by an interdomain disulfide bond and its effect on antibody affinity.” International journal of molecular sciences vol. 12, 1 1-11. 23 Dec. 2010, doi:10.3390/ijms12010001; Duan, Ye et al. “A novel disulfide-stabilized single-chain variable antibody fragment against rabies virus G protein with enhanced in vivo neutralizing potency.” Molecular immunology vol. 51, 2 (2012): 188-96. Doi:10.1016/j.molimm.2012.03.015; Eve E. Weatherill, et al., Towards a universal disulphide stabilised single chain Fv format: importance of interchain disulphide bond location and VL-VH orientation, Protein Engineering, Design and Selection, Volume 25, Issue 7, July 2012, Pages 321-329, https://doi.org/10.1093/protein/gzs021); the entire contents of each of which is incorporated by reference herein for all purposes).

In some embodiments, the introduction of the cysteine in the VH region of the scFv and the VL region of the scFv does not substantially affect the binding affinity of the scFv for its cognate antigen. In some embodiments, a cysteine is introduced into a framework region of the VH region of the scFv; and a cysteine is introduced into a framework region of the VL region of the scFv.

In some embodiments, a cysteine is introduced at amino acid position 97, 98, 99, 100, 101, 102, 103, 104, 105, or 106 of the VH region of the scFv, numbering according to Kabat; and a cysteine is introduced at amino acid position 40, 41, 42, 43, 44, 45, 46, or 47 of the VL region of the scFv, numbering according to Kabat.

In some embodiments, a cysteine is introduced into a framework region of the VH region of the scFv; and a cysteine is introduced into a framework region of the VL region of the scFv. In some embodiments, a cysteine is introduced at amino acid position 100 of the VH region of the scFv, numbering according to Kabat; and a cysteine is introduced at amino acid position 44 or 45 of the VL region of the scFv, numbering according to Kabat. In some embodiments, a cysteine is introduced into a framework region of the VH region of the scFv; and a cysteine is introduced into a framework region of the VL region of the scFv. In some embodiments, a cysteine is introduced at amino acid position 100 of the VH region of the scFv, numbering according to Kabat; and a cysteine is introduced at amino acid position 44 of the VL region of the scFv, numbering according to Kabat. In some embodiments, a cysteine is introduced into a framework region of the VH region of the scFv; and a cysteine is introduced into a framework region of the VL region of the scFv. In some embodiments, a cysteine is introduced at amino acid position 100 of the VH region of the scFv, numbering according to Kabat; and a cysteine is introduced at amino acid position 45 of the VL region of the scFv, numbering according to Kabat.

5.2.2 Human T-Cell C-Stimulatory Antigen (hTCSA) Binding Domains

The multispecific proteins described herein comprise an antigen binding domain that specifically binds a human T-Cell C-Stimulatory Antigen (hTCSA). As described above, hTCSAs are proteins expressed on the surface of human T-cells that are not part of the T-cell receptor complex, that enhance T-cell activation when bound by their cognate ligand. hTCSAs are known in the art. Exemplary hTCSAs include, but are not limited to, hCD28, hCD2, hCD137 (also known as 41BB), human CD27, human CD278 (also known as ICOS), human CD134 (also known as OX40), or human CD40. In some embodiments, the hTCSA is hCD28. In some embodiments, the hTCSA is hCD2. In some embodiments, the hTCSA is h41BB.

### 5.2.2.1 CD28 Binding Domains

CD28 is a co-stimulatory transmembrane protein expressed on the surface of T-cells, including naïve T-cells. CD28 functions in e.g., T-cell activation, the induction of T-cell proliferation, T-cell cytokine production, and the promotion of T-cell survival. CD28 is the receptor for CD80 and CD86 proteins, cell surface transmembrane proteins expressed by antigen presenting cells.

The amino acid sequence of a reference immature hCD28 polypeptide (signal peptide at the N-terminus) and mature hCD28 polypeptide (no signal peptide) is set forth in SEQ ID NOS: 1 and 2 respectively. See Table 1 herein.

TABLE 1
The Amino Acid Sequence of Reference hCD28 Polypeptides

| | | SEQ ID |
|---|---|---|
| Description | Amino Acid Sequence | NO |
| hCD28 | MLRLLLALNLFPSIQVTGNKILVKQSPMLVAYDNAVNLSCKY | 1 |
| Immature-Signal | SYNLFSREFRASLHKGLDSAVEVCVVYGNYSQQLQVYSKTGF | |
| Peptide at | NCDGKLGNESVTFYLQNLYVNQTDIYFCKIEVMYPPPYLDNE | |
| N-terminus | KSNGTIIHVKGKHLCPSPLFPGPSKPFWVLVVVGGVLACYSL | |
| UniProt ID: | LVTVAFIIFWVRSKRSRLLHSDYMNMTPRRPGPTRKHYQPYA | |
| P10747 | PPRDFAAYRS | |
| hCD28 | NKILVKQSPMLVAYDNAVNLSCKYSYNLFSREFRASLHKGLD | |
| Mature-No Signal | SAVEVCVVYGNYSQQLQVYSKTGFNCDGKLGNESVTFYLQNL | |
| Peptide | YVNQTDIYFCKIEVMYPPPYLDNEKSNGTIIHVKGKHLCPSP | 2 |
| UniProt ID: | LFPGPSKPFWVLVVVGGVLACYSLLVTVAFIIFWVRSKRSRL | |
| P10747 | LHSDYMNMTPRRPGPTRKHYQPYAPPRDFAAYRS | |

In some embodiments, multispecific proteins described herein comprise an antigen binding domain that specifically binds hCD28, also referred to herein as a hCD28 binding domain or an anti-hCD28 antibody (or functional fragment or variant thereof). In particular embodiments, the hCD28 binding domain is a scFv (see, e.g., § 5.2.1). In particular embodiments, the multispecific protein or polypeptide is monovalent for hCD28.

hCD28 binding domains that can be employed in multispecific proteins described herein are known in the art, and include, for example, those described in, e.g., U.S. Pat. Nos. 7,585,960, 9,562,098, 7,723,482, 7,939,638B2, U.S. Pat. Nos. 9,908,937, 7,723,482B2, WO2021155071, WO2023164510A1, WO2023155845A1, WO2023143547A1, WO2023114701A2, WO2017103003A1, WO2011101791A1, WO2022253867A1, WO2022216915A1, and WO2020127628A1, US20230265218A1, WO2022094299, US20220089766A1, the entire contents of each of which is incorporated by reference herein for all purposes.

Exemplary hCD28 binding domains that can be employed in multispecific proteins described herein are described in, see, e.g., § 5.2.2, Table 2.

Exemplary hCD28 binding domains known in the art that can be employed in fusion proteins described herein also, include, TGN1412.

The amino acid sequence of exemplary hCD28 binding domains that can be utilized in the multispecific proteins described herein is provided in Table 2. The CDRs of the hCD28 binding domains in Table 2, are denoted according to Kabat. A person of ordinary skill in the art would be able to determine the CDRs as defined by another scheme, e.g., Chothia, IMGT, using ordinary methods known in the art.

TABLE 2
The Amino Acid Sequence of Exemplary hCD28 Binding Domains

| | | SEQ ID | |
|---|---|---|---|
| Description | | NO | Amino Acid Sequence |
| TGN1412 | VH CDR1 | 3 | SYYIH |
| | VH CDR2 | 4 | CIYPGNVNTNYNEKFKD |
| | VH CDR3 | 5 | SHYGLDWNFDV |
| | VL CDR1 | 6 | HASQNIYVWLN |
| | VL CDR2 | 7 | KASNLHT |
| | VL CDR3 | 8 | QQGQTYPYT |
| | VH | 9 | QVQLVQSGAEVKKPGASVKVSCKASGYTFTSYYIHWV |
| | | | RQAPGQGLEWIGCIYPGNVNTNYNEKFKDRATLTVDT |
| | | | SISTAYMELSRLRSDDTAVYFCTRSHYGLDWNFDVWG |
| | | | QGTTVTVSS |
| | VL | 10 | DIQMTQSPSSLSASVGDRVTITCHASQNIYVWLNWYQ |
| | | | QKPGKAPKLLIYKASNLHTGVPSRFSGSGSGTDFTLT |
| | | | ISSLQPEDFATYYCQQGQTYPYTFGGGTKVEIK |
| S1 | VH CDR1 | 11 | DYGVH |
| | VH CDR2 | 12 | VIWAGGGTNYNP SLKS |
| | VH CDR3 | 13 | DKGYSYYYSMDY |
| | VL CDR1 | 14 | RASESVEYYVTSLMQ |
| | VL CDR2 | 15 | AASNVES |
| | VL CDR3 | 16 | QQSRKVPYT |
| | VH-C | 17 | QVQLQESGPGLVKPSQTLSLTCTVSGFSLSDYGVHWV |
| | | | RQPPGKCLEWLGVIWAGGGTNYNPSLKSRKTISKDTS |
| | | | KNQVSLKLSSVTAADTAVYYCARDKGYSYYYSMDYWG |
| | | | QGTTVTVSS |
| | VH | 18 | QVQLQESGPGLVKPSQTLSLTCTVSGFSLSDYGVHWV |
| | | | RQPPGKGLEWLGVIWAGGGTNYNPSLKSRKTISKDTS |
| | | | KNQVSLKLSSVTAADTAVYYCARDKGYSYYYSMDYWG |
| | | | QGTTVTVSS |
| | VL-C | 19 | DIVLTQSPASLAVSPGQRATITCRASESVEYYVTSLM |
| | | | QWYQQKPGQPPKLLIFAASNVESGVPARFSGSGSGTD |
| | | | FTLTINPVEANDVANYYCQQSRKVPYTFGCGTKLEIK |
| | VL | 20 | DIVLTQSPASLAVSPGQRATITCRASESVEYYVTSLM |
| | | | QWYQQKPGQPPKLLIFAASNVESGVPARFSGSGSGTD |
| | | | FTLTINPVEANDVANYYCQQSRKVPYTFGGGTKLEIK |
| CD28.3 | VH CDR1 | 327 | EYIIH |
| | VH CDR2 | 328 | WFYPGSNDIQYNAQFKG |
| | VH CDR3 | 329 | RDDFSGYDALPY |
| | VL CDR1 | 330 | KTNENIYSNLA |
| | VL CDR2 | 331 | AATHLVE |
| | VL CDR3 | 332 | QHFWGTPCT |
| | VH | 333 | VQLQQSGAELKKPGASVKVSCKASGYTFTEYIIHWIK |
| | | | LRSGQGLEWIGWFYPGSNDIQYNAQFKGKATLTADKS |
| | | | SSTVYMELTGLTPEDSAVYFCARRDDFSGYDALPYWG |
| | | | QGTLVTVSA |
| | VL-A | 334 | DIQMTQSPSSLSASVGDRVTITCKTNENIYSNLAWYQ |
| | | | QKDGKSPQLLIYAATHLVEGVPSRFSGSGSGTQYSLT |
| | | | ISSLQPEDFGNYYCQHFWGTPCTFGGGTKLEIKR |
| | VL-B | 335 | DIQMTQSPSSLSASVGDRVTITCKTNENIYSNLAWYQ |
| | | | QKDGKSPQLLIYAATHLVEGVPSRFSGSGSGTQYSLT |
| | | | ISSLQPEDFGNYYCQHFWGTPATFGGGTKLEIKR |
| | VL-C | 336 | DIQMTQSPSSLSASVGDRVTITCKTNENIYSNLAWYQ |
| | | | QKDGKSPQLLIYAATHLVEGVPSRFSGSGSGTQYSLT |
| | | | ISSLQPEDFGNYYCQHFWGTPNTFGGGTKLEIKR |

In some embodiments, the hCD28 binding domain comprises a hCD28 binding domain provided in Table 2.

In some embodiments, the hCD28 binding domain comprises a VH that comprises: VH CDR1, VH CDR2, and VH CDR3.

In some embodiments, the amino acid sequence of VH CDR1 comprises or consists of the amino acid sequence of a VH CDR1 of a VH set forth in Table 2, or the amino acid sequence of a VH CDR1 of a VH set forth in Table 2 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 of a VH set forth in Table 2, or the amino acid sequence of a VH CDR2 of a VH set forth in Table 2 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 of a VH set forth in Table 2, or the amino acid sequence of a VH CDR3 of a VH set forth in Table 2 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 of a VH set forth in Table 2, or the amino acid sequence of a VH CDR1 of a VH set forth in Table 2 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 2, or the amino acid sequence of a VH CDR2 of a VH set forth in Table 2 comprising or consisting of 1, 2, or 3 amino acid modifications (e.g., substitution, deletion, addition, etc.); and the amino acid sequence of VH CDR3 comprises or consists of the amino acid sequence of a VH CDR3 of a VH set forth in Table 2, or the amino acid sequence of a VH CDR3 of a VH set forth in Table 2 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 2, or the amino acid sequence of a VH CDR1 set forth in Table 2 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 set forth in Table 2, or the amino acid sequence of a VH CDR2 set forth in Table 2 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 set forth in Table 2, or the amino acid sequence of a VH CDR3 set forth in Table 2 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 2, or the amino acid sequence of a VH CDR1 set forth in Table 2 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 2, or the amino acid sequence of a VH CDR2 set forth in Table 2 comprising or consisting of 1, 2, or 3 amino acid modifications (e.g., substitution, deletion, addition, etc.); and the amino acid sequence of VH CDR3 comprises or consists of the amino acid sequence of a VH CDR3 set forth in Table 2, or the amino acid sequence of a VH CDR3 set forth in Table 2 comprising or consisting of 1, 2, or 3 amino acid modifications (e.g., substitution, deletion, addition, etc.).

In some embodiments, the hCD28 binding domain comprises a VL that comprises: VL CDR1, VL CDR2, and VL CDR3.

In some embodiments, the amino acid sequence of VL CDR1 comprises or consists of the amino acid sequence of a VL CDR1 of a VL set forth in Table 2, or the amino acid sequence of a VL CDR1 of a VL set forth in Table 2 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 of a VL set forth in Table 2, or the amino acid sequence of a VL CDR2 of a VL set forth in Table 2 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 of a VL set forth in Table 2, or the amino acid sequence of a VL CDR3 of a VL set forth in Table 2 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 of a VL set forth in Table 2, or the amino acid sequence of a VL CDR1 of a VL set forth in Table 2 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 2, or the amino acid sequence of a VL CDR2 of a VL set forth in Table 2 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 2, or the amino acid sequence of a VL CDR3 of a VL set forth in Table 2 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 2, or the amino acid sequence of a VL CDR1 set forth in Table 2 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 set forth in Table 2, or the amino acid sequence of a VL CDR2 set forth in Table 2 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 set forth in Table 2, or the amino acid sequence of a VL CDR3 set forth in Table 2 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 2, or the amino acid sequence of a VL CDR1 set forth in Table 2 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 2, or the amino acid sequence of a VL CDR2 set forth in Table 2 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 2, or the amino acid sequence of a VL CDR3 set forth in Table 2 comprising or consisting of 1, 2, or 3 amino acid modifications (e.g., substitution, deletion, addition, etc.).

In some embodiments, the hCD28 binding domain comprises a VH that comprises: VH CDR1, VH CDR2, and VH CDR3; and a VL that comprises: VL CDR1, VL CDR2, and VL CDR3.

In some embodiments, the amino acid sequence of VH CDR1 comprises or consists of the amino acid sequence of a VH CDR1 of a VH set forth in Table 2, or the amino acid sequence of a VH CDR1 of a VH set forth in Table 2 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 2, or the amino acid sequence of a VH CDR2 of a VH set forth in Table 2 comprising or consisting of 1, 2, or 3 amino acid modifications (e.g., substitution, deletion, addition, etc.); the amino acid sequence of VH CDR3 comprises or consists of the amino acid sequence of a VH CDR3 of a VH set forth in Table 2, or the amino acid sequence of a VH CDR3 of a VH set forth in Table 2 comprising or consisting of 1, 2, or 3 amino acid modifications (e.g., substitution, deletion, addition, etc.); the amino acid sequence of VL CDR1 comprises or consists of the amino acid sequence of a VL CDR1 of a VL set forth in Table 2, or the amino acid sequence of a VL CDR1 of a VL set forth in Table 2 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 2, or the amino acid sequence of a VL CDR2 of a VL set forth in Table 2 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 2, or the amino acid sequence of a VL CDR3 of a VL set forth in Table 2 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 2, or the amino acid sequence of a VH CDR1 set forth in Table 2 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 2, or the amino acid sequence of a VH CDR2 set forth in Table 2 comprising or consisting of 1, 2, or 3 amino acid modifications (e.g., substitution, deletion, addition, etc.); the amino acid sequence of VH CDR3 comprises or consists of the amino acid sequence of a VH CDR3 set forth in Table 2, or the amino acid sequence of a VH CDR3 set forth in Table 2 comprising or consisting of 1, 2, or 3 amino acid modifications (e.g., substitution, deletion, addition, etc.); the amino acid sequence of VL CDR1 comprises or consists of the amino acid sequence of a VL CDR1 set forth in Table 2, or the amino acid sequence CDR1 of a VL set forth in Table 2 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 2, or the amino acid sequence of a VL CDR2 set forth in Table 2 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 2, or the amino acid sequence of a VL CDR3 set forth in Table 2 comprising or consisting of 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 2. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 2. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 2; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 2.

In some embodiments, the amino acid sequence of VH CDR1 comprises or consists of the amino acid sequence set forth in SEQ ID NO: 3, or the amino acid sequence set forth in SEQ ID NO: 3 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 4, or the amino acid sequence set forth in SEQ ID NO: 4 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 5, or the amino acid sequence set forth in SEQ ID NO: 5 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 3, or the amino acid sequence set forth in SEQ ID NO: 3 comprising 1, 2, or 3 amino acid modifications (e.g., substitution, deletion, addition, etc.); the amino acid sequence of VH CDR2 comprises or consists of the amino acid sequence set forth in SEQ ID NO: 4, or the amino acid sequence set forth in SEQ ID NO: 4 comprising 1, 2, or 3 amino acid modifications (e.g., substitution, deletion, addition, etc.); and the amino acid sequence of VH CDR3 comprises or consists of the amino acid sequence set forth in SEQ ID NO: 5, or the amino acid sequence set forth in SEQ ID NO: 5 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 6, or the amino acid sequence set forth in SEQ ID NO: 6 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 7, or the amino acid sequence set forth in SEQ ID NO: 7 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 8, or the amino acid sequence set forth in SEQ ID NO: 8 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 6, or the amino acid sequence set forth in SEQ ID NO: 6 comprising 1, 2, or 3 amino acid modifications (e.g., substitution, deletion, addition, etc.); the amino acid sequence of VL CDR2 comprises or consists of the amino acid sequence set forth in SEQ ID NO: 7, or the amino acid sequence set forth in SEQ ID NO: 7 comprising 1, 2, or 3 amino acid modifications (e.g., substitution, deletion, addition, etc.); and the amino acid sequence of VL CDR3 comprises or consists of the amino acid sequence set forth in SEQ ID NO: 8, or the amino acid sequence set forth in SEQ ID NO: 8 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 3, or the amino acid sequence set forth in SEQ ID NO: 3 comprising 1, 2, or 3 amino acid modifications (e.g., substitution, deletion, addition, etc.); the amino acid sequence of VH CDR2 comprises or consists of the amino acid sequence set forth in SEQ ID NO: 4, or the amino acid sequence set forth in SEQ ID NO: 4 comprising 1, 2, or 3 amino acid modifications (e.g., substitution, deletion, addition, etc.); the amino acid sequence of VH CDR3 comprises or consists of the amino acid sequence set forth in SEQ ID NO: 5, or the amino acid sequence set forth in SEQ ID NO: 5 comprising 1, 2, or 3 amino acid modifications (e.g., substitution, deletion, addition, etc.); the amino acid sequence of VL CDR1 comprises or consists of the amino acid sequence set forth in SEQ ID NO: 6, or the amino acid sequence set forth in SEQ ID NO: 6 comprising 1, 2, or 3 amino acid modifications (e.g., substitution, deletion, addition, etc.); the amino acid sequence of VL CDR2 comprises or consists of the amino acid sequence set forth in SEQ ID NO: 7, or the amino acid sequence set forth in SEQ ID NO: 7 comprising 1, 2, or 3 amino acid modifications (e.g., substitution, deletion, addition, etc.); and the amino acid sequence of VL CDR3 comprises or consists of the amino acid sequence set forth in SEQ ID NO: 8, or the amino acid sequence set forth in SEQ ID NO: 8 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10.

In some embodiments, the amino acid sequence of VH CDR1 comprises or consists of the amino acid sequence set forth in SEQ ID NO: 11, or the amino acid sequence set forth in SEQ ID NO: 11 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 12, or the amino acid sequence set forth in SEQ ID NO: 12 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 13, or the amino acid sequence set forth in SEQ ID NO: 13 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 11, or the amino acid sequence set forth in SEQ ID NO: 11 comprising 1, 2, or 3 amino acid modifications (e.g., substitution, deletion, addition, etc.); the amino acid sequence of VH CDR2 comprises or consists of the amino acid sequence set forth in SEQ ID NO: 12, or the amino acid sequence set forth in SEQ ID NO: 12 comprising 1, 2, or 3 amino acid modifications (e.g., substitution, deletion, addition, etc.); and the amino acid sequence of VH CDR3 comprises or consists of the amino acid sequence set forth in SEQ ID NO: 13, or the amino acid sequence set forth in SEQ ID NO: 13 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 14, or the amino acid sequence set forth in SEQ ID NO: 14 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 15, or the amino acid sequence set forth in SEQ ID NO: 15 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 16, or the amino acid sequence set forth in SEQ ID NO: 16 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 14, or the amino acid sequence set forth in SEQ ID NO: 14 comprising 1, 2, or 3 amino acid modifications (e.g., substitution, deletion, addition, etc.); the amino acid sequence of VL CDR2 comprises or consists of the amino acid sequence set forth in SEQ ID NO: 15, or the amino acid sequence set forth in SEQ ID NO: 15 comprising 1, 2, or 3 amino acid modifications (e.g., substitution, deletion, addition, etc.); and the amino acid sequence of VL CDR3 comprises or consists of the amino acid sequence set forth in SEQ ID NO: 16, or the amino acid sequence set forth in SEQ ID NO: 16 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 11, or the amino acid sequence set forth in SEQ ID NO: 11 comprising 1, 2, or 3 amino acid modifications (e.g., substitution, deletion, addition, etc.); the amino acid sequence of VH CDR2 comprises or consists of the amino acid sequence set forth in SEQ ID NO: 12, or the amino acid sequence set forth in SEQ ID NO: 12 comprising 1, 2, or 3 amino acid modifications (e.g., substitution, deletion, addition, etc.); the amino acid sequence of VH CDR3 comprises or consists of the amino acid sequence set forth in SEQ ID NO: 13, or the amino acid sequence set forth in SEQ ID NO: 13 comprising 1, 2, or 3 amino acid modifications (e.g., substitution, deletion, addition, etc.); the amino acid sequence of VL CDR1 comprises or consists of the amino acid sequence set forth in SEQ ID NO: 14, or the amino acid sequence set forth in SEQ ID NO: 14 comprising 1, 2, or 3 amino acid modifications (e.g., substitution, deletion, addition, etc.); the amino acid sequence of VL CDR2 comprises or consists of the amino acid sequence set forth in SEQ ID NO: 15, or the amino acid sequence set forth in SEQ ID NO: 15 comprising 1, 2, or 3 amino acid modifications (e.g., substitution, deletion, addition, etc.); and the amino acid sequence of VL CDR3 comprises or consists of the amino acid sequence set forth in SEQ ID NO: 16, or the amino acid sequence set forth in SEQ ID NO: 16 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 19. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 19.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 18. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 20. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 18; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 20.

### 5.2.2.2 CD2 Binding Domains

CD2 is a co-stimulatory transmembrane protein expressed on the surface of T-cells and NK cells. CD28 functions in e.g., the formation and organization of the immunological synapse that is formed between T cells and antigen-presenting cells upon cell-cell conjugation and associated intracellular signaling. CD2 is the receptor for LFA3, a cell surface protein expressed by antigen presenting cells.

The amino acid sequence of a reference immature hCD2 polypeptide (signal peptide at the N-terminus) and mature hCD2 polypeptide (no signal peptide) is set forth in SEQ ID NO: 37 and 38, respectively. See Table 16, herein.

TABLE 16
The Amino Acid Sequence of Reference hCD2 Polypeptides

| | | SEQ ID |
|---|---|---|
| Description | Amino Acid Sequence | NO |
| hCD2 | MSFPCKFVASFLLIFNVSSKGAVSKEITNALETWGALGQDINLDIP | 37 |
| (Immature-Signal | SFQMSDDIDDIKWEKTSDKKKIAQFRKEKETFKEKDTYKLFKNGTL | |
| Peptide at | KIKHLKTDDQDIYKVSIYDTKGKNVLEKIFDLKIQERVSKPKISWT | |
| N-terminus) | CINTTLTCEVMNGTDPELNLYQDGKHLKLSQRVITHKWTTSLSAKF | |
| UniProt ID: | KCTAGNKVSKESSVEPVSCPEKGLDIYLIIGICGGGSLLMVFVALL | |
| P06729 | VFYITKRKKQRSRRNDEELETRAHRVATEERGRKPHQIPASTPQNP | |
| | ATSQHPPPPPGHRSQAPSHRPPPPGHRVQHQPQKRPPAPSGTQVHQ | |
| | QKGPPLPRPRVQPKPPHGAAENSLSPSSN | |
| hCD2 | KEITNALETWGALGQDINLDIPSFQMSDDIDDIKWEKTSDKKKIAQ | 38 |
| (Mature-No | FRKEKETFKEKDTYKLFKNGTLKIKHLKTDDQDIYKVSIYDTKGKN | |
| Signal | VLEKIFDLKIQERVSKPKISWTCINTTLTCEVMNGTDPELNLYQDG | |
| Peptide) | KHLKLSQRVITHKWTTSLSAKFKCTAGNKVSKESSVEPVSCPEKGL | |
| UniProt ID: | DIYLIIGICGGGSLLMVFVALLVFYITKRKKQRSRRNDEELETRAH | |
| P06729 | RVATEERGRKPHQIPASTPQNPATSQHPPPPPGHRSQAPSHRPPPP | |
| | GHRVQHQPQKRPPAPSGTQVHQQKGPPLPRPRVQPKPPHGAAENSL | |
| | SPSSN | |

In some embodiments, multispecific proteins described herein comprise an antigen binding domain that specifically binds hCD2, also referred to herein as a hCD2 binding domain or an anti-hCD2 antibody (or functional fragment or variant thereof). In particular embodiments, the hCD2 binding domain is a scFv (see, e.g., § 5.2.1). In particular embodiments, the multispecific protein or polypeptide is monovalent for hCD28.

Exemplary hCD2 binding domains that can be employed in multispecific proteins described herein are described in § 5.2.2, Table 17.

Exemplary hCD2 binding domains known in the art that can be employed in fusion proteins described herein also, include, e.g., siplizumab, LO-CD2b, humanized versions of LO-CD2b (see, e.g., WO1999003502).

hCD2 binding domains that can be employed in multispecific proteins described herein are known in the art, and include, for example, those described in, e.g., U.S. Pat. No. 5,656,438A, EP1003552B1, WO2022067089, WO2023126445A1, WO2023126445A1, WO2022157272A1, WO2021259927A2, US20210260212A1, U.S. Pat. No. 7,678,582B2, U.S. Pat. No. 7,250,167B2, U.S. Pat. No. 7,332,157B2, U.S. Pat. No. 6,384,198B1, and WO1999003502, the entire contents of each of which is incorporated by reference herein for all purposes.

The amino acid sequence of exemplary hCD2 binding domains that can be utilized in the multispecific proteins described herein is provided in Table 17. The CDRs of the hCD2 binding domains in Table 17, are denoted according to Kabat. A person of ordinary skill in the art would be able to determine the CDRs as defined by another scheme, e.g., Chothia, IMGT, using ordinary methods known in the art.

TABLE 17
The Amino Acid Sequence of Exemplary hCD2 Binding Domains

| | | SEQ ID | |
|---|---|---|---|
| Description | | NO | Amino Acid Sequence |
| Anti-CD2 | VH CDR1 | 60 | GYYMH |
| | VH CDR2 | 85 | RINPNSGGTNYAQKFQG |
| | VH CDR3 | 86 | GRTEYIVVAEGFDY |
| | VL CDR1 | 87 | RSSQSLVYSDGNTHLN |
| | VL CDR2 | 88 | KVSNRDS |
| | VL CDR3 | 89 | MQGTHWPYT |
| | VH | 90 | QVQLVQSGAEVKKPGASVKVSCKASGYTFTGYYMHWVRQAP |
| | | | GQGLEWMGRINPNSGGTNYAQKFQGRVTMTRDTSISTAYME |
| | | | LSRLRSDDTAVYYCARGRTEYIVVAEGFDYWGQGTLVTVSS |
| | VL | 91 | DVVMTQSPLSLPVTLGQPASISCRSSQSLVYSDGNTHLNWF |
| | | | QQRPGQSPRRLIYKVSNRDSGVPDRFSGSGSGTDFTLKISR |
| | | | VEAEDVGVYYCMQGTHWPYTFGQGTKLEIK |
| Anti-CD2 A | VH CDR1 | 337 | EYYMY |
| | VH CDR2 | 338 | RIDPEDGSIDYVEKFKK |
| | VH CDR3 | 339 | GKFNYRFAY |
| | VL CDR1 | 340 | RSSQSLLHSSGNTYLN |
| | VL CDR2 | 341 | LVSKLES |
| | VL CDR3 | 342 | MQFTHYPYT |
| | VH-A | 343 | QVQLVQSGAEVQRPGASVKVSCKASGYIFTEYYMYWVRQAP |
| | | | GQGLELVGRIDPEDGSIDYVEKFKKKVTLTADTSSSTAYME |
| | | | LSSLTSDDTAVYYCARGKFNYRFAYWGQGTLVTVSS |
| | VL-A | 344 | DVVMTQSPPSLLVTLGQPASISCRSSQSLLHSSGNTYLNWL |
| | | | LQRPGQSPQPLIYLVSKLESGVPDRFSGSGSGTDFTLKISG |
| | | | VEAEDVGVYYCMQFTHYPYTFGQGTKLEIK |
| | VH-B | 345 | EVQLQQSGPELQRPGASVKLSCKASGYIFTEYYMYWVKQRP |
| | | | KQQLELVGRIDPEDGSIDYVEKFKKKATLTADTSSNTAYMQ |
| | | | LSSLTSEDTATYFCARGKFNYRFAYWGQGTLVTVSS |
| | VL-B | 346 | DVLTQTPPTLLATIGQSVSISCRSSQSLLHSSGNTYLNWLL |
| | | | QRTGQSPQPLIYLVSKLESGVPNRFSGSGSGTDFTLKISGV |
| | | | EAEDLGVYYCMQFTHYPYTFGAGTKLELK |
| | VH-C | 347 | QVQLVQSGAEVKKPGASVKVSCKASGYTFTEYYMYWVRQAP |
| | | | GQGLELMGRIDPEDGSIDYVEKFKKKVTLTADTSSSTAYME |
| | | | LSSLISDDTAVYYCARGKFNYRFAYWGQGTLVTVSS |
| | VL-C | 348 | DVVMTQSPPSLLVTLGQPASISCRSSQSLLHSSGNTYLNWL |
| | | | LQRPGQSPQPLIYLVSKLESGVPDRFSGSGSGTDFTLKISG |
| | | | VEAEDVGVYYCMQFTHYPYTFGQGTKLEIK |
| | VH-D | 349 | EVQLVESGGGLVQPGGSLRLSCAASGFTFSDYWMSWVRQAP |
| | | | GKRLEWIGHIKYDGSYTNYAPSLKNRFTISRDNAKNSLYLQ |
| | | | MNSLRSEDTAVYYCAREAPGAASYWGQGTLVTVSS |
| | VL-D | 350 | DVVLTQTPLSLSVTPGQPASISCRSSQSLVHSNGNTYLEWF |
| | | | LQKPGQSPQLLIYKVSNRFSGVPDRFSGSGSGSDFTLKISR |
| | | | VEAEDVGVYYCFQGTHDPYTFGQGTKVEIK |

In some embodiments, the hCD2 binding domain comprises a hCD2 binding domain provided in Table 17.

In some embodiments, the hCD2 binding domain comprises a VH that comprises: VH CDR1, VH CDR2, and VH CDR3.

In some embodiments, the amino acid sequence of VH CDR1 comprises or consists of the amino acid sequence of a VH CDR1 of a VH set forth in Table 17, or the amino acid sequence of a VH CDR1 of a VH set forth in Table 17 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 of a VH set forth in Table 17, or the amino acid sequence of a VH CDR2 of a VH set forth in Table 17 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 of a VH set forth in Table 17, or the amino acid sequence of a VH CDR3 of a VH set forth in Table 17 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 of a VH set forth in Table 17, or the amino acid sequence of a VH CDR1 of a VH set forth in Table 17 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 17, or the amino acid sequence of a VH CDR2 of a VH set forth in Table 17 comprising or consisting of 1, 2, or 3 amino acid modifications (e.g., substitution, deletion, addition, etc.); and the amino acid sequence of VH CDR3 comprises or consists of the amino acid sequence of a VH CDR3 of a VH set forth in Table 17, or the amino acid sequence of a VH CDR3 of a VH set forth in Table 17 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 17, or the amino acid sequence of a VH CDR1 set forth in Table 17 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 set forth in Table 17, or the amino acid sequence of a VH CDR2 set forth in Table 17 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 set forth in Table 17, or the amino acid sequence of a VH CDR3 set forth in Table 17 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 17, or the amino acid sequence of a VH CDR1 set forth in Table 17 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 17, or the amino acid sequence of a VH CDR2 set forth in Table 17 comprising or consisting of 1, 2, or 3 amino acid modifications (e.g., substitution, deletion, addition, etc.); and the amino acid sequence of VH CDR3 comprises or consists of the amino acid sequence of a VH CDR3 set forth in Table 17, or the amino acid sequence of a VH CDR3 set forth in Table 17 comprising or consisting of 1, 2, or 3 amino acid modifications (e.g., substitution, deletion, addition, etc.).

In some embodiments, the hCD2 binding domain comprises a VL that comprises: VL CDR1, VL CDR2, and VL CDR3.

In some embodiments, the amino acid sequence of VL CDR1 comprises or consists of the amino acid sequence of a VL CDR1 of a VL set forth in Table 17, or the amino acid sequence of a VL CDR1 of a VL set forth in Table 17 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 of a VL set forth in Table 17, or the amino acid sequence of a VL CDR2 of a VL set forth in Table 17 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 of a VL set forth in Table 17, or the amino acid sequence of a VL CDR3 of a VL set forth in Table 2 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 of a VL set forth in Table 2, or the amino acid sequence of a VL CDR1 of a VL set forth in Table 17 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 17, or the amino acid sequence of a VL CDR2 of a VL set forth in Table 17 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 17, or the amino acid sequence of a VL CDR3 of a VL set forth in Table 17 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 17, or the amino acid sequence of a VL CDR1 set forth in Table 17 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 set forth in Table 17, or the amino acid sequence of a VL CDR2 set forth in Table 17 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 set forth in Table 17, or the amino acid sequence of a VL CDR3 set forth in Table 17 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 17, or the amino acid sequence of a VL CDR1 set forth in Table 17 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 17, or the amino acid sequence of a VL CDR2 set forth in Table 17 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 17, or the amino acid sequence of a VL CDR3 set forth in Table 17 comprising or consisting of 1, 2, or 3 amino acid modifications (e.g., substitution, deletion, addition, etc.).

In some embodiments, the hCD2 binding domain comprises a VH that comprises: VH CDR1, VH CDR2, and VH CDR3; and a VL that comprises: VL CDR1, VL CDR2, and VL CDR3.

In some embodiments, the amino acid sequence of VH CDR1 comprises or consists of the amino acid sequence of a VH CDR1 of a VH set forth in Table 17, or the amino acid sequence of a VH CDR1 of a VH set forth in Table 17 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 17, or the amino acid sequence of a VH CDR2 of a VH set forth in Table 17 comprising or consisting of 1, 2, or 3 amino acid modifications (e.g., substitution, deletion, addition, etc.); the amino acid sequence of VH CDR3 comprises or consists of the amino acid sequence of a VH CDR3 of a VH set forth in Table 17, or the amino acid sequence of a VH CDR3 of a VH set forth in Table 17 comprising or consisting of 1, 2, or 3 amino acid modifications (e.g., substitution, deletion, addition, etc.); the amino acid sequence of VL CDR1 comprises or consists of the amino acid sequence of a VL CDR1 of a VL set forth in Table 17, or the amino acid sequence of a VL CDR1 of a VL set forth in Table 17 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 17, or the amino acid sequence of a VL CDR2 of a VL set forth in Table 17 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 17, or the amino acid sequence of a VL CDR3 of a VL set forth in Table 17 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 17, or the amino acid sequence of a VH CDR1 set forth in Table 17 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 17, or the amino acid sequence of a VH CDR2 set forth in Table 17 comprising or consisting of 1, 2, or 3 amino acid modifications (e.g., substitution, deletion, addition, etc.); the amino acid sequence of VH CDR3 comprises or consists of the amino acid sequence of a VH CDR3 set forth in Table 17, or the amino acid sequence of a VH CDR3 set forth in Table 17 comprising or consisting of 1, 2, or 3 amino acid modifications (e.g., substitution, deletion, addition, etc.); the amino acid sequence of VL CDR1 comprises or consists of the amino acid sequence of a VL CDR1 set forth in Table 17, or the amino acid sequence CDR1 of a VL set forth in Table 17 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 17, or the amino acid sequence of a VL CDR2 set forth in Table 17 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 17, or the amino acid sequence of a VL CDR3 set forth in Table 17 comprising or consisting of 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 17. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 17. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 17; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 17.

In some embodiments, the amino acid sequence of VH CDR1 comprises or consists of the amino acid sequence set forth in SEQ ID NO: 60, or the amino acid sequence set forth in SEQ ID NO: 60 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 85, or the amino acid sequence set forth in SEQ ID NO: 85 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 86, or the amino acid sequence set forth in SEQ ID NO: 86 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 60, or the amino acid sequence set forth in SEQ ID NO: 60 comprising 1, 2, or 3 amino acid modifications (e.g., substitution, deletion, addition, etc.); the amino acid sequence of VH CDR2 comprises or consists of the amino acid sequence set forth in SEQ ID NO: 85, or the amino acid sequence set forth in SEQ ID NO: 85 comprising 1, 2, or 3 amino acid modifications (e.g., substitution, deletion, addition, etc.); and the amino acid sequence of VH CDR3 comprises or consists of the amino acid sequence set forth in SEQ ID NO: 86, or the amino acid sequence set forth in SEQ ID NO: 86 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 87, or the amino acid sequence set forth in SEQ ID NO: 87 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 88, or the amino acid sequence set forth in SEQ ID NO: 88 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 89, or the amino acid sequence set forth in SEQ ID NO: 89 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 87, or the amino acid sequence set forth in SEQ ID NO: 87 comprising 1, 2, or 3 amino acid modifications (e.g., substitution, deletion, addition, etc.); the amino acid sequence of VL CDR2 comprises or consists of the amino acid sequence set forth in SEQ ID NO: 88, or the amino acid sequence set forth in SEQ ID NO: 88 comprising 1, 2, or 3 amino acid modifications (e.g., substitution, deletion, addition, etc.); and the amino acid sequence of VL CDR3 comprises or consists of the amino acid sequence set forth in SEQ ID NO: 89, or the amino acid sequence set forth in SEQ ID NO: 89 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 60, or the amino acid sequence set forth in SEQ ID NO: 60 comprising 1, 2, or 3 amino acid modifications (e.g., substitution, deletion, addition, etc.); the amino acid sequence of VH CDR2 comprises or consists of the amino acid sequence set forth in SEQ ID NO: 85, or the amino acid sequence set forth in SEQ ID NO: 85 comprising 1, 2, or 3 amino acid modifications (e.g., substitution, deletion, addition, etc.); the amino acid sequence of VH CDR3 comprises or consists of the amino acid sequence set forth in SEQ ID NO: 86, or the amino acid sequence set forth in SEQ ID NO: 86 comprising 1, 2, or 3 amino acid modifications (e.g., substitution, deletion, addition, etc.); the amino acid sequence of VL CDR1 comprises or consists of the amino acid sequence set forth in SEQ ID NO: 87, or the amino acid sequence set forth in SEQ ID NO: 87 comprising 1, 2, or 3 amino acid modifications (e.g., substitution, deletion, addition, etc.); the amino acid sequence of VL CDR2 comprises or consists of the amino acid sequence set forth in SEQ ID NO: 88, or the amino acid sequence set forth in SEQ ID NO: 88 comprising 1, 2, or 3 amino acid modifications (e.g., substitution, deletion, addition, etc.); and the amino acid sequence of VL CDR3 comprises or consists of the amino acid sequence set forth in SEQ ID NO: 89, or the amino acid sequence set forth in SEQ ID NO: 89 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 90. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 91. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 90; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 91.

### 5.2.3 CD3 Binding Domains

The CD3 complex is a set of polypeptides that function together with the T-cell receptor (TCR) to activate T-cells. The CD3 complex comprises two CD3ε polypeptides, two CD3ζ polypeptides, a CD3δ polypeptide, and a CD3γ polypeptide. The intracellular tails of each of the CD3 chains (CD3ε, CD3ζ, CD3γ, and CD3δ) contains at least one single conserved motif known as an immunoreceptor tyrosine-based activation motif (ITAM), which is essential for the signaling capacity of the TCR.

The amino acid sequence of a reference immature hCD3F polypeptide and mature hCD3F polypeptide is set forth in SEQ ID NOS: 21 and 22, respectively. The amino acid sequence of a reference immature hCD3ζ polypeptide and mature hCD3ζ polypeptide is set forth in SEQ ID NOS: 23 and 24, respectively. The amino acid sequence of a reference immature hCD3γ polypeptide and mature hCD3γ polypeptide is set forth in SEQ ID NOS: 25 and 26, respectively. The amino acid sequence of a reference immature hCD3δ polypeptide and mature hCD3δ polypeptide is set forth in SEQ ID NOS: 27 and 28, respectively. See Table 3, herein.

TABLE 3
The Amino Acid Sequence of Reference hCD3 Polypeptides

| Description | Amino Acid Sequence | SEQ ID NO |
|---|---|---|
| hCD3ε | MQSGTHWRVLGLCLLSVGVWGQDGNEEMGGITQTPYKV | 21 |
| Immature- | SISGTTVILTCPQYPGSEILWQHNDKNIGGDEDDKNIG | |
| Signal | SDEDHLSLKEFSELEQSGYYVCYPRGSKPEDANFYLYL | |
| Peptide | RARVCENCMEMDVMSVATIVIVDICITGGLLLLVYYWS | |
| Underlined | KNRKAKAKPVTRGAGAGGRQRGQNKERPPPVPNPDYEP | |
| | IRKGQRDLYSGLNQRRI | |
| hCD3ε | DGNEEMGGITQTPYKVSISGTTVILTCPQYPGSEILWQ | 22 |
| Mature-No | HNDKNIGGDEDDKNIGSDEDHLSLKEFSELEQSGYYVC | |
| Signal | YPRGSKPEDANFYLYLRARVCENCMEMDVMSVATIVIV | |
| Peptide | DICITGGLLLLVYYWSKNRKAKAKPVTRGAGAGGRQRG | |
| | QNKERPPPVPNPDYEPIRKGQRDLYSGLNQRRI | |
| hCD3ζ | MKWKALFTAAILQAQLPITEAQSFGLLDPKLCYLLDGI | 23 |
| Immature- | LFIYGVILTALFLRVKFSRSADAPAYQQGQNQLYNELN | |
| Signal | LGRREEYDVLDKRRGRDPEMGGKPQRRKNPQEGLYNEL | |
| Peptide | QKDKMAEAYSEIGMKGERRRGKGHDGLYQGLSTATKDT | |
| Underlined | YDALHMQALPPR | |
| hCD3ζ | QSFGLLDPKLCYLLDGILFIYGVILTALFLRVKFSRSA | 24 |
| Mature-No | DAPAYQQGQNQLYNELNLGRREEYDVLDKRRGRDPEMG | |
| Signal | GKPQRRKNPQEGLYNELQKDKMAEAYSEIGMKGERRRG | |
| Peptide | KGHDGLYQGLSTATKDTYDALHMQALPPR | |
| hCD3γ | MEQGKGLAVLILAIILLQGTLAQSIKGNHLVKVYDYQE | 25 |
| Immature- | DGSVLLTCDAEAKNITWFKDGKMIGFLTEDKKKWNLGS | |
| Signal | NAKDPRGMYQCKGSQNKSKPLQVYYRMCQNCIELNAAT | |
| Peptide | ISGFLFAEIVSIFVLAVGVYFIAGQDGVRQSRASDKQT | |
| Underlined | LLPNDQLYQPLKDREDDQYSHLQGNQLRRN | |
| hCD3γ | QSIKGNHLVKVYDYQEDGSVLLTCDAEAKNITWFKDGK | 26 |
| Mature-No | MIGFLTEDKKKWNLGSNAKDPRGMYQCKGSQNKSKPLQ | |
| Signal | VYYRMCQNCIELNAATISGFLFAEIVSIFVLAVGVYFI | |
| Peptide | AGQDGVRQSRASDKQTLLPNDQLYQPLKDREDDQYSHL | |
| | QGNQLRRN | |
| hCD3δ | MEHSTFLSGLVLATLLSQVSPFKIPIEELEDRVFVNCN | 27 |
| Immature- | TSITWVEGTVGTLLSDITRLDLGKRILDPRGIYRCNGT | |
| Signal | DIYKDKESTVQVHYRMCQSCVELDPATVAGIIVTDVIA | |
| Peptide | TLLLALGVFCFAGHETGRLSGAADTQALLRNDQVYQPL | |
| Underlined | RDRDDAQYSHLGGNWARNK | |
| hCD3δ | FKIPIEELEDRVFVNCNTSITWVEGTVGTLLSDITRLD | 28 |
| Mature-No | LGKRILDPRGIYRCNGTDIYKDKESTVQVHYRMCQSCV | |
| Signal | ELDPATVAGIIVTDVIATLLLALGVFCFAGHETGRLSG | |
| Peptide | AADTQALLRNDQVYQPLRDRDDAQYSHLGGNWARNK | |

The multispecific proteins described herein comprise an antigen binding domain that specifically binds hCD3, also referred to herein as a hCD3 binding domain or an anti-hCD3 antibody (or functional fragment or variant thereof). In particular embodiments, the hCD3 binding domain is a scFv (see, e.g., § 5.2.1). In particular embodiments, the hCD3 binding domain is a Fab (see, e.g., § 5.2.1). In particular embodiments, the multispecific protein is monovalent for hCD3.

In some embodiments, the hCD3 binding domain specifically binds to hCD3c. In some embodiments, the hCD3 binding domain specifically binds to hCD3δ. In some embodiments, the hCD3 binding domain specifically binds to hCD3γ. In some embodiments, the hCD3 binding domain specifically binds to hCD3.

Exemplary hCD3 binding domains that can be employed in multispecific proteins described herein are described in § 5.2.3, Table 4.

hCD3 binding domains that can be employed in multispecific proteins described herein are known in the art, and include, for example, those described in, WO2023044402A1, WO2023014809A2, WO2020247929, U.S. Pat. No. 8,530,629, US20220041721A1, U.S. Pat. No. 7,820,166B2, U.S. Pat. No. 8,101,722, WO2021121215A1, US20220010015A1, WO2021240388A1, WO2021141996A1, WO2021063330A1, US20230212289A1, US20210054077A1, WO2020204708A1, WO2023044402A1, WO2020157210A1, US20210317213A1, WO2023014809A2, WO2023273914A1, WO2022170740A1, US20210269525A1, US20210277118A1, US20200048349A1, US20190382486A1, U.S. Ser. No. 11/203,646B2, US20210155694A1, US20200339686A1, US20200317779A1, US20200299384A1, US20200157217A1, US20200377594A1, US20200115449, WO2017053856A1, US20180273622A1, US20190284278A1, U.S. Pat. No. 9,914,777B2, U.S. Ser. No. 11/007,267B2, US20210054087A1, U.S. Ser. No. 10/066,015B2, U.S. Ser. No. 10/669,337B2, U.S. Pat. No. 9,611,325, US20230002487A1, U.S. Ser. No. 11/603,405B2, U.S. Ser. No. 11/639,388B2, U.S. Ser. No. 10/407,501B2, U.S. Ser. No. 10/640,572B2, U.S. Ser. No. 10/968,276B2, U.S. Ser. No. 10/000,567B2, U.S. Ser. No. 10/202,452B2, U.S. Ser. No. 10/150,812B2, U.S. Pat. No. 7,728,114B2, US20190359712A1, US20200048348A1, U.S. Ser. No. 10/066,016, the entire contents of each of which is incorporated by reference herein for all purposes.

Exemplary hCD3 binding domains known in the art that can be employed in fusion proteins and polypeptides described herein also, include, OKT3 (also known as muromonab), SP34, otelixizumab, teplizumab, visilizumab, catumaxomab, blinatumomab, and foralumab; and chimeric or humanized versions of any of the foregoing, e.g., a humanized version of OKT3, a humanized version of SP34. The anti-hCD3 antibody referred to herein as OKT3 (see, e.g., Table 4) specifically binds to hCD3. The anti-hCD3 antibody referred to herein as SP34 (see, e.g., Table 4) specifically binds to hCD3.

The amino acid sequence of exemplary hCD3 binding domains that can be utilized in the multispecific proteins described herein is provided in Table 4. The CDRs of the hCD3 binding domains in Table 4, are denoted according to Kabat. A person of ordinary skill in the art would be able to determine the CDRs as defined by another scheme, e.g., Chothia, IMGT, using ordinary methods known in the art.

TABLE 4
The Amino Acid Sequence of Exemplary hCD3 Binding Domains

| | | SEQ ID | |
|---|---|---|---|
| Description | | NO | Amino Acid Sequence |
| OKT3 | VH CDR1 | 29 | RYTMH |
| | VH CDR2 | 30 | YINPSRGYTNYNQKFKD |
| | VH CDR3 | 31 | YYDDHYCLDY |
| | VL CDR1 | 32 | SASSSVSYMN |
| | VL CDR2 | 33 | DTSKLAS |
| | VL CDR3 | 34 | QQWSSNPFT |
| | VH | 35 | QVQLVQSGGGVVQPGRSLRLSCKASGYTFTRYTMHWV |
| | | | RQAPGKGLEWIGYINPSRGYTNYNQKFKDRFTISRDN |
| | | | SKNTAFLQMDSLRPEDIGVYFCARYYDDHYSLDYWGQ |
| | | | GTPVTVSS |
| | VL | 36 | DIQMTQSPSSLSASVGDRVTITCSASSSVSYMNWYQQ |
| | | | TPGKAPKRWIYDTSKLASGVPSRFSGSGSGTDYTFTI |
| | | | SSLQPEDIATYYCQQWSSNPFTFGQGTKLQITR |
| SP34 | VH CDR1 | 39 | KYAMN |
| | VH CDR2 | 40 | RIRSKYNNYATYYADSVKD |
| | VH CDR3 | 41 | HGNFGNSYISYWAY |
| | VL CDR1 | 42 | GSSTGAVTSGYYPN |
| | VL CDR2 | 43 | GTKFLAP |
| | VL CDR3 | 44 | ALWYSNRWV |
| | VH | 45 | EVQLVESGGGLVQPGGSLKLSCAASGFTENKYAMNWV |
| | | | RQAPGKGLEWVARIRSKYNNYATYYADSVKDRFTISR |
| | | | DDSKNTAYLQMNNLKTEDTAVYYCVRHGNFGNSYISY |
| | | | WAYWGQGTLVTVSS |
| | VH-C | 46 | EVQLVESGGGLVQPGGSLKLSCAASGFTENKYAMNWV |
| | | | RQAPGKCLEWVARIRSKYNNYATYYADSVKDRFTISR |
| | | | DDSKNTAYLQMNNLKTEDTAVYYCVRHGNFGNSYISY |
| | | | WAYWGQGTLVTVSS |
| | VL | 47 | QTVVTQEPSLTVSPGGTVTLTCGSSTGAVTSGYYPNW |
| | | | VQQKPGQAPRGLIGGTKFLAPGTPARFSGSLLGGKAA |
| | | | LTLSGVQPEDEAEYYCALWYSNRWVFGGGTKLTVL |
| | VL-C | 48 | QTVVTQEPSLTVSPGGTVTLTCGSSTGAVTSGYYPNW |
| | | | VQQKPGQAPRGLIGGTKFLAPGTPARFSGSLLGGKAA |
| | | | LTLSGVQPEDEAEYYCALWYSNRWVFGCGTKLTVL |
| S2 | VH CDR1 | 49 | KAWMH |
| | VH CDR2 | 50 | QIKDKSNSYATYYADSVKG |
| | VH CDR3 | 51 | VYYALSPFDY |
| | VL CDR1 | 52 | KSSQSLVHNNANTYLS |
| | VL CDR2 | 53 | KVSNRFS |
| | VL CDR3 | 54 | GQGTQYPFT |
| | VH | 55 | QVQLVESGGGVVQPGRSLRLSCAASGFTFTKAWMHWV |
| | | | RQAPGKQLEWVAQIKDKSNSYATYYADSVKGRFTISR |
| | | | DDSKNTLYLQMNSLRAEDTAVYYCRGVYYALSPFDYW |
| | | | GQGTLVTVSS |
| | VL | 56 | DIVMTQTPLSLSVTPGQPASISCKSSQSLVHNNANTY |
| | | | LSWYLQKPGQSPQSLIYKVSNRFSGVPDRFSGSGSGT |
| | | | DFTLKISRVEAEDVGVYYCGQGTQYPFTFGSGTKVEI |
| | | | K |
| Anti-CD3 A | VH CDR1 | 351 | TYAMN |
| | VH CDR2 | 352 | RIRSKYNNYATYYADSVKD |
| | VH CDR3 | 353 | HGNFGNSYVSWFAY |
| | VL CDR1 | 354 | RSSTGAVTTSNYAN |
| | VL CDR2 | 355 | GTNKRAP |
| | VL CDR3 | 356 | ALWYSNLWV |
| | VH | 357 | EVQLVESGGGLVQPGGSLRLSCAASGFTFSTYAMNWV |
| | | | RQAPGKGLEWVARIRSKYNNYATYYADSVKDRFTISR |
| | | | DDSKNSLYLQMNSLRAEDTAVYYCARHGNFGNSYVSW |
| | | | FAYWGQGTLVTVSS |
| | VL | 358 | EIVLTQSPATLSLSPGERATLSCRSSTGAVTTSNYAN |
| | | | WVQQKPGQAPRGLIGGTNKRAPGVPARFSGSLSGTDA |
| | | | TLTISSLQPEDFAVYYCALWYSNLWVFGGGTKVEIK |
| Anti-CD3 B | VH CDR1 | 359 | NNNAAWS |
| | VH CDR2 | 360 | RTYYRSKWLYDYAVSVKS |
| | VH CDR3 | 361 | GYSSSFDY |
| | VL CDR1 | 362 | TGTSSNIGTYKFVS |
| | VL CDR2 | 363 | EVSKRPS |
| | VL CDR3 | 364 | VSYAGSGTLL |
| | VH | 365 | QVQLQQSGPRLVRPSQTLSLTCAISGDSVENNNAAWS |
| | | | WIRQSPSRGLEWLGRTYYRSKWLYDYAVSVKSRITVN |
| | | | PDTSRNQFTLQLNSVTPEDTALYYCARGYSSSFDYWG |
| | | | QGTLVTVSS |
| | VL | 366 | QSALTQPASVSGSPGQSITISCTGTSSNIGTYKFVSW |
| | | | YQQHPDKAPKVLLYEVSKRPSGVSSRFSGSKSGNTAS |
| | | | LTISGLQAEDQADYHCVSYAGSGTLLFGGGTKLTVL |

In some embodiments, the hCD3 binding domain comprises a hCD3 binding domain provided in Table 4.

In some embodiments, the hCD3 binding domain comprises a VH that comprises: VH CDR1, VH CDR2, and VH CDR3.

In some embodiments, the amino acid sequence of VH CDR1 comprises or consists of the amino acid sequence of a VH CDR1 of a VH set forth in Table 4, or the amino acid sequence of a VH CDR1 of a VH set forth in Table 4 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 of a VH set forth in Table 4, or the amino acid sequence of a VH CDR2 of a VH set forth in Table 4 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 of a VH set forth in Table 4, or the amino acid sequence of a VH CDR3 of a VH set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 of a VH set forth in Table 4, or the amino acid sequence of a VH CDR1 of a VH set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 4, or the amino acid sequence of a VH CDR2 of a VH set forth in Table 4 comprising or consisting of 1, 2, or 3 amino acid modifications (e.g., substitution, deletion, addition, etc.); and the amino acid sequence of VH CDR3 comprises or consists of the amino acid sequence of a VH CDR3 of a VH set forth in Table 4, or the amino acid sequence of a VH CDR3 of a VH set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 4, or the amino acid sequence of a VH CDR1 set forth in Table 4 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 set forth in Table 4, or the amino acid sequence of a VH CDR2 set forth in Table 4 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 set forth in Table 4, or the amino acid sequence of a VH CDR3 set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 4, or the amino acid sequence of a VH CDR1 set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 4, or the amino acid sequence of a VH CDR2 set forth in Table 4 comprising or consisting of 1, 2, or 3 amino acid modifications (e.g., substitution, deletion, addition, etc.); and the amino acid sequence of VH CDR3 comprises or consists of the amino acid sequence of a VH CDR3 set forth in Table 4, or the amino acid sequence of a VH CDR3 set forth in Table 4 comprising or consisting of 1, 2, or 3 amino acid modifications (e.g., substitution, deletion, addition, etc.).

In some embodiments, the hCD3 binding domain comprises a VL that comprises: VL CDR1, VL CDR2, and VL CDR3.

In some embodiments, the amino acid sequence of VL CDR1 comprises or consists of the amino acid sequence of a VL CDR1 of a VL set forth in Table 4, or the amino acid sequence of a VL CDR1 of a VL set forth in Table 4 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 of a VL set forth in Table 4, or the amino acid sequence of a VL CDR2 of a VL set forth in Table 4 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 of a VL set forth in Table 4, or the amino acid sequence of a VL CDR3 of a VL set forth in Table 4 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 of a VL set forth in Table 4, or the amino acid sequence of a VL CDR1 of a VL set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 4, or the amino acid sequence of a VL CDR2 of a VL set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 4, or the amino acid sequence of a VL CDR3 of a VL set forth in Table 4 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 4, or the amino acid sequence of a VL CDR1 set forth in Table 4 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 set forth in Table 4, or the amino acid sequence of a VL CDR2 set forth in Table 4 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 set forth in Table 4, or the amino acid sequence of a VL CDR3 set forth in Table 4 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 4, or the amino acid sequence of a VL CDR1 set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 4, or the amino acid sequence of a VL CDR2 set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 4, or the amino acid sequence of a VL CDR3 set forth in Table 4 comprising or consisting of 1, 2, or 3 amino acid modifications (e.g., substitution, deletion, addition, etc.).

In some embodiments, the hCD3 binding domain comprises a VH that comprises: VH CDR1, VH CDR2, and VH CDR3; and a VL that comprises: VL CDR1, VL CDR2, and VL CDR3.

In some embodiments, the amino acid sequence of VH CDR1 comprises or consists of the amino acid sequence of a VH CDR1 of a VH set forth in Table 4, or the amino acid sequence of a VH CDR1 of a VH set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 4, or the amino acid sequence of a VH CDR2 of a VH set forth in Table 4 comprising or consisting of 1, 2, or 3 amino acid modifications (e.g., substitution, deletion, addition, etc.); the amino acid sequence of VH CDR3 comprises or consists of the amino acid sequence of a VH CDR3 of a VH set forth in Table 4, or the amino acid sequence of a VH CDR3 of a VH set forth in Table 4 comprising or consisting of 1, 2, or 3 amino acid modifications (e.g., substitution, deletion, addition, etc.); the amino acid sequence of VL CDR1 comprises or consists of the amino acid sequence of a VL CDR1 of a VL set forth in Table 4, or the amino acid sequence of a VL CDR1 of a VL set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 4, or the amino acid sequence of a VL CDR2 of a VL set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 4, or the amino acid sequence of a VL CDR3 of a VL set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 4, or the amino acid sequence of a VH CDR1 set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 4, or the amino acid sequence of a VH CDR2 set forth in Table 4 comprising or consisting of 1, 2, or 3 amino acid modifications (e.g., substitution, deletion, addition, etc.); the amino acid sequence of VH CDR3 comprises or consists of the amino acid sequence of a VH CDR3 set forth in Table 4, or the amino acid sequence of a VH CDR3 set forth in Table 4 comprising or consisting of 1, 2, or 3 amino acid modifications (e.g., substitution, deletion, addition, etc.); the amino acid sequence of VL CDR1 comprises or consists of the amino acid sequence of a VL CDR1 set forth in Table 4, or the amino acid sequence CDR1 of a VL set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 4, or the amino acid sequence of a VL CDR2 set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 4, or the amino acid sequence of a VL CDR3 set forth in Table 4 comprising or consisting of 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 4. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 4. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 4; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 4.

In some embodiments, the amino acid sequence of VH CDR1 comprises or consists of the amino acid sequence set forth in SEQ ID NO: 29, or the amino acid sequence set forth in SEQ ID NO: 29 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 30, or the amino acid sequence set forth in SEQ ID NO: 30 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 31, or the amino acid sequence set forth in SEQ ID NO: 31 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 29, or the amino acid sequence set forth in SEQ ID NO: 29 comprising 1, 2, or 3 amino acid modifications (e.g., substitution, deletion, addition, etc.); the amino acid sequence of VH CDR2 comprises or consists of the amino acid sequence set forth in SEQ ID NO: 30, or the amino acid sequence set forth in SEQ ID NO: 30 comprising 1, 2, or 3 amino acid modifications (e.g., substitution, deletion, addition, etc.); and the amino acid sequence of VH CDR3 comprises or consists of the amino acid sequence set forth in SEQ ID NO: 31, or the amino acid sequence set forth in SEQ ID NO: 31 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 32, or the amino acid sequence set forth in SEQ ID NO: 32 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 33, or the amino acid sequence set forth in SEQ ID NO: 33 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 34, or the amino acid sequence set forth in SEQ ID NO: 34 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 32, or the amino acid sequence set forth in SEQ ID NO: 32 comprising 1, 2, or 3 amino acid modifications (e.g., substitution, deletion, addition, etc.); the amino acid sequence of VL CDR2 comprises or consists of the amino acid sequence set forth in SEQ ID NO: 33, or the amino acid sequence set forth in SEQ ID NO: 33 comprising 1, 2, or 3 amino acid modifications (e.g., substitution, deletion, addition, etc.); and the amino acid sequence of VL CDR3 comprises or consists of the amino acid sequence set forth in SEQ ID NO: 34, or the amino acid sequence set forth in SEQ ID NO: 34 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 29, or the amino acid sequence set forth in SEQ ID NO: 29 comprising 1, 2, or 3 amino acid modifications (e.g., substitution, deletion, addition, etc.); the amino acid sequence of VH CDR2 comprises or consists of the amino acid sequence set forth in SEQ ID NO: 30, or the amino acid sequence set forth in SEQ ID NO: 30 comprising 1, 2, or 3 amino acid modifications (e.g., substitution, deletion, addition, etc.); the amino acid sequence of VH CDR3 comprises or consists of the amino acid sequence set forth in SEQ ID NO: 31, or the amino acid sequence set forth in SEQ ID NO: 31 comprising 1, 2, or 3 amino acid modifications (e.g., substitution, deletion, addition, etc.); the amino acid sequence of VL CDR1 comprises or consists of the amino acid sequence set forth in SEQ ID NO: 32, or the amino acid sequence set forth in SEQ ID NO: 32 comprising 1, 2, or 3 amino acid modifications (e.g., substitution, deletion, addition, etc.); the amino acid sequence of VL CDR2 comprises or consists of the amino acid sequence set forth in SEQ ID NO: 33, or the amino acid sequence set forth in SEQ ID NO: 33 comprising 1, 2, or 3 amino acid modifications (e.g., substitution, deletion, addition, etc.); and the amino acid sequence of VL CDR3 comprises or consists of the amino acid sequence set forth in SEQ ID NO: 34, or the amino acid sequence set forth in SEQ ID NO: 34 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 35. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 36. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 35; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 36.

In some embodiments, the amino acid sequence of VH CDR1 comprises or consists of the amino acid sequence set forth in SEQ ID NO: 39, or the amino acid sequence set forth in SEQ ID NO: 39 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 40, or the amino acid sequence set forth in SEQ ID NO: 40 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 41, or the amino acid sequence set forth in SEQ ID NO: 41 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 39, or the amino acid sequence set forth in SEQ ID NO: 39 comprising 1, 2, or 3 amino acid modifications (e.g., substitution, deletion, addition, etc.); the amino acid sequence of VH CDR2 comprises or consists of the amino acid sequence set forth in SEQ ID NO: 40, or the amino acid sequence set forth in SEQ ID NO: 40 comprising 1, 2, or 3 amino acid modifications (e.g., substitution, deletion, addition, etc.); and the amino acid sequence of VH CDR3 comprises or consists of the amino acid sequence set forth in SEQ ID NO: 41, or the amino acid sequence set forth in SEQ ID NO: 41 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 42, or the amino acid sequence set forth in SEQ ID NO: 42 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 43, or the amino acid sequence set forth in SEQ ID NO: 43 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 42, or the amino acid sequence set forth in SEQ ID NO: 42 comprising 1, 2, or 3 amino acid modifications (e.g., substitution, deletion, addition, etc.); the amino acid sequence of VL CDR2 comprises or consists of the amino acid sequence set forth in SEQ ID NO: 43, or the amino acid sequence set forth in SEQ ID NO: 43 comprising 1, 2, or 3 amino acid modifications (e.g., substitution, deletion, addition, etc.); and the amino acid sequence of VL CDR3 comprises or consists of the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 39, or the amino acid sequence set forth in SEQ ID NO: 39 comprising 1, 2, or 3 amino acid modifications (e.g., substitution, deletion, addition, etc.); the amino acid sequence of VH CDR2 comprises or consists of the amino acid sequence set forth in SEQ ID NO: 40, or the amino acid sequence set forth in SEQ ID NO: 40 comprising 1, 2, or 3 amino acid modifications (e.g., substitution, deletion, addition, etc.); the amino acid sequence of VH CDR3 comprises or consists of the amino acid sequence set forth in SEQ ID NO: 41, or the amino acid sequence set forth in SEQ ID NO: 41 comprising 1, 2, or 3 amino acid modifications (e.g., substitution, deletion, addition, etc.); the amino acid sequence of VL CDR1 comprises or consists of the amino acid sequence set forth in SEQ ID NO: 42, or the amino acid sequence set forth in SEQ ID NO: 42 comprising 1, 2, or 3 amino acid modifications (e.g., substitution, deletion, addition, etc.); the amino acid sequence of VL CDR2 comprises or consists of the amino acid sequence set forth in SEQ ID NO: 43, or the amino acid sequence set forth in SEQ ID NO: 43 comprising 1, 2, or 3 amino acid modifications (e.g., substitution, deletion, addition, etc.); and the amino acid sequence of VL CDR3 comprises or consists of the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45; and the VH comprises a cysteine is introduced into a framework region of the VH region (see, e.g., § 5.2.1.5). In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47 and the VL comprises a cysteine is introduced into a framework region of the VL region (see, e.g., § 5.2.1.5). In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45; and the VH comprises a cysteine is introduced into a framework region of the VH region (see, e.g., § 5.2.1.5); and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47; and the VL comprises a cysteine is introduced into a framework region of the VL region (see, e.g., § 5.2.1.5).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46; and the VH comprises a cysteine is introduced into a framework region of the VH region (see, e.g., § 5.2.1.5). In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48 and the VL comprises a cysteine is introduced into a framework region of the VL region (see, e.g., § 5.2.1.5). In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46; and the VH comprises a cysteine is introduced into a framework region of the VH region (see, e.g., § 5.2.1.5); and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48; and the VL comprises a cysteine is introduced into a framework region of the VL region (see, e.g., § 5.2.1.5).

In some embodiments, the amino acid sequence of VH CDR1 comprises or consists of the amino acid sequence set forth in SEQ ID NO: 49, or the amino acid sequence set forth in SEQ ID NO: 49 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 50, or the amino acid sequence set forth in SEQ ID NO: 50 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 51, or the amino acid sequence set forth in SEQ ID NO: 51 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 49, or the amino acid sequence set forth in SEQ ID NO: 49 comprising 1, 2, or 3 amino acid modifications (e.g., substitution, deletion, addition, etc.); the amino acid sequence of VH CDR2 comprises or consists of the amino acid sequence set forth in SEQ ID NO: 50, or the amino acid sequence set forth in SEQ ID NO: 50 comprising 1, 2, or 3 amino acid modifications (e.g., substitution, deletion, addition, etc.); and the amino acid sequence of VH CDR3 comprises or consists of the amino acid sequence set forth in SEQ ID NO: 51, or the amino acid sequence set forth in SEQ ID NO: 51 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 52, or the amino acid sequence set forth in SEQ ID NO: 52 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 53, or the amino acid sequence set forth in SEQ ID NO: 53 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 54, or the amino acid sequence set forth in SEQ ID NO: 54 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 52, or the amino acid sequence set forth in SEQ ID NO: 52 comprising 1, 2, or 3 amino acid modifications (e.g., substitution, deletion, addition, etc.); the amino acid sequence of VL CDR2 comprises or consists of the amino acid sequence set forth in SEQ ID NO: 53, or the amino acid sequence set forth in SEQ ID NO: 53 comprising 1, 2, or 3 amino acid modifications (e.g., substitution, deletion, addition, etc.); and the amino acid sequence of VL CDR3 comprises or consists of the amino acid sequence set forth in SEQ ID NO: 54, or the amino acid sequence set forth in SEQ ID NO: 54 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 49, or the amino acid sequence set forth in SEQ ID NO: 49 comprising 1, 2, or 3 amino acid modifications (e.g., substitution, deletion, addition, etc.); the amino acid sequence of VH CDR2 comprises or consists of the amino acid sequence set forth in SEQ ID NO: 50, or the amino acid sequence set forth in SEQ ID NO: 50 comprising 1, 2, or 3 amino acid modifications (e.g., substitution, deletion, addition, etc.); the amino acid sequence of VH CDR3 comprises or consists of the amino acid sequence set forth in SEQ ID NO: 51, or the amino acid sequence set forth in SEQ ID NO: 51 comprising 1, 2, or 3 amino acid modifications (e.g., substitution, deletion, addition, etc.); the amino acid sequence of VL CDR1 comprises or consists of the amino acid sequence set forth in SEQ ID NO: 52, or the amino acid sequence set forth in SEQ ID NO: 52 comprising 1, 2, or 3 amino acid modifications (e.g., substitution, deletion, addition, etc.); the amino acid sequence of VL CDR2 comprises or consists of the amino acid sequence set forth in SEQ ID NO: 53, or the amino acid sequence set forth in SEQ ID NO: 53 comprising 1, 2, or 3 amino acid modifications (e.g., substitution, deletion, addition, etc.); and the amino acid sequence of VL CDR3 comprises or consists of the amino acid sequence set forth in SEQ ID NO: 54, or the amino acid sequence set forth in SEQ ID NO: 54 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 55. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 56. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 55; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 56.

### 5.2.4 Tumor Associated Antigen Binding Domains

The multispecific proteins described herein comprise a binding domain that specifically binds to a human tumor associated antigen (hTAA) (also referred to herein as tumor associated antigen (hTAA) binding domains and anti-tumor associated antigen (hTAA) antibodies) (or functional fragments or variants there).

In some embodiments, the tumor associated antigen is a tumor associated antigen of a solid tumor. In some embodiments, the solid tumor is of the breast, ovary, endometrium, uterus, cervix, anus, prostate, rectum, kidney, bladder, colon, liver, pancreas, thyroid, thymus, lung, bronchus, skin, brain, spinal cord, head, neck, lip, or oral cavity.

In some embodiments, the tumor associated antigen is mesothelin (MSLN), prostate specific membrane antigen (PMSA), epidermal growth factor receptor (EGFR), Human Epidermal Growth Factor Receptor 2 (HER2), CD19; membrane spanning 4-domains A1 (MS4A1; CD20); CD22 (SIGLEC2); CD27 (TNFRSF7); TNFRSF8 (CD30); CD33 (SIGLEC3); CD37; CD38; CD40 (TNFRSF5), CD44; CD47; CD48 (SLAMF2); CD52; CD70 (TNFSF7; CD27L); 5′-nucleotidase ecto (NT5E; CD73), ectonucleoside triphosphate diphosphohydrolase 1 (CD39), CD74; CD79B; CD80; CD86; interleukin 3 receptor subunit alpha (IL3RA), prominin 1 (PROM1; CD133); TNFRSF9 (CD137); syndecan 1 (SDC1; CD138); CD200 molecule (CD200); alpha fetoprotein (AFP), BAG cochaperone 6 (BAG6); MET proto-oncogene, receptor tyrosine kinase (MET); KIT proto-oncogene, receptor tyrosine kinase (KIT); C-type lectin domain family 12 member A (CLEC12A; CD371); C-type lectin domain containing 9A (CLEC9A; CD370); cadherin 3 (CDH3); carbonic anhydrase 6 (CAVI); carbonic anhydrase 9 (CAIX); carcinoembryonic antigen related cell adhesion molecule 3 (CEACAM3); carcinoembryonic antigen related cell adhesion molecule 5 (CEACAM5); carcinoembryonic antigen related cell adhesion molecule 6 (CEACAM6); chorionic somatomammotropin hormone 1 (CSH1); coagulation factor III, tissue factor (F3); collectin subfamily member 10 (COLEC10; CLL1); delta like canonical Notch ligand 3 (DLL3); ectonucleotide pyrophosphatase/phosphodiesterase 3 (ENPP3); ephrin A1 (EFNA1); epidermal growth factor receptor (EGFR; ERBB; HER1); EGFR variant III (EGFRvIII); EPH receptor A2 (EPHA2); epithelial cell adhesion molecule (EPCAM); erb-b2 receptor tyrosine kinase 2 (ERBB2; HER-2/neu); fibroblast activation protein alpha (FAP); fibroblast growth factor receptor 2 (FGFR2); fibroblast growth factor receptor 3 (FGFR3); folate hydrolase 1 (FOLH1); folate receptor 1 (FOLR1); GD2 ganglioside; glycoprotein NMB (GPNMB; osteoactivin); guanylate cyclase 2C (GUCY2C); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G); MHC class I polypeptide-related sequence A (MICA); MHC class I polypeptide-related sequence B (MICB); integrin subunit beta 7 (ITGB7); leukocyte immunoglobulin like receptor B1 (LILRB1; ILT2); leukocyte immunoglobulin like receptor B2 (LILRB2; ILT4); LY6/PLAUR domain containing 3 (LYPD3); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member All (MAGEA11); MAGE family member C1 (MAGEC1); MAGE family member C2 (MAGEC2); MAGE family member C3 (MAGEC3); MAGE family member D1 (MAGED1); MAGE family member D2 (MAGED2); mesothelin (MSLN); mucin 1 (MUC1) and splice variants thereof (e.g., including MUC1/A, C, D, X, Y, Z and REP); mucin 16 (MUC16; CA125); natural killer cell cytotoxicity receptor 3 ligand 1 (NCR3LG1; B7-H6); necdin, MAGE family member (NDN); nectin cell adhesion molecule 2 (NECTIN2); nectin cell adhesion molecule 4 (NECTIN4); SLIT and NTRK like family member 6 (SLITRK6); promyelocytic leukemia (PML); protein tyrosine kinase 7 (inactive) (PTK7); Poliovirus receptor (PVR) cell adhesion molecule (PVR); SLAM family member 6 (SLAMF6); SLAM family member 7 (SLAMF7); sialic acid binding Ig like lectin 7 (SIGLEC7); sialic acid binding Ig like lectin 9 (SIGLEC9); sialic acid binding Ig like lectin 10 (SIGLEC10); signal regulatory protein alpha (SIRPA) solute carrier family 34 (sodium phosphate), member 2 (SLC34A2); solute carrier family 39 member 6 (SLC39A6); STEAP family member 1 (STEAP1); suppression of tumorigenicity 2 (ST2); TNF receptor superfamily member 4 (TNFRSF4; OX40); TNF superfamily member 9 (TNFSF9; 4-1BB-L, CD137L); TNFRSF10A (DR4, TRAILR1); TNFRSF10B (DR5, TRAILR2); TNFRSF13B (BAFF); TNFRSF17 (BCMA); TNFRSF18 (GITR); transferrin (TF); transforming growth factor beta 1 (TGFB1) and isoforms thereof; triggering receptor expressed on myeloid cells 1 (TREM1); triggering receptor expressed on myeloid cells 2 (TREM2); trophoblast glycoprotein (TPBG); trophinin (TRO); tumor associated calcium signal transducer 2 (TACSTD2); Fucosyl GM1; sialyl Lewis adhesion molecule (sLe); and Lewis Y antigen.

In some embodiments, the tumor associated antigen is MSLN, PMSA, EGFR, or HER2. In some embodiments, the tumor associated antigen is human MSLN (hMSLN), human EGFR (hEGFR), human HER2 (hHER2), or human prostate specific membrane antigen (hPSMA).

The amino acid sequence of a reference mature hMSLN polypeptide is set forth in SEQ ID NO: 57. The amino acid sequence of a reference mature hEGFR polypeptide is set forth in SEQ ID NO: 58. The amino acid sequence of a reference mature hHER2 polypeptide is set forth in SEQ ID NO: 59. The amino acid sequence of a reference mature hPSMA polypeptide is set forth in SEQ ID NO: 92. Different isoforms of the proteins set forth in Table 5 may be more highly expressed by abnormal (e.g., cancer) cells relative to the expression of the reference protein. See Table 5, herein.

TABLE 5
The Amino Acid Sequence of Reference Exemplary Human Tumor
associated antigen Polypeptides

| Description | Amino Acid Sequence | SEQ ID NO |
|---|---|---|
| hMSLN | LAGETGQEAAPLDGVLANPPNISSLSPRQLLGFPCAEV | 57 |
| (Mature-No | SGLSTERVRELAVALAQKNVKLSTEQLRCLAHRLSEPP | |
| Signal | EDLDALPLDLLLFLNPDAFSGPQACTRFFSRITKANVD | |
| Peptide) | LLPRGAPERQRLLPAALACWGVRGSLLSEADVRALGGL | |
| | ACDLPGRFVAESAEVLLPRLVSCPGPLDQDQQEAARAA | |
| | LQGGGPPYGPPSTWSVSTMDALRGLLPVLGQPIIRSIP | |
| | QGIVAAWRQRSSRDPSWRQPERTILRPRFRREVEKTAC | |
| | PSGKKAREIDESLIFYKKWELEACVDAALLATQMDRVN | |
| | AIPFTYEQLDVLKHKLDELYPQGYPESVIQHLGYLFLK | |
| | MSPEDIRKWNVTSLETLKALLEVNKGHEMSPQAPRRPL | |
| | PQVATLIDRFVKGRGQLDKDTLDTLTAFYPGYLCSLSP | |
| | EELSSVPPSSIWAVRPQDLDTCDPRQLDVLYPKARLAF | |
| | QNMNGSEYFVKIQSFLGGAPTEDLKALSQQNVSMDLAT | |
| | FMKLRTDAVLPLTVAEVQKLLGPHVEGLKAEERHRPVR | |
| | DWILRQRQDDLDTLGLGLQGGIPNGYLVLDLSMQEALS | |
| hEGFR | LEEKKVCQGTSNKLTQLGTFEDHFLSLQRMENNCEVVL | 58 |
| (Mature-No | GNLEITYVQRNYDLSFLKTIQEVAGYVLIALNTVERIP | |
| Signal | LENLQIIRGNMYYENSYALAVLSNYDANKTGLKELPMR | |
| Peptide) | NLQEILHGAVRFSNNPALCNVESIQWRDIVSSDFLSNM | |
| | SMDFQNHLGSCQKCDPSCPNGSCWGAGEENCQKLTKII | |
| | CAQQCSGRCRGKSPSDCCHNQCAAGCTGPRESDCLVCR | |
| | KFRDEATCKDTCPPLMLYNPTTYQMDVNPEGKYSFGAT | |
| | CVKKCPRNYVVTDHGSCVRACGADSYEMEEDGVRKCKK | |
| | CEGPCRKVCNGIGIGEFKDSLSINATNIKHFKNCTSIS | |
| | GDLHILPVAFRGDSFTHTPPLDPQELDILKTVKEITGF | |
| | LLIQAWPENRTDLHAFENLEIIRGRTKQHGQFSLAVVS | |
| | LNITSLGLRSLKEISDGDVIISGNKNLCYANTINWKKL | |
| | FGTSGQKTKIISNRGENSCKATGQVCHALCSPEGCWGP | |
| | EPRDCVSCRNVSRGRECVDKCNLLEGEPREFVENSECI | |
| | QCHPECLPQAMNITCTGRGPDNCIQCAHYIDGPHCVKT | |
| | CPAGVMGENNTLVWKYADAGHVCHLCHPNCTYGCTGPG | |
| | LEGCPTNGPKIPSIATGMVGALLLLLVVALGIGLFMRR | |
| | RHIVRKRTLRRLLQERELVEPLTPSGEAPNQALLRILK | |
| | ETEFKKIKVLGSGAFGTVYKGLWIPEGEKVKIPVAIKE | |
| | LREATSPKANKEILDEAYVMASVDNPHVCRLLGICLTS | |
| | TVQLITQLMPFGCLLDYVREHKDNIGSQYLLNWCVQIA | |
| | KGMNYLEDRRLVHRDLAARNVLVKTPQHVKITDFGLAK | |
| | LLGAEEKEYHAEGGKVPIKWMALESILHRIYTHQSDVW | |
| | SYGVTVWELMTFGSKPYDGIPASEISSILEKGERLPQP | |
| | PICTIDVYMIMVKCWMIDADSRPKFRELIIEFSKMARD | |
| | PQRYLVIQGDERMHLPSPTDSNFYRALMDEEDMDDVVD | |
| | ADEYLIPQQGFFSSPSTSRTPLLSSLSATSNNSTVACI | |
| | DRNGLQSCPIKEDSFLQRYSSDPTGALTEDSIDDTFLP | |
| | VPEYINQSVPKRPAGSVQNPVYHNQPLNPAPSRDPHYQ | |
| | DPHSTAVGNPEYLNTVQPTCVNSTFDSPAHWAQKGSHQ | |
| | ISLDNPDYQQDFFPKEAKPNGIFKGSTAENAEYLRVAP | |
| | QSSEFIGA | |
| hHER2 | TQVCTGTDMKLRLPASPETHLDMLRHLYQGCQVVQGNL | 59 |
| (Mature-No | ELTYLPTNASLSFLQDIQEVQGYVLIAHNQVRQVPLQR | |
| Signal | LRIVRGTQLFEDNYALAVLDNGDPLNNTTPVTGASPGG | |
| Peptide) | LRELQLRSLTEILKGGVLIQRNPQLCYQDTILWKDIFH | |
| | KNNQLALTLIDTNRSRACHPCSPMCKGSRCWGESSEDC | |
| | QSLTRTVCAGGCARCKGPLPTDCCHEQCAAGCTGPKHS | |
| | DCLACLHFNHSGICELHCPALVTYNTDTFESMPNPEGR | |
| | YTFGASCVTACPYNYLSTDVGSCTLVCPLHNQEVTAED | |
| | GTQRCEKCSKPCARVCYGLGMEHLREVRAVTSANIQEF | |
| | AGCKKIFGSLAFLPESEDGDPASNTAPLQPEQLQVFET | |
| | LEEITGYLYISAWPDSLPDLSVFQNLQVIRGRILHNGA | |
| | YSLTLQGLGISWLGLRSLRELGSGLALIHHNTHLCFVH | |
| | TVPWDQLFRNPHQALLHTANRPEDECVGEGLACHQLCA | |
| | RGHCWGPGPTQCVNCSQFLRGQECVEECRVLQGLPREY | |
| | VNARHCLPCHPECQPQNGSVTCFGPEADQCVACAHYKD | |
| | PPFCVARCPSGVKPDLSYMPIWKFPDEEGACQPCPINC | |
| | THSCVDLDDKGCPAEQRASPLTSIISAVVGILLVVVLG | |
| | VVFGILIKRRQQKIRKYTMRRLLQETELVEPLTPSGAM | |
| | PNQAQMRILKETELRKVKVLGSGAFGTVYKGIWIPDGE | |
| | NVKIPVAIKVLRENTSPKANKEILDEAYVMAGVGSPYV | |
| | SRLLGICLTSTVQLVTQLMPYGCLLDHVRENRGRLGSQ | |
| | DLLNWCMQIAKGMSYLEDVRLVHRDLAARNVLVKSPNH | |
| | VKITDFGLARLLDIDETEYHADGGKVPIKWMALESILR | |
| | RRFTHQSDVWSYGVTVWELMTFGAKPYDGIPAREIPDL | |
| | LEKGERLPQPPICTIDVYMIMVKCWMIDSECRPRFREL | |
| | VSEFSRMARDPQRFVVIQNEDLGPASPLDSTFYRSLLE | |
| | DDDMGDLVDAEEYLVPQQGFFCPDPAPGAGGMVHHRHR | |
| | SSSTRSGGGDLTLGLEPSEEEAPRSPLAPSEGAGSDVF | |
| | DGDLGMGAAKGLQSLPTHDPSPLQRYSEDPTVPLPSET | |
| | DGYVAPLTCSPQPEYVNQPDVRPQPPSPREGPLPAARP | |
| | AGATLERPKTLSPGKNGVVKDVFAFGGAVENPEYLTPQ | |
| | GGAAPQPHPPPAFSPAFDNLYYWDQDPPERGAPPSTEK | |
| | GTPTAENPEYLGLDVPV | |
| hPSMA | MWNLLHETDSAVATARRPRWLCAGALVLAGGFFLLGEL | 92 |
| (Mature-No | FGWFIKSSNEATNITPKHNMKAFLDELKAENIKKFLYN | |
| Signal | FTQIPHLAGTEQNFQLAKQIQSQWKEFGLDSVELAHYD | |
| Peptide) | VLLSYPNKTHPNYISIINEDGNEIFNTSLFEPPPPGYE | |
| | NVSDIVPPFSAFSPQGMPEGDLVYVNYARTEDFFKLER | |
| | DMKINCSGKIVIARYGKVFRGNKVKNAQLAGAKGVILY | |
| | SDPADYFAPGVKSYPDGWNLPGGGVQRGNILNLNGAGD | |
| | PLTPGYPANEYAYRRGIAEAVGLPSIPVHPIGYYDAQK | |
| | LLEKMGGSAPPDSSWRGSLKVPYNVGPGFTGNFSTQKV | |
| | KMHIHSTNEVTRIYNVIGTLRGAVEPDRYVILGGHRDS | |
| | WVFGGIDPQSGAAVVHEIVRSFGTLKKEGWRPRRTILF | |
| | ASWDAEEFGLLGSTEWAEENSRLLQERGVAYINADSSI | |
| | EGNYTLRVDCTPLMYSLVHNLTKELKSPDEGFEGKSLY | |
| | ESWTKKSPSPEFSGMPRISKLGSGNDFEVFFQRLGIAS | |
| | GRARYTKNWETNKFSGYPLYHSVYETYELVEKFYDPMF | |
| | KYHLTVAQVRGGMVFELANSIVLPFDCRDYAVVLRKYA | |
| | DKIYSISMKHPQEMKTYSVSFDSLFSAVKNFTEIASKF | |
| | SERLQDFDKSNPIVLRMMNDQLMFLERAFIDPLGLPDR | |
| | PFYRHVIYAPSSHNKYAGESFPGIYDALFDIESKVDPS | |
| | KAWGEVKRQIYVAAFTVQAAAETLSEVA | |

The multispecific proteins described herein comprise an antigen binding domain that specifically binds a human tumor associated antigen, also referred to herein as a hTAA binding domain or an anti-hTAA antibody (or functional fragment or variant thereof). In particular embodiments, the hTAA binding domain is a full-length antibody, a Fab, or a hIgM CH2 mFab (see, e.g., § 5.2.1). In particular embodiments, the multispecific protein is monovalent for the hTAA. In particular embodiments, the multispecific protein is bivalent for the hTAA.

hHER2 binding domains that can be employed in multispecific proteins described herein are known in the art, and include, for example, those described in, WO2023028543A2, WO2023021319A1, WO2023014810A2, WO2022196697A1, WO2022152862A1, WO2022114163A1, US20230279144A1, US20230109218A1, US20220002436A1, US20210309759A1, WO2019160501A1, US20200399397A1, US20190241674A1, U.S. Pat. No. 9,555,112B2, US20170204194A1, U.S. Pat. No. 9,738,726B2, US20160053011A1, U.S. Ser. No. 10/174,116B2, WO2013160498A1, US20130243760A1, US20110217305A1, WO2011103700A1, WO2011084496A1, US20110158988A1, US20160130360A1, US20110117097A1, US20160060353A1, WO2001009187A2, U.S. Pat. No. 5,725,856A, and WO2023154780A1, the full contents of each of which are incorporated herein by reference for all purposes.

hEGFR binding domains that can be employed in multispecific proteins described herein are known in the art, and include, for example, those described in, WO2023172981A1, WO2023069888A1, WO2022272128A1, WO2022031935A1, WO2021003297A1, US20210115146A1, U.S. Ser. No. 10/927,176B2, US20170267765A1, WO2016044234A1, WO2015184403A2, WO2015066279A2, US20140271477A1, U.S. Pat. No. 9,132,192B2, U.S. Pat. No. 9,944,707B2, WO2013134743A1, U.S. Pat. No. 9,885,087B2, WO2012100384A1, US20130295086A1, US20140023662A1, U.S. Pat. No. 9,051,370B2, U.S. Pat. No. 9,029,513B2, WO2011140254A1, US20110117110A1, WO2011041319, US20210032366A1, US20100166744A1, WO2008140493A2, US20080274114A1, WO2005090407A1, U.S. Pat. No. 7,598,350B2, US20040033543A1, WO2002100348A2, and US20060193854A1, the full contents of each of which are incorporated herein by reference for all purposes.

hMSLN binding domains that can be employed in multispecific proteins described herein are known in the art, and include, for example, those described in, WO2023114980, U.S. Pat. Nos. 8,460,660, 7,081,518, WO2014031476, WO2019094482, WO2023020472A1, WO2023019396A1, WO2023115528A1, WO2023177974A2, WO2023081806A2, WO2023060157A1, US20230235081A1, WO2023006390A1, WO2022262859A1, WO2022242703A1, WO2022177913A1, WO2022121941A1, US20220249685A1, US20220267421A1, WO2019223579A1, WO2018060480A1, US20180002439A1, US20190112385A1, U.S. Ser. No. 10/851,175B2, U.S. Ser. No. 10/793,641B2, WO2014052064A1, U.S. Ser. No. 10/919,975B2, WO2010111282A1, WO2009120769A1, U.S. Pat. No. 9,023,351B2, WO2009045957A1, US20090202559A2, US20040142396A1, and US20050214304A1, the full contents of each of which are incorporated herein by reference for all purposes.

hPSMA binding domains that can be employed in multispecific proteins described herein are known in the art, and include, for example, those described in, WO2021123810A1, US20220411530A1, WO2020025564A1, U.S. Ser. No. 11/746,157B2, U.S. Ser. No. 11/414,497B2, U.S. Ser. No. 11/612,646B2, US20190023807A1, U.S. Ser. No. 11/059,903B2, US20230201366A1, U.S. Ser. No. 11/059,903B2, U.S. Ser. No. 10/100,126, U.S. Pat. Nos. 8,703,918B2, 6,107,090A, 8,470,330B2, US20090060908A1, U.S. Pat. No. 8,470,330B2, US20170037142A1, WO2023026236A1, WO2016145139A1, WO2022162518A2, US20230272110A1, US20150152187A1, U.S. Pat. No. 7,875,278B2, U.S. Ser. No. 11/612,646B2, U.S. Ser. No. 11/555,078B2, WO2020206330A1, US20210023094A1, EP1710256A1, US20070128671A1, the full contents of each of which are incorporated herein by reference for all purposes.

Exemplary hTAA binding domains that can be employed in multispecific proteins described herein are described in § 5.2.4, Table 6.

Exemplary hTAA binding domains known in the art that can be employed in fusion proteins described herein also, include, trastuzumab, cetuximab, or modified versions thereof.

The amino acid sequence of exemplary hTAA binding domains that can be utilized in the multispecific proteins described herein is provided in Table 6. The CDRs of the hTAA binding domains in Table 6, are denoted according to Kabat. A person of ordinary skill in the art would be able to determine the CDRs as defined by another scheme, e.g., Chothia, IMGT, using ordinary methods known in the art.

TABLE 6
The Amino Acid Sequence of Exemplary hTAA Binding Domains

| | | SEQ | |
|---|---|---|---|
| Description | | ID NO | Amino Acid Sequence |
| Anti-hHER2 | VH CDR1 | 61 | DTYIH |
| A | VH CDR2 | 62 | RIYPTNGYTRYADSVKG |
| | VH CDR3 | 63 | WGGDGFYAMDY |
| | VL CDR1 | 64 | RASQDVNTAVA |
| | VL CDR2 | 65 | SASFLYS |
| | VL CDR3 | 66 | QQHYTTPPT |
| | VH | 67 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSS |
| | VL | 68 | DIQMTQSPSSLSASVGDRVTITCRASQDVNTAVAWYQQKPGK |
| | | | APKLLIYSASFLYSGVPSRFSGSRSGTDFTLTISSLQPEDFA |
| | | | TYYCQQHYTTPPTFGQGTKVEIK |
| Anti-hHER2 | VH CDR1 | 367 | GFTFTDYT |
| B | VH CDR2 | 368 | VNPNSGGSIYNQRFK |
| | VH CDR3 | 369 | LGPSFYFDY |
| | VL CDR1 | 370 | KASQDVSIGVA |
| | VL CDR2 | 371 | ASYRYTG |
| | VL CDR3 | 372 | QQYYTYPYTF |
| | VH | 373 | EVQLVESGGGLVQPGGSLRLSCAASGFTFTDYTMDWVRQAPG |
| | | | KGLEWVADVNPNSGGSIYNQRFKGRFTLSVDRSKNTLYLQMN |
| | | | SLRAEDTAVYYCARNLGPSFYFDYWGQGTLVTVSS |
| | VL | 374 | DIQMTQSPSSLSASVGDRVTITCKASQDVSIGVAWYQQKPGK |
| | | | APKLLIYSASYRYTGVPSRFSGSGSGTDFTLTISSLQPEDFA |
| | | | TYYCQQYYIYPYTFGQGTKVEIKR |
| Anti-hHER2 | VH CDR1 | 375 | DYYMY |
| C | VH CDR2 | 376 | YINSGGGSTYYPDTVKG |
| | VH CDR3 | 377 | EALYDYDYAMDY |
| | VL CDR1 | 378 | KSSQSLLYSNGKTYLN |
| | VL CDR2 | 379 | LVSKLDS |
| | VL CDR3 | 380 | VQGTHFPLT |
| | VH | 381 | EVQLVESGGGLVQPGGSLRLSCAASGFTFSDYYMYWVRQAPG |
| | | | KGLEWVAYINSGGGSTYYPDTVKGRFTISRDNAKNSLYLQMN |
| | | | SLRAEDTAVYYCAREALYDYDYAMDYWGQGTTVTVSS |
| | VL | 382 | DIVMTQSPLSLSVTPGQPASISCKSSQSLLYSNGKTYLNWLL |
| | | | QKPGQSPQRLIYLVSKLDSGVPDRFSGSGSGTDFTLKISRVE |
| | | | AEDVGVYYCVQGTHFPLTFGGGTKVEIK |
| Anti-hEGFR | VH CDR1 | 69 | NYGVH |
| A | VH CDR2 | 70 | VIWSGGNTDYNTPFTS |
| | VH CDR3 | 71 | ALTYYDYEFAY |
| | VL CDR1 | 72 | RASQSIGTNIH |
| | VL CDR2 | 73 | YASESIS |
| | VL CDR3 | 74 | QQNNNWPTT |
| | VH | 75 | QVQLKQSGPGLVQPSQSLSITCTVSGFSLTNYGVHWVRQSPG |
| | | | KGLEWLGVIWSGGNTDYNTPFTSRLSINKDNSKSQVFFKMNS |
| | | | LQSNDTAIYYCARALTYYDYEFAYWGQGTLVTVSA |
| | VL | 76 | DILLTQSPVILSVSPGERVSFSCRASQSIGTNIHWYQQRING |
| | | | SPRLLIKYASESISGIPSRFSGSGSGTDFTLSINSVESEDIA |
| | | | DYYCQQNNNWPTTFGAGTKLELK |
| Anti-hEGFR | VH CDR1 | 272 | SGDYYWT |
| B | VH CDR2 | 273 | HIYYSGNTNYNPSLKS |
| | VH CDR3 | 274 | DRVTGAFDI |
| | VL CDR1 | 275 | QASQDISNYLN |
| | VL CDR2 | 276 | DASNLET |
| | VL CDR3 | 277 | QHFDHLPLA |
| | VH | 278 | QVQLQESGPGLVKPSETLSLTCTVSGGSVSSGDYYWTWIRQS |
| | | | PGKGLEWIGHIYYSGNTNYNPSLKSRLTISIDTSKTQFSLKL |
| | | | SSVTAADTAIYYCVRDRVTGAFDIWGQGTMVTVSS |
| | VL | 279 | DIQMTQSPSSLSASVGDRVTITCQASQDISNYLNWYQQKPGK |
| | | | APKLLIYDASNLETGVPSRFSGSGSGTDFTFTISSLQPEDIA |
| | | | TYFCQHFDHLPLAFGGGTKVEIK |
| Anti-hEGFR | VH CDR1 | 280 | TYGMH |
| C | VH CDR2 | 281 | VIWDDGSYKYYGDSVKG |
| | VH CDR3 | 282 | DGITMVRGVMKDYFDY |
| | VL CDR1 | 283 | RASQDISSALV |
| | VL CDR2 | 284 | DASSLES |
| | VL CDR3 | 285 | QQFNSYPLT |
| | VH | 286 | QVQLVESGGGVVQPGRSLRLSCAASGFTFSTYGMHWVRQAPG |
| | | | KGLEWVAVIWDDGSYKYYGDSVKGRFTISRDNSKNTLYLQMN |
| | | | SLRAEDTAVYYCARDGITMVRGVMKDYFDYWGQGTLVTVSS |
| | VL | 287 | AIQLTQSPSSLSASVGDRVTITCRASQDISSALVWYQQKPGK |
| | | | APKLLIYDASSLESGVPSRFSGSESGTDFTLTISSLQPEDFA |
| | | | TYYCQQFNSYPLTFGGGTKVEIK |
| Anti-hMSLN | VH CDR1 | 77 | SGSYYWS |
| (M912) | VH CDR2 | 78 | YIYYSGSTNYNPSLKS |
| | VH CDR3 | 79 | EGKNGAFDI |
| | VL CDR1 | 80 | RASQSISSYLN |
| | VL CDR2 | 81 | AASSLQS |
| | VL CDR3 | 82 | QQSYSTPLT |
| | VH | 83 | QVQLQESGPGLVKPSETLSLTCTVSGGSVSSGSYYWSWIRQP |
| | | | PGKGLEWIGYIYYSGSTNYNPSLKSRVTISVDTSKNQFSLKL |
| | | | SSVTAADTAVYYCAREGKNGAFDIWGQGTMVTVSS |
| | VL | 84 | DIQMTQSPSSLSASVGDRVTITCRASQSISSYLNWYQQKPGK |
| | | | APKLLIYAASSLQSGVPSGFSGSGSGTDFTLTISSLQPEDFA |
| | | | TYYCQQSYSTPLTFGGGTKVEIK |
| Anti-hMSLN | VH CDR1 | 288 | GYTMN |
| (MORab009) | VH CDR2 | 289 | LITPYNGASSYNQKFRG |
| | VH CDR3 | 290 | GGYDGRGFDY |
| | VL CDR1 | 291 | SASSSVSYMH |
| | VL CDR2 | 292 | DTSKLAS |
| | VL CDR3 | 293 | QQWSKHPLT |
| | VH | 294 | QVQLQQSGPELEKPGASVKISCKASGYSFTGYTMNWVKQSHG |
| | | | KSLEWIGLITPYNGASSYNQKFRGKATLTVDKSSSTAYMDLL |
| | | | SLTSEDSAVYFCARGGYDGRGFDYWGSGTPVTVSS |
| | VL | 295 | DIELTQSPAIMSASPGEKVTMTCSASSSVSYMHWYQQKSGTS |
| | | | PKRWIYDTSKLASGVPGRFSGSGSGNSYSLTISSVEAEDDAT |
| | | | YYCQQWSKHPLTFGSGTKVEIK |
| Anti-hMSLN | VH CDR1 | 383 | GYYMH |
| (FB6) | VH CDR2 | 384 | RINPYTGVPSYKHNFKD |
| | VH CDR3 | 385 | ELGGY |
| | VL CDR1 | 386 | RSSTGAVTTGNYPN |
| | VL CDR2 | 387 | GTNNRAP |
| | VL CDR3 | 388 | ALWFSSHWV |
| | VH | 389 | EVQLQQSGPVLVKPGASVKISCKASGYSFTGYYMHWVRQSLV |
| | | | KRLEWIGRINPYTGVPSYKHNFKDKASLTVDKSSSTAYMELH |
| | | | SLTSEDSAVYYCARELGGYWGQGTTLTVSS |
| | VL | 390 | QAVVTQESALTTSPGETVTLTCRSSTGAVTTGNYPNWVQEKP |
| | | | DHLFTGLIAGTNNRAPGVPARFSGSLIGDKAALTITGAQTED |
| | | | EAIYFCALWFSSHWVFGGGTKLTVL |
| Anti-hMSLN | VH CDR1 | 383 | GYYMH |
| (Humanized | VH CDR2 | 391 | RINPYTGVPSYKHKFQG |
| FB6 A) | VH CDR3 | 385 | ELGGY |
| | VL CDR1 | 392 | ASSTGAVTTGNYPN |
| | VL CDR2 | 387 | GTNNRAP |
| | VL CDR3 | 388 | ALWFSSHWV |
| | VH - A | 394 | EVQLVQSGAEVKKPGASVKVSCKASGYSFTGYYMHWVRQAPG |
| | | | QGLEWIGRINPYTGVPSYKHKFQGRVTLTVDKSTSTAYMELS |
| | | | SLRSEDTAVYYCARELGGYWGQGTTVTVSS |
| | VL - A | 395 | QAVVTQEPSLTVSPGGTVTLTCASSTGAVTTGNYPNWFQEKP |
| | | | GQAFRGLIAGTNNRAPWVPARFSGSLIGDKAALTLSGVQPED |
| | | | EAEYFCALWFSSHWVFGGGTKLTVL |
| | VH - B | 396 | QVQLVQSGAEVKKPGASVKVSCKASGYSFTGYYMHWVRQAPG |
| | | | QGLEWMGRINPYTGVPSYKHKFQGRVTMTVDKSTSTAYMELS |
| | | | SLRSEDTAVYYCARELGGYWGQGTTVTVSS |
| | VL - B | 395 | QAVVTQEPSLTVSPGGTVTLTCASSTGAVTTGNYPNWFQEKP |
| | | | GQAFRGLIAGTNNRAPWVPARFSGSLIGDKAALTLSGVQPED |
| | | | EAEYFCALWFSSHWVFGGGTKLTVL |
| Anti-hMSLN | VH CDR1 | 383 | GYYMH |
| (Humanized | VH CDR2 | 391 | RINPYTGVPSYKHKFQG |
| FB6 C) | VH CDR3 | 385 | ELGGY |
| | VL CDR1 | 392 | ASSTGAVTTGNYPN |
| | VL CDR2 | 393 | GTNNKAS |
| | VL CDR3 | 388 | ALWFSSHWV |
| | VH - A | 394 | EVQLVQSGAEVKKPGASVKVSCKASGYSFTGYYMHWVRQAPG |
| | | | QGLEWIGRINPYTGVPSYKHKFQGRVTLTVDKSTSTAYMELS |
| | | | SLRSEDTAVYYCARELGGYWGQGTTVTVSS |
| | VL - A | 397 | QAVVTQEPSLTVSPGGTVTLTCASSTGAVTTGNYPNWFQQKP |
| | | | GQAFRGLIAGTNNKASWTPARFSGSLLGDKAALTLSGVQPED |
| | | | EAEYYCALWFSSHWVFGGGTKLTVL |
| | VH - B | 396 | QVQLVQSGAEVKKPGASVKVSCKASGYSFTGYYMHWVRQAPG |
| | | | QGLEWMGRINPYTGVPSYKHKFQGRVTMTVDKSTSTAYMELS |
| | | | SLRSEDTAVYYCARELGGYWGQGTTVTVSS |
| | VL - B | 397 | QAVVTQEPSLTVSPGGTVTLTCASSTGAVTTGNYPNWFQQKP |
| | | | GQAFRGLIAGTNNKASWTPARFSGSLLGDKAALTLSGVQPED |
| | | | EAEYYCALWFSSHWVFGGGTKLTVL |
| Anti-hMSLN | VH CDR1 | 288 | GYTMN |
| (SS) | VH CDR2 | 289 | LITPYNGASSYNQKFRG |
| | VH CDR3 | 290 | GGYDGRGFDY |
| | VL CDR1 | 291 | SASSSVSYMH |
| | VL CDR2 | 292 | DTSKLAS |
| | VL CDR3 | 398 | QQWSGYPLT |
| | VH | 399 | QVQLQQSGPELEKPGASVKLSCKASGYSFTGYTMNWVKQSHG |
| | | | KSLEWIGLITPYNGASSYNQKFRGKATLTVDKSSSTAYMDLL |
| | | | SLTSEDSAVYFCARGGYDGRGFDYWGQGTTVTVSS |
| | VL | 400 | DIELTQSPAIMSASPGEKVTMTCSASSSVSYMHWYQQKSGTS |
| | | | PKRWIYDTSKLASGVPGRFSGSGSGNSYSLTISSVEAEDDAT |
| | | | YYCQQWSGYPLTFGAGTKLEIK |
| Anti-hMSLN | VH CDR1 | 401 | FYFYAC |
| (YP218) | VH CDR2 | 402 | CIYTAGSGSTYYASWAKG |
| | VH CDR3 | 403 | STANTRSTYYLNL |
| | VL CDR1 | 404 | QASQRISSYLS |
| | VL CDR2 | 405 | GASTLAS |
| | VL CDR3 | 406 | QSYAYFDSNNWHA |
| | VH | 407 | QQQLEESGGGLVKPEGSLTLTCKASGFDLGFYFYACWVRQAP |
| | | | GKGLEWIACIYTAGSGSTYYASWAKGRFTISKASSTTVTLQM |
| | | | TSLAAADTATYFCARSTANTRSTYYLNLWGPGTLVTVSS |
| | VL | 408 | DVVMTQTPASVSEPVGGTVTIKCQASQRISSYLSWYQQKPGQ |
| | | | RPKLLIFGASTLASGVPSRFKGSGSGTEYTLTISDLECADAA |
| | | | TYYCQSYAYFDSNNWHAFGGGTEVVV |
| Anti-hPSMA | VH CDR1 | 296 | SNWIG |
| A | VH CDR2 | 297 | IIYPGDSDTRYSPSFQG |
| | VH CDR3 | 298 | QTGFLWSSDL |
| | VL CDR1 | 299 | RASQDISSALA |
| | VL CDR2 | 300 | DASSLES |
| | VL CDR3 | 301 | QQFNSYPLT |
| | VH | 302 | EVQLVQSGAEVKKPGESLKISCKGSGYSFTSNWIGWVRQMPG |
| | | | KGLEWMGIIYPGDSDTRYSPSFQGQVTISADKSISTAYLQWS |
| | | | SLKASDTAMYYCARQTGFLWSSDLWGRGTLVTVSS |
| | VL | 303 | AIQLTQSPSSLSASVGDRVTITCRASQDISSALAWYQQKPGK |
| | | | APKLLIYDASSLESGVPSRFSGYGSGTDFTLTINSLQPEDFA |
| | | | TYYCQQFNSYPLTFGGGTKVEIK |
| Anti-hPSMA | VH CDR1 | 409 | SYWMS |
| B | VH CDR2 | 410 | NIKQDGSEKYYVDSVKG |
| | VH CDR3 | 411 | VWDYYYDSSGDAFDI |
| | VL CDR1 | 412 | RASQGISSWLA |
| | VL CDR2 | 413 | AASNLQS |
| | VL CDR3 | 414 | QQANSFPLT |
| | VH | 415 | QVQLVQSGGGLVQPGGSLRLSCAASGFTFSSYWMSWVRQAPG |
| | | | KGLEWVANIKQDGSEKYYVDSVKGRFTISRDNAKNSLYLQMN |
| | | | SLRAEDTAVYYCARVWDYYYDSSGDAFDIWGQGTMVTVSS |
| | VL | 416 | VIWMTQSPSSVSASVGDRVTITCRASQGISSWLAWYQQKPGK |
| | | | APKLLIYAASNLQSGVPSRFSGSGSGTDFTLTISSLQPEDFA |
| | | | TYYCQQANSFPLTFGGGTKVDIK |

In some embodiments, the hTAA binding domain comprises a hTAA binding domain provided in Table 6.

In some embodiments, the hTAA binding domain comprises a VH that comprises: VH CDR1, VH CDR2, and VH CDR3.

In some embodiments, the amino acid sequence of VH CDR1 comprises or consists of the amino acid sequence of a VH CDR1 of a VH set forth in Table 6, or the amino acid sequence of a VH CDR1 of a VH set forth in Table 6 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 of a VH set forth in Table 6, or the amino acid sequence of a VH CDR2 of a VH set forth in Table 6 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 of a VH set forth in Table 6, or the amino acid sequence of a VH CDR3 of a VH set forth in Table 6 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 of a VH set forth in Table 6, or the amino acid sequence of a VH CDR1 of a VH set forth in Table 6 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 6, or the amino acid sequence of a VH CDR2 of a VH set forth in Table 6 comprising or consisting of 1, 2, or 3 amino acid modifications (e.g., substitution, deletion, addition, etc.); and the amino acid sequence of VH CDR3 comprises or consists of the amino acid sequence of a VH CDR3 of a VH set forth in Table 6, or the amino acid sequence of a VH CDR3 of a VH set forth in Table 6 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 6, or the amino acid sequence of a VH CDR1 set forth in Table 6 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 set forth in Table 6, or the amino acid sequence of a VH CDR2 set forth in Table 6 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 set forth in Table 6, or the amino acid sequence of a VH CDR3 set forth in Table 6 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 6, or the amino acid sequence of a VH CDR1 set forth in Table 6 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 6, or the amino acid sequence of a VH CDR2 set forth in Table 6 comprising or consisting of 1, 2, or 3 amino acid modifications (e.g., substitution, deletion, addition, etc.); and the amino acid sequence of VH CDR3 comprises or consists of the amino acid sequence of a VH CDR3 set forth in Table 6, or the amino acid sequence of a VH CDR3 set forth in Table 6 comprising or consisting of 1, 2, or 3 amino acid modifications (e.g., substitution, deletion, addition, etc.).

In some embodiments, the hTAA binding domain comprises a VL that comprises: VL CDR1, VL CDR2, and VL CDR3.

In some embodiments, the amino acid sequence of VL CDR1 comprises or consists of the amino acid sequence of a VL CDR1 of a VL set forth in Table 6, or the amino acid sequence of a VL CDR1 of a VL set forth in Table 6 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 of a VL set forth in Table 6, or the amino acid sequence of a VL CDR2 of a VL set forth in Table 6 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 of a VL set forth in Table 6, or the amino acid sequence of a VL CDR3 of a VL set forth in Table 6 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 of a VL set forth in Table 6, or the amino acid sequence of a VL CDR1 of a VL set forth in Table 6 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 6, or the amino acid sequence of a VL CDR2 of a VL set forth in Table 6 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 6, or the amino acid sequence of a VL CDR3 of a VL set forth in Table 6 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 6, or the amino acid sequence of a VL CDR1 set forth in Table 6 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 set forth in Table 6, or the amino acid sequence of a VL CDR2 set forth in Table 6 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 set forth in Table 6, or the amino acid sequence of a VL CDR3 set forth in Table 6 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 6, or the amino acid sequence of a VL CDR1 set forth in Table 6 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 6, or the amino acid sequence of a VL CDR2 set forth in Table 6 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 6, or the amino acid sequence of a VL CDR3 set forth in Table 6 comprising or consisting of 1, 2, or 3 amino acid modifications (e.g., substitution, deletion, addition, etc.).

In some embodiments, the hTAA binding domain comprises a VH that comprises: VH CDR1, VH CDR2, and VH CDR3; and a VL that comprises: VL CDR1, VL CDR2, and VL CDR3.

In some embodiments, the amino acid sequence of VH CDR1 comprises or consists of the amino acid sequence of a VH CDR1 of a VH set forth in Table 6, or the amino acid sequence of a VH CDR1 of a VH set forth in Table 6 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 6, or the amino acid sequence of a VH CDR2 of a VH set forth in Table 6 comprising or consisting of 1, 2, or 3 amino acid modifications (e.g., substitution, deletion, addition, etc.); the amino acid sequence of VH CDR3 comprises or consists of the amino acid sequence of a VH CDR3 of a VH set forth in Table 6, or the amino acid sequence of a VH CDR3 of a VH set forth in Table 6 comprising or consisting of 1, 2, or 3 amino acid modifications (e.g., substitution, deletion, addition, etc.); the amino acid sequence of VL CDR1 comprises or consists of the amino acid sequence of a VL CDR1 of a VL set forth in Table 6, or the amino acid sequence of a VL CDR1 of a VL set forth in Table 6 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 6, or the amino acid sequence of a VL CDR2 of a VL set forth in Table 6 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 6, or the amino acid sequence of a VL CDR3 of a VL set forth in Table 6 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 6, or the amino acid sequence of a VH CDR1 set forth in Table 6 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 6, or the amino acid sequence of a VH CDR2 set forth in Table 6 comprising or consisting of 1, 2, or 3 amino acid modifications (e.g., substitution, deletion, addition, etc.); the amino acid sequence of VH CDR3 comprises or consists of the amino acid sequence of a VH CDR3 set forth in Table 6, or the amino acid sequence of a VH CDR3 set forth in Table 6 comprising or consisting of 1, 2, or 3 amino acid modifications (e.g., substitution, deletion, addition, etc.); the amino acid sequence of VL CDR1 comprises or consists of the amino acid sequence of a VL CDR1 set forth in Table 6, or the amino acid sequence CDR1 of a VL set forth in Table 6 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 6, or the amino acid sequence of a VL CDR2 set forth in Table 6 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 6, or the amino acid sequence of a VL CDR3 set forth in Table 6 comprising or consisting of 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 6. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 6. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 6; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 6.

In some embodiments, the amino acid sequence of VH CDR1 comprises or consists of the amino acid sequence set forth in SEQ ID NO: 61, or the amino acid sequence set forth in SEQ ID NO: 61 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 62, or the amino acid sequence set forth in SEQ ID NO: 62 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 63, or the amino acid sequence set forth in SEQ ID NO: 63 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 61, or the amino acid sequence set forth in SEQ ID NO: 61 comprising 1, 2, or 3 amino acid modifications (e.g., substitution, deletion, addition, etc.); the amino acid sequence of VH CDR2 comprises or consists of the amino acid sequence set forth in SEQ ID NO: 62, or the amino acid sequence set forth in SEQ ID NO: 62 comprising 1, 2, or 3 amino acid modifications (e.g., substitution, deletion, addition, etc.); and the amino acid sequence of VH CDR3 comprises or consists of the amino acid sequence set forth in SEQ ID NO: 63, or the amino acid sequence set forth in SEQ ID NO: 63 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 64, or the amino acid sequence set forth in SEQ ID NO: 64 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 65, or the amino acid sequence set forth in SEQ ID NO: 65 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 66, or the amino acid sequence set forth in SEQ ID NO: 66 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 64, or the amino acid sequence set forth in SEQ ID NO: 64 comprising 1, 2, or 3 amino acid modifications (e.g., substitution, deletion, addition, etc.); the amino acid sequence of VL CDR2 comprises or consists of the amino acid sequence set forth in SEQ ID NO: 65, or the amino acid sequence set forth in SEQ ID NO: 65 comprising 1, 2, or 3 amino acid modifications (e.g., substitution, deletion, addition, etc.); and the amino acid sequence of VL CDR3 comprises or consists of the amino acid sequence set forth in SEQ ID NO: 66, or the amino acid sequence set forth in SEQ ID NO: 66 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 61, or the amino acid sequence set forth in SEQ ID NO: 61 comprising 1, 2, or 3 amino acid modifications (e.g., substitution, deletion, addition, etc.); the amino acid sequence of VH CDR2 comprises or consists of the amino acid sequence set forth in SEQ ID NO: 62, or the amino acid sequence set forth in SEQ ID NO: 62 comprising 1, 2, or 3 amino acid modifications (e.g., substitution, deletion, addition, etc.); the amino acid sequence of VH CDR3 comprises or consists of the amino acid sequence set forth in SEQ ID NO: 63, or the amino acid sequence set forth in SEQ ID NO: 63 comprising 1, 2, or 3 amino acid modifications (e.g., substitution, deletion, addition, etc.); the amino acid sequence of VL CDR1 comprises or consists of the amino acid sequence set forth in SEQ ID NO: 64, or the amino acid sequence set forth in SEQ ID NO: 64 comprising 1, 2, or 3 amino acid modifications (e.g., substitution, deletion, addition, etc.); the amino acid sequence of VL CDR2 comprises or consists of the amino acid sequence set forth in SEQ ID NO: 65, or the amino acid sequence set forth in SEQ ID NO: 65 comprising 1, 2, or 3 amino acid modifications (e.g., substitution, deletion, addition, etc.); and the amino acid sequence of VL CDR3 comprises or consists of the amino acid sequence set forth in SEQ ID NO: 66, or the amino acid sequence set forth in SEQ ID NO: 66 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 67. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 68. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 67; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 68.

In some embodiments, the amino acid sequence of VH CDR1 comprises or consists of the amino acid sequence set forth in SEQ ID NO: 69, or the amino acid sequence set forth in SEQ ID NO: 69 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 70, or the amino acid sequence set forth in SEQ ID NO: 70 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 71, or the amino acid sequence set forth in SEQ ID NO: 71 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 69, or the amino acid sequence set forth in SEQ ID NO: 69 comprising 1, 2, or 3 amino acid modifications (e.g., substitution, deletion, addition, etc.); the amino acid sequence of VH CDR2 comprises or consists of the amino acid sequence set forth in SEQ ID NO: 70, or the amino acid sequence set forth in SEQ ID NO: 70 comprising 1, 2, or 3 amino acid modifications (e.g., substitution, deletion, addition, etc.); and the amino acid sequence of VH CDR3 comprises or consists of the amino acid sequence set forth in SEQ ID NO: 71, or the amino acid sequence set forth in SEQ ID NO: 71 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 72, or the amino acid sequence set forth in SEQ ID NO: 72 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 73, or the amino acid sequence set forth in SEQ ID NO: 73 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 74, or the amino acid sequence set forth in SEQ ID NO: 74 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 72, or the amino acid sequence set forth in SEQ ID NO: 72 comprising 1, 2, or 3 amino acid modifications (e.g., substitution, deletion, addition, etc.); the amino acid sequence of VL CDR2 comprises or consists of the amino acid sequence set forth in SEQ ID NO: 73, or the amino acid sequence set forth in SEQ ID NO: 73 comprising 1, 2, or 3 amino acid modifications (e.g., substitution, deletion, addition, etc.); and the amino acid sequence of VL CDR3 comprises or consists of the amino acid sequence set forth in SEQ ID NO: 74, or the amino acid sequence set forth in SEQ ID NO: 74 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 69, or the amino acid sequence set forth in SEQ ID NO: 69 comprising 1, 2, or 3 amino acid modifications (e.g., substitution, deletion, addition, etc.); the amino acid sequence of VH CDR2 comprises or consists of the amino acid sequence set forth in SEQ ID NO: 70, or the amino acid sequence set forth in SEQ ID NO: 70 comprising 1, 2, or 3 amino acid modifications (e.g., substitution, deletion, addition, etc.); the amino acid sequence of VH CDR3 comprises or consists of the amino acid sequence set forth in SEQ ID NO: 71, or the amino acid sequence set forth in SEQ ID NO: 71 comprising 1, 2, or 3 amino acid modifications (e.g., substitution, deletion, addition, etc.); the amino acid sequence of VL CDR1 comprises or consists of the amino acid sequence set forth in SEQ ID NO: 72, or the amino acid sequence set forth in SEQ ID NO: 72 comprising 1, 2, or 3 amino acid modifications (e.g., substitution, deletion, addition, etc.); the amino acid sequence of VL CDR2 comprises or consists of the amino acid sequence set forth in SEQ ID NO: 73, or the amino acid sequence set forth in SEQ ID NO: 73 comprising 1, 2, or 3 amino acid modifications (e.g., substitution, deletion, addition, etc.); and the amino acid sequence of VL CDR3 comprises or consists of the amino acid sequence set forth in SEQ ID NO: 74, or the amino acid sequence set forth in SEQ ID NO: 74 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 75. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 76. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 75; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 76.

In some embodiments, the amino acid sequence of VH CDR1 comprises or consists of the amino acid sequence set forth in SEQ ID NO: 272, or the amino acid sequence set forth in SEQ ID NO: 272 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 273, or the amino acid sequence set forth in SEQ ID NO: 273 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 274, or the amino acid sequence set forth in SEQ ID NO: 274 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 272, or the amino acid sequence set forth in SEQ ID NO: 272 comprising 1, 2, or 3 amino acid modifications (e.g., substitution, deletion, addition, etc.); the amino acid sequence of VH CDR2 comprises or consists of the amino acid sequence set forth in SEQ ID NO: 273, or the amino acid sequence set forth in SEQ ID NO: 273 comprising 1, 2, or 3 amino acid modifications (e.g., substitution, deletion, addition, etc.); and the amino acid sequence of VH CDR3 comprises or consists of the amino acid sequence set forth in SEQ ID NO: 274, or the amino acid sequence set forth in SEQ ID NO: 274 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 275, or the amino acid sequence set forth in SEQ ID NO: 275 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 276, or the amino acid sequence set forth in SEQ ID NO: 276 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 277, or the amino acid sequence set forth in SEQ ID NO: 277 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 275, or the amino acid sequence set forth in SEQ ID NO: 275 comprising 1, 2, or 3 amino acid modifications (e.g., substitution, deletion, addition, etc.); the amino acid sequence of VL CDR2 comprises or consists of the amino acid sequence set forth in SEQ ID NO: 276, or the amino acid sequence set forth in SEQ ID NO: 276 comprising 1, 2, or 3 amino acid modifications (e.g., substitution, deletion, addition, etc.); and the amino acid sequence of VL CDR3 comprises or consists of the amino acid sequence set forth in SEQ ID NO: 277, or the amino acid sequence set forth in SEQ ID NO: 277 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 272, or the amino acid sequence set forth in SEQ ID NO: 272 comprising 1, 2, or 3 amino acid modifications (e.g., substitution, deletion, addition, etc.); the amino acid sequence of VH CDR2 comprises or consists of the amino acid sequence set forth in SEQ ID NO: 273, or the amino acid sequence set forth in SEQ ID NO: 273 comprising 1, 2, or 3 amino acid modifications (e.g., substitution, deletion, addition, etc.); the amino acid sequence of VH CDR3 comprises or consists of the amino acid sequence set forth in SEQ ID NO: 274, or the amino acid sequence set forth in SEQ ID NO: 274 comprising 1, 2, or 3 amino acid modifications (e.g., substitution, deletion, addition, etc.); the amino acid sequence of VL CDR1 comprises or consists of the amino acid sequence set forth in SEQ ID NO: 275, or the amino acid sequence set forth in SEQ ID NO: 275 comprising 1, 2, or 3 amino acid modifications (e.g., substitution, deletion, addition, etc.); the amino acid sequence of VL CDR2 comprises or consists of the amino acid sequence set forth in SEQ ID NO: 276, or the amino acid sequence set forth in SEQ ID NO: 276 comprising 1, 2, or 3 amino acid modifications (e.g., substitution, deletion, addition, etc.); and the amino acid sequence of VL CDR3 comprises or consists of the amino acid sequence set forth in SEQ ID NO: 277, or the amino acid sequence set forth in SEQ ID NO: 277 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 278. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 279. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 278; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 279.

In some embodiments, the amino acid sequence of VH CDR1 comprises or consists of the amino acid sequence set forth in SEQ ID NO: 280, or the amino acid sequence set forth in SEQ ID NO: 280 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 281, or the amino acid sequence set forth in SEQ ID NO: 281 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 282, or the amino acid sequence set forth in SEQ ID NO: 282 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 280, or the amino acid sequence set forth in SEQ ID NO: 280 comprising 1, 2, or 3 amino acid modifications (e.g., substitution, deletion, addition, etc.); the amino acid sequence of VH CDR2 comprises or consists of the amino acid sequence set forth in SEQ ID NO: 281, or the amino acid sequence set forth in SEQ ID NO: 281 comprising 1, 2, or 3 amino acid modifications (e.g., substitution, deletion, addition, etc.); and the amino acid sequence of VH CDR3 comprises or consists of the amino acid sequence set forth in SEQ ID NO: 282, or the amino acid sequence set forth in SEQ ID NO: 282 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 283, or the amino acid sequence set forth in SEQ ID NO: 283 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 284, or the amino acid sequence set forth in SEQ ID NO: 284 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 285, or the amino acid sequence set forth in SEQ ID NO: 285 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 283, or the amino acid sequence set forth in SEQ ID NO: 283 comprising 1, 2, or 3 amino acid modifications (e.g., substitution, deletion, addition, etc.); the amino acid sequence of VL CDR2 comprises or consists of the amino acid sequence set forth in SEQ ID NO: 284, or the amino acid sequence set forth in SEQ ID NO: 284 comprising 1, 2, or 3 amino acid modifications (e.g., substitution, deletion, addition, etc.); and the amino acid sequence of VL CDR3 comprises or consists of the amino acid sequence set forth in SEQ ID NO: 285, or the amino acid sequence set forth in SEQ ID NO: 285 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 280, or the amino acid sequence set forth in SEQ ID NO: 280 comprising 1, 2, or 3 amino acid modifications (e.g., substitution, deletion, addition, etc.); the amino acid sequence of VH CDR2 comprises or consists of the amino acid sequence set forth in SEQ ID NO: 281, or the amino acid sequence set forth in SEQ ID NO: 281 comprising 1, 2, or 3 amino acid modifications (e.g., substitution, deletion, addition, etc.); the amino acid sequence of VH CDR3 comprises or consists of the amino acid sequence set forth in SEQ ID NO: 282, or the amino acid sequence set forth in SEQ ID NO: 282 comprising 1, 2, or 3 amino acid modifications (e.g., substitution, deletion, addition, etc.); the amino acid sequence of VL CDR1 comprises or consists of the amino acid sequence set forth in SEQ ID NO: 283, or the amino acid sequence set forth in SEQ ID NO: 283 comprising 1, 2, or 3 amino acid modifications (e.g., substitution, deletion, addition, etc.); the amino acid sequence of VL CDR2 comprises or consists of the amino acid sequence set forth in SEQ ID NO: 284, or the amino acid sequence set forth in SEQ ID NO: 284 comprising 1, 2, or 3 amino acid modifications (e.g., substitution, deletion, addition, etc.); and the amino acid sequence of VL CDR3 comprises or consists of the amino acid sequence set forth in SEQ ID NO: 285, or the amino acid sequence set forth in SEQ ID NO: 285 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 286. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 287. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 286; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 287.

In some embodiments, the amino acid sequence of VH CDR1 comprises or consists of the amino acid sequence set forth in SEQ ID NO: 288, or the amino acid sequence set forth in SEQ ID NO: 288 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 289, or the amino acid sequence set forth in SEQ ID NO: 289 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 290, or the amino acid sequence set forth in SEQ ID NO: 290 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 288, or the amino acid sequence set forth in SEQ ID NO: 288 comprising 1, 2, or 3 amino acid modifications (e.g., substitution, deletion, addition, etc.); the amino acid sequence of VH CDR2 comprises or consists of the amino acid sequence set forth in SEQ ID NO: 289, or the amino acid sequence set forth in SEQ ID NO: 289 comprising 1, 2, or 3 amino acid modifications (e.g., substitution, deletion, addition, etc.); and the amino acid sequence of VH CDR3 comprises or consists of the amino acid sequence set forth in SEQ ID NO: 290, or the amino acid sequence set forth in SEQ ID NO: 290 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 291, or the amino acid sequence set forth in SEQ ID NO: 291 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 292, or the amino acid sequence set forth in SEQ ID NO: 292 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 293, or the amino acid sequence set forth in SEQ ID NO: 293 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 291, or the amino acid sequence set forth in SEQ ID NO: 291 comprising 1, 2, or 3 amino acid modifications (e.g., substitution, deletion, addition, etc.); the amino acid sequence of VL CDR2 comprises or consists of the amino acid sequence set forth in SEQ ID NO: 292, or the amino acid sequence set forth in SEQ ID NO: 292 comprising 1, 2, or 3 amino acid modifications (e.g., substitution, deletion, addition, etc.); and the amino acid sequence of VL CDR3 comprises or consists of the amino acid sequence set forth in SEQ ID NO: 293, or the amino acid sequence set forth in SEQ ID NO: 293 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 288, or the amino acid sequence set forth in SEQ ID NO: 288 comprising 1, 2, or 3 amino acid modifications (e.g., substitution, deletion, addition, etc.); the amino acid sequence of VH CDR2 comprises or consists of the amino acid sequence set forth in SEQ ID NO: 289, or the amino acid sequence set forth in SEQ ID NO: 289 comprising 1, 2, or 3 amino acid modifications (e.g., substitution, deletion, addition, etc.); the amino acid sequence of VH CDR3 comprises or consists of the amino acid sequence set forth in SEQ ID NO: 290, or the amino acid sequence set forth in SEQ ID NO: 290 comprising 1, 2, or 3 amino acid modifications (e.g., substitution, deletion, addition, etc.); the amino acid sequence of VL CDR1 comprises or consists of the amino acid sequence set forth in SEQ ID NO: 291, or the amino acid sequence set forth in SEQ ID NO: 291 comprising 1, 2, or 3 amino acid modifications (e.g., substitution, deletion, addition, etc.); the amino acid sequence of VL CDR2 comprises or consists of the amino acid sequence set forth in SEQ ID NO: 292, or the amino acid sequence set forth in SEQ ID NO: 292 comprising 1, 2, or 3 amino acid modifications (e.g., substitution, deletion, addition, etc.); and the amino acid sequence of VL CDR3 comprises or consists of the amino acid sequence set forth in SEQ ID NO: 293, or the amino acid sequence set forth in SEQ ID NO: 293 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 294. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 295. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 294; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 295.

In some embodiments, the amino acid sequence of VH CDR1 comprises or consists of the amino acid sequence set forth in SEQ ID NO: 296, or the amino acid sequence set forth in SEQ ID NO: 296 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 297, or the amino acid sequence set forth in SEQ ID NO: 297 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 298, or the amino acid sequence set forth in SEQ ID NO: 298 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 296, or the amino acid sequence set forth in SEQ ID NO: 296 comprising 1, 2, or 3 amino acid modifications (e.g., substitution, deletion, addition, etc.); the amino acid sequence of VH CDR2 comprises or consists of the amino acid sequence set forth in SEQ ID NO: 297, or the amino acid sequence set forth in SEQ ID NO: 297 comprising 1, 2, or 3 amino acid modifications (e.g., substitution, deletion, addition, etc.); and the amino acid sequence of VH CDR3 comprises or consists of the amino acid sequence set forth in SEQ ID NO: 298, or the amino acid sequence set forth in SEQ ID NO: 298 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 299, or the amino acid sequence set forth in SEQ ID NO: 299 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 300, or the amino acid sequence set forth in SEQ ID NO: 300 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 301, or the amino acid sequence set forth in SEQ ID NO: 301 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 299, or the amino acid sequence set forth in SEQ ID NO: 299 comprising 1, 2, or 3 amino acid modifications (e.g., substitution, deletion, addition, etc.); the amino acid sequence of VL CDR2 comprises or consists of the amino acid sequence set forth in SEQ ID NO: 300, or the amino acid sequence set forth in SEQ ID NO: 300 comprising 1, 2, or 3 amino acid modifications (e.g., substitution, deletion, addition, etc.); and the amino acid sequence of VL CDR3 comprises or consists of the amino acid sequence set forth in SEQ ID NO: 301, or the amino acid sequence set forth in SEQ ID NO: 301 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 296, or the amino acid sequence set forth in SEQ ID NO: 296 comprising 1, 2, or 3 amino acid modifications (e.g., substitution, deletion, addition, etc.); the amino acid sequence of VH CDR2 comprises or consists of the amino acid sequence set forth in SEQ ID NO: 297, or the amino acid sequence set forth in SEQ ID NO: 297 comprising 1, 2, or 3 amino acid modifications (e.g., substitution, deletion, addition, etc.); the amino acid sequence of VH CDR3 comprises or consists of the amino acid sequence set forth in SEQ ID NO: 298, or the amino acid sequence set forth in SEQ ID NO: 298 comprising 1, 2, or 3 amino acid modifications (e.g., substitution, deletion, addition, etc.); the amino acid sequence of VL CDR1 comprises or consists of the amino acid sequence set forth in SEQ ID NO: 299, or the amino acid sequence set forth in SEQ ID NO: 299 comprising 1, 2, or 3 amino acid modifications (e.g., substitution, deletion, addition, etc.); the amino acid sequence of VL CDR2 comprises or consists of the amino acid sequence set forth in SEQ ID NO: 300, or the amino acid sequence set forth in SEQ ID NO: 300 comprising 1, 2, or 3 amino acid modifications (e.g., substitution, deletion, addition, etc.); and the amino acid sequence of VL CDR3 comprises or consists of the amino acid sequence set forth in SEQ ID NO: 301, or the amino acid sequence set forth in SEQ ID NO: 301 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 302. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 303. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 302; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 303.

In some embodiments, the amino acid sequence of VH CDR1 comprises or consists of the amino acid sequence set forth in SEQ ID NO: 77, or the amino acid sequence set forth in SEQ ID NO: 77 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 78, or the amino acid sequence set forth in SEQ ID NO: 78 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 79, or the amino acid sequence set forth in SEQ ID NO: 79 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 77, or the amino acid sequence set forth in SEQ ID NO: 77 comprising 1, 2, or 3 amino acid modifications (e.g., substitution, deletion, addition, etc.); the amino acid sequence of VH CDR2 comprises or consists of the amino acid sequence set forth in SEQ ID NO: 78, or the amino acid sequence set forth in SEQ ID NO: 78 comprising 1, 2, or 3 amino acid modifications (e.g., substitution, deletion, addition, etc.); and the amino acid sequence of VH CDR3 comprises or consists of the amino acid sequence set forth in SEQ ID NO: 79, or the amino acid sequence set forth in SEQ ID NO: 79 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 80, or the amino acid sequence set forth in SEQ ID NO: 80 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 81, or the amino acid sequence set forth in SEQ ID NO: 81 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 82, or the amino acid sequence set forth in SEQ ID NO: 82 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 80, or the amino acid sequence set forth in SEQ ID NO: 80 comprising 1, 2, or 3 amino acid modifications (e.g., substitution, deletion, addition, etc.); the amino acid sequence of VL CDR2 comprises or consists of the amino acid sequence set forth in SEQ ID NO: 81, or the amino acid sequence set forth in SEQ ID NO: 81 comprising 1, 2, or 3 amino acid modifications (e.g., substitution, deletion, addition, etc.); and the amino acid sequence of VL CDR3 comprises or consists of the amino acid sequence set forth in SEQ ID NO: 82, or the amino acid sequence set forth in SEQ ID NO: 82 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 77, or the amino acid sequence set forth in SEQ ID NO: 77 comprising 1, 2, or 3 amino acid modifications (e.g., substitution, deletion, addition, etc.); the amino acid sequence of VH CDR2 comprises or consists of the amino acid sequence set forth in SEQ ID NO: 78, or the amino acid sequence set forth in SEQ ID NO: 78 comprising 1, 2, or 3 amino acid modifications (e.g., substitution, deletion, addition, etc.); the amino acid sequence of VH CDR3 comprises or consists of the amino acid sequence set forth in SEQ ID NO: 79, or the amino acid sequence set forth in SEQ ID NO: 79 comprising 1, 2, or 3 amino acid modifications (e.g., substitution, deletion, addition, etc.); the amino acid sequence of VL CDR1 comprises or consists of the amino acid sequence set forth in SEQ ID NO: 80, or the amino acid sequence set forth in SEQ ID NO: 80 comprising 1, 2, or 3 amino acid modifications (e.g., substitution, deletion, addition, etc.); the amino acid sequence of VL CDR2 comprises or consists of the amino acid sequence set forth in SEQ ID NO: 81, or the amino acid sequence set forth in SEQ ID NO: 81 comprising 1, 2, or 3 amino acid modifications (e.g., substitution, deletion, addition, etc.); and the amino acid sequence of VL CDR3 comprises or consists of the amino acid sequence set forth in SEQ ID NO: 82, or the amino acid sequence set forth in SEQ ID NO: 82 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 83. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 84. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 83; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 84.

### 5.2.5 Affinity of Antigen Binding Domains

Binding affinity of any of the multispecific proteins described herein can be measured by standard assays known in the art and described herein (see, e.g., §§ 6.2, 6.3, 6.4). For example, binding affinity can be measured by ELISA. Binding affinity can also be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated herein by reference for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip.

Other suitable assays for measuring the binding of a multispecific protein described herein for one or more of its cognate antigens include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.2.6 Signal Peptides

In some embodiments, the multispecific protein (or one or more polypeptide thereof) comprises a signal peptide operably connected to e.g., the N-terminus, of said multispecific protein (or one or more polypeptide thereof). In some embodiments, each polypeptide chain of a multi-polypeptide multispecific protein described herein comprises a signal peptide (e.g., at the N-terminus of the polypeptide). Commonly used signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art. The amino acid sequence of exemplary signal peptides is provided in Table 7.

TABLE 7
The amino acid sequence of exemplary signal
peptides

| Description | Amino Acid Sequence | SEQ ID NO |
|---|---|---|
| hIL-2 | MYRMQLLSCIALSLALVTNS | 93 |
| hOSM | MGVLLTQRTLLSLVLALLFPSMASM | 94 |
| hCTRB1 | MASLWLLSCFSLVGAAFG | 95 |
| hTRY2 | MNLLLILTFVAAAVA | 96 |
| hINS | MALWMRLLPLLALLALWGPDPAAA | 97 |

In some embodiments, the amino acid sequence of the signal peptide comprises or consists of the amino acid sequence of any one of the signal peptides set forth in Table 7. In some embodiments, the amino acid sequence of the signal peptide comprises or consists of the amino acid sequence of any one of the signal peptides set forth in Table 7, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence of any one of the signal peptides set forth in Table 7, comprising 1, 2, or 3 amino acid variations (e.g., substitutions, deletions, additions). In some embodiments, the amino acid sequence of the signal peptide comprises or consists of the amino acid sequence of any one of the signal peptides set forth in Table 7, and further comprises 1 or more but less than 15% (less than 12%, less than 10%, less than 8%), amino acid substitutions. In some embodiments, the amino acid sequence of the signal peptide comprises or consists of the amino acid sequence of any one of the signal peptides set forth in Table 7, comprising 1, 2, or 3 amino acid substitutions.

In some embodiments, the amino acid sequence of the signal peptide comprises or consists of the amino acid sequence of any one of SEQ ID NOS: 93-97. In some embodiments, the amino acid sequence of the signal peptide comprises or consists of the amino acid sequence of any one of SEQ ID NOS: 93-97, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence of any one of SEQ ID NOS: 93-97, comprising 1, 2, or 3 amino acid variations (e.g., substitutions, deletions, additions). In some embodiments, the amino acid sequence of the signal peptide comprises or consists of the amino acid sequence of any one of SEQ ID NOS: 93-97, and further comprises 1 or more but less than 15% (less than 12%, less than 10%, less than 8%), amino acid substitutions. In some embodiments, the amino acid sequence of the signal peptide comprises or consists of the amino acid sequence of any one of SEQ ID NOS: 93-97, comprising 1, 2, or 3 amino acid substitutions.

### 5.2.7 Ig Constant Regions

As descried herein, the multispecific proteins (or one or more polypeptide thereof) described herein comprise one or more hIg heavy chain constant regions (e.g., a CH1 region, a hinge region, a CH2 region, a CH3 region, an Fc region) (see, e.g., § 5.2.1). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the multispecific protein (or one or more polypeptide thereof) comprises a Fc region (see, e.g., § 5.2.1). In some embodiments, the Fc region is part of a full-length antibody. In some embodiments, the Fc region comprises or consists of at least a portion of a hinge region, a CH2 region, and a CH3 region. In some embodiments, the Fc region comprises or consists of a hinge region, a CH2 region, and a CH3 region. In some embodiments, the Fc region comprises or consists of at least a portion of a hIgG hinge region, a hIgG CH2 region, and a hIgG CH3 region. In some embodiments, the Fc region comprises or consists of a hIgG hinge region, a hIgG CH2 region, and a hIgG CH3 region. In some embodiments, the Fc region comprises or consists of at least a portion of a hIgG1 hinge region, a hIgG1 CH2 region, and a hIgG1 CH3 region. In some embodiments, the Fc region comprises or consists of a hIgG1 hinge region, a hIgG1 CH2 region, and a hIgG1 CH3 region. In some embodiments, the Fc region comprises or consists of at least a portion of a hIgG4 hinge region, a hIgG4 CH2 region, and a hIgG4 CH3 region. In some embodiments, the Fc region comprises or consists of a hIgG4 hinge region, a hIgG4 CH2 region, and a hIgG4 CH3 region.

The amino acid sequence of exemplary reference hIgG1 and hIgG4 heavy chain constant regions, which can be incorporated in one or more of the embodiments described herein (e.g., multispecific proteins (or one or more polypeptide thereof)), is provided in Table 8.

TABLE 8
The Amino Acid Sequence of Exemplary hIg heavy chain constant region
components

| | | SEQ ID |
|---|---|---|
| Description | Amino Acid Sequence | NO |
| hIgG1 CH1 Region | ASTKGPSVFPLAPSSKSTSGGTAALGCLVKDYFPEPVTVSWN | 98 |
| | SGALTSGVHTFPAVLQSSGLYSLSSVVTVPSSSLGTQTYICN | |
| | VNHKPSNTKVDKKV | |
| hIgG1 Hinge Region | EPKSCDKTHTCP | 99 |
| hIgG1 CH2 Region | PCPAPELLGGPSVFLFPPKPKDTLMISRTPEVTCVVVDVSHE | 100 |
| | DPEVKFNWYVDGVEVHNAKTKPREEQYNSTYRVVSVLTVLHQ | |
| | DWLNGKEYKCKVSNKALPAPIEKTISKAK | |
| hIgG1 CH3 Region | GQPREPQVYTLPPSRDELTKNQVSLTCLVKGFYPSDIAVEWE | 101 |
| With C-terminal Lysine | SNGQPENNYKTTPPVLDSDGSFFLYSKLTVDKSRWQQGNVFS | |
| | CSVMHEALHNHYTQKSLSLSPGK | |
| hIgG1 CH3 Region | GQPREPQVYTLPPSRDELTKNQVSLTCLVKGFYPSDIAVEWE | 102 |
| Without C-terminal | SNGQPENNYKTTPPVLDSDGSFFLYSKLTVDKSRWQQGNVFS | |
| Lysine | CSVMHEALHNHYTQKSLSLSPG | |
| hIgG1 CH2 Region + | PCPAPELLGGPSVFLFPPKPKDTLMISRTPEVTCVVVDVSHE | 103 |
| CH3 Region | DPEVKFNWYVDGVEVHNAKTKPREEQYNSTYRVVSVLTVLHQ | |
| With C-terminal Lysine | DWLNGKEYKCKVSNKALPAPIEKTISKAKGQPREPQVYTLPP | |
| | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPGK | |
| hIgG1 CH2 Region + | PCPAPELLGGPSVFLFPPKPKDTLMISRTPEVTCVVVDVSHE | 104 |
| CH3 Region | DPEVKFNWYVDGVEVHNAKTKPREEQYNSTYRVVSVLTVLHQ | |
| Without C-terminal | DWLNGKEYKCKVSNKALPAPIEKTISKAKGQPREPQVYTLPP | |
| Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPG | |
| hIgG1 Partial Hinge | TCPPCPAPELLGGPSVFLFPPKPKDTLMISRTPEVTCVVVDV | 105 |
| Region + CH2 Region + | SHEDPEVKFNWYVDGVEVHNAKTKPREEQYNSTYRVVSVLTV | |
| CH3 Region | LHQDWLNGKEYKCKVSNKALPAPIEKTISKAKGQPREPQVYT | |
| With C-terminal Lysine | LPPSRDELTKNQVSLTCLVKGFYPSDIAVEWESNGQPENNYK | |
| | TTPPVLDSDGSFFLYSKLTVDKSRWQQGNVFSCSVMHEALHN | |
| | HYTQKSLSLSPGK | |
| hIgG1 Partial Hinge | TCPPCPAPELLGGPSVFLFPPKPKDTLMISRTPEVTCVVVDV | 106 |
| Region + CH2 Region + | SHEDPEVKFNWYVDGVEVHNAKTKPREEQYNSTYRVVSVLTV | |
| CH3 Region | LHQDWLNGKEYKCKVSNKALPAPIEKTISKAKGQPREPQVYT | |
| Without C-terminal | LPPSRDELTKNQVSLTCLVKGFYPSDIAVEWESNGQPENNYK | |
| Lysine | TTPPVLDSDGSFFLYSKLTVDKSRWQQGNVFSCSVMHEALHN | |
| | HYTQKSLSLSPG | |
| hIgG1 Hinge Region + | EPKSCDKTHTCPPCPAPELLGGPSVFLFPPKPKDTLMISRTP | 107 |
| CH2 Region + CH3 | EVTCVVVDVSHEDPEVKFNWYVDGVEVHNAKTKPREEQYNST | |
| Region | YRVVSVLTVLHQDWLNGKEYKCKVSNKALPAPIEKTISKAKG | |
| With C-terminal Lysine | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| hIgG1 Hinge Region + | EPKSCDKTHTCPPCPAPELLGGPSVFLFPPKPKDTLMISRTP | 108 |
| CH2 Region + CH3 | EVTCVVVDVSHEDPEVKFNWYVDGVEVHNAKTKPREEQYNST | |
| Region | YRVVSVLTVLHQDWLNGKEYKCKVSNKALPAPIEKTISKAKG | |
| Without C-terminal | QPREPQVYTLPPSRDELTKNQVSLTCLVKGFYPSDIAVEWES | |
| Lysine | NGQPENNYKTTPPVLDSDGSFFLYSKLTVDKSRWQQGNVFSC | |
| | SVMHEALHNHYTQKSLSLSPG | |
| hIgG1 CH1+ Hinge | ASTKGPSVFPLAPSSKSTSGGTAALGCLVKDYFPEPVTVSWN | 109 |
| Region + CH2 Region + | SGALTSGVHTFPAVLQSSGLYSLSSVVTVPSSSLGTQTYICN | |
| CH3 Region | VNHKPSNTKVDKKVEPKSCDKTHTCPPCPAPELLGGPSVFLF | |
| With C-terminal Lysine | PPKPKDTLMISRTPEVTCVVVDVSHEDPEVKFNWYVDGVEVH | |
| | NAKTKPREEQYNSTYRVVSVLTVLHQDWLNGKEYKCKVSNKA | |
| | LPAPIEKTISKAKGQPREPQVYTLPPSRDELTKNQVSLTCLV | |
| | KGFYPSDIAVEWESNGQPENNYKTTPPVLDSDGSFFLYSKLT | |
| | VDKSRWQQGNVFSCSVMHEALHNHYTQKSLSLSPGK | |
| hIgG1 CH1 + Hinge | ASTKGPSVFPLAPSSKSTSGGTAALGCLVKDYFPEPVTVSWN | 110 |
| Region + CH2 Region + | SGALTSGVHTFPAVLQSSGLYSLSSVVTVPSSSLGTQTYICN | |
| CH3 Region | VNHKPSNTKVDKKVEPKSCDKTHTCPPCPAPELLGGPSVFLF | |
| Without C-terminal | PPKPKDTLMISRTPEVTCVVVDVSHEDPEVKFNWYVDGVEVH | |
| Lysine | NAKTKPREEQYNSTYRVVSVLTVLHQDWLNGKEYKCKVSNKA | |
| | LPAPIEKTISKAKGQPREPQVYTLPPSRDELTKNQVSLTCLV | |
| | KGFYPSDIAVEWESNGQPENNYKTTPPVLDSDGSFFLYSKLT | |
| | VDKSRWQQGNVFSCSVMHEALHNHYTQKSLSLSPG | |
| hIgG4 CH1 Region | ASTKGPSVFPLAPCSRSTSESTAALGCLVKDYFPEPVTVSWN | 111 |
| | SGALTSGVHTFPAVLQSSGLYSLSSVVTVPSSSLGTKTYTCN | |
| | VDHKPSNTKVDKRV | |
| hIgG4 Hinge Region | ESKYGPPCPSCP | 112 |
| hIgG4 CH2 Region | APEFLGGPSVFLFPPKPKDTLMISRTPEVTCVVVDVSQEDPE | 113 |
| | VQFNWYVDGVEVHNAKTKPREEQFNSTYRVVSVLTVLHQDWL | |
| | NGKEYKCKVSNKGLPSSIEKTISKAK | |
| hIgG4 CH3 Region | GQPREPQVYTLPPSQEEMTKNQVSLTCLVKGFYPSDIAVEWE | 114 |
| With C-terminal Lysine | SNGQPENNYKTTPPVLDSDGSFFLYSRLTVDKSRWQEGNVFS | |
| | CSVMHEALHNHYTQKSLSLSLGK | |
| hIgG4 CH3 Region | GQPREPQVYTLPPSQEEMTKNQVSLTCLVKGFYPSDIAVEWE | 115 |
| Without C-terminal | SNGQPENNYKTTPPVLDSDGSFFLYSRLTVDKSRWQEGNVFS | |
| Lysine | CSVMHEALHNHYTQKSLSLSLG | |
| hIgG4 CH2 Region + | APEFLGGPSVFLFPPKPKDTLMISRTPEVTCVVVDVSQEDPE | 116 |
| CH3 Region | VQFNWYVDGVEVHNAKTKPREEQFNSTYRVVSVLTVLHQDWL | |
| With C-terminal Lysine | NGKEYKCKVSNKGLPSSIEKTISKAKGQPREPQVYTLPPSQE | |
| | EMTKNQVSLTCLVKGFYPSDIAVEWESNGQPENNYKTTPPVL | |
| | DSDGSFFLYSRLTVDKSRWQEGNVFSCSVMHEALHNHYTQKS | |
| | LSLSLGK | |
| hIgG4 CH2 Region + | APEFLGGPSVFLFPPKPKDTLMISRTPEVTCVVVDVSQEDPE | 117 |
| CH3 Region | VQFNWYVDGVEVHNAKTKPREEQFNSTYRVVSVLTVLHQDWL | |
| Without C-terminal | NGKEYKCKVSNKGLPSSIEKTISKAKGQPREPQVYTLPPSQE | |
| Lysine | EMTKNQVSLTCLVKGFYPSDIAVEWESNGQPENNYKTTPPVL | |
| | DSDGSFFLYSRLTVDKSRWQEGNVFSCSVMHEALHNHYTQKS | |
| | LSLSLG | |
| hIgG4 Partial Hinge | PCPSCPAPEFLGGPSVFLFPPKPKDTLMISRTPEVTCVVVDV | 118 |
| Region + CH2 Region + | SQEDPEVQFNWYVDGVEVHNAKTKPREEQFNSTYRVVSVLTV | |
| CH3 Region | LHQDWLNGKEYKCKVSNKGLPSSIEKTISKAKGQPREPQVYT | |
| With C-terminal Lysine | LPPSQEEMTKNQVSLTCLVKGFYPSDIAVEWESNGQPENNYK | |
| | TTPPVLDSDGSFFLYSRLTVDKSRWQEGNVFSCSVMHEALHN | |
| | HYTQKSLSLSLGK | |
| hIgG4 Partial Hinge | PCPSCPAPEFLGGPSVFLFPPKPKDTLMISRTPEVTCVVVDV | 119 |
| Region + CH2 Region + | SQEDPEVQFNWYVDGVEVHNAKTKPREEQFNSTYRVVSVLTV | |
| CH3 Region | LHQDWLNGKEYKCKVSNKGLPSSIEKTISKAKGQPREPQVYT | |
| Without C-terminal | LPPSQEEMTKNQVSLTCLVKGFYPSDIAVEWESNGQPENNYK | |
| Lysine | TTPPVLDSDGSFFLYSRLTVDKSRWQEGNVFSCSVMHEALHN | |
| | HYTQKSLSLSLG | |
| hIgG4 Hinge Region + | ESKYGPPCPSCPAPEFLGGPSVFLFPPKPKDTLMISRTPEVT | 120 |
| CH2 Region + CH3 | CVVVDVSQEDPEVQFNWYVDGVEVHNAKTKPREEQFNSTYRV | |
| Region | VSVLTVLHQDWLNGKEYKCKVSNKGLPSSIEKTISKAKGQPR | |
| With C-terminal Lysine | EPQVYTLPPSQEEMTKNQVSLTCLVKGFYPSDIAVEWESNGQ | |
| | PENNYKTTPPVLDSDGSFFLYSRLTVDKSRWQEGNVFSCSVM | |
| | HEALHNHYTQKSLSLSLGK | |
| hIgG4 Hinge Region + | ESKYGPPCPSCPAPEFLGGPSVFLFPPKPKDTLMISRTPEVT | 121 |
| CH2 Region + CH3 | CVVVDVSQEDPEVQFNWYVDGVEVHNAKTKPREEQFNSTYRV | |
| Region | VSVLTVLHQDWLNGKEYKCKVSNKGLPSSIEKTISKAKGQPR | |
| Without C-terminal | EPQVYTLPPSQEEMTKNQVSLTCLVKGFYPSDIAVEWESNGQ | |
| Lysine | PENNYKTTPPVLDSDGSFFLYSRLTVDKSRWQEGNVFSCSVM | |
| | HEALHNHYTQKSLSLSLG | |
| hIgG4 CH1 + Hinge | ASTKGPSVFPLAPCSRSTSESTAALGCLVKDYFPEPVTVSWN | 122 |
| Region + CH2 Region + | SGALTSGVHTFPAVLQSSGLYSLSSVVTVPSSSLGTKTYTCN | |
| CH3 Region | VDHKPSNTKVDKRVESKYGPPCPSCPAPEFLGGPSVFLFPPK | |
| | PKDTLMISRTPEVTCVVVDVSQEDPEVQFNWYVDGVEVHNAK | |
| With C-terminal Lysine | TKPREEQFNSTYRVVSVLTVLHQDWLNGKEYKCKVSNKGLPS | |
| | SIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| hIgG4 CH1 + Hinge | ASTKGPSVFPLAPCSRSTSESTAALGCLVKDYFPEPVTVSWN | 123 |
| Region + CH2 Region + | SGALTSGVHTFPAVLQSSGLYSLSSVVTVPSSSLGTKTYTCN | |
| CH3 Region | VDHKPSNTKVDKRVESKYGPPCPSCPAPEFLGGPSVFLFPPK | |
| Without C-terminal | PKDTLMISRTPEVTCVVVDVSQEDPEVQFNWYVDGVEVHNAK | |
| Lysine | TKPREEQFNSTYRVVSVLTVLHQDWLNGKEYKCKVSNKGLPS | |
| | SIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLG | |
| Ig light chain kappa | RTVAAPSVFIFPPSDEQLKSGTASVVCLLNNFYPREAKVQWK | 124 |
| constant region (κCL) | VDNALQSGNSQESVTEQDSKDSTYSLSSTLILSKADYEKHKV | |
| | YACEVTHQGLSSPVTKSFNRGEC | |
| Ig light chain kappa | GQPKANPTVTLFPPSSEELQANKATLVCLISDFYPGAVTVAW | 125 |
| constant region (λCL) | KADGSPVKAGVETTKPSKQSNNKYAASSYLSLTPEQWKSHRS | |
| | YSCQVTHEGSTVEKTVAPTECS | |
| hIgM CH2 | PTLFPLVSCENSNPSSTVAVGCLAQDFLPDSITFSWKYNQSQ | 126 |
| | KISSTRGFPSVLRGGKYAATSQVLLPSKDVMQGTDEHVC | |

In some embodiments, any one of the Ig constant regions set forth in Table 8 comprising a CH3 region further lacks the C-terminal lysine. In some embodiments, any one of the Ig constant regions set forth in Table 8 comprising a CH3 region further lacks the C-terminal glycine and lysine.

In some embodiments, the multispecific protein (or one or more polypeptide thereof) comprises one or more hIg constant regions, wherein the amino acid sequence of the one or more hIg constant regions comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 8. In some embodiments, the amino acid sequence of the one or more hIg constant regions comprises or consists of an amino acid sequence set forth in Table 8.

In some embodiments, the amino acid sequence of the one or more hIg constant regions comprises or consists of an amino acid sequence set forth in Table 8, and further comprises 1 or more but less than 15% (less than 12%, less than 10%, less than 8%), amino acid variations (e.g., amino acid substitutions, deletions, or additions). In some embodiments, the amino acid sequence of the one or more hIg constant regions comprises or consists of an amino acid sequence set forth in Table 8, comprising or consisting of at least about 1, 2, 3, 4, 5, 6, 7, 8, 9, 10, or more amino acid modifications (e.g., amino acid substitutions, deletions, or additions). In some embodiments, the amino acid sequence of the one or more hIg constant regions comprises or consists of an amino acid sequence set forth in Table 8, comprising or consisting of about 1, 2, 3, 4, 5, 6, 7, 8, 9, 10, or more amino acid modifications (e.g., amino acid substitutions, deletions, or additions). In some embodiments, the amino acid sequence of the one or more hIg constant regions comprises or consists of an amino acid sequence set forth in Table 8, comprising or consisting of about no more than 1, 2, 3, 4, 5, 6, 7, 8, 9, 10, or more amino acid modifications (e.g., amino acid substitutions, deletions, or additions).

In some embodiments, the amino acid sequence of the one or more hIg constant regions comprises or consists of an amino acid sequence set forth in Table 8, and further comprises 1 or more but less than 15% (less than 12%, less than 10%, less than 8%), amino acid substitutions. In some embodiments, the amino acid sequence of the one or more hIg constant regions comprises or consists of an amino acid sequence set forth in Table 8, comprising or consisting of at least about 1, 2, 3, 4, 5, 6, 7, 8, 9, 10, or more amino acid substitutions. In some embodiments, the amino acid sequence of the one or more hIg constant regions comprises or consists of an amino acid sequence set forth in Table 8, comprising or consisting of about 1, 2, 3, 4, 5, 6, 7, 8, 9, 10, or more amino acid substitutions. In some embodiments, the amino acid sequence of the one or more hIg constant regions comprises or consists of an amino acid sequence set forth in Table 8, comprising or consisting of about no more than 1, 2, 3, 4, 5, 6, 7, 8, 9, 10, or more amino acid substitutions.

In some embodiments, the amino acid sequence of the one or more hIg constant regions comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 103-110 or 116-123. In some embodiments, the amino acid sequence of the one or more hIg constant regions comprises or consists of the amino acid sequence of any one of SEQ ID NOS: 103-110 or 116-123.

In some embodiments, the amino acid sequence of the one or more hIg constant regions comprises or consists of the amino acid sequence of any one of SEQ ID NOS: 103-110 or 116-123, and further comprises 1 or more but less than 15% (less than 12%, less than 10%, less than 8%), amino acid modifications (e.g., amino acid substitutions, deletions, or additions). In some embodiments, the amino acid sequence of the one or more hIg constant regions comprises or consists of the amino acid sequence of any one of SEQ ID NOS: 103-110 or 116-123, comprising or consisting at least about 1, 2, 3, 4, 5, 6, 7, 8, 9, 10, or more amino acid modifications (e.g., amino acid substitutions, deletions, or additions). In some embodiments, the amino acid sequence of the one or more hIg constant regions comprises or consists of the amino acid sequence of any one of SEQ ID NOS: 103-110 or 116-123, comprising or consisting about 1, 2, 3, 4, 5, 6, 7, 8, 9, or 10 amino acid modifications (e.g., amino acid substitutions, deletions, or additions). In some embodiments, the amino acid sequence of the one or more hIg constant regions comprises or consists of the amino acid sequence of any one of SEQ ID NOS: 103-110 or 116-123, comprising or consisting of no more than about 1, 2, 3, 4, 5, 6, 7, 8, 9, or 10 amino acid modifications (e.g., amino acid substitutions, deletions, or additions).

In some embodiments, the amino acid sequence of the one or more hIg constant regions comprises or consists of the amino acid sequence of any one of SEQ ID NOS: 103-110 or 116-123, and further comprises 1 or more but less than 15% (less than 12%, less than 10%, less than 8%), amino acid substitutions. In some embodiments, the amino acid sequence of the one or more hIg constant regions comprises or consists of the amino acid sequence of any one of SEQ ID NOS: 103-110 or 116-123, comprising or consisting at least about 1, 2, 3, 4, 5, 6, 7, 8, 9, 10, or more amino acid substitutions. In some embodiments, the amino acid sequence of the one or more hIg constant regions comprises or consists of the amino acid sequence of any one of SEQ ID NOS: 103-110 or 116-123, comprising or consisting about 1, 2, 3, 4, 5, 6, 7, 8, 9, or 10 amino acid substitutions. In some embodiments, the amino acid sequence of the one or more hIg constant regions comprises or consists of the amino acid sequence of any one of SEQ ID NOS: 103-110 or 116-123, comprising or consisting of no more than about 1, 2, 3, 4, 5, 6, 7, 8, 9, or 10 amino acid substitutions.

### 5.2.7.1 Ig Effector Function

As described herein, in some embodiments, the multispecific protein (or one or more polypeptide thereof) comprises a Fc region (see, e.g., §§ 5.2.7, 5.2.7.1, 5.2.7.2, 5.2.7.3). In some embodiments, the Fc region of a fusion protein or polypeptide described herein exhibits a decrease in one or more Fc effector function relative to a reference (e.g., wild type) Fc region. Exemplary Fc effector functions include, but are not limited to, antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

Standard in vitro and/or in vivo assays known in the art can be conducted to evaluate Fc effector function, including, any one or more of ADCC, CDC, ADCP, Fc receptor (e.g., Fcγ receptor) binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (⁵¹Cr) assay. Briefly, ⁵¹Cr is pre-loaded into target cells, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide flurophore (Europium). See, e.g., Parekh, Bhavin S et al. “Development and validation of an antibody-dependent cell-mediated cytotoxicity-reporter gene assay.” mAbs vol. 4,3 (2012): 310-8. Doi:10.4161/mabs.19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a fusion protein described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of a hIg fusion protein or polypeptide described herein to bind C1q (or bind with less affinity than a reference fusion protein) and hence lack (or have decreased) CDC activity. The binding of a hIg fusion protein or polypeptide described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127(6):681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi:10.3791/3588; the entire contents of each of which is incorporated by reference herein.

Binding of a hIg fusion protein or polypeptide described herein to a Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4″ Ed., Lippincott-Raven, Philadelphia (1999), the entire contents of which is incorporated by reference herein for all purposes.

In some embodiments, the Fc region of a multispecific protein (or one or more polypeptide thereof) described herein is modified (e.g., comprises one or more amino acid modification (e.g., one or more amino acid substitution, deletion, addition, etc.)) (referred to herein as a “modified Fc region”), relative to the amino acid sequence of a reference Fc region (e.g., a wild type Fc region, e.g., SEQ ID NO: 107 or 108). In some embodiments, the one or more amino acid modification (e.g., the one or more amino acid substitution, deletion, addition, etc.)) decreases or abolishes one or more Fc effector function, relative to a reference Fc that does not comprise the modification (e.g., the one or more modification (e.g., the one or more amino acid substitution, deletion, addition, etc.)).

In some embodiments, the multispecific protein (or one or more polypeptide thereof) comprising a modified Fc region exhibits no detectable or decreased ADCC compared to a reference multispecific protein (or one or more polypeptide thereof) that does not comprise the Fc region modification (e.g., the one or more amino acid modification (e.g., one or more amino acid substitution, deletion, or addition)). In some embodiments, the multispecific protein (or one or more polypeptide thereof) comprising a modified Fc region exhibits no detectable or decreased CDC compared to a reference multispecific protein (or one or more polypeptide thereof) that does not comprise the Fc region modification (e.g., the one or more amino acid modification (e.g., one or more amino acid substitution, deletion, or addition)). In some embodiments, the multispecific protein (or one or more polypeptide thereof) comprising a modified Fc region exhibits no detectable or decreased ADCP compared to a reference multispecific protein (or one or more polypeptide thereof) that does not comprise the Fc region modification (e.g., the one or more modification (e.g., one or more amino acid substitution, deletion, or addition)). In some embodiments, the multispecific protein (or one or more polypeptide thereof) comprising a modified Fc region exhibits decreased or no detectable specific binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) compared to a reference multispecific protein (or one or more polypeptide thereof) that does not comprise the Fc region modification (e.g., the one or more modification (e.g., one or more amino acid substitution, deletion, or addition)). In some embodiments, the multispecific protein (or one or more polypeptide thereof) comprising a modified Fc region exhibits decreased or no detectable specific binding affinity to FcγRI, FcγIIa, and/or FcγIIIa compared to a reference multispecific protein (or one or more polypeptide thereof) that does not comprise the Fc region modification (e.g., the one or more modification (e.g., one or more amino acid substitution, deletion, or addition)). In some embodiments, the multispecific protein (or polypeptide thereof) or polypeptide comprising a modified Fc exhibits decreased or no detectable specific binding affinity to FcγRI compared to a reference multispecific protein (or one or more polypeptide thereof) that does not comprise the Fc modification (e.g., the one or more modification (e.g., one or more amino acid substitution, deletion, or addition)). In some embodiments, the multispecific protein (or one or more polypeptide thereof) comprising a modified Fc exhibits decreased or no detectable specific binding affinity to FcγIIa compared to a reference multispecific protein (or polypeptide thereof) or polypeptide that does not comprise the Fc region modification (e.g., the one or more modification (e.g., one or more amino acid substitution, deletion, or addition)). In some embodiments, the multispecific protein (or one or more polypeptide thereof) comprising a modified Fc region exhibits decreased or no detectable specific binding affinity to FcγIIIa compared to a reference multispecific protein (or one or more polypeptide thereof) that does not comprise the Fc region modification (e.g., the one or more modification (e.g., one or more amino acid substitution, deletion, or addition)). In some embodiments, the multispecific protein (or one or more polypeptide thereof) comprising a modified Fc region exhibits decreased or no detectable specific binding affinity to C1q compared to a reference multispecific protein (or one or more polypeptide thereof) that does not comprise the Fc region modification (e.g., the one or more modification (e.g., one or more amino acid substitution, deletion, or addition)).

In some embodiments, the multispecific protein (or one or more polypeptide thereof) comprising an Fc region exhibits no detectable ADCC. In some embodiments, the multispecific protein (or one or more polypeptide thereof) comprising an Fc region exhibits no detectable CDC. In some embodiments, the multispecific protein (or one or more polypeptide thereof) comprising an Fc region exhibits no detectable ADCP. In some embodiments, the multispecific protein (or one or more polypeptide thereof) comprising an Fc region exhibits no detectable specific binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In some embodiments, the multispecific protein (or one or more polypeptide thereof) comprising an Fc region exhibits no detectable specific binding affinity to FcγRI, FcγIIa, and/or. In some embodiments, the multispecific protein (or polypeptide thereof) comprising an Fc exhibits no detectable specific binding affinity to FcγRI. In some embodiments, the multispecific protein (or one or more polypeptide thereof) comprising an Fc exhibits no detectable specific binding affinity to FcγIIa. In some embodiments, the multispecific protein (or one or more polypeptide thereof) comprising an Fc region exhibits no detectable specific binding affinity to FcγIIIa. In some embodiments, the multispecific protein (or one or more polypeptide thereof) comprising an Fc region exhibits no detectable specific binding affinity to C1q.

Amino acid substitutions that decrease or abolish one or more Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated by reference herein for all purposes, see more particularly for example, e.g., Table 3 of Saunders.

In some embodiments, the modified Fc comprises a hIgG1 Fc region comprising one or more amino acid modifications (e.g., one or more amino acid substitutions). In some embodiments, the hIgG1 Fc region comprises an amino acid substitution at amino acid positions L234, L235, and/or P329, EU numbering according to Kabat. In some embodiments, the hIgG1 Fc region comprises the following amino acid substitutions L234A and/or L235A, EU numbering according to Kabat. In some embodiments, the hIgG1 Fc region comprises the following amino acid substitutions L234A, L235A, and P329G, EU numbering according to Kabat. In some embodiments, the hIgG1 Fc region comprises the following amino acid substitutions L234A, L235A, and P329A, EU numbering according to Kabat.

In some embodiments, the modified Fc region comprises a hIg4 Fc region comprising one or more amino acid modifications (e.g., one or more amino acid substitutions). In some embodiments, the hIgG4 Fc region comprises an amino acid substitution at amino acid positions S228, F234, and/or L235, EU numbering according to Kabat. In some embodiments, the hIgG4 Fe region comprises the following amino acid substitutions S228P, F234A, and/or L235A, EU numbering according to Kabat. In some embodiments, the hIgG4 Fc region comprises the following amino acid substitutions S228P, F234A, and/or L235E, EU numbering according to Kabat. In some embodiments, the hIgG4 Fc comprises the following amino acid substitutions S228P and/or L235E, EU numbering according to Kabat.

The amino acid sequence of exemplary modified Fc regions that are known in the art to exhibit a decrease in one more effector function is provided in Table 9.

TABLE 9
The Amino Acid Sequence of Exemplary Modified Fc Regions

| Description | Amino Acid Sequence | SEQ ID NO |
|---|---|---|
| hIgG1 CH2 Region + | PCPAPEAAGGPSVFLFPPKPKDTLMISRTPEVTCVVVD | 127 |
| CH3 Region | VSHEDPEVKFNWYVDGVEVHNAKTKPREEQYNSTYRVV | |
| L234A/L235A | SVLTVLHQDWLNGKEYKCKVSNKALPAPIEKTISKAKG | |
| With C-terminal Lysine | QPREPQVYTLPPSRDELTKNQVSLTCLVKGFYPSDIAV | |
| | EWESNGQPENNYKTTPPVLDSDGSFFLYSKLTVDKSRW | |
| | QQGNVFSCSVMHEALHNHYTQKSLSLSPGK | |
| hIgG1 CH2 Region + | PCPAPEAAGGPSVFLFPPKPKDTLMISRTPEVTCVVVD | 128 |
| CH3 Region | VSHEDPEVKFNWYVDGVEVHNAKTKPREEQYNSTYRVV | |
| L234A/L235A | SVLTVLHQDWLNGKEYKCKVSNKALPAPIEKTISKAKG | |
| Without C-terminal | QPREPQVYTLPPSRDELTKNQVSLTCLVKGFYPSDIAV | |
| Lysine | EWESNGQPENNYKTTPPVLDSDGSFFLYSKLTVDKSRW | |
| | QQGNVFSCSVMHEALHNHYTQKSLSLSPG | |
| hIgG1 CH2 Region + | PCPAPEAAGGPSVFLFPPKPKDTLMISRTPEVTCVVVD | 129 |
| CH3 Region | VSHEDPEVKFNWYVDGVEVHNAKTKPREEQYNSTYRVV | |
| L234A/L235A/P329A | SVLTVLHQDWLNGKEYKCKVSNKALAAPIEKTISKAKG | |
| With C-terminal Lysine | QPREPQVYTLPPSRDELTKNQVSLTCLVKGFYPSDIAV | |
| | EWESNGQPENNYKTTPPVLDSDGSFFLYSKLTVDKSRW | |
| | QQGNVFSCSVMHEALHNHYTQKSLSLSPGK | |
| hIgG1 CH2 Region + | PCPAPEAAGGPSVFLFPPKPKDTLMISRTPEVTCVVVD | 130 |
| CH3 Region | VSHEDPEVKFNWYVDGVEVHNAKTKPREEQYNSTYRVV | |
| L234A/L235A/P329A | SVLTVLHQDWLNGKEYKCKVSNKALAAPIEKTISKAKG | |
| Without C-terminal | QPREPQVYTLPPSRDELTKNQVSLTCLVKGFYPSDIAV | |
| Lysine | EWESNGQPENNYKTTPPVLDSDGSFFLYSKLTVDKSRW | |
| | QQGNVFSCSVMHEALHNHYTQKSLSLSPG | |
| hIgG1 CH2 Region + | PCPAPEAAGGPSVFLFPPKPKDTLMISRTPEVTCVVVD | 131 |
| CH3 Region | VSHEDPEVKFNWYVDGVEVHNAKTKPREEQYNSTYRVV | |
| L234A/L235A/P329G | SVLTVLHQDWLNGKEYKCKVSNKALGAPIEKTISKAKG | |
| With C-terminal Lysine | QPREPQVYTLPPSRDELTKNQVSLTCLVKGFYPSDIAV | |
| | EWESNGQPENNYKTTPPVLDSDGSFFLYSKLTVDKSRW | |
| | QQGNVFSCSVMHEALHNHYTQKSLSLSPGK | |
| hIgG1 CH2 Region + | PCPAPEAAGGPSVFLFPPKPKDTLMISRTPEVTCVVVD | 132 |
| CH3 Region | VSHEDPEVKFNWYVDGVEVHNAKTKPREEQYNSTYRVV | |
| L234A/L235A/P329G | SVLTVLHQDWLNGKEYKCKVSNKALGAPIEKTISKAKG | |
| Without C-terminal | QPREPQVYTLPPSRDELTKNQVSLTCLVKGFYPSDIAV | |
| Lysine | EWESNGQPENNYKTTPPVLDSDGSFFLYSKLTVDKSRW | |
| | QQGNVFSCSVMHEALHNHYTQKSLSLSPG | |
| hIgG1 Partial Hinge | TCPPCPAPEAAGGPSVFLFPPKPKDTLMISRTPEVTCV | 133 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| L234A/L235A | AKGQPREPQVYTLPPSRDELTKNQVSLTCLVKGFYPSD | |
| With C-terminal Lysine | IAVEWESNGQPENNYKTTPPVLDSDGSFFLYSKLTVDK | |
| | SRWQQGNVFSCSVMHEALHNHYTQKSLSLSPGK | |
| hIgG1 Partial Hinge | TCPPCPAPEAAGGPSVFLFPPKPKDTLMISRTPEVTCV | 134 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| L234A/L235A | AKGQPREPQVYTLPPSRDELTKNQVSLTCLVKGFYPSD | |
| Without C-terminal | IAVEWESNGQPENNYKTTPPVLDSDGSFFLYSKLTVDK | |
| Lysine | SRWQQGNVFSCSVMHEALHNHYTQKSLSLSPG | |
| hIgG1 Partial Hinge | TCPPCPAPEAAGGPSVFLFPPKPKDTLMISRTPEVTCV | 135 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALAAPIEKTISK | |
| L234A/L235A/P329A | AKGQPREPQVYTLPPSRDELTKNQVSLTCLVKGFYPSD | |
| With C-terminal Lysine | IAVEWESNGQPENNYKTTPPVLDSDGSFFLYSKLTVDK | |
| | SRWQQGNVFSCSVMHEALHNHYTQKSLSLSPGK | |
| hIgG1 Partial Hinge | TCPPCPAPEAAGGPSVFLFPPKPKDTLMISRTPEVTCV | 136 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALAAPIEKTISK | |
| L234A/L235A/P329A | AKGQPREPQVYTLPPSRDELTKNQVSLTCLVKGFYPSD | |
| Without C-terminal | IAVEWESNGQPENNYKTTPPVLDSDGSFFLYSKLTVDK | |
| Lysine | SRWQQGNVFSCSVMHEALHNHYTQKSLSLSPG | |
| hIgG1 Partial Hinge | TCPPCPAPEAAGGPSVFLFPPKPKDTLMISRTPEVTCV | 137 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALGAPIEKTISK | |
| L234A/L235A/P329G | AKGQPREPQVYTLPPSRDELTKNQVSLTCLVKGFYPSD | |
| With C-terminal Lysine | IAVEWESNGQPENNYKTTPPVLDSDGSFFLYSKLTVDK | |
| | SRWQQGNVFSCSVMHEALHNHYTQKSLSLSPGK | |
| hIgG1 Partial Hinge | TCPPCPAPEAAGGPSVFLFPPKPKDTLMISRTPEVTCV | 138 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALGAPIEKTISK | |
| L234A/L235A/P329G | AKGQPREPQVYTLPPSRDELTKNQVSLTCLVKGFYPSD | |
| Without C-terminal | IAVEWESNGQPENNYKTTPPVLDSDGSFFLYSKLTVDK | |
| Lysine | SRWQQGNVFSCSVMHEALHNHYTQKSLSLSPG | |
| hIgG1 Hinge Region + | EPKSCDKTHTCPPCPAPEAAGGPSVFLFPPKPKDTLMI | 139 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| L234A/L235A | APIEKTISKAKGQPREPQVYTLPPSRDELTKNQVSLTC | |
| With C-terminal Lysine | LVKGFYPSDIAVEWESNGQPENNYKTTPPVLDSDGSFF | |
| | LYSKLTVDKSRWQQGNVFSCSVMHEALHNHYTQKSLSL | |
| | SPGK | |
| hIgG1 Hinge Region + | EPKSCDKTHTCPPCPAPEAAGGPSVFLFPPKPKDTLMI | 140 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| L234A/L235A | APIEKTISKAKGQPREPQVYTLPPSRDELTKNQVSLTC | |
| Without C-terminal | LVKGFYPSDIAVEWESNGQPENNYKTTPPVLDSDGSFF | |
| Lysine | LYSKLTVDKSRWQQGNVFSCSVMHEALHNHYTQKSLSL | |
| | SPG | |
| hIgG1 Hinge Region + | EPKSCDKTHTCPPCPAPEAAGGPSVFLFPPKPKDTLMI | 141 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| L234A/L235A/P329A | APIEKTISKAKGQPREPQVYTLPPSRDELTKNQVSLTC | |
| With C-terminal Lysine | LVKGFYPSDIAVEWESNGQPENNYKTTPPVLDSDGSFF | |
| | LYSKLTVDKSRWQQGNVFSCSVMHEALHNHYTQKSLSL | |
| | SPGK | |
| hIgG1 Hinge Region + | EPKSCDKTHTCPPCPAPEAAGGPSVFLFPPKPKDTLMI | 142 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| L234A/L235A/P329A | APIEKTISKAKGQPREPQVYTLPPSRDELTKNQVSLTC | |
| Without C-terminal | LVKGFYPSDIAVEWESNGQPENNYKTTPPVLDSDGSFF | |
| Lysine | LYSKLTVDKSRWQQGNVFSCSVMHEALHNHYTQKSLSL | |
| | SPG | |
| hIgG1 Hinge Region + | EPKSCDKTHTCPPCPAPEAAGGPSVFLFPPKPKDTLMI | 143 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| L234A/L235A/P329G | APIEKTISKAKGQPREPQVYTLPPSRDELTKNQVSLTC | |
| With C-terminal Lysine | LVKGFYPSDIAVEWESNGQPENNYKTTPPVLDSDGSFF | |
| | LYSKLTVDKSRWQQGNVFSCSVMHEALHNHYTQKSLSL | |
| | SPGK | |
| hIgG1 Hinge Region + | EPKSCDKTHTCPPCPAPEAAGGPSVFLFPPKPKDTLMI | 144 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| L234A/L235A/P329G | APIEKTISKAKGQPREPQVYTLPPSRDELTKNQVSLTC | |
| Without C-terminal | LVKGFYPSDIAVEWESNGQPENNYKTTPPVLDSDGSFF | |
| Lysine | LYSKLTVDKSRWQQGNVFSCSVMHEALHNHYTQKSLSL | |
| | SPG | |
| hIgG4 CH2 Region + | APEAAGGPSVFLFPPKPKDTLMISRTPEVTCVVVDVSQ | 145 |
| CH3 Region | EDPEVQFNWYVDGVEVHNAKTKPREEQFNSTYRVVSVL | |
| S228P/F234A/L235A | TVLHQDWLNGKEYKCKVSNKGLPSSIEKTISKAKGQPR | |
| With C-terminal Lysine | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGK | |
| hIgG4 CH2 Region + | APEAAGGPSVFLFPPKPKDTLMISRTPEVTCVVVDVSQ | 146 |
| CH3 Region | EDPEVQFNWYVDGVEVHNAKTKPREEQFNSTYRVVSVL | |
| S228P/F234A/L235A | TVLHQDWLNGKEYKCKVSNKGLPSSIEKTISKAKGQPR | |
| Without C-terminal | EPQVYTLPPSQEEMTKNQVSLTCLVKGFYPSDIAVEWE | |
| Lysine | SNGQPENNYKTTPPVLDSDGSFFLYSRLTVDKSRWQEG | |
| | NVFSCSVMHEALHNHYTQKSLSLSLG | |
| hIgG4 Partial Hinge | PCPSCPAPEAAGGPSVFLFPPKPKDTLMISRTPEVTCV | 147 |
| Region + CH2 Region + | VVDVSQEDPEVQFNWYVDGVEVHNAKTKPREEQFNSTY | |
| CH3 Region | RVVSVLTVLHQDWLNGKEYKCKVSNKGLPSSIEKTISK | |
| S228P/F234A/L235A | AKGQPREPQVYTLPPSQEEMTKNQVSLTCLVKGFYPSD | |
| With C-terminal Lysine | IAVEWESNGQPENNYKTTPPVLDSDGSFFLYSRLTVDK | |
| | SRWQEGNVFSCSVMHEALHNHYTQKSLSLSLGK | |
| hIgG4 Partial Hinge | PCPPCPAPEAAGGPSVFLFPPKPKDTLMISRTPEVTCV | 148 |
| Region + CH2 Region + | VVDVSQEDPEVQFNWYVDGVEVHNAKTKPREEQFNSTY | |
| CH3 Region | RVVSVLTVLHQDWLNGKEYKCKVSNKGLPSSIEKTISK | |
| S228P/F234A/L235A | AKGQPREPQVYTLPPSQEEMTKNQVSLTCLVKGFYPSD | |
| Without C-terminal | IAVEWESNGQPENNYKTTPPVLDSDGSFFLYSRLTVDK | |
| Lysine | SRWQEGNVFSCSVMHEALHNHYTQKSLSLSLG | |
| hIgG4 Hinge Region + | ESKYGPPCPPCPAPEAAGGPSVFLFPPKPKDTLMISRT | 149 |
| CH2 Region + CH3 | PEVTCVVVDVSQEDPEVQFNWYVDGVEVHNAKTKPREE | |
| Region | QFNSTYRVVSVLTVLHQDWLNGKEYKCKVSNKGLPSSI | |
| S228P/F234A/L235A | EKTISKAKGQPREPQVYTLPPSQEEMTKNQVSLTCLVK | |
| With C-terminal Lysine | GFYPSDIAVEWESNGQPENNYKTTPPVLDSDGSFFLYS | |
| | RLTVDKSRWQEGNVFSCSVMHEALHNHYTQKSLSLSLG | |
| | K | |
| hIgG4 Hinge Region + | ESKYGPPCPPCPAPEAAGGPSVFLFPPKPKDTLMISRT | 150 |
| CH2 Region + CH3 | PEVTCVVVDVSQEDPEVQFNWYVDGVEVHNAKTKPREE | |
| Region | QFNSTYRVVSVLTVLHQDWLNGKEYKCKVSNKGLPSSI | |
| S228P/F234A/L235A | EKTISKAKGQPREPQVYTLPPSQEEMTKNQVSLTCLVK | |
| Without C-terminal | GFYPSDIAVEWESNGQPENNYKTTPPVLDSDGSFFLYS | |
| Lysine | RLTVDKSRWQEGNVFSCSVMHEALHNHYTQKSLSLSLG | |
| hIgG4 Hinge Region + | AESKYGPPCPPCPAPEAAGGPSVFLFPPKPKDTLMISR | 151 |
| CH2 Region + CH3 | TPEVTCVVVDVSQEDPEVQFNWYVDGVEVHNAKTKPRE | |
| Region | EQFNSTYRVVSVLTVLHQDWLNGKEYKCKVSNKGLPSS | |
| (Modified) | IEKTISKAKGQPREPQVYTLPPSQEEMTKNQVSLTCLV | |
| S228P/F234A/L235A | KGFYPSDIAVEWESNGQPENNYKTTPPVLDSDGSFFLY | |
| With C-terminal Lysine | SRLTVDKSRWQEGNVFSCSVMHEALHNHYTQKSLSLSL | |
| | GK | |
| hIgG4 Hinge Region + | AESKYGPPCPPCPAPEAAGGPSVFLFPPKPKDTLMISR | 152 |
| CH2 Region + CH3 | TPEVTCVVVDVSQEDPEVQFNWYVDGVEVHNAKTKPRE | |
| Region | EQFNSTYRVVSVLTVLHQDWLNGKEYKCKVSNKGLPSS | |
| (Modified) | IEKTISKAKGQPREPQVYTLPPSQEEMTKNQVSLTCLV | |
| S228P/F234A/L235A | KGFYPSDIAVEWESNGQPENNYKTTPPVLDSDGSFFLY | |
| Without C-terminal | SRLTVDKSRWQEGNVFSCSVMHEALHNHYTQKSLSLSL | |
| Lysine | G | |

In some embodiments, the amino acid sequence of the modified hIgG1 Fc region comprises or consists of an A amino acid at position L234 and/or an A amino acid at position L235, EU numbering according to Kabat. In some embodiments, the amino acid sequence of the modified hIgG1 Fc region comprises or consists of an A amino acid at position L234 and an A amino acid at position L235, EU numbering according to Kabat. In some embodiments, the amino acid sequence of the modified hIgG1 Fc region comprises or consists of an A or G amino acid at position P329, EU numbering according to Kabat.

In some embodiments, the amino acid sequence of the modified hIgG1 Fc region comprises or consists of an A amino acid at position L234; an A amino acid at position L235; and an A or G amino acid at position P329 EU numbering according to Kabat. In some embodiments, the amino acid sequence of the modified hIgG1 Fc region comprises or consists of an A amino acid at position L234; an A amino acid at position L235; and an A amino acid at position P329 EU numbering according to Kabat. In some embodiments, the amino acid sequence of the modified hIgG1 Fc region comprises or consists of an A amino acid at position L234; an A amino acid at position L235; and a G amino acid at position P329 EU numbering according to Kabat.

In some embodiments, the amino acid sequence of the modified hIgG1 Fc region comprises or consists of an A amino acid at position L234, an A amino acid at position L235, and/or an A or G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9. In some embodiments, the amino acid sequence of the modified hIgG1 Fc region comprises or consists of an A amino acid at position L234 and/or an A amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9. In some embodiments, the amino acid sequence of the modified hIgG1 Fc region comprises or consists of an A amino acid at position L234 and an A amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9. In some embodiments, the amino acid sequence of the modified hIgG1 Fc region comprises or consists of an A amino acid at position L234, an A amino acid at position L235, and an A or G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9. In some embodiments, the amino acid sequence of the modified hIgG1 Fc region comprises or consists of an A amino acid at position L234, an A amino acid at position L235, and an A amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9. In some embodiments, the amino acid sequence of the modified hIgG1 Fc region comprises or consists of an A amino acid at position L234, an A amino acid at position L235, and a G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9.

In some embodiments, the amino acid sequence of the modified hIgG1 Fc region comprises or consists of an A amino acid at position L234, an A amino acid at position L235, and/or an A or G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 127-144. In some embodiments, the amino acid sequence of the modified hIgG1 Fc region comprises or consists of an A amino acid at position L234 and/or an A amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 127-144. In some embodiments, the amino acid sequence of the modified hIgG1 Fc region comprises or consists of an A amino acid at position L234 and an A amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 127-144. In some embodiments, the amino acid sequence of the modified hIgG1 Fc region comprises or consists of an A amino acid at position L234, an A amino acid at position L235, and an A or G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 127-144. In some embodiments, the amino acid sequence of the modified hIgG1 Fc region comprises or consists of an A amino acid at position L234, an A amino acid at position L235, and an A amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 127-144. In some embodiments, the amino acid sequence of the modified hIgG1 Fc region comprises or consists of an A amino acid at position L234, an A amino acid at position L235, and a G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 127-144.

In some embodiments, the amino acid sequence of the modified hIgG1 Fc region comprises or consists of an A amino acid at position L234, an A amino acid at position L235, and/or an A or G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 145-152. In some embodiments, the amino acid sequence of the modified hIgG1 Fc region comprises or consists of an A amino acid at position L234 and/or an A amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 145-152. In some embodiments, the amino acid sequence of the modified hIgG1 Fc region comprises or consists of an A amino acid at position L234 and an A amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 145-152. In some embodiments, the amino acid sequence of the modified hIgG1 Fc region comprises or consists of an A amino acid at position L234, an A amino acid at position L235, and an A or G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 145-152. In some embodiments, the amino acid sequence of the modified hIgG1 Fc region comprises or consists of an A amino acid at position L234, an A amino acid at position L235, and an A amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 145-152. In some embodiments, the amino acid sequence of the modified hIgG1 Fc region comprises or consists of an A amino acid at position L234, an A amino acid at position L235, and a G amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 145-152.

### 5.2.7.2 Promotion of Heterodimerization

As described herein, in some embodiments, the multispecific protein (or one or more polypeptide thereof) comprises a first and second Fc region (see, e.g., §§ 5.2.7, 5.2.7.1, 5.2.7.2, 5.2.7.3). In some embodiments, the first Ig Fc region and the second Ig Fc region each comprise one or more amino acid modifications relative to each other to promote heterodimerization. IgG derived heterodimeric formats can be generated by methods known in the art, e.g., by forced heavy chain heterodimerization. Forced heavy chain heterodimerization can be obtained using known methods in the art, e.g., knob-in-hole or strand exchange engineered domains (SEED), see, e.g., Ji-Hee et al., “Immunoglobulin Fc Heterodimer Platform Technology: From Design to Applications in Therapeutic Antibodies and Proteins” Frontiers in Immunology, v7 (article 394) (2016) DOI=10.3389/fimmu.2016.00394 (hereinafter “Ji-Hee 2016”), the entire contents of which is incorporated by reference herein for all purposes.

In some embodiments, an interface of the first and the second Ig Fc regions is modified, e.g., introduction of an amino acid substitution, to increase heterodimerization, e.g., relative to a non-modified interface, e.g., a naturally occurring interface. For example, dimerization of the first and second Ig Fc regions can be enhanced by providing an Ig Fc interface of a first and a second Fc region with one or more of: a paired protuberance-cavity (“knob-in-hole”), an electrostatic interaction, or a strand-exchange, such that a greater ratio of heteromultimer to homomultimer forms, e.g., relative to a non-modified interface.

Knob-in-Hole amino acid pairing modifications are known in the art, and described in e.g., U.S. Pat. Nos. 5,731,116; 7,476,724; Ji-Hee 2016; and Ridgway, J. “‘Knobs-into-holes’ engineering of antibody CH3 domains for heavy chain heterodimerization” et al. Prot. Engineering 9(7): 617-621 (1996), the full contents of each of which is incorporated by reference herein. Generally, Knob-in-Hole comprises 1) introducing one or more amino acid substitutions in the CH3 domain of one or both of the first and second subject Ig Fc regions to promote heterodimerization; and 2) combining the modified Ig Fc regions under conditions that promote heterodimerization. “Knobs” are typically created by substituting a small amino acid in a parental Ig Fc region with a larger amino acid (e.g., T366Y or T366W); “holes” are created by substituting a larger residue in a parental Ig Fc region with a smaller amino acid (e.g., Y407T, T366S, 11368A, or Y407V). Exemplary Knob-in-Hole mutations include S354C, T366W in the “knob” Ig Fc region and Y349C, T366S, L368A, Y407V in the “hole” Ig Fc region. Other exemplary Knob-in-Hole mutations, which can be incorporated into any one or more of the embodiments, are provided in Table 10, with additional exemplary optional stabilizing Ig Fc cysteine mutations.

TABLE 10
Exemplary Knob-in-hole and Stabilizing Cysteine Modifications

| | | |
|---|---|---|
| Amino Acid Position | | |
| (EU numbering | Knob Ig Fc Amino | Hole Ig Fc Amino |
| according to Kabat) | Acid Substitution | Acid Substitution |

Knob-in Hole Amio Acid Substitutions

| | | |
|---|---|---|
| T366 | T366W | T366S |
| L368 | — | L368A |
| Y407 | — | Y407V |

Stabilizing Cysteine Amino Acid Substitutions

| | | |
|---|---|---|
| S354 | S354C | — |
| Y349 | — | Y349C |

The amino acid sequence of exemplary Fc regions that are known in the art to promote heterodimerization is provided in Table 11.

TABLE 11
The Amino Acid Sequence of Exemplary Pairs of Modified Heterodimeric Fc
Regions

| Description | Amino Acid Sequence | SEQ ID NO |
|---|---|---|
| hIgG1 CH2 Region + | PCPAPELLGGPSVFLFPPKPKDTLMISRTPEVTCVVVD | 153 |
| CH3 Region | VSHEDPEVKFNWYVDGVEVHNAKTKPREEQYNSTYRVV | |
| Knob T366W | SVLTVLHQDWLNGKEYKCKVSNKALPAPIEKTISKAKG | |
| With C-terminal Lysine | QPREPQVYTLPPSRDELTKNQVSLWCLVKGFYPSDIAV | |
| | EWESNGQPENNYKTTPPVLDSDGSFFLYSKLTVDKSRW | |
| | QQGNVFSCSVMHEALHNHYTQKSLSLSPGK | |
| hIgG1 CH2 Region + | PCPAPELLGGPSVFLFPPKPKDTLMISRTPEVTCVVVD | 154 |
| CH3 Region | VSHEDPEVKFNWYVDGVEVHNAKTKPREEQYNSTYRVV | |
| Knob T366W | SVLTVLHQDWLNGKEYKCKVSNKALPAPIEKTISKAKG | |
| Without C-terminal | QPREPQVYTLPPSRDELTKNQVSLWCLVKGFYPSDIAV | |
| Lysine | EWESNGQPENNYKTTPPVLDSDGSFFLYSKLTVDKSRW | |
| | QQGNVFSCSVMHEALHNHYTQKSLSLSPG | |
| hIgG1 Partial Hinge | TCPPCPAPELLGGPSVFLFPPKPKDTLMISRTPEVTCV | 155 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Knob T366W | AKGQPREPQVYTLPPSRDELTKNQVSLWCLVKGFYPSD | |
| With C-terminal Lysine | IAVEWESNGQPENNYKTTPPVLDSDGSFFLYSKLTVDK | |
| | SRWQQGNVFSCSVMHEALHNHYTQKSLSLSPGK | |
| hIgG1 Partial Hinge | TCPPCPAPELLGGPSVFLFPPKPKDTLMISRTPEVTCV | 156 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Knob T366W | AKGQPREPQVYTLPPSRDELTKNQVSLWCLVKGFYPSD | |
| Without C-terminal | IAVEWESNGQPENNYKTTPPVLDSDGSFFLYSKLTVDK | |
| Lysine | SRWQQGNVFSCSVMHEALHNHYTQKSLSLSPG | |
| hIgG1 Hinge Region + | EPKSCDKTHTCPPCPAPELLGGPSVFLFPPKPKDTLMI | 157 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Knob T366W | APIEKTISKAKGQPREPQVYTLPPSRDELTKNQVSLWC | |
| With C-terminal Lysine | LVKGFYPSDIAVEWESNGQPENNYKTTPPVLDSDGSFF | |
| | LYSKLTVDKSRWQQGNVFSCSVMHEALHNHYTQKSLSL | |
| | SPGK | |
| hIgG1 Hinge Region + | EPKSCDKTHTCPPCPAPELLGGPSVFLFPPKPKDTLMI | 158 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Knob T366W | APIEKTISKAKGQPREPQVYTLPPSRDELTKNQVSLWC | |
| Without C-terminal | LVKGFYPSDIAVEWESNGQPENNYKTTPPVLDSDGSFF | |
| Lysine | LYSKLTVDKSRWQQGNVFSCSVMHEALHNHYTQKSLSL | |
| | SPG | |
| hIgG1 CH1+ Hinge | ASTKGPSVFPLAPSSKSTSGGTAALGCLVKDYFPEPVT | 159 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W | PELLGGPSVFLFPPKPKDTLMISRTPEVTCVVVDVSHE | |
| With C-terminal Lysine | DPEVKFNWYVDGVEVHNAKTKPREEQYNSTYRVVSVLT | |
| | VLHQDWLNGKEYKCKVSNKALPAPIEKTISKAKGQPRE | |
| | PQVYTLPPCRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 160 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W | PELLGGPSVFLFPPKPKDTLMISRTPEVTCVVVDVSHE | |
| Without C-terminal | DPEVKFNWYVDGVEVHNAKTKPREEQYNSTYRVVSVLT | |
| Lysine | VLHQDWLNGKEYKCKVSNKALPAPIEKTISKAKGQPRE | |
| | PQVYTLPPSRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPG | |
| hIgG1 CH2 Region + | PCPAPELLGGPSVFLFPPKPKDTLMISRTPEVTCVVVD | 161 |
| CH3 Region | VSHEDPEVKFNWYVDGVEVHNAKTKPREEQYNSTYRVV | |
| Knob T366W/S354C | SVLTVLHQDWLNGKEYKCKVSNKALPAPIEKTISKAKG | |
| With C-terminal Lysine | QPREPQVYTLPPCRDELTKNQVSLWCLVKGFYPSDIAV | |
| | EWESNGQPENNYKTTPPVLDSDGSFFLYSKLTVDKSRW | |
| | QQGNVFSCSVMHEALHNHYTQKSLSLSPGK | |
| hIgG1 CH2 Region + | PCPAPELLGGPSVFLFPPKPKDTLMISRTPEVTCVVVD | 162 |
| CH3 Region | VSHEDPEVKFNWYVDGVEVHNAKTKPREEQYNSTYRVV | |
| Knob T366W/S354C | SVLTVLHQDWLNGKEYKCKVSNKALPAPIEKTISKAKG | |
| Without C-terminal | QPREPQVYTLPPCRDELTKNQVSLWCLVKGFYPSDIAV | |
| Lysine | EWESNGQPENNYKTTPPVLDSDGSFFLYSKLTVDKSRW | |
| | QQGNVFSCSVMHEALHNHYTQKSLSLSPG | |
| hIgG1 Partial Hinge | TCPPCPAPELLGGPSVFLFPPKPKDTLMISRTPEVTCV | 163 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Knob T366W/S354C | AKGQPREPQVYTLPPCRDELTKNQVSLWCLVKGFYPSD | |
| With C-terminal Lysine | IAVEWESNGQPENNYKTTPPVLDSDGSFFLYSKLTVDK | |
| | SRWQQGNVFSCSVMHEALHNHYTQKSLSLSPGK | |
| hIgG1 Partial Hinge | TCPPCPAPELLGGPSVFLFPPKPKDTLMISRTPEVTCV | 164 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Knob T366W/S354C | AKGQPREPQVYTLPPCRDELTKNQVSLWCLVKGFYPSD | |
| Without C-terminal | IAVEWESNGQPENNYKTTPPVLDSDGSFFLYSKLTVDK | |
| Lysine | SRWQQGNVFSCSVMHEALHNHYTQKSLSLSPG | |
| hIgG1 Hinge Region + | EPKSCDKTHTCPPCPAPELLGGPSVFLFPPKPKDTLMI | 165 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Knob T366W/S354C | APIEKTISKAKGQPREPQVYTLPPCRDELTKNQVSLWC | |
| With C-terminal Lysine | LVKGFYPSDIAVEWESNGQPENNYKTTPPVLDSDGSFF | |
| | LYSKLTVDKSRWQQGNVFSCSVMHEALHNHYTQKSLSL | |
| | SPGK | |
| hIgG1 Hinge Region + | EPKSCDKTHTCPPCPAPELLGGPSVFLFPPKPKDTLMI | 166 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Knob T366W/S354C | APIEKTISKAKGQPREPQVYTLPPCRDELTKNQVSLWC | |
| Without C-terminal | LVKGFYPSDIAVEWESNGQPENNYKTTPPVLDSDGSFF | |
| Lysine | LYSKLTVDKSRWQQGNVFSCSVMHEALHNHYTQKSLSL | |
| | SPG | |
| hIgG1 CH1+ Hinge | ASTKGPSVFPLAPSSKSTSGGTAALGCLVKDYFPEPVT | 167 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W/S354C | PELLGGPSVFLFPPKPKDTLMISRTPEVTCVVVDVSHE | |
| With C-terminal Lysine | DPEVKFNWYVDGVEVHNAKTKPREEQYNSTYRVVSVLT | |
| | VLHQDWLNGKEYKCKVSNKALPAPIEKTISKAKGQPRE | |
| | PQVYTLPPCRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 168 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W/S354C | PELLGGPSVFLFPPKPKDTLMISRTPEVTCVVVDVSHE | |
| Without C-terminal | DPEVKFNWYVDGVEVHNAKTKPREEQYNSTYRVVSVLT | |
| Lysine | VLHQDWLNGKEYKCKVSNKALPAPIEKTISKAKGQPRE | |
| | PQVYTLPPCRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPG | |
| hIgG1 CH2 Region + | PCPAPELLGGPSVFLFPPKPKDTLMISRTPEVTCVVVD | 169 |
| CH3 Region | VSHEDPEVKFNWYVDGVEVHNAKTKPREEQYNSTYRVV | |
| Hole | SVLTVLHQDWLNGKEYKCKVSNKALPAPIEKTISKAKG | |
| T366S/L368A/407V | QPREPQVYTLPPSRDELTKNQVSLSCAVKGFYPSDIAV | |
| With C-terminal Lysine | EWESNGQPENNYKTTPPVLDSDGSFFLVSKLTVDKSRW | |
| | QQGNVFSCSVMHEALHNHYTQKSLSLSPGK | |
| hIgG1 CH2 Region + | PCPAPELLGGPSVFLFPPKPKDTLMISRTPEVTCVVVD | 170 |
| CH3 Region | VSHEDPEVKFNWYVDGVEVHNAKTKPREEQYNSTYRVV | |
| Hole | SVLTVLHQDWLNGKEYKCKVSNKALPAPIEKTISKAKG | |
| T366S/L368A/407V | QPREPQVYTLPPSRDELTKNQVSLSCAVKGFYPSDIAV | |
| Without C-terminal | EWESNGQPENNYKTTPPVLDSDGSFFLVSKLTVDKSRW | |
| Lysine | QQGNVFSCSVMHEALHNHYTQKSLSLSPG | |
| hIgG1 Partial Hinge | TCPPCPAPELLGGPSVFLFPPKPKDTLMISRTPEVTCV | 171 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Hole | AKGQPREPQVYTLPPSRDELTKNQVSLSCAVKGFYPSD | |
| T366S/L368A/407V | IAVEWESNGQPENNYKTTPPVLDSDGSFFLVSKLTVDK | |
| With C-terminal Lysine | SRWQQGNVFSCSVMHEALHNHYTQKSLSLSPGK | |
| hIgG1 Partial Hinge | TCPPCPAPELLGGPSVFLFPPKPKDTLMISRTPEVTCV | 172 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Hole | AKGQPREPQVYTLPPSRDELTKNQVSLSCAVKGFYPSD | |
| T366S/L368A/407V | IAVEWESNGQPENNYKTTPPVLDSDGSFFLVSKLTVDK | |
| Without C-terminal | SRWQQGNVFSCSVMHEALHNHYTQKSLSLSPG | |
| Lysine | | |
| hIgG1 Hinge Region + | EPKSCDKTHTCPPCPAPELLGGPSVFLFPPKPKDTLMI | 173 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Hole | APIEKTISKAKGQPREPQVYTLPPSRDELTKNQVSLSC | |
| T366S/L368A/407V | AVKGFYPSDIAVEWESNGQPENNYKTTPPVLDSDGSFF | |
| With C-terminal Lysine | LVSKLTVDKSRWQQGNVFSCSVMHEALHNHYTQKSLSL | |
| | SPGK | |
| hIgG1 Hinge Region + | EPKSCDKTHTCPPCPAPELLGGPSVFLFPPKPKDTLMI | 174 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Hole | APIEKTISKAKGQPREPQVYTLPPSRDELTKNQVSLSC | |
| T366S/L368A/407V | AVKGFYPSDIAVEWESNGQPENNYKTTPPVLDSDGSFF | |
| Without C-terminal | LVSKLTVDKSRWQQGNVFSCSVMHEALHNHYTQKSLSL | |
| Lysine | SPG | |
| hIgG1 CH1+ Hinge | ASTKGPSVFPLAPSSKSTSGGTAALGCLVKDYFPEPVT | 175 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Hole | PELLGGPSVFLFPPKPKDTLMISRTPEVTCVVVDVSHE | |
| T366S/L368A/407V | DPEVKFNWYVDGVEVHNAKTKPREEQYNSTYRVVSVLT | |
| With C-terminal Lysine | VLHQDWLNGKEYKCKVSNKALPAPIEKTISKAKGQPRE | |
| | PQVYTLPPSRDELTKNQVSLSCAVKGFYPSDIAVEWES | |
| | NGQPENNYKTTPPVLDSDGSFFLVSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 176 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Hole | PELLGGPSVFLFPPKPKDTLMISRTPEVTCVVVDVSHE | |
| T366S/L368A/407V | DPEVKFNWYVDGVEVHNAKTKPREEQYNSTYRVVSVLT | |
| Without C-terminal | VLHQDWLNGKEYKCKVSNKALPAPIEKTISKAKGQPRE | |
| Lysine | PQVYTLPPSRDELTKNQVSLSCAVKGFYPSDIAVEWES | |
| | NGQPENNYKTTPPVLDSDGSFFLVSKLTVDKSRWQQGN | |
| | VFSCSVMHEALHNHYTQKSLSLSPG | |
| hIgG1 CH2 Region + | PCPAPELLGGPSVFLFPPKPKDTLMISRTPEVTCVVVD | 177 |
| CH3 Region | VSHEDPEVKFNWYVDGVEVHNAKTKPREEQYNSTYRVV | |
| Hole | SVLTVLHQDWLNGKEYKCKVSNKALPAPIEKTISKAKG | |
| T366S/L368A/407V/ | QPREPQVYTLPPSRDELTKNQVSLSCAVKGFYPSDIAV | |
| Y349C | EWESNGQPENNYKTTPPVLDSDGSFFLVSKLTVDKSRW | |
| With C-terminal Lysine | QQGNVFSCSVMHEALHNHYTQKSLSLSPGK | |
| hIgG1 CH2 Region + | PCPAPELLGGPSVFLFPPKPKDTLMISRTPEVTCVVVD | 178 |
| CH3 Region | VSHEDPEVKFNWYVDGVEVHNAKTKPREEQYNSTYRVV | |
| Hole | SVLTVLHQDWLNGKEYKCKVSNKALPAPIEKTISKAKG | |
| T366S/L368A/407V/ | QPREPQVYTLPPSRDELTKNQVSLSCAVKGFYPSDIAV | |
| Y349C | EWESNGQPENNYKTTPPVLDSDGSFFLVSKLTVDKSRW | |
| Without C-terminal | QQGNVFSCSVMHEALHNHYTQKSLSLSPG | |
| Lysine | | |
| hIgG1 Partial Hinge | TCPPCPAPELLGGPSVFLFPPKPKDTLMISRTPEVTCV | 179 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Hole | AKGQPREPQVCTLPPSRDELTKNQVSLSCAVKGFYPSD | |
| T366S/L368A/407V/ | IAVEWESNGQPENNYKTTPPVLDSDGSFFLVSKLTVDK | |
| Y349C | SRWQQGNVFSCSVMHEALHNHYTQKSLSLSPGK | |
| With C-terminal Lysine | | |
| hIgG1 Partial Hinge | TCPPCPAPELLGGPSVFLFPPKPKDTLMISRTPEVTCV | 180 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALPAPIEKTISK | |
| Hole | AKGQPREPQVCTLPPSRDELTKNQVSLSCAVKGFYPSD | |
| T366S/L368A/407V/ | IAVEWESNGQPENNYKTTPPVLDSDGSFFLVSKLTVDK | |
| Y349C | SRWQQGNVFSCSVMHEALHNHYTQKSLSLSPG | |
| Without C-terminal | | |
| Lysine | | |
| hIgG1 Hinge Region + | EPKSCDKTHTCPPCPAPELLGGPSVFLFPPKPKDTLMI | 181 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Hole | APIEKTISKAKGQPREPQVCTLPPSRDELTKNQVSLSC | |
| T366S/L368A/407V/ | AVKGFYPSDIAVEWESNGQPENNYKTTPPVLDSDGSFF | |
| Y349C | LVSKLTVDKSRWQQGNVFSCSVMHEALHNHYTQKSLSL | |
| With C-terminal Lysine | SPGK | |
| hIgG1 Hinge Region + | EPKSCDKTHTCPPCPAPELLGGPSVFLFPPKPKDTLMI | 182 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALP | |
| Hole | APIEKTISKAKGQPREPQVCTLPPSRDELTKNQVSLSC | |
| T366S/L368A/407V/ | AVKGFYPSDIAVEWESNGQPENNYKTTPPVLDSDGSFF | |
| Y349C | LVSKLTVDKSRWQQGNVFSCSVMHEALHNHYTQKSLSL | |
| Without C-terminal | SPG | |
| Lysine | | |
| hIgG1 CH1+ Hinge | ASTKGPSVFPLAPSSKSTSGGTAALGCLVKDYFPEPVT | 183 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Hole | PELLGGPSVFLFPPKPKDTLMISRTPEVTCVVVDVSHE | |
| T366S/L368A/407V/ | DPEVKFNWYVDGVEVHNAKTKPREEQYNSTYRVVSVLT | |
| Y349C | VLHQDWLNGKEYKCKVSNKALPAPIEKTISKAKGQPRE | |
| With C-terminal Lysine | PQVCTLPPSRDELTKNQVSLSCAVKGFYPSDIAVEWES | |
| | NGQPENNYKTTPPVLDSDGSFFLVSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 184 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Hole | PELLGGPSVFLFPPKPKDTLMISRTPEVTCVVVDVSHE | |
| T366S/L368A/407V/ | DPEVKFNWYVDGVEVHNAKTKPREEQYNSTYRVVSVLT | |
| Y349C | VLHQDWLNGKEYKCKVSNKALPAPIEKTISKAKGQPRE | |
| Without C-terminal | PQVCTLPPSRDELTKNQVSLSCAVKGFYPSDIAVEWES | |
| Lysine | NGQPENNYKTTPPVLDSDGSFFLVSKLTVDKSRWQQGN | |
| | VFSCSVMHEALHNHYTQKSLSLSPG | |

As described herein, in some embodiments, the multispecific protein (or one or more polypeptide thereof) comprises a first Fc region and a second Ig Fc region.

In some embodiments, the amino acid sequence of the first Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Ig Fc region (e.g., a reference Ig Fc region set forth in Table 8). In some embodiments, the amino acid sequence of the first hIg further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Ig Fc region (e.g., a reference Ig Fc region set forth in Table 8).

In some embodiments, the amino acid sequence of the first Fc region comprises each of the following amino acid substitutions: T366W and a S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Ig Fc region (e.g., a reference Ig Fc region set forth in Table 8).

In some embodiments, the amino acid sequence of the second Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Ig Fc region (e.g., a reference Ig Fc region set forth in Table 8). In some embodiments, the amino acid sequence of the second hIg further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Ig Fc region (e.g., a reference Ig Fc region set forth in Table 8).

In some embodiments, the amino acid sequence of the second Fc region comprises each of the following amino acid substitutions: T366W and a S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Ig Fc region (e.g., a reference Ig Fc region set forth in Table 8).

In some embodiments, the amino acid sequence of the first Ig Fc region comprises a W amino acid at position T366, EU numbering according to Kabat; and the amino acid sequence of the second Ig Fc region comprises a S amino acid at position T366, an A amino acid at position L368, and a V amino acid at position Y407, EU numbering according to Kabat.

In some embodiments, the amino acid sequence of the first Ig Fc region comprises a W amino acid at position T366 and a C amino acid at position S354, EU numbering according to Kabat; and the amino acid sequence of the second Ig Fc region comprises a S amino acid at position T366, an A amino acid at position L368, a V amino acid at position Y407, and a C amino acid at position Y349, EU numbering according to Kabat.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 153-160; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 169-176.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 161-168; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 177-184.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 153; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 169.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 154; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 170.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 155; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 171.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 156; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 172.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 157; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 173.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 158; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 174.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 159; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 175.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 160; and the amino acid sequence of the second Ig Fc region comprises a S amino acid at position T366, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 176.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 161; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 177.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 162; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 178.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 163; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 179.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 164; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 180.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 165; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 181.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 166; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 182.

In some embodiments, the amino acid sequence of the first Fc region comprises a W amino acid at position T366 and a C amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 167; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 183.

In some embodiments, the amino acid sequence of the first Fc region comprises a W amino acid at position T366 and a C amino acid at position 5354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 168; and the amino acid sequence of the second Ig Fc region comprises a S amino acid at position T366, an A amino acid at position L368, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 184.

### 5.2.7.3 Exemplary Modified Fc Regions

As described herein, in some embodiments, the multispecific protein (or one or more polypeptide thereof) comprises a first and second Fc region (see, e.g., §§ 5.2.7, 5.2.7.1, 5.2.7.2, 5.2.7.3). In some embodiments, the first Ig Fc region and the second Ig Fc region each comprise multiple amino acid modifications described herein, e.g., one or more amino acid modification that decreases or abolishes one or more Fc effector functions (e.g., antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) (see, e.g., § 5.2.7.1); and one or more amino acid modification that promote heterodimerization of the first and second Fc regions (see, e.g., § 5.2.7.2).

In some embodiments, the first and second Fc region each comprise one or more amino acid modification that decreases or abolishes one or more Fc effector functions (e.g., antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) (see, e.g., § 5.2.7.1); and one or more amino acid modification that promote heterodimerization of the first and second Fc regions (see, e.g., § 5.2.7.2).

TABLE 12
The Amino Acid Sequence of Exemplary Modified Fc Regions

| Description | Amino Acid Sequence | SEQ ID NO |
|---|---|---|
| hIgG1 CH2 Region + | PCPAPEAAGGPSVFLFPPKPKDTLMISRTPEVTCVVVD | 185 |
| CH3 Region | VSHEDPEVKFNWYVDGVEVHNAKTKPREEQYNSTYRVV | |
| Knob T366W/S354C | SVLTVLHQDWLNGKEYKCKVSNKALAAPIEKTISKAKG | |
| L234A/L235A/P329A | QPREPQVYTLPPCRDELTKNQVSLWCLVKGFYPSDIAV | |
| With C-terminal Lysine | EWESNGQPENNYKTTPPVLDSDGSFFLYSKLTVDKSRW | |
| | QQGNVFSCSVMHEALHNHYTQKSLSLSPGK | |
| hIgG1 CH2 Region + | PCPAPEAAGGPSVFLFPPKPKDTLMISRTPEVTCVVVD | 186 |
| CH3 Region | VSHEDPEVKFNWYVDGVEVHNAKTKPREEQYNSTYRVV | |
| Knob T366W/S354C | SVLTVLHQDWLNGKEYKCKVSNKALAAPIEKTISKAKG | |
| L234A/L235A/P329A | QPREPQVYTLPPCRDELTKNQVSLWCLVKGFYPSDIAV | |
| Without C-terminal | EWESNGQPENNYKTTPPVLDSDGSFFLYSKLTVDKSRW | |
| Lysine | QQGNVFSCSVMHEALHNHYTQKSLSLSPG | |
| hIgG1 Partial Hinge | TCPPCPAPEAAGGPSVFLFPPKPKDTLMISRTPEVTCV | 187 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALAAPIEKTISK | |
| Knob T366W/S354C | AKGQPREPQVYTLPPCRDELTKNQVSLWCLVKGFYPSD | |
| L234A/L235A/P329A | IAVEWESNGQPENNYKTTPPVLDSDGSFFLYSKLTVDK | |
| With C-terminal Lysine | SRWQQGNVFSCSVMHEALHNHYTQKSLSLSPGK | |
| hIgG1 Partial Hinge | TCPPCPAPEAAGGPSVFLFPPKPKDTLMISRTPEVTCV | 188 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALAAPIEKTISK | |
| Knob T366W/S354C | AKGQPREPQVYTLPPCRDELTKNQVSLWCLVKGFYPSD | |
| L234A/L235A/P329A | IAVEWESNGQPENNYKTTPPVLDSDGSFFLYSKLTVDK | |
| Without C-terminal | SRWQQGNVFSCSVMHEALHNHYTQKSLSLSPG | |
| Lysine | | |
| hIgG1 Hinge Region + | EPKSCDKTHTCPPCPAPEAAGGPSVFLFPPKPKDTLMI | 189 |
| CH2 Region + CH3 | SRTPEVTCVVVDVSHEDPEVKENWYVDGVEVHNAKTKP | |
| Region | REEQYNSTYRVVSVLTVLHQDWLNGKEYKCKVSNKALA | |
| Knob T366W/S354C | APIEKTISKAKGQPREPQVYTLPPCRDELTKNQVSLWC | |
| L234A/L235A/P329A | LVKGFYPSDIAVEWESNGQPENNYKTTPPVLDSDGSFF | |
| With C-terminal Lysine | LYSKLTVDKSRWQQGNVFSCSVMHEALHNHYTQKSLSL | |
| | SPGK | |
| hIgG1 Hinge Region + | EPKSCDKTHTCPPCPAPEAAGGPSVFLFPPKPKDTLMI | 190 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALA | |
| Knob T366W/S354C | APIEKTISKAKGQPREPQVYTLPPCRDELTKNQVSLWC | |
| L234A/L235A/P329A | LVKGFYPSDIAVEWESNGQPENNYKTTPPVLDSDGSFF | |
| Without C-terminal | LYSKLTVDKSRWQQGNVFSCSVMHEALHNHYTQKSLSL | |
| Lysine | SPG | |
| hIgG1 CH1+ Hinge | ASTKGPSVFPLAPSSKSTSGGTAALGCLVKDYFPEPVT | 191 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W/S354C | PEAAGGPSVFLFPPKPKDTLMISRTPEVTCVVVDVSHE | |
| L234A/L235A/P329A | DPEVKFNWYVDGVEVHNAKTKPREEQYNSTYRVVSVLT | |
| With C-terminal Lysine | VLHQDWLNGKEYKCKVSNKALAAPIEKTISKAKGQPRE | |
| | PQVYTLPPCRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 192 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W/S354C | PEAAGGPSVFLFPPKPKDTLMISRTPEVTCVVVDVSHE | |
| L234A/L235A/P329A | DPEVKFNWYVDGVEVHNAKTKPREEQYNSTYRVVSVLT | |
| Without C-terminal | VLHQDWLNGKEYKCKVSNKALAAPIEKTISKAKGQPRE | |
| Lysine | PQVYTLPPCRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPG | |
| hIgG1 CH2 Region + | PCPAPEAAGGPSVFLFPPKPKDTLMISRTPEVTCVVVD | 193 |
| CH3 Region | VSHEDPEVKFNWYVDGVEVHNAKTKPREEQYNSTYRVV | |
| Hole | SVLTVLHQDWLNGKEYKCKVSNKALAAPIEKTISKAKG | |
| T366S/L368A/407V/ | QPREPQVYTLPPSRDELTKNQVSLSCAVKGFYPSDIAV | |
| Y349C | EWESNGQPENNYKTTPPVLDSDGSFFLVSKLTVDKSRW | |
| L234A/L235A/P329A | QQGNVFSCSVMHEALHNHYTQKSLSLSPGK | |
| With C-terminal Lysine | | |
| hIgG1 CH2 Region + | PCPAPEAAGGPSVFLFPPKPKDTLMISRTPEVTCVVVD | 194 |
| CH3 Region | VSHEDPEVKFNWYVDGVEVHNAKTKPREEQYNSTYRVV | |
| Hole | SVLTVLHQDWLNGKEYKCKVSNKALAAPIEKTISKAKG | |
| T366S/L368A/407V/ | QPREPQVYTLPPSRDELTKNQVSLSCAVKGFYPSDIAV | |
| Y349C | EWESNGQPENNYKTTPPVLDSDGSFFLVSKLTVDKSRW | |
| L234A/L235A/P329A | QQGNVFSCSVMHEALHNHYTQKSLSLSPG | |
| Without C-terminal | | |
| Lysine | | |
| hIgG1 Partial Hinge | TCPPCPAPEAAGGPSVFLFPPKPKDTLMISRTPEVTCV | 195 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALAAPIEKTISK | |
| Hole | AKGQPREPQVCTLPPSRDELTKNQVSLSCAVKGFYPSD | |
| T366S/L368A/407V/ | IAVEWESNGQPENNYKTTPPVLDSDGSFFLVSKLTVDK | |
| Y349C | SRWQQGNVFSCSVMHEALHNHYTQKSLSLSPGK | |
| L234A/L235A/P329A | | |
| With C-terminal Lysine | | |
| hIgG1 Partial Hinge | TCPPCPAPEAAGGPSVFLFPPKPKDTLMISRTPEVTCV | 196 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALAAPIEKTISK | |
| Hole | AKGQPREPQVCTLPPSRDELTKNQVSLSCAVKGFYPSD | |
| T366S/L368A/407V/ | IAVEWESNGQPENNYKTTPPVLDSDGSFFLVSKLTVDK | |
| Y349C | SRWQQGNVFSCSVMHEALHNHYTQKSLSLSPG | |
| L234A/L235A/P329A | | |
| Without C-terminal | | |
| Lysine | | |
| hIgG1 Hinge Region + | EPKSCDKTHTCPPCPAPEAAGGPSVFLFPPKPKDTLMI | 197 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALA | |
| Hole | APIEKTISKAKGQPREPQVCTLPPSRDELTKNQVSLSC | |
| T366S/L368A/407V/ | AVKGFYPSDIAVEWESNGQPENNYKTTPPVLDSDGSFF | |
| Y349C | LVSKLTVDKSRWQQGNVFSCSVMHEALHNHYTQKSLSL | |
| L234A/L235A/P329A | SPGK | |
| With C-terminal Lysine | | |
| hIgG1 Hinge Region + | EPKSCDKTHTCPPCPAPEAAGGPSVFLFPPKPKDTLMI | 198 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALA | |
| Hole | APIEKTISKAKGQPREPQVCTLPPSRDELTKNQVSLSC | |
| T366S/L368A/407V/ | AVKGFYPSDIAVEWESNGQPENNYKTTPPVLDSDGSFF | |
| Y349C | LVSKLTVDKSRWQQGNVFSCSVMHEALHNHYTQKSLSL | |
| L234A/L235A/P329A | SPG | |
| Without C-terminal | | |
| Lysine | | |
| hIgG1 CH1+ Hinge | ASTKGPSVFPLAPSSKSTSGGTAALGCLVKDYFPEPVT | 199 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Hole | PEAAGGPSVFLFPPKPKDTLMISRTPEVTCVVVDVSHE | |
| T366S/L368A/407V/ | DPEVKFNWYVDGVEVHNAKTKPREEQYNSTYRVVSVLT | |
| Y349C | VLHQDWLNGKEYKCKVSNKALAAPIEKTISKAKGQPRE | |
| L234A/L235A/P329A | PQVCTLPPSRDELTKNQVSLSCAVKGFYPSDIAVEWES | |
| With C-terminal Lysine | NGQPENNYKTTPPVLDSDGSFFLVSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 200 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| L234A/L235A/P329A | PEAAGGPSVFLFPPKPKDTLMISRTPEVTCVVVDVSHE | |
| Hole | DPEVKFNWYVDGVEVHNAKTKPREEQYNSTYRVVSVLT | |
| T366S/L368A/407V/ | VLHQDWLNGKEYKCKVSNKALAAPIEKTISKAKGQPRE | |
| Y349C | PQVCTLPPSRDELTKNQVSLSCAVKGFYPSDIAVEWES | |
| L234A/L235A/P329A | NGQPENNYKTTPPVLDSDGSFFLVSKLTVDKSRWQQGN | |
| Without C-terminal | VFSCSVMHEALHNHYTQKSLSLSPG | |
| Lysine | | |
| hIgG1 CH2 Region + | PCPAPEAAGGPSVFLFPPKPKDTLMISRTPEVTCVVVD | 201 |
| CH3 Region | VSHEDPEVKFNWYVDGVEVHNAKTKPREEQYNSTYRVV | |
| Knob T366W/S354C | SVLTVLHQDWLNGKEYKCKVSNKALGAPIEKTISKAKG | |
| L234A/L235A/P329A | QPREPQVYTLPPCRDELTKNQVSLWCLVKGFYPSDIAV | |
| With C-terminal Lysine | EWESNGQPENNYKTTPPVLDSDGSFFLYSKLTVDKSRW | |
| | QQGNVFSCSVMHEALHNHYTQKSLSLSPGK | |
| hIgG1 CH2 Region + | PCPAPEAAGGPSVFLFPPKPKDTLMISRTPEVTCVVVD | 202 |
| CH3 Region | VSHEDPEVKFNWYVDGVEVHNAKTKPREEQYNSTYRVV | |
| Knob T366W/S354C | SVLTVLHQDWLNGKEYKCKVSNKALGAPIEKTISKAKG | |
| L234A/L235A/P329A | QPREPQVYTLPPCRDELTKNQVSLWCLVKGFYPSDIAV | |
| Without C-terminal | EWESNGQPENNYKTTPPVLDSDGSFFLYSKLTVDKSRW | |
| Lysine | QQGNVFSCSVMHEALHNHYTQKSLSLSPG | |
| hIgG1 Partial Hinge | TCPPCPAPEAAGGPSVFLFPPKPKDTLMISRTPEVTCV | 203 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALGAPIEKTISK | |
| Knob T366W/S354C | AKGQPREPQVYTLPPCRDELTKNQVSLWCLVKGFYPSD | |
| L234A/L235A/P329A | IAVEWESNGQPENNYKTTPPVLDSDGSFFLYSKLTVDK | |
| With C-terminal Lysine | SRWQQGNVFSCSVMHEALHNHYTQKSLSLSPGK | |
| hIgG1 Partial Hinge | TCPPCPAPEAAGGPSVFLFPPKPKDTLMISRTPEVTCV | 204 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALGAPIEKTISK | |
| Knob T366W/S354C | AKGQPREPQVYTLPPCRDELTKNQVSLWCLVKGFYPSD | |
| L234A/L235A/P329A | IAVEWESNGQPENNYKTTPPVLDSDGSFFLYSKLTVDK | |
| Without C-terminal | SRWQQGNVFSCSVMHEALHNHYTQKSLSLSPG | |
| Lysine | | |
| hIgG1 Hinge Region + | EPKSCDKTHTCPPCPAPEAAGGPSVFLFPPKPKDTLMI | 205 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALG | |
| Knob T366W/S354C | APIEKTISKAKGQPREPQVYTLPPCRDELTKNQVSLWC | |
| L234A/L235A/P329A | LVKGFYPSDIAVEWESNGQPENNYKTTPPVLDSDGSFF | |
| With C-terminal Lysine | LYSKLTVDKSRWQQGNVFSCSVMHEALHNHYTQKSLSL | |
| | SPGK | |
| hIgG1 Hinge Region + | EPKSCDKTHTCPPCPAPEAAGGPSVFLFPPKPKDTLMI | 206 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALG | |
| Knob T366W/S354C | APIEKTISKAKGQPREPQVYTLPPCRDELTKNQVSLWC | |
| L234A/L235A/P329A | LVKGFYPSDIAVEWESNGQPENNYKTTPPVLDSDGSFF | |
| Without C-terminal | LYSKLTVDKSRWQQGNVFSCSVMHEALHNHYTQKSLSL | |
| Lysine | SPG | |
| hIgG1 CH1+ Hinge | ASTKGPSVFPLAPSSKSTSGGTAALGCLVKDYFPEPVT | 207 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W/S354C | PEAAGGPSVFLFPPKPKDTLMISRTPEVTCVVVDVSHE | |
| L234A/L235A/P329A | DPEVKFNWYVDGVEVHNAKTKPREEQYNSTYRVVSVLT | |
| With C-terminal Lysine | VLHQDWLNGKEYKCKVSNKALGAPIEKTISKAKGQPRE | |
| | PQVYTLPPCRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 208 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Knob T366W/S354C | PEAAGGPSVFLFPPKPKDTLMISRTPEVTCVVVDVSHE | |
| L234A/L235A/P329A | DPEVKFNWYVDGVEVHNAKTKPREEQYNSTYRVVSVLT | |
| Without C-terminal | VLHQDWLNGKEYKCKVSNKALGAPIEKTISKAKGQPRE | |
| Lysine | PQVYTLPPCRDELTKNQVSLWCLVKGFYPSDIAVEWES | |
| | NGQPENNYKTTPPVLDSDGSFFLYSKLTVDKSRWQQGN | |
| | VFSCSVMHEALHNHYTQKSLSLSPG | |
| hIgG1 CH2 Region + | PCPAPEAAGGPSVFLFPPKPKDTLMISRTPEVTCVVVD | 209 |
| CH3 Region | VSHEDPEVKFNWYVDGVEVHNAKTKPREEQYNSTYRVV | |
| Hole | SVLTVLHQDWLNGKEYKCKVSNKALGAPIEKTISKAKG | |
| T366S/L368A/407V/ | QPREPQVYTLPPSRDELTKNQVSLSCAVKGFYPSDIAV | |
| Y349C | EWESNGQPENNYKTTPPVLDSDGSFFLVSKLTVDKSRW | |
| L234A/L235A/P329A | QQGNVFSCSVMHEALHNHYTQKSLSLSPGK | |
| With C-terminal Lysine | | |
| hIgG1 CH2 Region + | PCPAPEAAGGPSVFLFPPKPKDTLMISRTPEVTCVVVD | 210 |
| CH3 Region | VSHEDPEVKFNWYVDGVEVHNAKTKPREEQYNSTYRVV | |
| Hole | SVLTVLHQDWLNGKEYKCKVSNKALGAPIEKTISKAKG | |
| T366S/L368A/407V/ | QPREPQVYTLPPSRDELTKNQVSLSCAVKGFYPSDIAV | |
| Y349C | EWESNGQPENNYKTTPPVLDSDGSFFLVSKLTVDKSRW | |
| L234A/L235A/P329A | QQGNVFSCSVMHEALHNHYTQKSLSLSPG | |
| Without C-terminal | | |
| Lysine | | |
| hIgG1 Partial Hinge | TCPPCPAPEAAGGPSVFLFPPKPKDTLMISRTPEVTCV | 211 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALGAPIEKTISK | |
| Hole | AKGQPREPQVCTLPPSRDELTKNQVSLSCAVKGFYPSD | |
| T366S/L368A/407V/ | IAVEWESNGQPENNYKTTPPVLDSDGSFFLVSKLTVDK | |
| Y349C | SRWQQGNVFSCSVMHEALHNHYTQKSLSLSPGK | |
| L234A/L235A/P329A | | |
| With C-terminal Lysine | | |
| hIgG1 Partial Hinge | TCPPCPAPEAAGGPSVFLFPPKPKDTLMISRTPEVTCV | 212 |
| Region + CH2 Region + | VVDVSHEDPEVKFNWYVDGVEVHNAKTKPREEQYNSTY | |
| CH3 Region | RVVSVLTVLHQDWLNGKEYKCKVSNKALGAPIEKTISK | |
| Hole | AKGQPREPQVCTLPPSRDELTKNQVSLSCAVKGFYPSD | |
| T366S/L368A/407V/ | IAVEWESNGQPENNYKTTPPVLDSDGSFFLVSKLTVDK | |
| Y349C | SRWQQGNVFSCSVMHEALHNHYTQKSLSLSPG | |
| L234A/L235A/P329A | | |
| Without C-terminal | | |
| Lysine | | |
| hIgG1 Hinge Region + | EPKSCDKTHTCPPCPAPEAAGGPSVFLFPPKPKDTLMI | 213 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALG | |
| Hole | APIEKTISKAKGQPREPQVCTLPPSRDELTKNQVSLSC | |
| T366S/L368A/407V/ | AVKGFYPSDIAVEWESNGQPENNYKTTPPVLDSDGSFF | |
| Y349C | LVSKLTVDKSRWQQGNVFSCSVMHEALHNHYTQKSLSL | |
| L234A/L235A/P329A | SPGK | |
| With C-terminal Lysine | | |
| hIgG1 Hinge Region + | EPKSCDKTHTCPPCPAPEAAGGPSVFLFPPKPKDTLMI | 214 |
| CH2 Region + CH3 | SRTPEVTCVVVDVSHEDPEVKFNWYVDGVEVHNAKTKP | |
| Region | REEQYNSTYRVVSVLTVLHQDWLNGKEYKCKVSNKALG | |
| Hole | APIEKTISKAKGQPREPQVCTLPPSRDELTKNQVSLSC | |
| T366S/L368A/407V/ | AVKGFYPSDIAVEWESNGQPENNYKTTPPVLDSDGSFF | |
| Y349C | LVSKLTVDKSRWQQGNVFSCSVMHEALHNHYTQKSLSL | |
| L234A/L235A/P329A | SPG | |
| Without C-terminal | | |
| Lysine | | |
| hIgG1 CH1+ Hinge | ASTKGPSVFPLAPSSKSTSGGTAALGCLVKDYFPEPVT | 215 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| Hole | PEAAGGPSVFLFPPKPKDTLMISRTPEVTCVVVDVSHE | |
| T366S/L368A/407V/ | DPEVKFNWYVDGVEVHNAKTKPREEQYNSTYRVVSVLT | |
| Y349C | VLHQDWLNGKEYKCKVSNKALGAPIEKTISKAKGQPRE | |
| L234A/L235A/P329A | PQVCTLPPSRDELTKNQVSLSCAVKGFYPSDIAVEWES | |
| With C-terminal Lysine | NGQPENNYKTTPPVLDSDGSFFLVSKLTVDKSRWQQGN | |
| | VFSCSVMHEALHNHYTQKSLSLSPGK | |
| hIgG1 CH1 + Hinge | ASTKGPSVFPLAPSSKSTSGGTAALGCLVKDYFPEPVT | 216 |
| Region + CH2 Region + | VSWNSGALTSGVHTFPAVLQSSGLYSLSSVVTVPSSSL | |
| CH3 Region | GTQTYICNVNHKPSNTKVDKKVEPKSCDKTHTCPPCPA | |
| L234A/L235A/P329A | PEAAGGPSVFLFPPKPKDTLMISRTPEVTCVVVDVSHE | |
| Hole | DPEVKFNWYVDGVEVHNAKTKPREEQYNSTYRVVSVLT | |
| T366S/L368A/407V/ | VLHQDWLNGKEYKCKVSNKALGAPIEKTISKAKGQPRE | |
| Y349C | PQVCTLPPSRDELTKNQVSLSCAVKGFYPSDIAVEWES | |
| L234A/L235A/P329A | NGQPENNYKTTPPVLDSDGSFFLVSKLTVDKSRWQQGN | |
| Without C-terminal | VFSCSVMHEALHNHYTQKSLSLSPG | |
| Lysine | | |

In some embodiments, the amino acid sequence of the first Fc region comprises a W amino acid residue at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 185-192; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 193-200.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 201-208; and the amino acid sequence of the second Ig Fc region comprises a S amino acid at position T366, an A amino acid at position L368, a V amino acid at position Y407, and a C amino acid at position Y349, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 209-216.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 185; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 193.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 186; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 194.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 187; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 195.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 188; and the amino acid sequence of the second Ig Fc region comprises a S amino acid at position T366, a C amino acid residue at position Y349, an L amino acid residue at position L234, an L amino acid residue at position L235, and an A amino acid residue at position P329, an A amino acid at position L368, and a V amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 196.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 189; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 197.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 190; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 198.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 191; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 199.

In some embodiments, the amino acid sequence of the first Fc region comprises a W amino acid at position T366, a C amino acid residue at position S354, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 192; and the amino acid sequence of the second Ig Fc region comprises a S amino acid at position T366, an A amino acid at position L368, and a V amino acid at position Y407, a C amino acid residue at position Y349, an L amino acid residue at position L234, an L amino acid residue at position L235, and an A amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 200.

In some embodiments, the amino acid sequence of the first Fc region comprises a W amino acid at position T366, a C amino acid residue at position S354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 201; and the amino acid sequence of the second Ig Fc region comprises a S amino acid at position T366, an A amino acid at position L368, a C amino acid residue at position Y349, an L amino acid residue at position L234, an L amino acid residue at position L235, and an G amino acid residue at position P329, a V amino acid at position Y407, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 209.

In some embodiments, the amino acid sequence of the first Fc region comprises a W amino acid at position T366, a C amino acid residue at position S354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 202; and the amino acid sequence of the second Ig Fc region comprises a S amino acid at position T366, an A amino acid at position L368, a V amino acid at position Y407, a C amino acid residue at position Y349, an L amino acid residue at position L234, an L amino acid residue at position L235, and an G amino acid residue at position P329, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 210.

In some embodiments, the amino acid sequence of the first Fc region comprises a W amino acid at position T366, a C amino acid residue at position S354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 203; and the amino acid sequence of the second Ig Fc region comprises a S amino acid at position T366, an A amino acid at position L368, a V amino acid at position Y407, a C amino acid residue at position Y349, an L amino acid residue at position L234, an L amino acid residue at position L235, and an G amino acid residue at position P329, and a C amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 211.

In some embodiments, the amino acid sequence of the first Fc region comprises a W amino acid at position T366, a C amino acid residue at position 5354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 204; and the amino acid sequence of the second Ig Fc region comprises a S amino acid at position T366, an A amino acid at position L368, a V amino acid at position Y407, and a C amino acid at position Y349, a C amino acid residue at position Y349, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 212.

In some embodiments, the amino acid sequence of the first Fc region comprises a W amino acid at position T366, a C amino acid residue at position S354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 205; and the amino acid sequence of the second Ig Fc region comprises a S amino acid at position T366, an A amino acid at position L368, a V amino acid at position Y407, and a C amino acid at position Y349, a C amino acid residue at position Y349, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 213.

In some embodiments, the amino acid sequence of the first Fc region comprises a W amino acid at position T366, a C amino acid residue at position S354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 206; and the amino acid sequence of the second Ig Fc region comprises a S amino acid at position T366, an A amino acid at position L368, a V amino acid at position Y407, and a C amino acid at position Y349, a C amino acid residue at position Y349, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 214.

In some embodiments, the amino acid sequence of the first Fc region comprises a W amino acid at position T366, a C amino acid residue at position S354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 207; and the amino acid sequence of the second Ig Fc region comprises a S amino acid at position T366, an A amino acid at position L368, a V amino acid at position Y407, and a C amino acid at position Y349, a C amino acid residue at position Y349, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 215.

In some embodiments, the amino acid sequence of the first Fc region comprises a W amino acid at position T366, a C amino acid residue at position S354, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 208; and the amino acid sequence of the second Ig Fc region comprises a S amino acid at position T366, an A amino acid at position L368, a V amino acid at position Y407, and a C amino acid at position Y349, a C amino acid residue at position Y349, an L amino acid residue at position L234, an L amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 216.

### 5.2.8 Linkers

As described herein, peptide linkers can be used to operably connect a first component of a fusion protein or polypeptide thereof described herein to a second component (see, e.g., § 5.2.1). For example, in Format BCA405 (see, e.g., § 5.2.1.1), described herein a peptide linker can be utilized to operably connect the first scFv to the full-length antibody (more particularly to the C-terminus of the CH3 of the first Fc region of the full-length antibody).

In some embodiments, the peptide linker is one or any combination of a non-cleavable linker, a flexible linker, a rigid linker, a helical linker, and/or a non-helical linker.

In some embodiments, the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises or consists of glycine, serine, or both glycine and serine amino acid residues. In some embodiments, the amino acid sequence of the peptide linker comprises or consists of glycine, serine, and proline amino acid residues.

The amino acid sequence of exemplary peptide linkers, which can be incorporated in one or more of the embodiments described herein (e.g., multispecific proteins (and polypeptides thereof)), is set provided in Table 13.

TABLE 13
The Amino Acid Sequence of Exemplary Peptide
Linker

| | | SEQ |
|---|---|---|
| | | ID |
| Description | Amino Acid Sequence | NO |
| Linker A | GGGS | 217 |
| Linker B | GGGSGGGS | 218 |
| Linker C | GGGSGGGSGGGS | 219 |
| Linker D | GGGSGGGSGGGSGGGS | 220 |
| Linker E | GGGGS | 221 |
| Linker F | GGGGSGGGGS | 222 |
| Linker G | GGGGSGGGGSGGGGS | 223 |
| Linker H | GGGGSGGGGSGGGGSGGGGS | 224 |
| Linker I | GGGGGGGS | 225 |
| Linker J | GGGGGGGSGGGGGGGS | 226 |
| Linker K | GGGGGGGSGGGGGGGSGGGGGGGS | 227 |
| Linker L | GGGGGGGSGGGGGGGSGGGGGGGSGGGGGGGS | 228 |
| Linker M | SGGGG | 229 |
| Linker N | SGGGGSGGGG | 230 |
| Linker O | SGGGGSGGGGSGGGG | 231 |
| Linker P | SGGGGSGGGGSGGGGSGGGG | 232 |
| Linker Q | GGGGGGS | 233 |
| Linker R | GGGGGGSGGGGGGS | 234 |
| Linker S | GGGGGGSGGGGGGSGGGGGGS | 235 |
| Linker T | GGGGGGSGGGGGGSGGGGGGSGGGGGGS | 236 |
| Linker U | DKTHTCPPCP | 318 |
| Linker V | EPKSCDGGGGGGGGS | 319 |

In some embodiments, the amino acid sequence of the peptide linker comprises or consists of the amino acid sequence of any one of the linkers set forth in Table 13. In some embodiments, the amino acid sequence of the peptide linker comprises or consists of the amino acid sequence of any one of the linkers set forth in Table 13, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of the linkers set forth in Table 13, comprising 1, 2, or 3 amino acid variations (e.g., substitutions, deletions, additions). In some embodiments, the amino acid sequence of the peptide linker comprises or consists of the amino acid sequence of any one of the linkers set forth in Table 13, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 13, comprising 1, 2, or 3 amino acid substitutions.

In some embodiments, the amino acid sequence of the peptide linker comprises or consists of the amino acid sequence of any one of SEQ ID NOS: 217-236 or 318-319. In some embodiments, the amino acid sequence of the peptide linker comprises or consists of the amino acid sequence of any one of SEQ ID NOS: 217-236 or 318-319, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of SEQ ID NOS: 217-236 or 318-319, comprising 1, 2, or 3 amino acid variations (e.g., substitutions, deletions, additions). In some embodiments, the amino acid sequence of the peptide linker comprises or consists of the amino acid sequence of any one of SEQ ID NOS: 217-236 or 318-319, and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 217-236 or 318-319, comprising 1, 2, or 3 amino acid substitutions.

In some embodiments, the amino acid sequence of the peptide linker comprises or consists of the amino acid sequence of SEQ ID NO: 223, and further comprises 1 or more but less than 15% (less than 12%, less than 10%, less than 8%), amino acid substitutions. In some embodiments, the amino acid sequence of the peptide linker comprises or consists of the amino acid sequence of SEQ ID NO: 223. In some embodiments, the amino acid sequence of the peptide linker comprises or consists of the amino acid sequence of any one of SEQ ID NO: 223, comprising 1, 2, or 3 amino acid variations (e.g., substitutions, additions, deletions). In some embodiments, the amino acid sequence of the peptide linker comprises or consists of the amino acid sequence of any one of SEQ ID NO: 223, comprising 1, 2, or 3 amino acid substitutions.

### 5.2.9 Exemplary Multispecific Proteins and Polypeptides Thereof

The amino acid sequence of exemplary multispecific proteins (MFP-1-4) (and polypeptides thereof) described herein is provided in Table 14. The fusion proteins (and polypeptides thereof) provided in Table 14 are exemplary only, and not intended to be limiting.

TABLE 14
The Amino Acid Sequence of Exemplary Multispecific proteins & Polypeptides
Thereof

| | | | SEQ |
|---|---|---|---|
| Description | | Amino Acid Sequence | ID NO |
| BCA405 | Heavy Chain | EVQLVESGGGLVQPGGSLRLSCAASGFNIKDTYIHWVRQ | 237 |
| αhHER2/hCD3/ | (HC)(Knob) | APGKGLEWVARIYPTNGYTRYADSVKGRFTISADTSKNT | |
| hCD28 | αHER2 | AYLQMNSLRAEDTAVYYCSRWGGDGFYAMDYWGQGTLVT | |
| | αCD3 scFv | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDKTHTCPPCPA | |
| | | PEAAGGPSVFLFPPKPKDTLMISRTPEVTCVVVDVSHED | |
| | | PEVKFNWYVDGVEVHNAKTKPREEQYNSTYRVVSVLTVL | |
| | | HQDWLNGKEYKCKVSNKALAAPIEKTISKAKGQPREPQV | |
| | | YTLPPCRDELTKNQVSLWCLVKGFYPSDIAVEWESNGQP | |
| | | ENNYKTTPPVLDSDGSFFLYSKLTVDKSRWQQGNVFSCS | |
| | | VMHEALHNHYTQKSLSLSPGKGGGGSGGGGSGGGGSEVQ | |
| | | LVESGGGLVQPGGSLKLSCAASGFTFNKYAMNWVRQAPG | |
| | | KGLEWVARIRSKYNNYATYYADSVKDRFTISRDDSKNTA | |
| | | YLQMNNLKTEDTAVYYCVRHGNFGNSYISYWAYWGQGTL | |
| | | VTVSSGGGGSGGGGSGGGGSQTVVTQEPSLTVSPGGTVT | |
| | | LTCGSSTGAVTSGYYPNWVQQKPGQAPRGLIGGTKFLAP | |
| | | GTPARFSGSLLGGKAALTLSGVQPEDEAEYYCALWYSNR | |
| | | WVFGGGTKLTVL | |
| | HC (Hole) | EVQLVESGGGLVQPGGSLRLSCAASGFNIKDTYIHWVRQ | 238 |
| | αHER2 | APGKGLEWVARIYPTNGYTRYADSVKGRFTISADTSKNT | |
| | αCD28 scFv | AYLQMNSLRAEDTAVYYCSRWGGDGFYAMDYWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDKTHTCPPCPA | |
| | | PEAAGGPSVFLFPPKPKDTLMISRTPEVTCVVVDVSHED | |
| | | PEVKFNWYVDGVEVHNAKTKPREEQYNSTYRVVSVLTVL | |
| | | HQDWLNGKEYKCKVSNKALAAPIEKTISKAKGQPREPQV | |
| | | CTLPPSRDELTKNQVSLSCAVKGFYPSDIAVEWESNGQP | |
| | | ENNYKTTPPVLDSDGSFFLVSKLTVDKSRWQQGNVFSCS | |
| | | VMHEALHNHYTQKSLSLSPGKGGGGSGGGGSGGGGSQVQ | |
| | | LVQSGAEVKKPGASVKVSCKASGYTFTSYYIHWVRQAPG | |
| | | QGLEWIGCIYPGNVNTNYNEKFKDRATLTVDTSISTAYM | |
| | | ELSRLRSDDTAVYFCTRSHYGLDWNFDVWGQGTTVTVSS | |
| | | GGGGSGGGGSGGGGSDIQMTQSPSSLSASVGDRVTITCH | |
| | | ASQNIYVWLNWYQQKPGKAPKLLIYKASNLHTGVPSRFS | |
| | | GSGSGTDFTLTISSLQPEDFATYYCQQGQTYPYTFGGGT | |
| | | KVEIK | |
| | Light Chain | DIQMTQSPSSLSASVGDRVTITCRASQDVNTAVAWYQQK | 239 |
| | (LC) | PGKAPKLLIYSASFLYSGVPSRFSGSRSGTDFTLTISSL | |
| | αHER2 | QPEDFATYYCQQHYTTPPTFGQGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA406 | HC (Knob) | QTVVTQEPSLTVSPGGTVTLTCGSSTGAVTSGYYPNWVQ | 240 |
| αhHER2/hCD3/ | αCD3 scFv | QKPGQAPRGLIGGTKFLAPGTPARFSGSLLGGKAALTLS | |
| hCD28 | αHER2 | GVQPEDEAEYYCALWYSNRWVFGGGTKLTVLGGGGSGGG | |
| | | GSGGGGSEVQLVESGGGLVQPGGSLKLSCAASGFTFNKY | |
| | | AMNWVRQAPGKGLEWVARIRSKYNNYATYYADSVKDRFT | |
| | | ISRDDSKNTAYLQMNNLKTEDTAVYYCVRHGNFGNSYIS | |
| | | YWAYWGQGTLVTVSSASTKGPEVQLVESGGGLVQPGGSL | |
| | | RLSCAASGFNIKDTYIHWVRQAPGKGLEWVARIYPTNGY | |
| | | TRYADSVKGRFTISADTSKNTAYLQMNSLRAEDTAVYYC | |
| | | SRWGGDGFYAMDYWGQGTLVTVSSASTKGPSVFPLAPSS | |
| | | KSTSGGTAALGCLVKDYFPEPVTVSWNSGALTSGVHTFP | |
| | | AVLQSSGLYSLSSVVTVPSSSLGTQTYICNVNHKPSNTK | |
| | | VDKKVEPKSCDKTHTCPPCPAPEAAGGPSVFLFPPKPKD | |
| | | TLMISRTPEVTCVVVDVSHEDPEVKFNWYVDGVEVHNAK | |
| | | TKPREEQYNSTYRVVSVLTVLHQDWLNGKEYKCKVSNKA | |
| | | LAAPIEKTISKAKGQPREPQVYTLPPCRDELTKNQVSLW | |
| | | CLVKGFYPSDIAVEWESNGQPENNYKTTPPVLDSDGSFF | |
| | | LYSKLTVDKSRWQQGNVFSCSVMHEALHNHYTQKSLSLS | |
| | | PGK | |
| | HC (Hole) | DIQMTQSPSSLSASVGDRVTITCHASQNIYVWLNWYQQK | 241 |
| | αCD28 scFv | PGKAPKLLIYKASNLHTGVPSRFSGSGSGTDFTLTISSL | |
| | αHER2 | QPEDFATYYCQQGQTYPYTFGGGTKVEIKGGGGSGGGGS | |
| | | GGGGSQVQLVQSGAEVKKPGASVKVSCKASGYTFTSYYI | |
| | | HWVRQAPGQGLEWIGCIYPGNVNTNYNEKFKDRATLTVD | |
| | | TSISTAYMELSRLRSDDTAVYFCTRSHYGLDWNFDVWGQ | |
| | | GTTVTVSSASTKGPEVQLVESGGGLVQPGGSLRLSCAAS | |
| | | GFNIKDTYIHWVRQAPGKGLEWVARIYPTNGYTRYADSV | |
| | | KGRFTISADTSKNTAYLQMNSLRAEDTAVYYCSRWGGDG | |
| | | FYAMDYWGQGTLVTVSSASTKGPSVFPLAPSSKSTSGGT | |
| | | AALGCLVKDYFPEPVTVSWNSGALTSGVHTFPAVLQSSG | |
| | | LYSLSSVVTVPSSSLGTQTYICNVNHKPSNTKVDKKVEP | |
| | | KSCDKTHTCPPCPAPEAAGGPSVFLFPPKPKDTLMISRT | |
| | | PEVTCVVVDVSHEDPEVKFNWYVDGVEVHNAKTKPREEQ | |
| | | YNSTYRVVSVLTVLHQDWLNGKEYKCKVSNKALAAPIEK | |
| | | TISKAKGQPREPQVCTLPPSRDELTKNQVSLSCAVKGFY | |
| | | PSDIAVEWESNGQPENNYKTTPPVLDSDGSFFLVSKLTV | |
| | | DKSRWQQGNVFSCSVMHEALHNHYTQKSLSLSPGK | |
| | LC | DIQMTQSPSSLSASVGDRVTITCRASQDVNTAVAWYQQK | 239 |
| | αHER2 | PGKAPKLLIYSASFLYSGVPSRFSGSRSGTDFTLTISSL | |
| | | QPEDFATYYCQQHYTTPPTFGQGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA424 | HC (Knob) | EVQLVESGGGLVQPGGSLRLSCAASGFNIKDTYIHWVRQ | 242 |
| αhHER2/hCD3/ | αHER2 | APGKGLEWVARIYPTNGYTRYADSVKGRFTISADTSKNT | |
| hCD28 | αCD28 scFv | AYLQMNSLRAEDTAVYYCSRWGGDGFYAMDYWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDGGGGSGGGGS | |
| | | QVQLVQSGAEVKKPGASVKVSCKASGYTFTSYYIHWVRQ | |
| | | APGQCLEWIGCIYPGNVNTNYNEKFKDRATLTVDTSIST | |
| | | AYMELSRLRSDDTAVYFCTRSHYGLDWNFDVWGQGTTVT | |
| | | VSSGGGGSGGGGSGGGGSDIQMTQSPSSLSASVGDRVTI | |
| | | TCHASQNIYVWLNWYQQKPGKAPKLLIYKASNLHTGVPS | |
| | | RFSGSGSGTDFTLTISSLQPEDFATYYCQQGQTYPYTFG | |
| | | CGTKVEIKEPKSSDKTHTCPPCPAPEAAGGPSVFLFPPK | |
| | | PKDTLMISRTPEVTCVVVDVSHEDPEVKFNWYVDGVEVH | |
| | | NAKTKPREEQYNSTYRVVSVLTVLHQDWLNGKEYKCKVS | |
| | | NKALAAPIEKTISKAKGQPREPQVYTLPPCRDELTKNQV | |
| | | SLWCLVKGFYPSDIAVEWESNGQPENNYKTTPPVLDSDG | |
| | | SFFLYSKLTVDKSRWQQGNVFSCSVMHEALHNHYTQKSL | |
| | | SLSPGK | |
| | HC (Hole) | EVQLVESGGGLVQPGGSLRLSCAASGFNIKDTYIHWVRQ | 243 |
| | αHER2 | APGKGLEWVARIYPTNGYTRYADSVKGRFTISADTSKNT | |
| | αCD3 scFv | AYLQMNSLRAEDTAVYYCSRWGGDGFYAMDYWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDGGGGSGGGGS | |
| | | EVQLVESGGGLVQPGGSLKLSCAASGFTENKYAMNWVRQ | |
| | | APGKCLEWVARIRSKYNNYATYYADSVKDRFTISRDDSK | |
| | | NTAYLQMNNLKTEDTAVYYCVRHGNFGNSYISYWAYWGQ | |
| | | GTLVTVSSGGGGSGGGGSGGGGSQTVVTQEPSLTVSPGG | |
| | | TVTLTCGSSTGAVTSGYYPNWVQQKPGQAPRGLIGGTKF | |
| | | LAPGTPARFSGSLLGGKAALTLSGVQPEDEAEYYCALWY | |
| | | SNRWVFGCGTKLTVLEPKSSDKTHTCPPCPAPEAAGGPS | |
| | | VFLFPPKPKDTLMISRTPEVTCVVVDVSHEDPEVKENWY | |
| | | VDGVEVHNAKTKPREEQYNSTYRVVSVLTVLHQDWLNGK | |
| | | EYKCKVSNKALAAPIEKTISKAKGQPREPQVCTLPPSRD | |
| | | ELTKNQVSLSCAVKGFYPSDIAVEWESNGQPENNYKTTP | |
| | | PVLDSDGSFFLVSKLTVDKSRWQQGNVFSCSVMHEALHN | |
| | | HYTQKSLSLSPGK | |
| | LC | DIQMTQSPSSLSASVGDRVTITCRASQDVNTAVAWYQQK | 239 |
| | αHER2 | PGKAPKLLIYSASFLYSGVPSRFSGSRSGTDFTLTISSL | |
| | | QPEDFATYYCQQHYTTPPTFGQGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA418 | HC (Knob) | QVQLVQSGAEVKKPGASVKVSCKASGYTFTSYYIHWVRQ | 244 |
| αhHER2/hCD3/ | αCD28 ScFv | APGQCLEWIGCIYPGNVNTNYNEKFKDRATLTVDTSIST | |
| hCD28 | αCD3 | AYMELSRLRSDDTAVYFCTRSHYGLDWNFDVWGQGTTVT | |
| | | VSSGGGGSGGGGSGGGGSDIQMTQSPSSLSASVGDRVTI | |
| | | TCHASQNIYVWLNWYQQKPGKAPKLLIYKASNLHTGVPS | |
| | | RFSGSGSGTDFTLTISSLQPEDFATYYCQQGQTYPYTFG | |
| | | CGTKVEIKGGGGSGGGGSEVQLVESGGGLVQPGGSLKLS | |
| | | CAASGFTENKYAMNWVRQAPGKGLEWVARIRSKYNNYAT | |
| | | YYADSVKDRFTISRDDSKNTAYLQMNNLKTEDTAVYYCV | |
| | | RHGNFGNSYISYWAYWGQGTLVTVSSASTKGPSVFPLAP | |
| | | SSKSTSGGTAALGCLVKDYFPEPVTVSWNSGALTSGVHT | |
| | | FPAVLQSSGLYSLSSVVTVPSSSLGTQTYICNVNHKPSN | |
| | | TKVDKKVEPKSCDKTHTCPPCPAPEAAGGPSVFLFPPKP | |
| | | KDTLMISRTPEVTCVVVDVSHEDPEVKFNWYVDGVEVHN | |
| | | AKTKPREEQYNSTYRVVSVLTVLHQDWLNGKEYKCKVSN | |
| | | KALAAPIEKTISKAKGQPREPQVYTLPPCRDELTKNQVS | |
| | | LWCLVKGFYPSDIAVEWESNGQPENNYKTTPPVLDSDGS | |
| | | FFLYSKLTVDKSRWQQGNVFSCSVMHEALHNHYTQKSLS | |
| | | LSPGK | |
| | HC Hole | EVQLVESGGGLVQPGGSLRLSCAASGFNIKDTYIHWVRQ | 245 |
| | αHER2 | APGKGLEWVARIYPTNGYTRYADSVKGRFTISADTSKNT | |
| | | AYLQMNSLRAEDTAVYYCSRWGGDGFYAMDYWGQGTLVT | |
| | | VSSASTELPPKVSVFVPPRDGFFGNPRKSELICEATGFS | |
| | | PRQIQVSWLREGKQVGSGVTTDQVQAEAKESGPTTYDVT | |
| | | STLTIKESDWLGQSMFTCRVDHRGLTFQQSASSMCDKTH | |
| | | TCPPCPAPEAAGGPSVFLFPPKPKDTLMISRTPEVTCVV | |
| | | VDVSHEDPEVKFNWYVDGVEVHNAKTKPREEQYNSTYRV | |
| | | VSVLTVLHQDWLNGKEYKCKVSNKALAAPIEKTISKAKG | |
| | | QPREPQVCTLPPSRDELTKNQVSLSCAVKGFYPSDIAVE | |
| | | WESNGQPENNYKTTPPVLDSDGSFFLVSKLTVDKSRWQQ | |
| | | GNVFSCSVMHEALHNHYTQKSLSLSPGK | |
| | LC αCD3 | QTVVTQEPSLTVSPGGTVTLTCGSSTGAVTSGYYPNWVQ | 246 |
| | | QKPGQAPRGLIGGTKFLAPGTPARFSGSLLGGKAALTLS | |
| | | GVQPEDEAEYYCALWYSNRWVFGGGTKLTVLRTVAAPSV | |
| | | FIFPPSDEQLKSGTASVVCLLNNFYPREAKVQWKVDNAL | |
| | | QSGNSQESVTEQDSKDSTYSLSSTLTLSKADYEKHKVYA | |
| | | CEVTHQGLSSPVTKSFNRGEC | |
| | LC IgM | DIQMTQSPSSLSASVGDRVTITCRASQDVNTAVAWYQQK | 247 |
| | CH2 αHER2 | PGKAPKLLIYSASFLYSGVPSRFSGSRSGTDFTLTISSL | |
| | | QPEDFATYYCQQHYTTPPTFGQGTKVEIKRTAELPPKVS | |
| | | VFVPPRDGFFGNPRKSKLICKATGFSPRQIQVSWLREGK | |
| | | QVGSGVTTKQVQAEAKESGPTTYKVTSTLTIKESDWLGQ | |
| | | SMFTCRVDHRGLTFQQSASSMC | |
| BCA405.S | HC (Knob) | EVQLVESGGGLVQPGGSLRLSCAASGFNIKDTYIHWVRQ | 248 |
| αhHER2/hCD3/ | αHER2 | APGKGLEWVARIYPTNGYTRYADSVKGRFTISADTSKNT | |
| hCD28 | αCD3 scFv | AYLQMNSLRAEDTAVYYCSRWGGDGFYAMDYWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDKTHTCPPCPA | |
| | | PEAAGGPSVFLFPPKPKDTLMISRTPEVTCVVVDVSHED | |
| | | PEVKFNWYVDGVEVHNAKTKPREEQYNSTYRVVSVLTVL | |
| | | HQDWLNGKEYKCKVSNKALAAPIEKTISKAKGQPREPQV | |
| | | YTLPPCRDELTKNQVSLWCLVKGFYPSDIAVEWESNGQP | |
| | | ENNYKTTPPVLDSDGSFFLYSKLTVDKSRWQQGNVFSCS | |
| | | VMHEALHNHYTQKSLSLSPGKGGGGSGGGGSGGGGSQVQ | |
| | | LVESGGGVVQPGRSLRLSCAASGFTFTKAWMHWVRQAPG | |
| | | KQLEWVAQIKDKSNSYATYYADSVKGRFTISRDDSKNTL | |
| | | YLQMNSLRAEDTAVYYCRGVYYALSPFDYWGQGTLVTVS | |
| | | SGGGGSGGGGSGGGGSDIVMTQTPLSLSVTPGQPASISC | |
| | | KSSQSLVHNNANTYLSWYLQKPGQSPQSLIYKVSNRFSG | |
| | | VPDRFSGSGSGTDFTLKISRVEAEDVGVYYCGQGTQYPF | |
| | | TFGSGTKVEIK | |
| | HC (Hole) | EVQLVESGGGLVQPGGSLRLSCAASGFNIKDTYIHWVRQ | 249 |
| | αHER2 | APGKGLEWVARIYPTNGYTRYADSVKGRFTISADTSKNT | |
| | αCD28 scFv | AYLQMNSLRAEDTAVYYCSRWGGDGFYAMDYWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDKTHTCPPCPA | |
| | | PEAAGGPSVFLFPPKPKDTLMISRTPEVTCVVVDVSHED | |
| | | PEVKFNWYVDGVEVHNAKTKPREEQYNSTYRVVSVLTVL | |
| | | HQDWLNGKEYKCKVSNKALAAPIEKTISKAKGQPREPQV | |
| | | CTLPPSRDELTKNQVSLSCAVKGFYPSDIAVEWESNGQP | |
| | | ENNYKTTPPVLDSDGSFFLVSKLTVDKSRWQQGNVFSCS | |
| | | VMHEALHNHYTQKSLSLSPGKGGGGSGGGGSGGGGSQVQ | |
| | | LQESGPGLVKPSQTLSLTCTVSGFSLSDYGVHWV'RQPP | |
| | | GKGLEWLGVIWAGGGTNYNPSLKSRKTISKDTSKNQVSL | |
| | | KLSSVTAADTAVYYCARDKGYSYYYSMDYWGQGTTVTVS | |
| | | SGGGGSGGGGSGGGGSDIVLTQSPASLAVSPGQRATITC | |
| | | RASESVEYYVTSLMQWYQQKPGQPPKLLIFAASNVESGV | |
| | | PARFSGSGSGTDFTLTINPVEANDVANYYCQQSRKVPYT | |
| | | FGQGTKLEIK | |
| | LC | DIQMTQSPSSLSASVGDRVTITCRASQDVNTAVAWYQQK | 239 |
| | αHER2 | PGKAPKLLIYSASFLYSGVPSRFSGSRSGTDFTLTISSL | |
| | | QPEDFATYYCQQHYTTPPTFGQGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA406.S | HC (Knob) | DIVMTQTPLSLSVTPGQPASISCKSSQSLVHNNANTYLS | 250 |
| αhHER2/hCD3/ | αCD3 scFv | WYLQKPGQSPQSLIYKVSNRFSGVPDRFSGSGSGTDFTL | |
| hCD28 | αHER2 | KISRVEAEDVGVYYCGQGTQYPFTFGSGTKVEIKGGGGS | |
| | | GGGGSGGGGSQVQLVESGGGVVQPGRSLRLSCAASGFTF | |
| | | TKAWMHWVRQAPGKQLEWVAQIKDKSNSYATYYADSVKG | |
| | | RFTISRDDSKNTLYLQMNSLRAEDTAVYYCRGVYYALSP | |
| | | FDYWGQGTLVTVSSASTKGPEVQLVESGGGLVQPGGSLR | |
| | | LSCAASGFNIKDTYIHWVRQAPGKGLEWVARIYPTNGYT | |
| | | RYADSVKGRFTISADTSKNTAYLQMNSLRAEDTAVYYCS | |
| | | RWGGDGFYAMDYWGQGTLVTVSSASTKGPSVFPLAPSSK | |
| | | STSGGTAALGCLVKDYFPEPVTVSWNSGALTSGVHTFPA | |
| | | VLQSSGLYSLSSVVTVPSSSLGTQTYICNVNHKPSNTKV | |
| | | DKKVEPKSCDKTHTCPPCPAPEAAGGPSVFLFPPKPKDT | |
| | | LMISRTPEVTCVVVDVSHEDPEVKFNWYVDGVEVHNAKT | |
| | | KPREEQYNSTYRVVSVLTVLHQDWLNGKEYKCKVSNKAL | |
| | | AAPIEKTISKAKGQPREPQVYTLPPCRDELTKNQVSLWC | |
| | | LVKGFYPSDIAVEWESNGQPENNYKTTPPVLDSDGSFFL | |
| | | YSKLTVDKSRWQQGNVFSCSVMHEALHNHYTQKSLSLSP | |
| | | GK | |
| | HC (Hole) | DIVLTQSPASLAVSPGQRATITCRASESVEYYVTSLMQW | 251 |
| | αCD28 scFv | YQQKPGQPPKLLIFAASNVESGVPARFSGSGSGTDFTLT | |
| | αHER2 | INPVEANDVANYYCQQSRKVPYTFGQGTKLEIKGGGGSG | |
| | | GGGSGGGGSQVQLQESGPGLVKPSQTLSLTCTVSGFSLS | |
| | | DYGVHWV'RQPPGKGLEWLGVIWAGGGTNYNPSLKSRKT | |
| | | ISKDTSKNQVSLKLSSVTAADTAVYYCARDKGYSYYYSM | |
| | | DYWGQGTTVTVSSASTKGPEVQLVESGGGLVQPGGSLRL | |
| | | SCAASGFNIKDTYIHWVRQAPGKGLEWVARIYPTNGYTR | |
| | | YADSVKGRFTISADTSKNTAYLQMNSLRAEDTAVYYCSR | |
| | | WGGDGFYAMDYWGQGTLVTVSSASTKGPSVFPLAPSSKS | |
| | | TSGGTAALGCLVKDYFPEPVTVSWNSGALTSGVHTFPAV | |
| | | LQSSGLYSLSSVVTVPSSSLGTQTYICNVNHKPSNTKVD | |
| | | KKVEPKSCDKTHTCPPCPAPEAAGGPSVFLFPPKPKDTL | |
| | | MISRTPEVTCVVVDVSHEDPEVKFNWYVDGVEVHNAKTK | |
| | | PREEQYNSTYRVVSVLTVLHQDWLNGKEYKCKVSNKALA | |
| | | APIEKTISKAKGQPREPQVCTLPPSRDELTKNQVSLSCA | |
| | | VKGFYPSDIAVEWESNGQPENNYKTTPPVLDSDGSFFLV | |
| | | SKLTVDKSRWQQGNVFSCSVMHEALHNHYTQKSLSLSPG | |
| | | K | |
| | LC | DIQMTQSPSSLSASVGDRVTITCRASQDVNTAVAWYQQK | 239 |
| | αHER2 | PGKAPKLLIYSASFLYSGVPSRFSGSRSGTDFTLTISSL | |
| | | QPEDFATYYCQQHYTTPPTFGQGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA424.S | HC (Knob) | EVQLVESGGGLVQPGGSLRLSCAASGFNIKDTYIHWVRQ | 252 |
| αhHER2/hCD3/ | αHER2 | APGKGLEWVARIYPTNGYTRYADSVKGRFTISADTSKNT | |
| hCD28 | αCD28 scFv | AYLQMNSLRAEDTAVYYCSRWGGDGFYAMDYWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDGGGGSGGGGS | |
| | | QVQLQESGPGLVKPSQTLSLTCTVSGFSLSDYGVHWVRQ | |
| | | PPGKCLEWLGVIWAGGGTNYNPSLKSRKTISKDTSKNQV | |
| | | SLKLSSVTAADTAVYYCARDKGYSYYYSMDYWGQGTTVT | |
| | | VSSGGGGSGGGGSGGGGSDIVLTQSPASLAVSPGQRATI | |
| | | TCRASESVEYYVTSLMQWYQQKPGQPPKLLIFAASNVES | |
| | | GVPARFSGSGSGTDFTLTINPVEANDVANYYCQQSRKVP | |
| | | YTFGCGTKLEIKEPKSSDKTHTCPPCPAPEAAGGPSVFL | |
| | | FPPKPKDTLMISRTPEVTCVVVDVSHEDPEVKFNWYVDG | |
| | | VEVHNAKTKPREEQYNSTYRVVSVLTVLHQDWLNGKEYK | |
| | | CKVSNKALAAPIEKTISKAKGQPREPQVYTLPPCRDELT | |
| | | KNQVSLWCLVKGFYPSDIAVEWESNGQPENNYKTTPPVL | |
| | | DSDGSFFLYSKLTVDKSRWQQGNVFSCSVMHEALHNHYT | |
| | | QKSLSLSPGK | |
| | HC (Hole) | EVQLVESGGGLVQPGGSLRLSCAASGFNIKDTYIHWVRQ | 253 |
| | αHER2 | APGKGLEWVARIYPTNGYTRYADSVKGRFTISADTSKNT | |
| | αCD3 scFv | AYLQMNSLRAEDTAVYYCSRWGGDGFYAMDYWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDGGGGSGGGGS | |
| | | QVQLVESGGGVVQPGRSLRLSCAASGFTFTKAWMHWVRQ | |
| | | APGKCLEWVAQIKDKSNSYATYYADSVKGRFTISRDDSK | |
| | | NTLYLQMNSLRAEDTAVYYCRGVYYALSPFDYWGQGTLV | |
| | | TVSSGGGGSGGGGSGGGGSDIVMTQTPLSLSVTPGQPAS | |
| | | ISCKSSQSLVHNNANTYLSWYLQKPGQSPQSLIYKVSNR | |
| | | FSGVPDRFSGSGSGTDFTLKISRVEAEDVGVYYCGQGTQ | |
| | | YPFTFGCGTKVEIKEPKSSDKTHTCPPCPAPEAAGGPSV | |
| | | FLFPPKPKDTLMISRTPEVTCVVVDVSHEDPEVKFNWYV | |
| | | DGVEVHNAKTKPREEQYNSTYRVVSVLTVLHQDWLNGKE | |
| | | YKCKVSNKALAAPIEKTISKAKGQPREPQVCTLPPSRDE | |
| | | LTKNQVSLSCAVKGFYPSDIAVEWESNGQPENNYKTTPP | |
| | | VLDSDGSFFLVSKLTVDKSRWQQGNVFSCSVMHEALHNH | |
| | | YTQKSLSLSPGK | |
| | LC | DIQMTQSPSSLSASVGDRVTITCRASQDVNTAVAWYQQK | 239 |
| | αHER2 | PGKAPKLLIYSASFLYSGVPSRFSGSRSGTDFTLTISSL | |
| | | QPEDFATYYCQQHYTTPPTFGQGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA405.EG | HC (Knob) | QVQLKQSGPGLVQPSQSLSITCTVSGFSLTNYGVHWVRQ | 254 |
| αhEGFR/hCD3/ | αEGFR | SPGKGLEWLGVIWSGGNTDYNTPFTSRLSINKDNSKSQV | |
| hCD28 | αCD3 scFv | FFKMNSLQSNDTAIYYCARALTYYDYEFAYWGQGTLVTV | |
| | | SAASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDKTHTCPPCPAP | |
| | | EAAGGPSVFLFPPKPKDTLMISRTPEVTCVVVDVSHEDP | |
| | | EVKFNWYVDGVEVHNAKTKPREEQYNSTYRVVSVLTVLH | |
| | | QDWLNGKEYKCKVSNKALAAPIEKTISKAKGQPREPQVY | |
| | | TLPPCRDELTKNQVSLWCLVKGFYPSDIAVEWESNGQPE | |
| | | NNYKTTPPVLDSDGSFFLYSKLTVDKSRWQQGNVFSCSV | |
| | | MHEALHNHYTQKSLSLSPGKGGGGSGGGGSGGGGSEVQL | |
| | | VESGGGLVQPGGSLKLSCAASGFTFNKYAMNWVRQAPGK | |
| | | GLEWVARIRSKYNNYATYYADSVKDRFTISRDDSKNTAY | |
| | | LQMNNLKTEDTAVYYCVRHGNFGNSYISYWAYWGQGTLV | |
| | | TVSSGGGGSGGGGSGGGGSQTVVTQEPSLTVSPGGTVTL | |
| | | TCGSSTGAVTSGYYPNWVQQKPGQAPRGLIGGTKFLAPG | |
| | | TPARFSGSLLGGKAALTLSGVQPEDEAEYYCALWYSNRW | |
| | | VFGGGTKLTVL | |
| | HC (Hole) | QVQLKQSGPGLVQPSQSLSITCTVSGFSLTNYGVHWVRQ | 255 |
| | αEGFR | SPGKGLEWLGVIWSGGNTDYNTPFTSRLSINKDNSKSQV | |
| | αCD28 scFv | FFKMNSLQSNDTAIYYCARALTYYDYEFAYWGQGTLVTV | |
| | | SAASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDKTHTCPPCPAP | |
| | | EAAGGPSVFLFPPKPKDTLMISRTPEVTCVVVDVSHEDP | |
| | | EVKFNWYVDGVEVHNAKTKPREEQYNSTYRVVSVLTVLH | |
| | | QDWLNGKEYKCKVSNKALAAPIEKTISKAKGQPREPQVC | |
| | | TLPPSRDELTKNQVSLSCAVKGFYPSDIAVEWESNGQPE | |
| | | NNYKTTPPVLDSDGSFFLVSKLTVDKSRWQQGNVFSCSV | |
| | | MHEALHNHYTQKSLSLSPGKGGGGSGGGGSGGGGSQVQL | |
| | | VQSGAEVKKPGASVKVSCKASGYTFTSYYIHWVRQAPGQ | |
| | | GLEWIGCIYPGNVNTNYNEKFKDRATLTVDTSISTAYME | |
| | | LSRLRSDDTAVYFCTRSHYGLDWNFDVWGQGTTVTVSSG | |
| | | GGGSGGGGSGGGGSDIQMTQSPSSLSASVGDRVTITCHA | |
| | | SQNIYVWLNWYQQKPGKAPKLLIYKASNLHTGVPSRFSG | |
| | | SGSGTDFTLTISSLQPEDFATYYCQQGQTYPYTFGGGTK | |
| | | VEIK | |
| | LC | DILLTQSPVILSVSPGERVSFSCRASQSIGTNIHWYQQR | 256 |
| | αEGFR | TNGSPRLLIKYASESISGIPSRFSGSGSGTDFTLSINSV | |
| | | ESEDIADYYCQQNNNWPTTFGAGTKLELKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA406.EG | HC (Knob) | QTVVTQEPSLTVSPGGTVTLTCGSSTGAVTSGYYPNWVQ | 257 |
| αhEGFR/hCD3/ | αCD3 scFv | QKPGQAPRGLIGGTKFLAPGTPARFSGSLLGGKAALTLS | |
| hCD28 | αEGFR | GVQPEDEAEYYCALWYSNRWVFGGGTKLTVLGGGGSGGG | |
| | | GSGGGGSEVQLVESGGGLVQPGGSLKLSCAASGFTFNKY | |
| | | AMNWVRQAPGKGLEWVARIRSKYNNYATYYADSVKDRFT | |
| | | ISRDDSKNTAYLQMNNLKTEDTAVYYCVRHGNFGNSYIS | |
| | | YWAYWGQGTLVTVSSASTKGPQVQLKQSGPGLVQPSQSL | |
| | | SITCTVSGFSLTNYGVHWVRQSPGKGLEWLGVIWSGGNT | |
| | | DYNTPFTSRLSINKDNSKSQVFFKMNSLQSNDTAIYYCA | |
| | | RALTYYDYEFAYWGQGTLVTVSAASTKGPSVFPLAPSSK | |
| | | STSGGTAALGCLVKDYFPEPVTVSWNSGALTSGVHTFPA | |
| | | VLQSSGLYSLSSVVTVPSSSLGTQTYICNVNHKPSNTKV | |
| | | DKKVEPKSCDKTHTCPPCPAPEAAGGPSVFLFPPKPKDT | |
| | | LMISRTPEVTCVVVDVSHEDPEVKFNWYVDGVEVHNAKT | |
| | | KPREEQYNSTYRVVSVLTVLHQDWLNGKEYKCKVSNKAL | |
| | | AAPIEKTISKAKGQPREPQVYTLPPCRDELTKNQVSLWC | |
| | | LVKGFYPSDIAVEWESNGQPENNYKTTPPVLDSDGSFFL | |
| | | YSKLTVDKSRWQQGNVFSCSVMHEALHNHYTQKSLSLSP | |
| | | GK | |
| | HC (Hole) | DIQMTQSPSSLSASVGDRVTITCHASQNIYVWLNWYQQK | 258 |
| | αCD28 scFv | PGKAPKLLIYKASNLHTGVPSRFSGSGSGTDFTLTISSL | |
| | αEGFR | QPEDFATYYCQQGQTYPYTFGGGTKVEIKGGGGSGGGGS | |
| | | GGGGSQVQLVQSGAEVKKPGASVKVSCKASGYTFTSYYI | |
| | | HWVRQAPGQGLEWIGCIYPGNVNTNYNEKFKDRATLTVD | |
| | | TSISTAYMELSRLRSDDTAVYFCTRSHYGLDWNFDVWGQ | |
| | | GTTVTVSSASTKGPQVQLKQSGPGLVQPSQSLSITCTVS | |
| | | GFSLTNYGVHWVRQSPGKGLEWLGVIWSGGNTDYNTPFT | |
| | | SRLSINKDNSKSQVFFKMNSLQSNDTAIYYCARALTYYD | |
| | | YEFAYWGQGTLVTVSAASTKGPSVFPLAPSSKSTSGGTA | |
| | | ALGCLVKDYFPEPVTVSWNSGALTSGVHTFPAVLQSSGL | |
| | | YSLSSVVTVPSSSLGTQTYICNVNHKPSNTKVDKKVEPK | |
| | | SCDKTHTCPPCPAPEAAGGPSVFLFPPKPKDTLMISRTP | |
| | | EVTCVVVDVSHEDPEVKFNWYVDGVEVHNAKTKPREEQY | |
| | | NSTYRVVSVLTVLHQDWLNGKEYKCKVSNKALAAPIEKT | |
| | | ISKAKGQPREPQVCTLPPSRDELTKNQVSLSCAVKGFYP | |
| | | SDIAVEWESNGQPENNYKTTPPVLDSDGSFFLVSKLTVD | |
| | | KSRWQQGNVFSCSVMHEALHNHYTQKSLSLSPGK | |
| | LC | DILLTQSPVILSVSPGERVSFSCRASQSIGTNIHWYQQR | 256 |
| | αEGFR | TNGSPRLLIKYASESISGIPSRFSGSGSGTDFTLSINSV | |
| | | ESEDIADYYCQQNNNWPTTFGAGTKLELKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA424.EG | HC (Knob) | QVQLKQSGPGLVQPSQSLSITCTVSGFSLTNYGVHWVRQ | 259 |
| αhEGFR/hCD3/ | αEGFR | SPGKGLEWLGVIWSGGNTDYNTPFTSRLSINKDNSKSQV | |
| hCD28 | αCD28 scFv | FFKMNSLQSNDTAIYYCARALTYYDYEFAYWGQGTLVTV | |
| | | SAASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDGGGGSGGGGSQ | |
| | | VQLVQSGAEVKKPGASVKVSCKASGYTFTSYYIHWVRQA | |
| | | PGQCLEWIGCIYPGNVNTNYNEKFKDRATLTVDTSISTA | |
| | | YMELSRLRSDDTAVYFCTRSHYGLDWNFDVWGQGTTVTV | |
| | | SSGGGGSGGGGSGGGGSDIQMTQSPSSLSASVGDRVTIT | |
| | | CHASQNIYVWLNWYQQKPGKAPKLLIYKASNLHTGVPSR | |
| | | FSGSGSGTDFTLTISSLQPEDFATYYCQQGQTYPYTFGC | |
| | | GTKVEIKEPKSSDKTHTCPPCPAPEAAGGPSVFLFPPKP | |
| | | KDTLMISRTPEVTCVVVDVSHEDPEVKFNWYVDGVEVHN | |
| | | AKTKPREEQYNSTYRVVSVLTVLHQDWLNGKEYKCKVSN | |
| | | KALAAPIEKTISKAKGQPREPQVYTLPPCRDELTKNQVS | |
| | | LWCLVKGFYPSDIAVEWESNGQPENNYKTTPPVLDSDGS | |
| | | FFLYSKLTVDKSRWQQGNVFSCSVMHEALHNHYTQKSLS | |
| | | LSPGK | |
| | HC (Hole) | QVQLKQSGPGLVQPSQSLSITCTVSGFSLTNYGVHWVRQ | 260 |
| | αEGFR | SPGKGLEWLGVIWSGGNTDYNTPFTSRLSINKDNSKSQV | |
| | αCD3 scFv | FFKMNSLQSNDTAIYYCARALTYYDYEFAYWGQGTLVTV | |
| | | SAASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDGGGGSGGGGSE | |
| | | VQLVESGGGLVQPGGSLKLSCAASGFTFNKYAMNWVRQA | |
| | | PGKCLEWVARIRSKYNNYATYYADSVKDRFTISRDDSKN | |
| | | TAYLQMNNLKTEDTAVYYCVRHGNFGNSYISYWAYWGQG | |
| | | TLVTVSSGGGGSGGGGSGGGGSQTVVTQEPSLTVSPGGT | |
| | | VTLTCGSSTGAVTSGYYPNWVQQKPGQAPRGLIGGTKFL | |
| | | APGTPARFSGSLLGGKAALTLSGVQPEDEAEYYCALWYS | |
| | | NRWVFGCGTKLTVLEPKSSDKTHTCPPCPAPEAAGGPSV | |
| | | FLFPPKPKDTLMISRTPEVTCVVVDVSHEDPEVKFNWYV | |
| | | DGVEVHNAKTKPREEQYNSTYRVVSVLTVLHQDWLNGKE | |
| | | YKCKVSNKALAAPIEKTISKAKGQPREPQVCTLPPSRDE | |
| | | LTKNQVSLSCAVKGFYPSDIAVEWESNGQPENNYKTTPP | |
| | | VLDSDGSFFLVSKLTVDKSRWQQGNVFSCSVMHEALHNH | |
| | | YTQKSLSLSPGK | |
| | LC | DILLTQSPVILSVSPGERVSFSCRASQSIGTNIHWYQQR | 256 |
| | αEGFR | TNGSPRLLIKYASESISGIPSRFSGSGSGTDFTLSINSV | |
| | | ESEDIADYYCQQNNNWPTTFGAGTKLELKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA605 | HC (Knob) | QVQLQESGPGLVKPSETLSLTCTVSGGSVSSGSYYWSWI | 304 |
| (M912) | αMSLN | RQPPGKGLEWIGYIYYSGSTNYNPSLKSRVTISVDTSKN | |
| (405 Format) | αCD3 scFv | QFSLKLSSVTAADTAVYYCAREGKNGAFDIWGQGTMVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDKTHTCPPCPAP | |
| | | EAAGGPSVFLFPPKPKDTLMISRTPEVTCVVVDVSHEDP | |
| | | EVKFNWYVDGVEVHNAKTKPREEQYNSTYRVVSVLTVLH | |
| | | QDWLNGKEYKCKVSNKALAAPIEKTISKAKGQPREPQVY | |
| | | TLPPCRDELTKNQVSLWCLVKGFYPSDIAVEWESNGQPE | |
| | | NNYKTTPPVLDSDGSFFLYSKLTVDKSRWQQGNVFSCSV | |
| | | MHEALHNHYTQKSLSLSPGKGGGGSGGGGSGGGGSEVQL | |
| | | VESGGGLVQPGGSLKLSCAASGFTFNKYAMNWVRQAPGK | |
| | | CLEWVARIRSKYNNYATYYADSVKDRFTISRDDSKNTAY | |
| | | LQMNNLKTEDTAVYYCVRHGNFGNSYISYWAYWGQGTLV | |
| | | TVSSGGGGSGGGGSGGGGSQTVVTQEPSLTVSPGGTVTL | |
| | | TCGSSTGAVTSGYYPNWVQQKPGQAPRGLIGGTKFLAPG | |
| | | TPARFSGSLLGGKAALTLSGVQPEDEAEYYCALWYSNRW | |
| | | VFGCGTKLTVL | |
| | HC (Hole) | QVQLQESGPGLVKPSETLSLTCTVSGGSVSSGSYYWSWI | 305 |
| | αMSLN | RQPPGKGLEWIGYIYYSGSTNYNPSLKSRVTISVDTSKN | |
| | αCD28 scFv | QFSLKLSSVTAADTAVYYCAREGKNGAFDIWGQGTMVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDKTHTCPPCPAP | |
| | | EAAGGPSVFLFPPKPKDTLMISRTPEVTCVVVDVSHEDP | |
| | | EVKFNWYVDGVEVHNAKTKPREEQYNSTYRVVSVLTVLH | |
| | | QDWLNGKEYKCKVSNKALAAPIEKTISKAKGQPREPQVC | |
| | | TLPPSRDELTKNQVSLSCAVKGFYPSDIAVEWESNGQPE | |
| | | NNYKTTPPVLDSDGSFFLVSKLTVDKSRWQQGNVFSCSV | |
| | | MHEALHNHYTQKSLSLSPGKGGGGSGGGGSGGGGSQVQL | |
| | | VQSGAEVKKPGASVKVSCKASGYTFTSYYIHWVRQAPGQ | |
| | | CLEWIGCIYPGNVNTNYNEKFKDRATLTVDTSISTAYME | |
| | | LSRLRSDDTAVYFCTRSHYGLDWNFDVWGQGTTVTVSSG | |
| | | GGGSGGGGSGGGGSDIQMTQSPSSLSASVGDRVTITCHA | |
| | | SQNIYVWLNWYQQKPGKAPKLLIYKASNLHTGVPSRFSG | |
| | | SGSGTDFTLTISSLQPEDFATYYCQQGQTYPYTFGCGTK | |
| | | VEIK | |
| | LC | DIQMTQSPSSLSASVGDRVTITCRASQSISSYLNWYQQK | 306 |
| | αMSLN | PGKAPKLLIYAASSLQSGVPSGFSGSGSGTDFTLTISSL | |
| | | QPEDFATYYCQQSYSTPLTFGGGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA606 | HC (Knob) | QTVVTQEPSLTVSPGGTVTLTCGSSTGAVTSGYYPNWVQ | 307 |
| (M912) | αCD3 scFv | QKPGQAPRGLIGGTKFLAPGTPARFSGSLLGGKAALTLS | |
| (406 Format) | αMSLN | GVQPEDEAEYYCALWYSNRWVFGCGTKLTVLGGGGSGGG | |
| | | GSGGGGSEVQLVESGGGLVQPGGSLKLSCAASGFTFNKY | |
| | | AMNWVRQAPGKCLEWVARIRSKYNNYATYYADSVKDRFT | |
| | | ISRDDSKNTAYLQMNNLKTEDTAVYYCVRHGNFGNSYIS | |
| | | YWAYWGQGTLVTVSSASTKGPQVQLQESGPGLVKPSETL | |
| | | SLTCTVSGGSVSSGSYYWSWIRQPPGKGLEWIGYIYYSG | |
| | | STNYNPSLKSRVTISVDTSKNQFSLKLSSVTAADTAVYY | |
| | | CAREGKNGAFDIWGQGTMVTVSSASTKGPSVFPLAPSSK | |
| | | STSGGTAALGCLVKDYFPEPVTVSWNSGALTSGVHTFPA | |
| | | VLQSSGLYSLSSVVTVPSSSLGTQTYICNVNHKPSNTKV | |
| | | DKKVEPKSCDKTHTCPPCPAPEAAGGPSVFLFPPKPKDT | |
| | | LMISRTPEVTCVVVDVSHEDPEVKFNWYVDGVEVHNAKT | |
| | | KPREEQYNSTYRVVSVLTVLHQDWLNGKEYKCKVSNKAL | |
| | | AAPIEKTISKAKGQPREPQVYTLPPCRDELTKNQVSLWC | |
| | | LVKGFYPSDIAVEWESNGQPENNYKTTPPVLDSDGSFFL | |
| | | YSKLTVDKSRWQQGNVFSCSVMHEALHNHYTQKSLSLSP | |
| | | GK | |
| | HC (Hole) | DIQMTQSPSSLSASVGDRVTITCHASQNIYVWLNWYQQK | 308 |
| | αCD28 scFv | PGKAPKLLIYKASNLHTGVPSRFSGSGSGTDFTLTISSL | |
| | αMSLN | QPEDFATYYCQQGQTYPYTFGCGTKVEIKGGGGSGGGGS | |
| | | GGGGSQVQLVQSGAEVKKPGASVKVSCKASGYTFTSYYI | |
| | | HWVRQAPGQCLEWIGCIYPGNVNTNYNEKFKDRATLTVD | |
| | | TSISTAYMELSRLRSDDTAVYFCTRSHYGLDWNFDVWGQ | |
| | | GTTVTVSSASTKGPQVQLQESGPGLVKPSETLSLTCTVS | |
| | | GGSVSSGSYYWSWIRQPPGKGLEWIGYIYYSGSTNYNPS | |
| | | LKSRVTISVDTSKNQFSLKLSSVTAADTAVYYCAREGKN | |
| | | GAFDIWGQGTMVTVSSASTKGPSVFPLAPSSKSTSGGTA | |
| | | ALGCLVKDYFPEPVTVSWNSGALTSGVHTFPAVLQSSGL | |
| | | YSLSSVVTVPSSSLGTQTYICNVNHKPSNTKVDKKVEPK | |
| | | SCDKTHTCPPCPAPEAAGGPSVFLFPPKPKDTLMISRTP | |
| | | EVTCVVVDVSHEDPEVKFNWYVDGVEVHNAKTKPREEQY | |
| | | NSTYRVVSVLTVLHQDWLNGKEYKCKVSNKALAAPIEKT | |
| | | ISKAKGQPREPQVCTLPPSRDELTKNQVSLSCAVKGFYP | |
| | | SDIAVEWESNGQPENNYKTTPPVLDSDGSFFLVSKLTVD | |
| | | KSRWQQGNVFSCSVMHEALHNHYTQKSLSLSPGK | |
| | LC | DIQMTQSPSSLSASVGDRVTITCRASQSISSYLNWYQQK | 306 |
| | αMSLN | PGKAPKLLIYAASSLQSGVPSGFSGSGSGTDFTLTISSL | |
| | | QPEDFATYYCQQSYSTPLTFGGGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA624 | HC (Knob) | QVQLQESGPGLVKPSETLSLTCTVSGGSVSSGSYYWSWI | 309 |
| (M912) | αMSLN | RQPPGKGLEWIGYIYYSGSTNYNPSLKSRVTISVDTSKN | |
| (424 Format) | αCD28 scFv | QFSLKLSSVTAADTAVYYCAREGKNGAFDIWGQGTMVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDGGGGSGGGGSQ | |
| | | VQLVQSGAEVKKPGASVKVSCKASGYTFTSYYIHWVRQA | |
| | | PGQCLEWIGCIYPGNVNTNYNEKFKDRATLTVDTSISTA | |
| | | YMELSRLRSDDTAVYFCTRSHYGLDWNFDVWGQGTTVTV | |
| | | SSGGGGSGGGGSGGGGSDIQMTQSPSSLSASVGDRVTIT | |
| | | CHASQNIYVWLNWYQQKPGKAPKLLIYKASNLHTGVPSR | |
| | | FSGSGSGTDFTLTISSLQPEDFATYYCQQGQTYPYTFGC | |
| | | GTKVEIKEPKSSDKTHTCPPCPAPEAAGGPSVFLFPPKP | |
| | | KDTLMISRTPEVTCVVVDVSHEDPEVKFNWYVDGVEVHN | |
| | | AKTKPREEQYNSTYRVVSVLTVLHQDWLNGKEYKCKVSN | |
| | | KALAAPIEKTISKAKGQPREPQVYTLPPCRDELTKNQVS | |
| | | LWCLVKGFYPSDIAVEWESNGQPENNYKTTPPVLDSDGS | |
| | | FFLYSKLTVDKSRWQQGNVFSCSVMHEALHNHYTQKSLS | |
| | | LSPGK | |
| | HC (Hole) | QVQLQESGPGLVKPSETLSLTCTVSGGSVSSGSYYWSWI | 310 |
| | αMSLN | RQPPGKGLEWIGYIYYSGSTNYNPSLKSRVTISVDTSKN | |
| | αCD3 scFv | QFSLKLSSVTAADTAVYYCAREGKNGAFDIWGQGTMVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDGGGGSGGGGSE | |
| | | VQLVESGGGLVQPGGSLKLSCAASGFTFNKYAMNWVRQA | |
| | | PGKCLEWVARIRSKYNNYATYYADSVKDRFTISRDDSKN | |
| | | TAYLQMNNLKTEDTAVYYCVRHGNFGNSYISYWAYWGQG | |
| | | TLVTVSSGGGGSGGGGSGGGGSQTVVTQEPSLTVSPGGT | |
| | | VTLTCGSSTGAVTSGYYPNWVQQKPGQAPRGLIGGTKFL | |
| | | APGTPARFSGSLLGGKAALTLSGVQPEDEAEYYCALWYS | |
| | | NRWVFGCGTKLTVLEPKSSDKTHTCPPCPAPEAAGGPSV | |
| | | FLFPPKPKDTLMISRTPEVTCVVVDVSHEDPEVKFNWYV | |
| | | DGVEVHNAKTKPREEQYNSTYRVVSVLTVLHQDWLNGKE | |
| | | YKCKVSNKALAAPIEKTISKAKGQPREPQVCTLPPSRDE | |
| | | LTKNQVSLSCAVKGFYPSDIAVEWESNGQPENNYKTTPP | |
| | | VLDSDGSFFLVSKLTVDKSRWQQGNVFSCSVMHEALHNH | |
| | | YTQKSLSLSPGK | |
| | LC | DIQMTQSPSSLSASVGDRVTITCRASQSISSYLNWYQQK | 306 |
| | αMSLN | PGKAPKLLIYAASSLQSGVPSGFSGSGSGTDFTLTISSL | |
| | | QPEDFATYYCQQSYSTPLTFGGGTKVEIKRTVAAPSVFI | |
| | | FPPSDEQLKSGTASVVCLLNNFYPREAKVQWKVDNALQS | |
| | | GNSQESVTEQDSKDSTYSLSSTLTLSKADYEKHKVYACE | |
| | | VTHQGLSSPVTKSFNRGEC | |
| BCA605 | HC (Knob) | QVQLQQSGPELEKPGASVKISCKASGYSFTGYTMNWVKQ | 311 |
| (MOR) | αMSLN | SHGKSLEWIGLITPYNGASSYNQKFRGKATLTVDKSSST | |
| (405 Format) | αCD3 scFv | AYMDLLSLTSEDSAVYFCARGGYDGRGFDYWGSGTPVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDKTHTCPPCPAP | |
| | | EAAGGPSVFLFPPKPKDTLMISRTPEVTCVVVDVSHEDP | |
| | | EVKFNWYVDGVEVHNAKTKPREEQYNSTYRVVSVLTVLH | |
| | | QDWLNGKEYKCKVSNKALAAPIEKTISKAKGQPREPQVY | |
| | | TLPPCRDELTKNQVSLWCLVKGFYPSDIAVEWESNGQPE | |
| | | NNYKTTPPVLDSDGSFFLYSKLTVDKSRWQQGNVFSCSV | |
| | | MHEALHNHYTQKSLSLSPGKGGGGSGGGGSGGGGSEVQL | |
| | | VESGGGLVQPGGSLKLSCAASGFTFNKYAMNWVRQAPGK | |
| | | CLEWVARIRSKYNNYATYYADSVKDRFTISRDDSKNTAY | |
| | | LQMNNLKTEDTAVYYCVRHGNFGNSYISYWAYWGQGTLV | |
| | | TVSSGGGGSGGGGSGGGGSQTVVTQEPSLTVSPGGTVTL | |
| | | TCGSSTGAVTSGYYPNWVQQKPGQAPRGLIGGTKFLAPG | |
| | | TPARFSGSLLGGKAALTLSGVQPEDEAEYYCALWYSNRW | |
| | | VFGCGTKLTVL | |
| | HC (Hole) | QVQLQQSGPELEKPGASVKISCKASGYSFTGYTMNWVKQ | 312 |
| | αMSLN | SHGKSLEWIGLITPYNGASSYNQKFRGKATLTVDKSSST | |
| | αCD28 scFv | AYMDLLSLTSEDSAVYFCARGGYDGRGFDYWGSGTPVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDKTHTCPPCPAP | |
| | | EAAGGPSVFLFPPKPKDTLMISRTPEVTCVVVDVSHEDP | |
| | | EVKFNWYVDGVEVHNAKTKPREEQYNSTYRVVSVLTVLH | |
| | | QDWLNGKEYKCKVSNKALAAPIEKTISKAKGQPREPQVC | |
| | | TLPPSRDELTKNQVSLSCAVKGFYPSDIAVEWESNGQPE | |
| | | NNYKTTPPVLDSDGSFFLVSKLTVDKSRWQQGNVFSCSV | |
| | | MHEALHNHYTQKSLSLSPGKGGGGSGGGGSGGGGSQVQL | |
| | | VQSGAEVKKPGASVKVSCKASGYTFTSYYIHWVRQAPGQ | |
| | | CLEWIGCIYPGNVNTNYNEKFKDRATLTVDTSISTAYME | |
| | | LSRLRSDDTAVYFCTRSHYGLDWNFDVWGQGTTVTVSSG | |
| | | GGGSGGGGSGGGGSDIQMTQSPSSLSASVGDRVTITCHA | |
| | | SQNIYVWLNWYQQKPGKAPKLLIYKASNLHTGVPSRFSG | |
| | | SGSGTDFTLTISSLQPEDFATYYCQQGQTYPYTFGCGTK | |
| | | VEIK | |
| | LC | DIELTQSPAIMSASPGEKVTMTCSASSSVSYMHWYQQKS | 313 |
| | αMSLN | GTSPKRWIYDTSKLASGVPGRFSGSGSGNSYSLTISSVE | |
| | | AEDDATYYCQQWSKHPLTFGSGTKVEIKRTVAAPSVFIF | |
| | | PPSDEQLKSGTASVVCLLNNFYPREAKVQWKVDNALQSG | |
| | | NSQESVTEQDSKDSTYSLSSTLTLSKADYEKHKVYACEV | |
| | | THQGLSSPVTKSFNRGEC | |
| BCA606 | HC (Knob) | QTVVTQEPSLTVSPGGTVTLTCGSSTGAVTSGYYPNWVQ | 314 |
| (MOR) | αCD3 scFv | QKPGQAPRGLIGGTKFLAPGTPARFSGSLLGGKAALTLS | |
| (406 Format) | αMSLN | GVQPEDEAEYYCALWYSNRWVFGCGTKLTVLGGGGSGGG | |
| | | GSGGGGSEVQLVESGGGLVQPGGSLKLSCAASGFTFNKY | |
| | | AMNWVRQAPGKCLEWVARIRSKYNNYATYYADSVKDRFT | |
| | | ISRDDSKNTAYLQMNNLKTEDTAVYYCVRHGNFGNSYIS | |
| | | YWAYWGQGTLVTVSSASTKGPQVQLQQSGPELEKPGASV | |
| | | KISCKASGYSFTGYTMNWVKQSHGKSLEWIGLITPYNGA | |
| | | SSYNQKFRGKATLTVDKSSSTAYMDLLSLTSEDSAVYFC | |
| | | ARGGYDGRGFDYWGSGTPVTVSSASTKGPSVFPLAPSSK | |
| | | STSGGTAALGCLVKDYFPEPVTVSWNSGALTSGVHTFPA | |
| | | VLQSSGLYSLSSVVTVPSSSLGTQTYICNVNHKPSNTKV | |
| | | DKKVEPKSCDKTHTCPPCPAPEAAGGPSVFLFPPKPKDT | |
| | | LMISRTPEVTCVVVDVSHEDPEVKFNWYVDGVEVHNAKT | |
| | | KPREEQYNSTYRVVSVLTVLHQDWLNGKEYKCKVSNKAL | |
| | | AAPIEKTISKAKGQPREPQVYTLPPCRDELTKNQVSLWC | |
| | | LVKGFYPSDIAVEWESNGQPENNYKTTPPVLDSDGSFFL | |
| | | YSKLTVDKSRWQQGNVFSCSVMHEALHNHYTQKSLSLSP | |
| | | GK | |
| | HC (Hole) | DIQMTQSPSSLSASVGDRVTITCHASQNIYVWLNWYQQK | 315 |
| | αCD28 scFv | PGKAPKLLIYKASNLHTGVPSRFSGSGSGTDFTLTISSL | |
| | αMSLN | QPEDFATYYCQQGQTYPYTFGCGTKVEIKGGGGSGGGGS | |
| | | GGGGSQVQLVQSGAEVKKPGASVKVSCKASGYTFTSYYI | |
| | | HWVRQAPGQCLEWIGCIYPGNVNTNYNEKFKDRATLTVD | |
| | | TSISTAYMELSRLRSDDTAVYFCTRSHYGLDWNFDVWGQ | |
| | | GTTVTVSSASTKGPQVQLQQSGPELEKPGASVKISCKAS | |
| | | GYSFTGYTMNWVKQSHGKSLEWIGLITPYNGASSYNQKF | |
| | | RGKATLTVDKSSSTAYMDLLSLTSEDSAVYFCARGGYDG | |
| | | RGFDYWGSGTPVTVSSASTKGPSVFPLAPSSKSTSGGTA | |
| | | ALGCLVKDYFPEPVTVSWNSGALTSGVHTFPAVLQSSGL | |
| | | YSLSSVVTVPSSSLGTQTYICNVNHKPSNTKVDKKVEPK | |
| | | SCDKTHTCPPCPAPEAAGGPSVFLFPPKPKDTLMISRTP | |
| | | EVTCVVVDVSHEDPEVKFNWYVDGVEVHNAKTKPREEQY | |
| | | NSTYRVVSVLTVLHQDWLNGKEYKCKVSNKALAAPIEKT | |
| | | ISKAKGQPREPQVCTLPPSRDELTKNQVSLSCAVKGFYP | |
| | | SDIAVEWESNGQPENNYKTTPPVLDSDGSFFLVSKLTVD | |
| | | KSRWQQGNVFSCSVMHEALHNHYTQKSLSLSPGK | |
| | LC | DIELTQSPAIMSASPGEKVTMTCSASSSVSYMHWYQQKS | 313 |
| | αMSLN | GTSPKRWIYDTSKLASGVPGRFSGSGSGNSYSLTISSVE | |
| | | AEDDATYYCQQWSKHPLTFGSGTKVEIKRTVAAPSVFIF | |
| | | PPSDEQLKSGTASVVCLLNNFYPREAKVQWKVDNALQSG | |
| | | NSQESVTEQDSKDSTYSLSSTLTLSKADYEKHKVYACEV | |
| | | THQGLSSPVTKSFNRGEC | |
| BCA624 | HC (Knob) | QVQLQQSGPELEKPGASVKISCKASGYSFTGYTMNWVKQ | 316 |
| (MOR) | αMSLN | SHGKSLEWIGLITPYNGASSYNQKFRGKATLTVDKSSST | |
| (424 Format) | αCD28 scFv | AYMDLLSLTSEDSAVYFCARGGYDGRGFDYWGSGTPVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDGGGGSGGGGSQ | |
| | | VQLVQSGAEVKKPGASVKVSCKASGYTFTSYYIHWVRQA | |
| | | PGQCLEWIGCIYPGNVNTNYNEKFKDRATLTVDTSISTA | |
| | | YMELSRLRSDDTAVYFCTRSHYGLDWNFDVWGQGTTVTV | |
| | | SSGGGGSGGGGSGGGGSDIQMTQSPSSLSASVGDRVTIT | |
| | | CHASQNIYVWLNWYQQKPGKAPKLLIYKASNLHTGVPSR | |
| | | FSGSGSGTDFTLTISSLQPEDFATYYCQQGQTYPYTFGC | |
| | | GTKVEIKEPKSSDKTHTCPPCPAPEAAGGPSVFLFPPKP | |
| | | KDTLMISRTPEVTCVVVDVSHEDPEVKFNWYVDGVEVHN | |
| | | AKTKPREEQYNSTYRVVSVLTVLHQDWLNGKEYKCKVSN | |
| | | KALAAPIEKTISKAKGQPREPQVYTLPPCRDELTKNQVS | |
| | | LWCLVKGFYPSDIAVEWESNGQPENNYKTTPPVLDSDGS | |
| | | FFLYSKLTVDKSRWQQGNVFSCSVMHEALHNHYTQKSLS | |
| | | LSPGK | |
| | HC (Hole) | QVQLQQSGPELEKPGASVKISCKASGYSFTGYTMNWVKQ | 317 |
| | αMSLN | SHGKSLEWIGLITPYNGASSYNQKFRGKATLTVDKSSST | |
| | αCD3 scFv | AYMDLLSLTSEDSAVYFCARGGYDGRGFDYWGSGTPVTV | |
| | | SSASTKGPSVFPLAPSSKSTSGGTAALGCLVKDYFPEPV | |
| | | TVSWNSGALTSGVHTFPAVLQSSGLYSLSSVVTVPSSSL | |
| | | GTQTYICNVNHKPSNTKVDKKVEPKSCDGGGGSGGGGSE | |
| | | VQLVESGGGLVQPGGSLKLSCAASGFTFNKYAMNWVRQA | |
| | | PGKCLEWVARIRSKYNNYATYYADSVKDRFTISRDDSKN | |
| | | TAYLQMNNLKTEDTAVYYCVRHGNFGNSYISYWAYWGQG | |
| | | TLVTVSSGGGGSGGGGSGGGGSQTVVTQEPSLTVSPGGT | |
| | | VTLTCGSSTGAVTSGYYPNWVQQKPGQAPRGLIGGTKFL | |
| | | APGTPARFSGSLLGGKAALTLSGVQPEDEAEYYCALWYS | |
| | | NRWVFGCGTKLTVLEPKSSDKTHTCPPCPAPEAAGGPSV | |
| | | FLFPPKPKDTLMISRTPEVTCVVVDVSHEDPEVKFNWYV | |
| | | DGVEVHNAKTKPREEQYNSTYRVVSVLTVLHQDWLNGKE | |
| | | YKCKVSNKALAAPIEKTISKAKGQPREPQVCTLPPSRDE | |
| | | LTKNQVSLSCAVKGFYPSDIAVEWESNGQPENNYKTTPP | |
| | | VLDSDGSFFLVSKLTVDKSRWQQGNVFSCSVMHEALHNH | |
| | | YTQKSLSLSPGK | |
| | LC | DIELTQSPAIMSASPGEKVTMTCSASSSVSYMHWYQQKS | 313 |
| | αMSLN | GTSPKRWIYDTSKLASGVPGRFSGSGSGNSYSLTISSVE | |
| | | AEDDATYYCQQWSKHPLTFGSGTKVEIKRTVAAPSVFIF | |
| | | PPSDEQLKSGTASVVCLLNNFYPREAKVQWKVDNALQSG | |
| | | NSQESVTEQDSKDSTYSLSSTLTLSKADYEKHKVYACEV | |
| | | THQGLSSPVTKSFNRGEC | |
| BCA605 | HC (Knob) | EVQLQQSGPVLVKPGASVKISCKASGYSFTGYYMHWVRQ | 320 |
| (FB6) | αMSLN | SLVKRLEWIGRINPYTGVPSYKHNFKDKASLTVDKSSST | |
| (405 Format) | αCD3 scFv | AYMELHSLTSEDSAVYYCARELGGYWGQGTTLTVSSAST | |
| | | KGPSVFPLAPSSKSTSGGTAALGCLVKDYFPEPVTVSWN | |
| | | SGALTSGVHTFPAVLQSSGLYSLSSVVTVPSSSLGTQTY | |
| | | ICNVNHKPSNTKVDKKVEPKSCDKTHTCPPCPAPEAAGG | |
| | | PSVFLFPPKPKDTLMISRTPEVTCVVVDVSHEDPEVKFN | |
| | | WYVDGVEVHNAKTKPREEQYNSTYRVVSVLTVLHQDWLN | |
| | | GKEYKCKVSNKALAAPIEKTISKAKGQPREPQVYTLPPC | |
| | | RDELTKNQVSLWCLVKGFYPSDIAVEWESNGQPENNYKT | |
| | | TPPVLDSDGSFFLYSKLTVDKSRWQQGNVFSCSVMHEAL | |
| | | HNHYTQKSLSLSPGKGGGGSGGGGSGGGGSEVQLVESGG | |
| | | GLVQPGGSLKLSCAASGFTFNKYAMNWVRQAPGKCLEWV | |
| | | ARIRSKYNNYATYYADSVKDRFTISRDDSKNTAYLQMNN | |
| | | LKTEDTAVYYCVRHGNFGNSYISYWAYWGQGTLVTVSSG | |
| | | GGGSGGGGSGGGGSQTVVTQEPSLTVSPGGTVTLTCGSS | |
| | | TGAVTSGYYPNWVQQKPGQAPRGLIGGTKFLAPGTPARF | |
| | | SGSLLGGKAALTLSGVQPEDEAEYYCALWYSNRWVFGCG | |
| | | TKLTVL | |
| | HC (Hole) | EVQLQQSGPVLVKPGASVKISCKASGYSFTGYYMHWVRQ | 321 |
| | αMSLN | SLVKRLEWIGRINPYTGVPSYKHNFKDKASLTVDKSSST | |
| | αCD28 scFv | AYMELHSLTSEDSAVYYCARELGGYWGQGTTLTVSSAST | |
| | | KGPSVFPLAPSSKSTSGGTAALGCLVKDYFPEPVTVSWN | |
| | | SGALTSGVHTFPAVLQSSGLYSLSSVVTVPSSSLGTQTY | |
| | | ICNVNHKPSNTKVDKKVEPKSCDKTHTCPPCPAPEAAGG | |
| | | PSVFLFPPKPKDTLMISRTPEVTCVVVDVSHEDPEVKFN | |
| | | WYVDGVEVHNAKTKPREEQYNSTYRVVSVLTVLHQDWLN | |
| | | GKEYKCKVSNKALAAPIEKTISKAKGQPREPQVCTLPPS | |
| | | RDELTKNQVSLSCAVKGFYPSDIAVEWESNGQPENNYKT | |
| | | TPPVLDSDGSFFLVSKLTVDKSRWQQGNVFSCSVMHEAL | |
| | | HNHYTQKSLSLSPGKGGGGSGGGGSGGGGSQVQLVQSGA | |
| | | EVKKPGASVKVSCKASGYTFTSYYIHWVRQAPGQCLEWI | |
| | | GCIYPGNVNTNYNEKFKDRATLTVDTSISTAYMELSRLR | |
| | | SDDTAVYFCTRSHYGLDWNFDVWGQGTTVTVSSGGGGSG | |
| | | GGGSGGGGSDIQMTQSPSSLSASVGDRVTITCHASQNIY | |
| | | VWLNWYQQKPGKAPKLLIYKASNLHTGVPSRFSGSGSGT | |
| | | DFTLTISSLQPEDFATYYCQQGQTYPYTFGCGTKVEIK | |
| | LC | QAVVTQESALTTSPGETVTLTCRSSTGAVTTGNYPNWVQ | 322 |
| | αMSLN | EKPDHLFTGLIAGTNNRAPGVPARFSGSLIGDKAALTIT | |
| | | GAQTEDEAIYFCALWFSSHWVFGGGTKLTVLRTVAAPSV | |
| | | FIFPPSDEQLKSGTASVVCLLNNFYPREAKVQWKVDNAL | |
| | | QSGNSQESVTEQDSKDSTYSLSSTLTLSKADYEKHKVYA | |
| | | CEVTHQGLSSPVTKSFNRGEC | |
| BCA606 | HC (Knob) | QTVVTQEPSLTVSPGGTVTLTCGSSTGAVTSGYYPNWVQ | 323 |
| (FB6) | αCD3 scFv | QKPGQAPRGLIGGTKFLAPGTPARFSGSLLGGKAALTLS | |
| (406 Format) | αMSLN | GVQPEDEAEYYCALWYSNRWVFGCGTKLTVLGGGGSGGG | |
| | | GSGGGGSEVQLVESGGGLVQPGGSLKLSCAASGFTFNKY | |
| | | AMNWVRQAPGKCLEWVARIRSKYNNYATYYADSVKDRFT | |
| | | ISRDDSKNTAYLQMNNLKTEDTAVYYCVRHGNFGNSYIS | |
| | | YWAYWGQGTLVTVSSASTKGPEVQLQQSGPVLVKPGASV | |
| | | KISCKASGYSFTGYYMHWVRQSLVKRLEWIGRINPYTGV | |
| | | PSYKHNFKDKASLTVDKSSSTAYMELHSLTSEDSAVYYC | |
| | | ARELGGYWGQGTTLTVSSASTKGPSVFPLAPSSKSTSGG | |
| | | TAALGCLVKDYFPEPVTVSWNSGALTSGVHTFPAVLQSS | |
| | | GLYSLSSVVTVPSSSLGTQTYICNVNHKPSNTKVDKKVE | |
| | | PKSCDKTHTCPPCPAPEAAGGPSVFLFPPKPKDTLMISR | |
| | | TPEVTCVVVDVSHEDPEVKFNWYVDGVEVHNAKTKPREE | |
| | | QYNSTYRVVSVLTVLHQDWLNGKEYKCKVSNKALAAPIE | |
| | | KTISKAKGQPREPQVYTLPPCRDELTKNQVSLWCLVKGF | |
| | | YPSDIAVEWESNGQPENNYKTTPPVLDSDGSFFLYSKLT | |
| | | VDKSRWQQGNVFSCSVMHEALHNHYTQKSLSLSPGK | |
| | HC (Hole) | DIQMTQSPSSLSASVGDRVTITCHASQNIYVWLNWYQQK | 324 |
| | αCD28 scFv | PGKAPKLLIYKASNLHTGVPSRFSGSGSGTDFTLTISSL | |
| | αMSLN | QPEDFATYYCQQGQTYPYTFGCGTKVEIKGGGGSGGGGS | |
| | | GGGGSQVQLVQSGAEVKKPGASVKVSCKASGYTFTSYYI | |
| | | HWVRQAPGQCLEWIGCIYPGNVNTNYNEKFKDRATLTVD | |
| | | TSISTAYMELSRLRSDDTAVYFCTRSHYGLDWNFDVWGQ | |
| | | GTTVTVSSASTKGPEVQLQQSGPVLVKPGASVKISCKAS | |
| | | GYSFTGYYMHWVRQSLVKRLEWIGRINPYTGVPSYKHNF | |
| | | KDKASLTVDKSSSTAYMELHSLTSEDSAVYYCARELGGY | |
| | | WGQGTTLTVSSASTKGPSVFPLAPSSKSTSGGTAALGCL | |
| | | VKDYFPEPVTVSWNSGALTSGVHTFPAVLQSSGLYSLSS | |
| | | VVTVPSSSLGTQTYICNVNHKPSNTKVDKKVEPKSCDKT | |
| | | HTCPPCPAPEAAGGPSVFLFPPKPKDTLMISRTPEVTCV | |
| | | VVDVSHEDPEVKFNWYVDGVEVHNAKTKPREEQYNSTYR | |
| | | VVSVLTVLHQDWLNGKEYKCKVSNKALAAPIEKTISKAK | |
| | | GQPREPQVCTLPPSRDELTKNQVSLSCAVKGFYPSDIAV | |
| | | EWESNGQPENNYKTTPPVLDSDGSFFLVSKLTVDKSRWQ | |
| | | QGNVFSCSVMHEALHNHYTQKSLSLSPGK | |
| | LC | QAVVTQESALTTSPGETVTLTCRSSTGAVTTGNYPNWVQ | 322 |
| | αMSLN | EKPDHLFTGLIAGTNNRAPGVPARFSGSLIGDKAALTIT | |
| | | GAQTEDEAIYFCALWFSSHWVFGGGTKLTVLRTVAAPSV | |
| | | FIFPPSDEQLKSGTASVVCLLNNFYPREAKVQWKVDNAL | |
| | | QSGNSQESVTEQDSKDSTYSLSSTLTLSKADYEKHKVYA | |
| | | CEVTHQGLSSPVTKSFNRGEC | |
| BCA624 | HC (Knob) | EVQLQQSGPVLVKPGASVKISCKASGYSFTGYYMHWVRQ | 325 |
| (FB6) | αMSLN | SLVKRLEWIGRINPYTGVPSYKHNFKDKASLTVDKSSST | |
| (424 Format) | αCD28 scFv | AYMELHSLTSEDSAVYYCARELGGYWGQGTTLTVSSAST | |
| | | KGPSVFPLAPSSKSTSGGTAALGCLVKDYFPEPVTVSWN | |
| | | SGALTSGVHTFPAVLQSSGLYSLSSVVTVPSSSLGTQTY | |
| | | ICNVNHKPSNTKVDKKVEPKSCDGGGGSGGGGSQVQLVQ | |
| | | SGAEVKKPGASVKVSCKASGYTFTSYYIHWVRQAPGQCL | |
| | | EWIGCIYPGNVNTNYNEKFKDRATLTVDTSISTAYMELS | |
| | | RLRSDDTAVYFCTRSHYGLDWNFDVWGQGTTVTVSSGGG | |
| | | GSGGGGSGGGGSDIQMTQSPSSLSASVGDRVTITCHASQ | |
| | | NIYVWLNWYQQKPGKAPKLLIYKASNLHTGVPSRFSGSG | |
| | | SGTDFTLTISSLQPEDFATYYCQQGQTYPYTFGCGTKVE | |
| | | IKEPKSSDKTHTCPPCPAPEAAGGPSVFLFPPKPKDTLM | |
| | | ISRTPEVTCVVVDVSHEDPEVKFNWYVDGVEVHNAKTKP | |
| | | REEQYNSTYRVVSVLTVLHQDWLNGKEYKCKVSNKALAA | |
| | | PIEKTISKAKGQPREPQVYTLPPCRDELTKNQVSLWCLV | |
| | | KGFYPSDIAVEWESNGQPENNYKTTPPVLDSDGSFFLYS | |
| | | KLTVDKSRWQQGNVFSCSVMHEALHNHYTQKSLSLSPGK | |
| | HC (Hole) | EVQLQQSGPVLVKPGASVKISCKASGYSFTGYYMHWVRQ | 326 |
| | αMSLN | SLVKRLEWIGRINPYTGVPSYKHNFKDKASLTVDKSSST | |
| | αCD3 scFv | AYMELHSLTSEDSAVYYCARELGGYWGQGTTLTVSSAST | |
| | | KGPSVFPLAPSSKSTSGGTAALGCLVKDYFPEPVTVSWN | |
| | | SGALTSGVHTFPAVLQSSGLYSLSSVVTVPSSSLGTQTY | |
| | | ICNVNHKPSNTKVDKKVEPKSCDGGGGSGGGGSEVQLVE | |
| | | SGGGLVQPGGSLKLSCAASGFTFNKYAMNWVRQAPGKCL | |
| | | EWVARIRSKYNNYATYYADSVKDRFTISRDDSKNTAYLQ | |
| | | MNNLKTEDTAVYYCVRHGNFGNSYISYWAYWGQGTLVTV | |
| | | SSGGGGSGGGGSGGGGSQTVVTQEPSLTVSPGGTVTLTC | |
| | | GSSTGAVTSGYYPNWVQQKPGQAPRGLIGGTKFLAPGTP | |
| | | ARFSGSLLGGKAALTLSGVQPEDEAEYYCALWYSNRWVF | |
| | | GCGTKLTVLEPKSSDKTHTCPPCPAPEAAGGPSVFLFPP | |
| | | KPKDTLMISRTPEVTCVVVDVSHEDPEVKFNWYVDGVEV | |
| | | HNAKTKPREEQYNSTYRVVSVLTVLHQDWLNGKEYKCKV | |
| | | SNKALAAPIEKTISKAKGQPREPQVCTLPPSRDELTKNQ | |
| | | VSLSCAVKGFYPSDIAVEWESNGQPENNYKTTPPVLDSD | |
| | | GSFFLVSKLTVDKSRWQQGNVFSCSVMHEALHNHYTQKS | |
| | | LSLSPGK | |
| | LC | QAVVTQESALTTSPGETVTLTCRSSTGAVTTGNYPNWVQ | 322 |
| | αMSLN | EKPDHLFTGLIAGTNNRAPGVPARFSGSLIGDKAALTIT | |
| | | GAQTEDEAIYFCALWFSSHWVFGGGTKLTVLRTVAAPSV | |
| | | FIFPPSDEQLKSGTASVVCLLNNFYPREAKVQWKVDNAL | |
| | | QSGNSQESVTEQDSKDSTYSLSSTLTLSKADYEKHKVYA | |
| | | CEVTHQGLSSPVTKSFNRGEC | |

In some embodiments, the amino acid sequence of the multispecific protein comprises or consists of the amino acid sequence of a multispecific protein set forth in Table 14. In some embodiments, the amino acid sequence of each polypeptide of the multispecific protein comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of the polypeptide set forth in Table 14. In some embodiments, the amino acid sequence of each polypeptide of the multispecific protein comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of one of the multispecific proteins set forth in Table 14. In some embodiments, the amino acid sequence of each polypeptide of the multispecific protein comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of one of the multispecific proteins set forth in Table 14: BCA405, BCA406, BCA424, BCA418, BCA405.S, BCA406.S, BCA424.S, BCA405.EG, BCA406.EG, BCA424.EG, BCA605(M912), BCA606(M912), BCA624(M912), BCA605(MOR), BCA606(MOR), BCA624(MOR), BCA605(FB6), BCA606(FB6), or BCA624(FB6).

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 237; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 238; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 240; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 241; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 242; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 243; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 244; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 245; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 246; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 247.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 248; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 249; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 250; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 251; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 252; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 253; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 239.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 254; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 255; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 256; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 256.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 257; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 258; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 256; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 256.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 259; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 260; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 256; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 256.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 304; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 305; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 306; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 306.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 307; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 308; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 306; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 306.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 309; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 310; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 306; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 306.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 311; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 312; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 313; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 313.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 314; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 315; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 313; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 313.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 316; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 317; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 313; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 313.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 320; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 321; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 322; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 322.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 323; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 324; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 322; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 322.

In some embodiments, the multispecific protein comprises or consists of four polypeptides, wherein the amino acid sequence of the first polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 325; the amino acid sequence of the second polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 326; the amino acid sequence of the third polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 322; the amino acid sequence of the fourth polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 322.

### 5.3 Methods of Making Multispecific Proteins & Polypeptides

The multispecific proteins (and polypeptides thereof) described herein may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the fusion protein (or one or more polypeptide thereof). Such general methods are common knowledge in the art. The expression vector typically contains an expression cassette that includes nucleic acid sequences capable of bringing about expression of the nucleic acid molecule encoding the protein or polypeptide of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein or polypeptide of interest are cultured under conditions conducive to expression of the nucleic acid molecule encoding the antigenic peptide or protein. Culture media is available from various vendors, and a suitable medium can be routinely chosen for a host cell to express a protein or polypeptide of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein or polypeptide may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein or polypeptide may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes. The multispecific proteins (and polypeptides thereof) described herein may be produced synthetically.

### 5.4 Polynucleotides

In one aspect, provided herein are polynucleotides (e.g., DNA, RNA) encoding a multispecific protein (or any (e.g., one or more)) polypeptide thereof) described herein. In some embodiments, the polynucleotide is a DNA polynucleotide or an RNA polynucleotide. In some embodiments, the polynucleotide is an mRNA polynucleotide.

In some embodiments, the polynucleotide is codon optimized. Codon optimization may be used to match codon frequencies in target and host organisms to ensure proper folding; bias guanosine (G) and/or cytosine content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.5 Vectors

In one aspect, provided herein are vectors comprising a polynucleotide (e.g., DNA, RNA) described herein (e.g., a polynucleotide encoding a multispecific protein (or one or more polypeptide thereof) described herein. In some embodiments, the vector is a viral vector. In some embodiments, the vector is a non-viral vector (e.g., a plasmid).

### 5.5.1 Non-Viral Vectors

In some embodiments, the vector is a non-viral vector. In some embodiments, the vector is a minicircle. In some embodiments, the vector is a plasmid. A person of ordinary skill in the art is aware of suitable plasmids for expression of the DNA of interest. For example, plasmid DNA may be generated to allow efficient production of the encoded endonucleases in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

### 5.5.2 Viral Vectors

In some embodiments, the nucleic acid molecules (e.g., DNA or RNA) encoding a multispecific protein (or one or more polypeptide thereof) described herein are contained in a viral vector. Thus, also provided herein are viral vectors comprising the nucleic acid molecules encoding a multispecific protein (or one or more polypeptide thereof) described herein. Such vectors can be easily manipulated by methods well known to the ordinary person of skill in the art. The vector used can be any vector that is suitable for cloning nucleic acids that can be used for transcription of the nucleic acid molecule of interest.

Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., an immunogenic peptide or protein described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™ pCR3.1™, pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation,). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

### 5.6 Carriers

In one aspect, provided herein are carriers comprising a multispecific protein (or one or more polypeptide thereof) described herein, a polynucleotide described herein (e.g., a polynucleotide encoding comprising a multispecific protein (or polypeptide thereof) described herein described herein, or a vector described herein (e.g., a vector comprising a polynucleotide described herein). Carriers include, but are not limited to, lipid-based carriers such as lipid nanoparticles (LNPs), liposomes, lipoplexes, or nanoliposomes. In some embodiments, the carrier is an LNP.

### 5.7 Host Cells

In one aspect, provided herein are host cells comprising a multispecific protein (or one or more polypeptide thereof) described herein, a polynucleotide described herein (e.g., a polynucleotide encoding a multispecific protein (or one or more polypeptide thereof) described herein, a vector described herein (e.g., a vector comprising a polynucleotide described herein), or a carrier described herein (e.g., a carrier comprising a multispecific protein (or one or more polypeptide thereof) described herein).

In some embodiments, the cell is a eukaryotic cell. In some embodiments, the cell is mammalian cell. In some embodiments, the cell is an animal cell. In some embodiments, the cell is a human cell. In some embodiments, the cell is in vitro. In some embodiments, the cell is in vivo. In some embodiments, the cell is ex vivo.

Standard methods known in the art can be utilized to deliver any one of the foregoing (e.g., immunomodulatory protein, fusion protein, vector, polynucleotide, carrier, etc.) into a cell (e.g., a host cell). Standard methods known in the art can be utilized to culture cells (e.g., host cells) in vitro or ex vivo.

### 5.8 Pharmaceutical Compositions

In one aspect, provided herein are pharmaceutical compositions comprising a multispecific protein (or one or more polypeptide thereof) described herein, a polynucleotide described herein, a vector described herein, a host cell described herein, or a carrier described herein, and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing a multispecific protein (or one or more polypeptide thereof) described herein, a polynucleotide described herein, a vector described herein, a host cell described herein, or a carrier described herein, and formulating it into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

A pharmaceutical composition may be formulated for any route of administration to a subject. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.9 Methods of Use

Provided herein are various methods of utilizing the multispecific proteins (and polypeptides thereof) described herein, polynucleotides described herein, vectors described herein, host cells described herein, carriers described herein, and pharmaceutical compositions described herein. In some embodiments, the methods comprise administration of a multispecific protein (or polypeptides thereof) described herein, a polynucleotide described herein, a vector described herein, a host cell described herein, a carrier described herein, or a pharmaceutical composition described herein to a subject. Exemplary subjects include mammals, e.g., humans, non-human mammals, e.g., non-human primates. In some embodiments, the subject is a human.

The dosage of a multispecific protein or polypeptide described herein, a polynucleotide described herein, a vector described herein, a host cell described herein, a carrier described herein, or a pharmaceutical composition described herein to be administered to a subject in accordance with any of the methods described herein can be determined in accordance with standard techniques known to those of ordinary skill in the art, including the route of administration, the age and weight of the subject, and the type (if any) adjuvant is used.

### 5.9.1 Methods of Delivery

In one aspect, provided herein are methods of delivering (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) to a subject, the method comprising administering to the subject the multispecific protein (or polypeptides thereof), the polynucleotide, the vector, the host cell, the carrier, or the pharmaceutical composition, to thereby deliver the multispecific fusion protein (or polypeptides thereof), the polynucleotide, the vector, the host cell, the carrier, or the pharmaceutical composition to the subject. In some embodiments, the multispecific fusion protein (or polypeptides thereof), the polynucleotide, the vector, the host cell, the carrier, or the pharmaceutical composition is administered to the subject in an amount and for a time sufficient to deliver the multispecific fusion protein (or polypeptides thereof), the polynucleotide, the vector, the host cell, the carrier, or the pharmaceutical composition to the subject.

In one aspect, provided herein are methods of delivering (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) to a subject, the method comprising introducing into the cell the multispecific protein (or polypeptides thereof), the polynucleotide, the vector, the host cell, the carrier, or the pharmaceutical composition, to thereby deliver the multispecific fusion protein (or polypeptides thereof), the polynucleotide, the vector, the host cell, the carrier, or the pharmaceutical composition to the cell. In some embodiments, the multispecific fusion protein (or polypeptides thereof), the polynucleotide, the vector, the host cell, the carrier, or the pharmaceutical composition is introduced to the cell in an amount and for a time sufficient to deliver the multispecific fusion protein (or polypeptides thereof), the polynucleotide, the vector, the host cell, the carrier, or the pharmaceutical composition to the cell.

### 5.9.2 Methods of Inducing an Immune Response

In one aspect, provided herein are methods of inducing an immune response in a subject in need thereof, comprising administering to the subject (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); to thereby induce an immune response in the subject. In some embodiments, the multispecific fusion protein (or polypeptides thereof), the polynucleotide, the vector, the host cell, the carrier, or the pharmaceutical composition is administered to the subject in an amount sufficient and for a sufficient time to induce an immune response in the subject.

In one aspect, provided herein is (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for use in a method of inducing an immune response in a subject (e.g., the method comprising administering to the subject the multispecific protein, the polynucleotide, the expression vector, the host cell, the carrier, or the pharmaceutical composition, to thereby induce an immune response in the subject). In some embodiments, the multispecific fusion protein (or polypeptides thereof), the polynucleotide, the vector, the host cell, the carrier, or the pharmaceutical composition is administered to the subject in an amount sufficient and for a sufficient time to induce an immune response in the subject.

In one aspect, provided herein is (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for use in the manufacture of a medicament for inducing an immune response in a subject (e.g., comprising administering to the subject the multispecific protein, the polynucleotide, the expression vector, the host cell, the carrier, or the pharmaceutical composition, to thereby induce an immune response in the subject). In some embodiments, the multispecific fusion protein (or polypeptides thereof), the polynucleotide, the vector, the host cell, the carrier, or the pharmaceutical composition is administered to the subject in an amount sufficient and for a sufficient time to induce an immune response in the subject.

In one aspect, provided herein is use of a (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for inducing an immune response in a subject (e.g., comprising administering to the subject the multispecific protein, the polynucleotide, the expression vector, the host cell, the carrier, or the pharmaceutical composition, to thereby induce an immune response in a subject). In some embodiments, the multispecific fusion protein (or polypeptides thereof), the polynucleotide, the vector, the host cell, the carrier, or the pharmaceutical composition is administered to the subject in an amount sufficient and for a sufficient time to induce an immune response in the subject.

An immune response in a subject can be measured by common methods known to those of skill in the art. For example, serological assays can be employed to detect a humoral response by measuring titers of anti-antigen (e.g., anti-immunomodulatory protein) IgG antibodies post administration. For example, an enzyme-linked immunosorbent assay (ELISA) is a standard laboratory test for detecting and quantifying antibodies well known to the person of skill in the art. Generally, blood is collected from a consenting subject, centrifuged, and the serum isolated according to standard techniques. The recombinant target antigen (e.g., immunomodulatory polypeptide or protein) is immobilized in microplate wells. The microplate is blocked by through the incubation with an irrelevant antigen (e.g., bovine serum albumin). The serum sample from the subject is prepared and added to the blocked wells to allow for binding of an antigen specific antibodies to the immobilized antigen. The bound antibodies are detected using a secondary tagged antibody that binds to the previously bound antibodies (e.g., anti-human IgG antibodies). See, e.g., Yannick G. et al. Humoral Responses and Serological Assays in SARS-CoV-2 Infections, Frontiers in Immunology, Vol 11 (2020) 10.3389/fimmu.2020.610688; Forgacs David et al., SARS-CoV-2 mRNA Vaccines Elicit Different Responses in Immunologically Naïve and Pre-Immune Humans; Front. Immunol., Vol 12 (27 Sep. 2021) https://doi.org/10.3389/fimmu.2021.728021, the entire contents of each of which is incorporated by reference herein for all purposes.

Cell based assays can also be utilized to detect a cell based immune response (e.g., T cell immune response). For example, antigen specific T cells (e.g., CD4+ or CD8+ T cells) can be measured using an enzyme-linked immunospot (ELISpot), an intracellular cytokine staining (ICS) assay, or an activation induced marker assay (AIM). Each of these assays is commonly used to detect cell based (e.g., T cell) immune responses to vaccines and well known to the person of ordinary skill in the art. See, e.g., Bowyer, Georgina et al. “Activation-induced Markers Detect Vaccine-Specific CD4+ T Cell Responses Not Measured by Assays Conventionally Used in Clinical Trials.” Vaccines vol. 6, 3 50. 31 Jul. 2018, doi:10.3390/vaccines6030050, the entire contents of which is incorporated by reference herein for all purposes.

### 5.9.3 Methods of Activating a T-Cell or Population of T Cells

In one aspect, provided herein are methods of activating a T-cell or population of T cells in a subject, comprising administering (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein or polypeptide described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) to the subject, to thereby activate a T-cell or population of T-cells in the subject. In some embodiments, the multispecific fusion protein (or polypeptides thereof), the polynucleotide, the vector, the host cell, the carrier, or the pharmaceutical composition is administered to the subject in an amount and for a time sufficient to activate a T-cell or population of T-cells in the subject.

In one aspect, provided herein is (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for use in a method of activating a T-cell or population of T cells in a subject (e.g., the method comprising administering to the subject the multispecific protein, the polynucleotide, the expression vector, the host cell, the carrier, or the pharmaceutical composition, to thereby activate a T-cell or population of T cells in the subject). In some embodiments, the multispecific fusion protein (or polypeptides thereof), the polynucleotide, the vector, the host cell, the carrier, or the pharmaceutical composition is administered to the subject in an amount and for a time sufficient to activate a T-cell or population of T-cells in the subject.

In one aspect, provided herein is (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for use in the manufacture of a medicament for activating a T-cell or population of T cells in a subject (e.g., comprising administering to the subject the multispecific protein, the polynucleotide, the expression vector, the host cell, the carrier, or the pharmaceutical composition, to thereby activate a T-cell or population of T cells in the subject). In some embodiments, the multispecific fusion protein (or polypeptides thereof), the polynucleotide, the vector, the host cell, the carrier, or the pharmaceutical composition is administered to the subject in an amount and for a time sufficient to activate a T-cell or population of T-cells in the subject.

In one aspect, provided herein is use of a (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for activating a T-cell or population of T cells in a subject (e.g., comprising administering to the subject the multispecific protein, the polynucleotide, the expression vector, the host cell, the carrier, or the pharmaceutical composition, to thereby activate a T-cell or population of T cells in a subject). In some embodiments, the multispecific fusion protein (or polypeptides thereof), the polynucleotide, the vector, the host cell, the carrier, or the pharmaceutical composition is administered to the subject in an amount and for a time sufficient to activate a T-cell or population of T-cells in the subject.

### 5.9.4 Methods of Preventing or Treating Cancer

In one aspect, provided herein are methods of preventing or treating cancer in a subject, the method comprising administering (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) to the subject, to thereby prevent or treat the cancer in the subject. In some embodiments, the multispecific fusion protein (or polypeptides thereof), the polynucleotide, the vector, the host cell, the carrier, or the pharmaceutical composition is administered to the subject in an amount and for a time sufficient to prevent or treat the cancer in the subject.

In one aspect, provided herein is (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for use in a method of preventing or treating a cancer in a subject, (e.g., the method comprising administering to the subject the multispecific protein, the polynucleotide, the expression vector, the host cell, the carrier, or the pharmaceutical composition, to thereby prevent or treat the cancer in the subject). In some embodiments, the multispecific fusion protein (or polypeptides thereof), the polynucleotide, the vector, the host cell, the carrier, or the pharmaceutical composition is administered to the subject in an amount and for a time sufficient to prevent or treat the cancer in the subject.

In one aspect, provided herein is (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for use in the manufacture of a medicament for the prevention or treatment of cancer in a subject (e.g., comprising administering to the subject the multispecific protein, the polynucleotide, the expression vector, the host cell, the carrier, or the pharmaceutical composition, to thereby prevent or treat the cancer in the subject). In some embodiments, the multispecific fusion protein (or polypeptides thereof), the polynucleotide, the vector, the host cell, the carrier, or the pharmaceutical composition is administered to the subject in an amount and for a time sufficient to prevent or treat the cancer in the subject.

In one aspect, provided herein is use of a (i) a multispecific protein (or polypeptides thereof) described herein; (ii) a polynucleotide described herein (e.g., a polynucleotide encoding a multispecific protein (or polypeptides thereof) described herein); (iii) a vector described herein (e.g., a vector comprising a polynucleotide described herein (e.g., a polynucleotide encoding a multispecific protein (or polypeptides thereof) described herein)); (iv) a host cell described herein (e.g., a host cell comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)); (v) a carrier described herein (e.g., a carrier a multispecific protein or polypeptide described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), or a host cell described herein); or (vi) a pharmaceutical composition described herein (e.g., a pharmaceutical composition comprising a multispecific protein (or polypeptides thereof) described herein, a polynucleotide described herein (e.g., a polynucleotide encoding a multispecific protein (or polypeptides thereof) described herein), a vector described herein (e.g., a vector comprising a polynucleotide described herein (e.g., a polynucleotide encoding a multispecific protein (or polypeptides thereof) described herein), a host cell described herein, or a carrier described herein (e.g., a carrier comprising a multispecific protein (or polypeptides thereof) described herein, a polynucleotide described herein (e.g., a polynucleotide encoding a multispecific protein (or polypeptides thereof) described herein), or a vector described herein (e.g., a vector comprising a polynucleotide described herein (e.g., a polynucleotide encoding a multispecific protein (or polypeptides thereof) described herein)) for the treatment or prevention of cancer in a subject (e.g., comprising administering to the subject the multispecific protein, the polynucleotide, the expression vector, the host cell, the carrier, or the pharmaceutical composition, to thereby prevent or treat the cancer in the subject). In some embodiments, the multispecific fusion protein (or polypeptides thereof), the polynucleotide, the vector, the host cell, the carrier, or the pharmaceutical composition is administered to the subject in an amount and for a time sufficient to prevent or treat the cancer in the subject.

In some embodiments, the cancer is a solid tumor. In some embodiments, the cancer is breast cancer, ovarian cancer, endometrial cancer, uterine cancer, cervical cancer, anal cancer, prostate cancer, rectal cancer, kidney cancer, bladder cancer, colon cancer, liver cancer, pancreatic cancer, thyroid cancer, thymus cancer, lung cancer, bronchus cancer, skin cancer, brain cancer, spinal cord cancer, head cancer, neck cancer, lip cancer, or oral cavity cancer.

### 5.10 Kits

In a one aspect, provided herein are kits comprising a multispecific protein (or polypeptides thereof) described herein, a polynucleotide described herein, a vector described herein, a host cell described herein, a carrier described herein, or a pharmaceutical composition described herein. In addition, the kit may comprise a liquid vehicle for solubilizing or diluting, and/or technical instructions. The technical instructions of the kit may contain information about administration and dosage and subject groups.

In some embodiments, the multispecific protein (or polypeptides thereof) described herein, the polynucleotide described herein, the vector described herein, the host cell described herein, the carrier described herein, or the pharmaceutical composition described herein is provided in a separate part of the kit, wherein the multispecific protein (or polypeptide thereof) described herein, the polynucleotide described herein, the vector described herein, the host cell described herein, the carrier described herein, or the pharmaceutical composition described herein is optionally lyophilized, spray-dried, or spray-freeze dried. The kit may further contain as a part a vehicle (e.g., buffer solution) for solubilizing the dried or lyophilized multispecific protein (or polypeptides thereof) described herein, polynucleotide described herein, vector described herein, host cell described herein, carrier described herein, or pharmaceutical composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container.

Any of the kits described herein may be used in any of the methods described herein (see, e.g., § 5.9).

### 6. EXAMPLES

### 6.1 Example 1. Design and Generation of Multispecific Proteins

A set of multispecific proteins specifically binding to hCD3, hCD8, and a hTAA (e.g., hHER2, hEGFR) were designed in four distinct formats: Format BCA405 (see, e.g., FIG. 1), Format BCA406 (see, e.g., FIG. 2), Format BCA424 (see, e.g., FIG. 3), or Format BCA418 (see, e.g., FIG. 4).

The multispecific proteins were generated using standard methods known in the art. Briefly, the sequences of each multispecific protein were codon optimized to obtain a DNA polynucleotide counterpart that was cloned into a suitable mammalian expression plasmid. The respective plasmids were transiently co-transfected into Expi-CHO-S cells. The transfected cells were grown for 7-10 days with appropriate feeding as per the manufacturer's protocol. The supernatants of the cell cultures were harvested and the multispecific proteins purified using protein A and size exclusion chromatography to obtain high purity intact proteins columns. The purified proteins were eluted in suitable buffer and subsequently diluted to working concentrations. In some experiments one or more reference construct was also assessed, including BCA401(αHER2 control), BCA402 (αCD28 IgG1 control), BCA402 (αCD28 IgG4 control), BCA403 (αCD3ε control), and/or BCA410 (trispecific antibody reference). Cetuximab was used an αEGFR control.

The amino acid sequence of each generated multispecific proteins, as well as certain control and reference proteins) is provided in Table 15.

TABLE 15
The Amino Acid Sequence of Exemplary Multispecific Proteins

| Description | | Amino Acid Sequence | SEQ ID NQ |
|---|---|---|---|
| BCA405 | Heavy Chain | EVQLVESGGGLVQPGGSLRLSCAASGFNIKDTYIH | 237 |
| | (HC)(Knob) | WVRQAPGKGLEWVARIYPTNGYTRYADSVKGRFTI | |
| | αHER2 | SADTSKNTAYLQMNSLRAEDTAVYYCSRWGGDGFY | |
| | αCD3 scFv | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPEAAGGPSVFL | |
| | | FPPKPKDTLMISRTPEVTCVVVDVSHEDPEVKFNW | |
| | | YVDGVEVHNAKTKPREEQYNSTYRVVSVLTVLHQD | |
| | | WLNGKEYKCKVSNKALAAPIEKTISKAKGQPREPQ | |
| | | VYTLPPCRDELTKNQVSLWCLVKGFYPSDIAVEWE | |
| | | SNGQPENNYKTTPPVLDSDGSFFLYSKLTVDKSRW | |
| | | QQGNVFSCSVMHEALHNHYTQKSLSLSPGKGGGGS | |
| | | GGGGSGGGGSEVQLVESGGGLVQPGGSLKLSCAAS | |
| | | GFTFNKYAMNWVRQAPGKGLEWVARIRSKYNNYAT | |
| | | YYADSVKDRFTISRDDSKNTAYLQMNNLKTEDTAV | |
| | | YYCVRHGNFGNSYISYWAYWGQGTLVTVSSGGGGS | |
| | | GGGGSGGGGSQTVVTQEPSLTVSPGGTVTLTCGSS | |
| | | TGAVTSGYYPNWVQQKPGQAPRGLIGGTKFLAPGT | |
| | | PARFSGSLLGGKAALTLSGVQPEDEAEYYCALWYS | |
| | | NRWVFGGGTKLTVL | |
| | HC (Hole) | EVQLVESGGGLVQPGGSLRLSCAASGFNIKDTYIH | 238 |
| | αHER2 | WVRQAPGKGLEWVARIYPTNGYTRYADSVKGRFTI | |
| | αCD28 scFv | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPEAAGGPSVFL | |
| | | FPPKPKDTLMISRTPEVTCVVVDVSHEDPEVKFNW | |
| | | YVDGVEVHNAKTKPREEQYNSTYRVVSVLTVLHQD | |
| | | WLNGKEYKCKVSNKALAAPIEKTISKAKGQPREPQ | |
| | | VCTLPPSRDELTKNQVSLSCAVKGFYPSDIAVEWE | |
| | | SNGQPENNYKTTPPVLDSDGSFFLVSKLTVDKSRW | |
| | | QQGNVFSCSVMHEALHNHYTQKSLSLSPGKGGGGS | |
| | | GGGGSGGGGSQVQLVQSGAEVKKPGASVKVSCKAS | |
| | | GYTFTSYYIHWVRQAPGQGLEWIGCIYPGNVNTNY | |
| | | NEKFKDRATLTVDTSISTAYMELSRLRSDDTAVYF | |
| | | CTRSHYGLDWNFDVWGQGTTVTVSSGGGGSGGGGS | |
| | | GGGGSDIQMTQSPSSLSASVGDRVTITCHASQNIY | |
| | | VWLNWYQQKPGKAPKLLIYKASNLHTGVPSRFSGS | |
| | | GSGTDFTLTISSLQPEDFATYYCQQGQTYPYTFGG | |
| | | GTKVEIK | |
| | Light Chain | DIQMTQSPSSLSASVGDRVTITCRASQDVNTAVAW | 239 |
| | (LC) | YQQKPGKAPKLLIYSASFLYSGVPSRFSGSRSGTD | |
| | αHER2 | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA406 | HC (Knob) | QTVVTQEPSLTVSPGGTVTLTCGSSTGAVTSGYYP | 240 |
| | αCD3 scFv | NWVQQKPGQAPRGLIGGTKFLAPGTPARFSGSLLG | |
| | αHER2 | GKAALTLSGVQPEDEAEYYCALWYSNRWVFGGGTK | |
| | | LTVLGGGGSGGGGSGGGGSEVQLVESGGGLVQPGG | |
| | | SLKLSCAASGFTFNKYAMNWVRQAPGKGLEWVARI | |
| | | RSKYNNYATYYADSVKDRFTISRDDSKNTAYLQMN | |
| | | NLKTEDTAVYYCVRHGNFGNSYISYWAYWGQGTLV | |
| | | TVSSASTKGPEVQLVESGGGLVQPGGSLRLSCAAS | |
| | | GFNIKDTYIHWVRQAPGKGLEWVARIYPTNGYTRY | |
| | | ADSVKGRFTISADTSKNTAYLQMNSLRAEDTAVYY | |
| | | CSRWGGDGFYAMDYWGQGTLVTVSSASTKGPSVFP | |
| | | LAPSSKSTSGGTAALGCLVKDYFPEPVTVSWNSGA | |
| | | LTSGVHTFPAVLQSSGLYSLSSVVTVPSSSLGTQT | |
| | | YICNVNHKPSNTKVDKKVEPKSCDKTHTCPPCPAP | |
| | | EAAGGPSVFLFPPKPKDTLMISRTPEVTCVVVDVS | |
| | | HEDPEVKFNWYVDGVEVHNAKTKPREEQYNSTYRV | |
| | | VSVLTVLHQDWLNGKEYKCKVSNKALAAPIEKTIS | |
| | | KAKGQPREPQVYTLPPCRDELTKNQVSLWCLVKGF | |
| | | YPSDIAVEWESNGQPENNYKTTPPVLDSDGSFFLY | |
| | | SKLTVDKSRWQQGNVFSCSVMHEALHNHYTQKSLS | |
| | | LSPGK | |
| | HC (Hole) | DIQMTQSPSSLSASVGDRVTITCHASQNIYVWLNW | 241 |
| | αCD28 scFv | YQQKPGKAPKLLIYKASNLHTGVPSRFSGSGSGTD | |
| | αHER2 | FTLTISSLQPEDFATYYCQQGQTYPYTFGGGTKVE | |
| | | IKGGGGSGGGGSGGGGSQVQLVQSGAEVKKPGASV | |
| | | KVSCKASGYTFTSYYIHWVRQAPGQGLEWIGCIYP | |
| | | GNVNTNYNEKFKDRATLTVDTSISTAYMELSRLRS | |
| | | DDTAVYFCTRSHYGLDWNFDVWGQGTTVTVSSAST | |
| | | KGPEVQLVESGGGLVQPGGSLRLSCAASGFNIKDT | |
| | | YIHWVRQAPGKGLEWVARIYPTNGYTRYADSVKGR | |
| | | FTISADTSKNTAYLQMNSLRAEDTAVYYCSRWGGD | |
| | | GFYAMDYWGQGTLVTVSSASTKGPSVFPLAPSSKS | |
| | | TSGGTAALGCLVKDYFPEPVTVSWNSGALTSGVHT | |
| | | FPAVLQSSGLYSLSSVVTVPSSSLGTQTYICNVNH | |
| | | KPSNTKVDKKVEPKSCDKTHTCPPCPAPEAAGGPS | |
| | | VFLFPPKPKDTLMISRTPEVTCVVVDVSHEDPEVK | |
| | | FNWYVDGVEVHNAKTKPREEQYNSTYRVVSVLTVL | |
| | | HQDWLNGKEYKCKVSNKALAAPIEKTISKAKGQPR | |
| | | EPQVCTLPPSRDELTKNQVSLSCAVKGFYPSDIAV | |
| | | EWESNGQPENNYKTTPPVLDSDGSFFLVSKLTVDK | |
| | | SRWQQGNVFSCSVMHEALHNHYTQKSLSLSPGK | |
| | LC | DIQMTQSPSSLSASVGDRVTITCRASQDVNTAVAW | 239 |
| | αHER2 | YQQKPGKAPKLLIYSASFLYSGVPSRFSGSRSGTD | |
| | | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA424 | HC (Knob) | EVQLVESGGGLVQPGGSLRLSCAASGFNIKDTYIH | 242 |
| | αHER2 | WVRQAPGKGLEWVARIYPTNGYTRYADSVKGRFTI | |
| | αCD28 scFv | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDGGGGSGGGGSQVQLVQSGAEV | |
| | | KKPGASVKVSCKASGYTFTSYYIHWVRQAPGQCLE | |
| | | WIGCIYPGNVNTNYNEKFKDRATLTVDTSISTAYM | |
| | | ELSRLRSDDTAVYFCTRSHYGLDWNFDVWGQGTTV | |
| | | TVSSGGGGSGGGGSGGGGSDIQMTQSPSSLSASVG | |
| | | DRVTITCHASQNIYVWLNWYQQKPGKAPKLLIYKA | |
| | | SNLHTGVPSRFSGSGSGTDFTLTISSLQPEDFATY | |
| | | YCQQGQTYPYTFGCGTKVEIKEPKSSDKTHTCPPC | |
| | | PAPEAAGGPSVFLFPPKPKDTLMISRTPEVTCVVV | |
| | | DVSHEDPEVKFNWYVDGVEVHNAKTKPREEQYNST | |
| | | YRVVSVLTVLHQDWLNGKEYKCKVSNKALAAPIEK | |
| | | TISKAKGQPREPQVYTLPPCRDELTKNQVSLWCLV | |
| | | KGFYPSDIAVEWESNGQPENNYKTTPPVLDSDGSF | |
| | | FLYSKLTVDKSRWQQGNVFSCSVMHEALHNHYTQK | |
| | | SLSLSPGK | |
| | HC (Hole) | EVQLVESGGGLVQPGGSLRLSCAASGFNIKDTYIH | 243 |
| | αHER2 | WVRQAPGKGLEWVARIYPTNGYTRYADSVKGRFTI | |
| | αCD3 scFv | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDGGGGSGGGGSEVQLVESGGGL | |
| | | VQPGGSLKLSCAASGFTFNKYAMNWVRQAPGKCLE | |
| | | WVARIRSKYNNYATYYADSVKDRFTISRDDSKNTA | |
| | | YLQMNNLKTEDTAVYYCVRHGNFGNSYISYWAYWG | |
| | | QGTLVTVSSGGGGSGGGGSGGGGSQTVVTQEPSLT | |
| | | VSPGGTVTLTCGSSTGAVTSGYYPNWVQQKPGQAP | |
| | | RGLIGGTKFLAPGTPARFSGSLLGGKAALTLSGVQ | |
| | | PEDEAEYYCALWYSNRWVFGCGTKLTVLEPKSSDK | |
| | | THTCPPCPAPEAAGGPSVFLFPPKPKDTLMISRTP | |
| | | EVTCVVVDVSHEDPEVKFNWYVDGVEVHNAKTKPR | |
| | | EEQYNSTYRVVSVLTVLHQDWLNGKEYKCKVSNKA | |
| | | LAAPIEKTISKAKGQPREPQVCTLPPSRDELTKNQ | |
| | | VSLSCAVKGFYPSDIAVEWESNGQPENNYKTTPPV | |
| | | LDSDGSFFLVSKLTVDKSRWQQGNVFSCSVMHEAL | |
| | | HNHYTQKSLSLSPGK | |
| | LC | DIQMTQSPSSLSASVGDRVTITCRASQDVNTAVAW | 239 |
| | αHER2 | YQQKPGKAPKLLIYSASFLYSGVPSRFSGSRSGTD | |
| | | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA418 | HC (Knob) | QVQLVQSGAEVKKPGASVKVSCKASGYTFTSYYIH | 244 |
| | αCD28 ScFv | WVRQAPGQCLEWIGCIYPGNVNTNYNEKFKDRATL | |
| | αCD3 | TVDTSISTAYMELSRLRSDDTAVYFCTRSHYGLDW | |
| | | NFDVWGQGTTVTVSSGGGGSGGGGSGGGGSDIQMT | |
| | | QSPSSLSASVGDRVTITCHASQNIYVWLNWYQQKP | |
| | | GKAPKLLIYKASNLHTGVPSRFSGSGSGTDFTLTI | |
| | | SSLQPEDFATYYCQQGQTYPYTFGCGTKVEIKGGG | |
| | | GSGGGGSEVQLVESGGGLVQPGGSLKLSCAASGFT | |
| | | FNKYAMNWVRQAPGKGLEWVARIRSKYNNYATYYA | |
| | | DSVKDRFTISRDDSKNTAYLQMNNLKTEDTAVYYC | |
| | | VRHGNFGNSYISYWAYWGQGTLVTVSSASTKGPSV | |
| | | FPLAPSSKSTSGGTAALGCLVKDYFPEPVTVSWNS | |
| | | GALTSGVHTFPAVLQSSGLYSLSSVVTVPSSSLGT | |
| | | QTYICNVNHKPSNTKVDKKVEPKSCDKTHTCPPCP | |
| | | APEAAGGPSVFLFPPKPKDTLMISRTPEVTCVVVD | |
| | | VSHEDPEVKFNWYVDGVEVHNAKTKPREEQYNSTY | |
| | | RVVSVLTVLHQDWLNGKEYKCKVSNKALAAPIEKT | |
| | | ISKAKGQPREPQVYTLPPCRDELTKNQVSLWCLVK | |
| | | GFYPSDIAVEWESNGQPENNYKTTPPVLDSDGSFF | |
| | | LYSKLTVDKSRWQQGNVFSCSVMHEALHNHYTQKS | |
| | | LSLSPGK | |
| | HC Hole | EVQLVESGGGLVQPGGSLRLSCAASGFNIKDTYIH | 245 |
| | αHER2 | WVRQAPGKGLEWVARIYPTNGYTRYADSVKGRFTI | |
| | | SADTSKNTAYLQMNSLRAEDTAVYYCSRWGGDGFY | |
| | | AMDYWGQGTLVTVSSASTELPPKVSVFVPPRDGFF | |
| | | GNPRKSELICEATGFSPRQIQVSWLREGKQVGSGV | |
| | | TTDQVQAEAKESGPTTYDVTSTLTIKESDWLGQSM | |
| | | FTCRVDHRGLTFQQSASSMCDKTHTCPPCPAPEAA | |
| | | GGPSVFLFPPKPKDTLMISRTPEVTCVVVDVSHED | |
| | | PEVKFNWYVDGVEVHNAKTKPREEQYNSTYRVVSV | |
| | | LTVLHQDWLNGKEYKCKVSNKALAAPIEKTISKAK | |
| | | GQPREPQVCTLPPSRDELTKNQVSLSCAVKGFYPS | |
| | | DIAVEWESNGQPENNYKTTPPVLDSDGSFFLVSKL | |
| | | TVDKSRWQQGNVFSCSVMHEALHNHYTQKSLSLSP | |
| | | GK | |
| | LC αCD3 | QTVVTQEPSLTVSPGGTVTLTCGSSTGAVTSGYYP | 246 |
| | | NWVQQKPGQAPRGLIGGTKFLAPGTPARFSGSLLG | |
| | | GKAALTLSGVQPEDEAEYYCALWYSNRWVFGGGTK | |
| | | LTVLRTVAAPSVFIFPPSDEQLKSGTASVVCLLNN | |
| | | FYPREAKVQWKVDNALQSGNSQESVTEQDSKDSTY | |
| | | SLSSTLTLSKADYEKHKVYACEVTHQGLSSPVTKS | |
| | | FNRGEC | |
| | LC IgM | DIQMTQSPSSLSASVGDRVTITCRASQDVNTAVAW | 247 |
| | CH2 αHER2 | YQQKPGKAPKLLIYSASFLYSGVPSRFSGSRSGTD | |
| | | FTLTISSLQPEDFATYYCQQHYTTPPTFGQGTKVE | |
| | | IKRTAELPPKVSVFVPPRDGFFGNPRKSKLICKAT | |
| | | GFSPRQIQVSWLREGKQVGSGVTTKQVQAEAKESG | |
| | | PTTYKVTSTLTIKESDWLGQSMFTCRVDHRGLTFQ | |
| | | QSASSMC | |
| BCA405.S | HC (Knob) | EVQLVESGGGLVQPGGSLRLSCAASGFNIKDTYIH | 248 |
| | αHER2 | WVRQAPGKGLEWVARIYPTNGYTRYADSVKGRFTI | |
| | αCD3 scFv | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPEAAGGPSVFL | |
| | | FPPKPKDTLMISRTPEVTCVVVDVSHEDPEVKFNW | |
| | | YVDGVEVHNAKTKPREEQYNSTYRVVSVLTVLHQD | |
| | | WLNGKEYKCKVSNKALAAPIEKTISKAKGQPREPQ | |
| | | VYTLPPCRDELTKNQVSLWCLVKGFYPSDIAVEWE | |
| | | SNGQPENNYKTTPPVLDSDGSFFLYSKLTVDKSRW | |
| | | QQGNVFSCSVMHEALHNHYTQKSLSLSPGKGGGGS | |
| | | GGGGSGGGGSQVQLVESGGGVVQPGRSLRLSCAAS | |
| | | GFTFTKAWMHWVRQAPGKQLEWVAQIKDKSNSYAT | |
| | | YYADSVKGRFTISRDDSKNTLYLQMNSLRAEDTAV | |
| | | YYCRGVYYALSPFDYWGQGTLVTVSSGGGGSGGGG | |
| | | SGGGGSDIVMTQTPLSLSVTPGQPASISCKSSQSL | |
| | | VHNNANTYLSWYLQKPGQSPQSLIYKVSNRFSGVP | |
| | | DRFSGSGSGTDFTLKISRVEAEDVGVYYCGQGTQY | |
| | | PFTFGSGTKVEIK | |
| | HC (Hole) | EVQLVESGGGLVQPGGSLRLSCAASGFNIKDTYIH | 249 |
| | αHER2 | WVRQAPGKGLEWVARIYPTNGYTRYADSVKGRFTI | |
| | αCD28 scFv | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPEAAGGPSVFL | |
| | | FPPKPKDTLMISRTPEVTCVVVDVSHEDPEVKFNW | |
| | | YVDGVEVHNAKTKPREEQYNSTYRVVSVLTVLHQD | |
| | | WLNGKEYKCKVSNKALAAPIEKTISKAKGQPREPQ | |
| | | VCTLPPSRDELTKNQVSLSCAVKGFYPSDIAVEWE | |
| | | SNGQPENNYKTTPPVLDSDGSFFLVSKLTVDKSRW | |
| | | QQGNVFSCSVMHEALHNHYTQKSLSLSPGKGGGGS | |
| | | GGGGSGGGGSQVQLQESGPGLVKPSQTLSLTCTVS | |
| | | GFSLSDYGVHWVRQPPGKGLEWLGVIWAGGGTNYN | |
| | | PSLKSRKTISKDTSKNQVSLKLSSVTAADTAVYYC | |
| | | ARDKGYSYYYSMDYWGQGTTVTVSSGGGGSGGGGS | |
| | | GGGGSDIVLTQSPASLAVSPGQRATITCRASESVE | |
| | | YYVTSLMQWYQQKPGQPPKLLIFAASNVESGVPAR | |
| | | FSGSGSGTDFTLTINPVEANDVANYYCQQSRKVPY | |
| | | TFGQGTKLEIK | |
| | LC | DIQMTQSPSSLSASVGDRVTITCRASQDVNTAVAW | 239 |
| | αHER2 | YQQKPGKAPKLLIYSASFLYSGVPSRFSGSRSGTD | |
| | | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA406.S | HC (Knob) | DIVMTQTPLSLSVTPGQPASISCKSSQSLVHNNAN | 250 |
| | αCD3 scFv | TYLSWYLQKPGQSPQSLIYKVSNRFSGVPDRFSGS | |
| | αHER2 | GSGTDFTLKISRVEAEDVGVYYCGQGTQYPFTFGS | |
| | | GTKVEIKGGGGSGGGGSGGGGSQVQLVESGGGVVQ | |
| | | PGRSLRLSCAASGFTFTKAWMHWVRQAPGKQLEWV | |
| | | AQIKDKSNSYATYYADSVKGRFTISRDDSKNTLYL | |
| | | QMNSLRAEDTAVYYCRGVYYALSPFDYWGQGTLVT | |
| | | VSSASTKGPEVQLVESGGGLVQPGGSLRLSCAASG | |
| | | FNIKDTYIHWVRQAPGKGLEWVARIYPTNGYTRYA | |
| | | DSVKGRFTISADTSKNTAYLQMNSLRAEDTAVYYC | |
| | | SRWGGDGFYAMDYWGQGTLVTVSSASTKGPSVFPL | |
| | | APSSKSTSGGTAALGCLVKDYFPEPVTVSWNSGAL | |
| | | TSGVHTFPAVLQSSGLYSLSSVVTVPSSSLGTQTY | |
| | | ICNVNHKPSNTKVDKKVEPKSCDKTHTCPPCPAPE | |
| | | AAGGPSVFLFPPKPKDTLMISRTPEVTCVVVDVSH | |
| | | EDPEVKFNWYVDGVEVHNAKTKPREEQYNSTYRVV | |
| | | SVLTVLHQDWLNGKEYKCKVSNKALAAPIEKTISK | |
| | | AKGQPREPQVYTLPPCRDELTKNQVSLWCLVKGFY | |
| | | PSDIAVEWESNGQPENNYKTTPPVLDSDGSFFLYS | |
| | | KLTVDKSRWQQGNVFSCSVMHEALHNHYTQKSLSL | |
| | | SPGK | |
| | HC (Hole) | DIVLTQSPASLAVSPGQRATITCRASESVEYYVTS | 251 |
| | αCD28 scFv | LMQWYQQKPGQPPKLLIFAASNVESGVPARFSGSG | |
| | αHER2 | SGTDFTLTINPVEANDVANYYCQQSRKVPYTFGQG | |
| | | TKLEIKGGGGSGGGGSGGGGSQVQLQESGPGLVKP | |
| | | SQTLSLTCTVSGFSLSDYGVHWV'RQPPGKGLEWL | |
| | | GVIWAGGGTNYNPSLKSRKTISKDTSKNQVSLKLS | |
| | | SVTAADTAVYYCARDKGYSYYYSMDYWGQGTTVTV | |
| | | SSASTKGPEVQLVESGGGLVQPGGSLRLSCAASGF | |
| | | NIKDTYIHWVRQAPGKGLEWVARIYPTNGYTRYAD | |
| | | SVKGRFTISADTSKNTAYLQMNSLRAEDTAVYYCS | |
| | | RWGGDGFYAMDYWGQGTLVTVSSASTKGPSVFPLA | |
| | | PSSKSTSGGTAALGCLVKDYFPEPVTVSWNSGALT | |
| | | SGVHTFPAVLQSSGLYSLSSVVTVPSSSLGTQTYI | |
| | | CNVNHKPSNTKVDKKVEPKSCDKTHTCPPCPAPEA | |
| | | AGGPSVFLFPPKPKDTLMISRTPEVTCVVVDVSHE | |
| | | DPEVKFNWYVDGVEVHNAKTKPREEQYNSTYRVVS | |
| | | VLTVLHQDWLNGKEYKCKVSNKALAAPIEKTISKA | |
| | | KGQPREPQVCTLPPSRDELTKNQVSLSCAVKGFYP | |
| | | SDIAVEWESNGQPENNYKTTPPVLDSDGSFFLVSK | |
| | | LTVDKSRWQQGNVFSCSVMHEALHNHYTQKSLSLS | |
| | | PGK | |
| | LC | DIQMTQSPSSLSASVGDRVTITCRASQDVNTAVAW | 239 |
| | αHER2 | YQQKPGKAPKLLIYSASFLYSGVPSRFSGSRSGTD | |
| | | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA424.S | HC (Knob) | EVQLVESGGGLVQPGGSLRLSCAASGFNIKDTYIH | 252 |
| | αHER2 | WVRQAPGKGLEWVARIYPTNGYTRYADSVKGRFTI | |
| | αCD28 scFv | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDGGGGSGGGGSQVQLQESGPGL | |
| | | VKPSQTLSLTCTVSGFSLSDYGVHWVRQPPGKCLE | |
| | | WLGVIWAGGGTNYNPSLKSRKTISKDTSKNQVSLK | |
| | | LSSVTAADTAVYYCARDKGYSYYYSMDYWGQGTTV | |
| | | TVSSGGGGSGGGGSGGGGSDIVLTQSPASLAVSPG | |
| | | QRATITCRASESVEYYVTSLMQWYQQKPGQPPKLL | |
| | | IFAASNVESGVPARFSGSGSGTDFTLTINPVEAND | |
| | | VANYYCQQSRKVPYTFGCGTKLEIKEPKSSDKTHT | |
| | | CPPCPAPEAAGGPSVFLFPPKPKDTLMISRTPEVT | |
| | | CVVVDVSHEDPEVKFNWYVDGVEVHNAKTKPREEQ | |
| | | YNSTYRVVSVLTVLHQDWLNGKEYKCKVSNKALAA | |
| | | PIEKTISKAKGQPREPQVYTLPPCRDELTKNQVSL | |
| | | WCLVKGFYPSDIAVEWESNGQPENNYKTTPPVLDS | |
| | | DGSFFLYSKLTVDKSRWQQGNVFSCSVMHEALHNH | |
| | | YTQKSLSLSPGK | |
| | HC (Hole) | EVQLVESGGGLVQPGGSLRLSCAASGFNIKDTYIH | 253 |
| | αHER2 | WVRQAPGKGLEWVARIYPTNGYTRYADSVKGRFTI | |
| | αCD3 scFv | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDGGGGSGGGGSQVQLVESGGGV | |
| | | VQPGRSLRLSCAASGFTFTKAWMHWVRQAPGKCLE | |
| | | WVAQIKDKSNSYATYYADSVKGRFTISRDDSKNTL | |
| | | YLQMNSLRAEDTAVYYCRGVYYALSPFDYWGQGTL | |
| | | VTVSSGGGGSGGGGSGGGGSDIVMTQTPLSLSVTP | |
| | | GQPASISCKSSQSLVHNNANTYLSWYLQKPGQSPQ | |
| | | SLIYKVSNRFSGVPDRFSGSGSGTDFTLKISRVEA | |
| | | EDVGVYYCGQGTQYPFTFGCGTKVEIKEPKSSDKT | |
| | | HTCPPCPAPEAAGGPSVFLFPPKPKDTLMISRTPE | |
| | | VTCVVVDVSHEDPEVKFNWYVDGVEVHNAKTKPRE | |
| | | EQYNSTYRVVSVLTVLHQDWLNGKEYKCKVSNKAL | |
| | | AAPIEKTISKAKGQPREPQVCTLPPSRDELTKNQV | |
| | | SLSCAVKGFYPSDIAVEWESNGQPENNYKTTPPVL | |
| | | DSDGSFFLVSKLTVDKSRWQQGNVFSCSVMHEALH | |
| | | NHYTQKSLSLSPGK | |
| | LC | DIQMTQSPSSLSASVGDRVTITCRASQDVNTAVAW | 239 |
| | αHER2 | YQQKPGKAPKLLIYSASFLYSGVPSRFSGSRSGTD | |
| | | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA405.EG | HC (Knob) | QVQLKQSGPGLVQPSQSLSITCTVSGFSLTNYGVH | 254 |
| | αEGFR | WVRQSPGKGLEWLGVIWSGGNTDYNTPFTSRLSIN | |
| | αCD3 scFv | KDNSKSQVFFKMNSLQSNDTAIYYCARALTYYDYE | |
| | | FAYWGQGTLVTVSAASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPEAAGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALAAPIEKTISKAKGQPREPQV | |
| | | YTLPPCRDELTKNQVSLWCLVKGFYPSDIAVEWES | |
| | | NGQPENNYKTTPPVLDSDGSFFLYSKLTVDKSRWQ | |
| | | QGNVFSCSVMHEALHNHYTQKSLSLSPGKGGGGSG | |
| | | GGGSGGGGSEVQLVESGGGLVQPGGSLKLSCAASG | |
| | | FTFNKYAMNWVRQAPGKGLEWVARIRSKYNNYATY | |
| | | YADSVKDRFTISRDDSKNTAYLQMNNLKTEDTAVY | |
| | | YCVRHGNFGNSYISYWAYWGQGTLVTVSSGGGGSG | |
| | | GGGSGGGGSQTVVTQEPSLTVSPGGTVTLTCGSST | |
| | | GAVTSGYYPNWVQQKPGQAPRGLIGGTKFLAPGTP | |
| | | ARFSGSLLGGKAALTLSGVQPEDEAEYYCALWYSN | |
| | | RWVFGGGTKLTVL | |
| | HC (Hole) | QVQLKQSGPGLVQPSQSLSITCTVSGFSLTNYGVH | 255 |
| | αEGFR | WVRQSPGKGLEWLGVIWSGGNTDYNTPFTSRLSIN | |
| | αCD28 scFv | KDNSKSQVFFKMNSLQSNDTAIYYCARALTYYDYE | |
| | | FAYWGQGTLVTVSAASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPEAAGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALAAPIEKTISKAKGQPREPQV | |
| | | CTLPPSRDELTKNQVSLSCAVKGFYPSDIAVEWES | |
| | | NGQPENNYKTTPPVLDSDGSFFLVSKLTVDKSRWQ | |
| | | QGNVFSCSVMHEALHNHYTQKSLSLSPGKGGGGSG | |
| | | GGGSGGGGSQVQLVQSGAEVKKPGASVKVSCKASG | |
| | | YTFTSYYIHWVRQAPGQGLEWIGCIYPGNVNTNYN | |
| | | EKFKDRATLTVDTSISTAYMELSRLRSDDTAVYFC | |
| | | TRSHYGLDWNFDVWGQGTTVTVSSGGGGSGGGGSG | |
| | | GGGSDIQMTQSPSSLSASVGDRVTITCHASQNIYV | |
| | | WLNWYQQKPGKAPKLLIYKASNLHTGVPSRFSGSG | |
| | | SGTDFTLTISSLQPEDFATYYCQQGQTYPYTFGGG | |
| | | TKVEIK | |
| | LC | DILLTQSPVILSVSPGERVSFSCRASQSIGTNIHW | 256 |
| | αEGFR | YQQRTNGSPRLLIKYASESISGIPSRFSGSGSGTD | |
| | | FTLSINSVESEDIADYYCQQNNNWPTTFGAGTKLE | |
| | | LKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA406.EG | HC (Knob) | QTVVTQEPSLTVSPGGTVTLTCGSSTGAVTSGYYP | 257 |
| | αCD3 scFv | NWVQQKPGQAPRGLIGGTKFLAPGTPARFSGSLLG | |
| | αEGFR | GKAALTLSGVQPEDEAEYYCALWYSNRWVFGGGTK | |
| | | LTVLGGGGSGGGGSGGGGSEVQLVESGGGLVQPGG | |
| | | SLKLSCAASGFTFNKYAMNWVRQAPGKGLEWVARI | |
| | | RSKYNNYATYYADSVKDRFTISRDDSKNTAYLQMN | |
| | | NLKTEDTAVYYCVRHGNFGNSYISYWAYWGQGTLV | |
| | | TVSSASTKGPQVQLKQSGPGLVQPSQSLSITCTVS | |
| | | GFSLTNYGVHWVRQSPGKGLEWLGVIWSGGNTDYN | |
| | | TPFTSRLSINKDNSKSQVFFKMNSLQSNDTAIYYC | |
| | | ARALTYYDYEFAYWGQGTLVTVSAASTKGPSVFPL | |
| | | APSSKSTSGGTAALGCLVKDYFPEPVTVSWNSGAL | |
| | | TSGVHTFPAVLQSSGLYSLSSVVTVPSSSLGTQTY | |
| | | ICNVNHKPSNTKVDKKVEPKSCDKTHTCPPCPAPE | |
| | | AAGGPSVFLFPPKPKDTLMISRTPEVTCVVVDVSH | |
| | | EDPEVKFNWYVDGVEVHNAKTKPREEQYNSTYRVV | |
| | | SVLTVLHQDWLNGKEYKCKVSNKALAAPIEKTISK | |
| | | AKGQPREPQVYTLPPCRDELTKNQVSLWCLVKGFY | |
| | | PSDIAVEWESNGQPENNYKTTPPVLDSDGSFFLYS | |
| | | KLTVDKSRWQQGNVFSCSVMHEALHNHYTQKSLSL | |
| | | SPGK | |
| | HC (Hole) | DIQMTQSPSSLSASVGDRVTITCHASQNIYVWLNW | 258 |
| | αCD28 scFv | YQQKPGKAPKLLIYKASNLHTGVPSRFSGSGSGTD | |
| | αEGFR | FTLTISSLQPEDFATYYCQQGQTYPYTFGGGTKVE | |
| | | IKGGGGSGGGGSGGGGSQVQLVQSGAEVKKPGASV | |
| | | KVSCKASGYTFTSYYIHWVRQAPGQGLEWIGCIYP | |
| | | GNVNTNYNEKFKDRATLTVDTSISTAYMELSRLRS | |
| | | DDTAVYFCTRSHYGLDWNFDVWGQGTTVTVSSAST | |
| | | KGPQVQLKQSGPGLVQPSQSLSITCTVSGFSLTNY | |
| | | GVHWVRQSPGKGLEWLGVIWSGGNTDYNTPFTSRL | |
| | | SINKDNSKSQVFFKMNSLQSNDTAIYYCARALTYY | |
| | | DYEFAYWGQGTLVTVSAASTKGPSVFPLAPSSKST | |
| | | SGGTAALGCLVKDYFPEPVTVSWNSGALTSGVHTF | |
| | | PAVLQSSGLYSLSSVVTVPSSSLGTQTYICNVNHK | |
| | | PSNTKVDKKVEPKSCDKTHTCPPCPAPEAAGGPSV | |
| | | FLFPPKPKDTLMISRTPEVTCVVVDVSHEDPEVKF | |
| | | NWYVDGVEVHNAKTKPREEQYNSTYRVVSVLTVLH | |
| | | QDWLNGKEYKCKVSNKALAAPIEKTISKAKGQPRE | |
| | | PQVCTLPPSRDELTKNQVSLSCAVKGFYPSDIAVE | |
| | | WESNGQPENNYKTTPPVLDSDGSFFLVSKLTVDKS | |
| | | RWQQGNVFSCSVMHEALHNHYTQKSLSLSPGK | |
| | LC | DILLTQSPVILSVSPGERVSFSCRASQSIGTNIHW | 256 |
| | αEGFR | YQQRTNGSPRLLIKYASESISGIPSRFSGSGSGTD | |
| | | FTLSINSVESEDIADYYCQQNNNWPTTFGAGTKLE | |
| | | LKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA424.EG | HC (Knob) | QVQLKQSGPGLVQPSQSLSITCTVSGFSLTNYGVH | 259 |
| | αEGFR | WVRQSPGKGLEWLGVIWSGGNTDYNTPFTSRLSIN | |
| | αCD28 scFv | KDNSKSQVFFKMNSLQSNDTAIYYCARALTYYDYE | |
| | | FAYWGQGTLVTVSAASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDGGGGSGGGGSQVQLVQSGAEVK | |
| | | KPGASVKVSCKASGYTFTSYYIHWVRQAPGQCLEW | |
| | | IGCIYPGNVNTNYNEKFKDRATLTVDTSISTAYME | |
| | | LSRLRSDDTAVYFCTRSHYGLDWNFDVWGQGTTVT | |
| | | VSSGGGGSGGGGSGGGGSDIQMTQSPSSLSASVGD | |
| | | RVTITCHASQNIYVWLNWYQQKPGKAPKLLIYKAS | |
| | | NLHTGVPSRFSGSGSGTDFTLTISSLQPEDFATYY | |
| | | CQQGQTYPYTFGCGTKVEIKEPKSSDKTHTCPPCP | |
| | | APEAAGGPSVFLFPPKPKDTLMISRTPEVTCVVVD | |
| | | VSHEDPEVKFNWYVDGVEVHNAKTKPREEQYNSTY | |
| | | RVVSVLTVLHQDWLNGKEYKCKVSNKALAAPIEKT | |
| | | ISKAKGQPREPQVYTLPPCRDELTKNQVSLWCLVK | |
| | | GFYPSDIAVEWESNGQPENNYKTTPPVLDSDGSFF | |
| | | LYSKLTVDKSRWQQGNVFSCSVMHEALHNHYTQKS | |
| | | LSLSPGK | |
| | HC (Hole) | QVQLKQSGPGLVQPSQSLSITCTVSGFSLTNYGVH | 260 |
| | αEGFR | WVRQSPGKGLEWLGVIWSGGNTDYNTPFTSRLSIN | |
| | αCD3 scFv | KDNSKSQVFFKMNSLQSNDTAIYYCARALTYYDYE | |
| | | FAYWGQGTLVTVSAASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDGGGGSGGGGSEVQLVESGGGLV | |
| | | QPGGSLKLSCAASGFTFNKYAMNWVRQAPGKCLEW | |
| | | VARIRSKYNNYATYYADSVKDRFTISRDDSKNTAY | |
| | | LQMNNLKTEDTAVYYCVRHGNFGNSYISYWAYWGQ | |
| | | GTLVTVSSGGGGSGGGGSGGGGSQTVVTQEPSLTV | |
| | | SPGGTVTLTCGSSTGAVTSGYYPNWVQQKPGQAPR | |
| | | GLIGGTKFLAPGTPARFSGSLLGGKAALTLSGVQP | |
| | | EDEAEYYCALWYSNRWVFGCGTKLTVLEPKSSDKT | |
| | | HTCPPCPAPEAAGGPSVFLFPPKPKDTLMISRTPE | |
| | | VTCVVVDVSHEDPEVKFNWYVDGVEVHNAKTKPRE | |
| | | EQYNSTYRVVSVLTVLHQDWLNGKEYKCKVSNKAL | |
| | | AAPIEKTISKAKGQPREPQVCTLPPSRDELTKNQV | |
| | | SLSCAVKGFYPSDIAVEWESNGQPENNYKTTPPVL | |
| | | DSDGSFFLVSKLTVDKSRWQQGNVFSCSVMHEALH | |
| | | NHYTQKSLSLSPGK | |
| | LC | DILLTQSPVILSVSPGERVSFSCRASQSIGTNIHW | 256 |
| | αEGFR | YQQRTNGSPRLLIKYASESISGIPSRFSGSGSGTD | |
| | | FTLSINSVESEDIADYYCQQNNNWPTTFGAGTKLE | |
| | | LKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA605 | HC (Knob) | QVQLQESGPGLVKPSETLSLTCTVSGGSVSSGSYY | 304 |
| (M912) | αMSLN | WSWIRQPPGKGLEWIGYIYYSGSTNYNPSLKSRVT | |
| (405 Format) | αCD3 scFv | ISVDTSKNQFSLKLSSVTAADTAVYYCAREGKNGA | |
| | | FDIWGQGTMVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPEAAGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALAAPIEKTISKAKGQPREPQV | |
| | | YTLPPCRDELTKNQVSLWCLVKGFYPSDIAVEWES | |
| | | NGQPENNYKTTPPVLDSDGSFFLYSKLTVDKSRWQ | |
| | | QGNVFSCSVMHEALHNHYTQKSLSLSPGKGGGGSG | |
| | | GGGSGGGGSEVQLVESGGGLVQPGGSLKLSCAASG | |
| | | FTFNKYAMNWVRQAPGKCLEWVARIRSKYNNYATY | |
| | | YADSVKDRFTISRDDSKNTAYLQMNNLKTEDTAVY | |
| | | YCVRHGNFGNSYISYWAYWGQGTLVTVSSGGGGSG | |
| | | GGGSGGGGSQTVVTQEPSLTVSPGGTVTLTCGSST | |
| | | GAVTSGYYPNWVQQKPGQAPRGLIGGTKFLAPGTP | |
| | | ARFSGSLLGGKAALTLSGVQPEDEAEYYCALWYSN | |
| | | RWVFGCGTKLTVL | |
| | HC (Hole) | QVQLQESGPGLVKPSETLSLTCTVSGGSVSSGSYY | 305 |
| | αMSLN | WSWIRQPPGKGLEWIGYIYYSGSTNYNPSLKSRVT | |
| | αCD28 scFv | ISVDTSKNQFSLKLSSVTAADTAVYYCAREGKNGA | |
| | | FDIWGQGTMVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPEAAGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALAAPIEKTISKAKGQPREPQV | |
| | | CTLPPSRDELTKNQVSLSCAVKGFYPSDIAVEWES | |
| | | NGQPENNYKTTPPVLDSDGSFFLVSKLTVDKSRWQ | |
| | | QGNVFSCSVMHEALHNHYTQKSLSLSPGKGGGGSG | |
| | | GGGSGGGGSQVQLVQSGAEVKKPGASVKVSCKASG | |
| | | YTFTSYYIHWVRQAPGQCLEWIGCIYPGNVNTNYN | |
| | | EKFKDRATLTVDTSISTAYMELSRLRSDDTAVYFC | |
| | | TRSHYGLDWNFDVWGQGTTVTVSSGGGGSGGGGSG | |
| | | GGGSDIQMTQSPSSLSASVGDRVTITCHASQNIYV | |
| | | WLNWYQQKPGKAPKLLIYKASNLHTGVPSRFSGSG | |
| | | SGTDFTLTISSLQPEDFATYYCQQGQTYPYTFGCG | |
| | | TKVEIK | |
| | LC | DIQMTQSPSSLSASVGDRVTITCRASQSISSYLNW | 306 |
| | αMSLN | YQQKPGKAPKLLIYAASSLQSGVPSGFSGSGSGTD | |
| | | FTLTISSLQPEDFATYYCQQSYSTPLTFGGGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA606 | HC (Knob) | QTVVTQEPSLTVSPGGTVTLTCGSSTGAVTSGYYP | 307 |
| (M912) | αCD3 scFv | NWVQQKPGQAPRGLIGGTKFLAPGTPARFSGSLLG | |
| (406 Format) | αMSLN | GKAALTLSGVQPEDEAEYYCALWYSNRWVFGCGTK | |
| | | LTVLGGGGSGGGGSGGGGSEVQLVESGGGLVQPGG | |
| | | SLKLSCAASGFTFNKYAMNWVRQAPGKCLEWVARI | |
| | | RSKYNNYATYYADSVKDRFTISRDDSKNTAYLQMN | |
| | | NLKTEDTAVYYCVRHGNFGNSYISYWAYWGQGTLV | |
| | | TVSSASTKGPQVQLQESGPGLVKPSETLSLTCTVS | |
| | | GGSVSSGSYYWSWIRQPPGKGLEWIGYIYYSGSTN | |
| | | YNPSLKSRVTISVDTSKNQFSLKLSSVTAADTAVY | |
| | | YCAREGKNGAFDIWGQGTMVTVSSASTKGPSVFPL | |
| | | APSSKSTSGGTAALGCLVKDYFPEPVTVSWNSGAL | |
| | | TSGVHTFPAVLQSSGLYSLSSVVTVPSSSLGTQTY | |
| | | ICNVNHKPSNTKVDKKVEPKSCDKTHTCPPCPAPE | |
| | | AAGGPSVFLFPPKPKDTLMISRTPEVTCVVVDVSH | |
| | | EDPEVKFNWYVDGVEVHNAKTKPREEQYNSTYRVV | |
| | | SVLTVLHQDWLNGKEYKCKVSNKALAAPIEKTISK | |
| | | AKGQPREPQVYTLPPCRDELTKNQVSLWCLVKGFY | |
| | | PSDIAVEWESNGQPENNYKTTPPVLDSDGSFFLYS | |
| | | KLTVDKSRWQQGNVFSCSVMHEALHNHYTQKSLSL | |
| | | SPGK | |
| | HC (Hole) | DIQMTQSPSSLSASVGDRVTITCHASQNIYVWLNW | 308 |
| | αCD28 scFv | YQQKPGKAPKLLIYKASNLHTGVPSRFSGSGSGTD | |
| | αMSLN | FTLTISSLQPEDFATYYCQQGQTYPYTFGCGTKVE | |
| | | IKGGGGSGGGGSGGGGSQVQLVQSGAEVKKPGASV | |
| | | KVSCKASGYTFTSYYIHWVRQAPGQCLEWIGCIYP | |
| | | GNVNTNYNEKFKDRATLTVDTSISTAYMELSRLRS | |
| | | DDTAVYFCTRSHYGLDWNFDVWGQGTTVTVSSAST | |
| | | KGPQVQLQESGPGLVKPSETLSLTCTVSGGSVSSG | |
| | | SYYWSWIRQPPGKGLEWIGYIYYSGSTNYNPSLKS | |
| | | RVTISVDTSKNQFSLKLSSVTAADTAVYYCAREGK | |
| | | NGAFDIWGQGTMVTVSSASTKGPSVFPLAPSSKST | |
| | | SGGTAALGCLVKDYFPEPVTVSWNSGALTSGVHTF | |
| | | PAVLQSSGLYSLSSVVTVPSSSLGTQTYICNVNHK | |
| | | PSNTKVDKKVEPKSCDKTHTCPPCPAPEAAGGPSV | |
| | | FLFPPKPKDTLMISRTPEVTCVVVDVSHEDPEVKF | |
| | | NWYVDGVEVHNAKTKPREEQYNSTYRVVSVLTVLH | |
| | | QDWLNGKEYKCKVSNKALAAPIEKTISKAKGQPRE | |
| | | PQVCTLPPSRDELTKNQVSLSCAVKGFYPSDIAVE | |
| | | WESNGQPENNYKTTPPVLDSDGSFFLVSKLTVDKS | |
| | | RWQQGNVFSCSVMHEALHNHYTQKSLSLSPGK | |
| | LC | DIQMTQSPSSLSASVGDRVTITCRASQSISSYLNW | 306 |
| | αMSLN | YQQKPGKAPKLLIYAASSLQSGVPSGFSGSGSGTD | |
| | | FTLTISSLQPEDFATYYCQQSYSTPLTFGGGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA624 | HC (Knob) | QVQLQESGPGLVKPSETLSLTCTVSGGSVSSGSYY | 309 |
| (M912) | αMSLN | WSWIRQPPGKGLEWIGYIYYSGSTNYNPSLKSRVT | |
| (424 Format) | αCD28 scFv | ISVDTSKNQFSLKLSSVTAADTAVYYCAREGKNGA | |
| | | FDIWGQGTMVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDGGGGSGGGGSQVQLVQSGAEVK | |
| | | KPGASVKVSCKASGYTFTSYYIHWVRQAPGQCLEW | |
| | | IGCIYPGNVNTNYNEKFKDRATLTVDTSISTAYME | |
| | | LSRLRSDDTAVYFCTRSHYGLDWNFDVWGQGTTVT | |
| | | VSSGGGGSGGGGSGGGGSDIQMTQSPSSLSASVGD | |
| | | RVTITCHASQNIYVWLNWYQQKPGKAPKLLIYKAS | |
| | | NLHTGVPSRFSGSGSGTDFTLTISSLQPEDFATYY | |
| | | CQQGQTYPYTFGCGTKVEIKEPKSSDKTHTCPPCP | |
| | | APEAAGGPSVFLFPPKPKDTLMISRTPEVTCVVVD | |
| | | VSHEDPEVKFNWYVDGVEVHNAKTKPREEQYNSTY | |
| | | RVVSVLTVLHQDWLNGKEYKCKVSNKALAAPIEKT | |
| | | ISKAKGQPREPQVYTLPPCRDELTKNQVSLWCLVK | |
| | | GFYPSDIAVEWESNGQPENNYKTTPPVLDSDGSFF | |
| | | LYSKLTVDKSRWQQGNVFSCSVMHEALHNHYTQKS | |
| | | LSLSPGK | |
| | HC (Hole) | QVQLQESGPGLVKPSETLSLTCTVSGGSVSSGSYY | 310 |
| | αMSLN | WSWIRQPPGKGLEWIGYIYYSGSTNYNPSLKSRVT | |
| | αCD3 scFv | ISVDTSKNQFSLKLSSVTAADTAVYYCAREGKNGA | |
| | | FDIWGQGTMVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDGGGGSGGGGSEVQLVESGGGLV | |
| | | QPGGSLKLSCAASGFTFNKYAMNWVRQAPGKCLEW | |
| | | VARIRSKYNNYATYYADSVKDRFTISRDDSKNTAY | |
| | | LQMNNLKTEDTAVYYCVRHGNFGNSYISYWAYWGQ | |
| | | GTLVTVSSGGGGSGGGGSGGGGSQTVVTQEPSLTV | |
| | | SPGGTVTLTCGSSTGAVTSGYYPNWVQQKPGQAPR | |
| | | GLIGGTKFLAPGTPARFSGSLLGGKAALTLSGVQP | |
| | | EDEAEYYCALWYSNRWVFGCGTKLTVLEPKSSDKT | |
| | | HTCPPCPAPEAAGGPSVFLFPPKPKDTLMISRTPE | |
| | | VTCVVVDVSHEDPEVKFNWYVDGVEVHNAKTKPRE | |
| | | EQYNSTYRVVSVLTVLHQDWLNGKEYKCKVSNKAL | |
| | | AAPIEKTISKAKGQPREPQVCTLPPSRDELTKNQV | |
| | | SLSCAVKGFYPSDIAVEWESNGQPENNYKTTPPVL | |
| | | DSDGSFFLVSKLTVDKSRWQQGNVFSCSVMHEALH | |
| | | NHYTQKSLSLSPGK | |
| | LC | DIQMTQSPSSLSASVGDRVTITCRASQSISSYLNW | 306 |
| | αMSLN | YQQKPGKAPKLLIYAASSLQSGVPSGFSGSGSGTD | |
| | | FTLTISSLQPEDFATYYCQQSYSTPLTFGGGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA605 | HC (Knob) | QVQLQQSGPELEKPGASVKISCKASGYSFTGYTMN | 311 |
| (MOR) | αMSLN | WVKQSHGKSLEWIGLITPYNGASSYNQKFRGKATL | |
| (405 Format) | αCD3 scFv | TVDKSSSTAYMDLLSLTSEDSAVYFCARGGYDGRG | |
| | | FDYWGSGTPVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPEAAGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALAAPIEKTISKAKGQPREPQV | |
| | | YTLPPCRDELTKNQVSLWCLVKGFYPSDIAVEWES | |
| | | NGQPENNYKTTPPVLDSDGSFFLYSKLTVDKSRWQ | |
| | | QGNVFSCSVMHEALHNHYTQKSLSLSPGKGGGGSG | |
| | | GGGSGGGGSEVQLVESGGGLVQPGGSLKLSCAASG | |
| | | FTFNKYAMNWVRQAPGKCLEWVARIRSKYNNYATY | |
| | | YADSVKDRFTISRDDSKNTAYLQMNNLKTEDTAVY | |
| | | YCVRHGNFGNSYISYWAYWGQGTLVTVSSGGGGSG | |
| | | GGGSGGGGSQTVVTQEPSLTVSPGGTVTLTCGSST | |
| | | GAVTSGYYPNWVQQKPGQAPRGLIGGTKFLAPGTP | |
| | | ARFSGSLLGGKAALTLSGVQPEDEAEYYCALWYSN | |
| | | RWVFGCGTKLTVL | |
| | HC (Hole) | QVQLQQSGPELEKPGASVKISCKASGYSFTGYTMN | 312 |
| | αMSLN | WVKQSHGKSLEWIGLITPYNGASSYNQKFRGKATL | |
| | αCD28 scFv | TVDKSSSTAYMDLLSLTSEDSAVYFCARGGYDGRG | |
| | | FDYWGSGTPVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPEAAGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALAAPIEKTISKAKGQPREPQV | |
| | | CTLPPSRDELTKNQVSLSCAVKGFYPSDIAVEWES | |
| | | NGQPENNYKTTPPVLDSDGSFFLVSKLTVDKSRWQ | |
| | | QGNVFSCSVMHEALHNHYTQKSLSLSPGKGGGGSG | |
| | | GGGSGGGGSQVQLVQSGAEVKKPGASVKVSCKASG | |
| | | YTFTSYYIHWVRQAPGQCLEWIGCIYPGNVNTNYN | |
| | | EKFKDRATLTVDTSISTAYMELSRLRSDDTAVYFC | |
| | | TRSHYGLDWNFDVWGQGTTVTVSSGGGGSGGGGSG | |
| | | GGGSDIQMTQSPSSLSASVGDRVTITCHASQNIYV | |
| | | WLNWYQQKPGKAPKLLIYKASNLHTGVPSRFSGSG | |
| | | SGTDFTLTISSLQPEDFATYYCQQGQTYPYTFGCG | |
| | | TKVEIK | |
| | LC | DIELTQSPAIMSASPGEKVTMTCSASSSVSYMHWY | 313 |
| | αMSLN | QQKSGTSPKRWIYDTSKLASGVPGRFSGSGSGNSY | |
| | | SLTISSVEAEDDATYYCQQWSKHPLTFGSGTKVEI | |
| | | KRTVAAPSVFIFPPSDEQLKSGTASVVCLLNNFYP | |
| | | REAKVQWKVDNALQSGNSQESVTEQDSKDSTYSLS | |
| | | STLTLSKADYEKHKVYACEVTHQGLSSPVTKSFNR | |
| | | GEC | |
| BCA606 | HC (Knob) | QTVVTQEPSLTVSPGGTVTLTCGSSTGAVTSGYYP | 314 |
| (MOR) | αCD3 scFv | NWVQQKPGQAPRGLIGGTKFLAPGTPARFSGSLLG | |
| (406 Format) | αMSLN | GKAALTLSGVQPEDEAEYYCALWYSNRWVFGCGTK | |
| | | LTVLGGGGSGGGGSGGGGSEVQLVESGGGLVQPGG | |
| | | SLKLSCAASGFTFNKYAMNWVRQAPGKCLEWVARI | |
| | | RSKYNNYATYYADSVKDRFTISRDDSKNTAYLQMN | |
| | | NLKTEDTAVYYCVRHGNFGNSYISYWAYWGQGTLV | |
| | | TVSSASTKGPQVQLQQSGPELEKPGASVKISCKAS | |
| | | GYSFTGYTMNWVKQSHGKSLEWIGLITPYNGASSY | |
| | | NQKFRGKATLTVDKSSSTAYMDLLSLTSEDSAVYF | |
| | | CARGGYDGRGFDYWGSGTPVTVSSASTKGPSVFPL | |
| | | APSSKSTSGGTAALGCLVKDYFPEPVTVSWNSGAL | |
| | | TSGVHTFPAVLQSSGLYSLSSVVTVPSSSLGTQTY | |
| | | ICNVNHKPSNTKVDKKVEPKSCDKTHTCPPCPAPE | |
| | | AAGGPSVFLFPPKPKDTLMISRTPEVTCVVVDVSH | |
| | | EDPEVKFNWYVDGVEVHNAKTKPREEQYNSTYRVV | |
| | | SVLTVLHQDWLNGKEYKCKVSNKALAAPIEKTISK | |
| | | AKGQPREPQVYTLPPCRDELTKNQVSLWCLVKGFY | |
| | | PSDIAVEWESNGQPENNYKTTPPVLDSDGSFFLYS | |
| | | KLTVDKSRWQQGNVFSCSVMHEALHNHYTQKSLSL | |
| | | SPGK | |
| | HC (Hole) | DIQMTQSPSSLSASVGDRVTITCHASQNIYVWLNW | 315 |
| | αCD28 scFv | YQQKPGKAPKLLIYKASNLHTGVPSRFSGSGSGTD | |
| | αMSLN | FTLTISSLQPEDFATYYCQQGQTYPYTFGCGTKVE | |
| | | IKGGGGSGGGGSGGGGSQVQLVQSGAEVKKPGASV | |
| | | KVSCKASGYTFTSYYIHWVRQAPGQCLEWIGCIYP | |
| | | GNVNTNYNEKFKDRATLTVDTSISTAYMELSRLRS | |
| | | DDTAVYFCTRSHYGLDWNFDVWGQGTTVTVSSAST | |
| | | KGPQVQLQQSGPELEKPGASVKISCKASGYSFTGY | |
| | | TMNWVKQSHGKSLEWIGLITPYNGASSYNQKFRGK | |
| | | ATLTVDKSSSTAYMDLLSLTSEDSAVYFCARGGYD | |
| | | GRGFDYWGSGTPVTVSSASTKGPSVFPLAPSSKST | |
| | | SGGTAALGCLVKDYFPEPVTVSWNSGALTSGVHTF | |
| | | PAVLQSSGLYSLSSVVTVPSSSLGTQTYICNVNHK | |
| | | PSNTKVDKKVEPKSCDKTHTCPPCPAPEAAGGPSV | |
| | | FLFPPKPKDTLMISRTPEVTCVVVDVSHEDPEVKF | |
| | | NWYVDGVEVHNAKTKPREEQYNSTYRVVSVLTVLH | |
| | | QDWLNGKEYKCKVSNKALAAPIEKTISKAKGQPRE | |
| | | PQVCTLPPSRDELTKNQVSLSCAVKGFYPSDIAVE | |
| | | WESNGQPENNYKTTPPVLDSDGSFFLVSKLTVDKS | |
| | | RWQQGNVFSCSVMHEALHNHYTQKSLSLSPGK | |
| | LC | DIELTQSPAIMSASPGEKVTMTCSASSSVSYMHWY | 313 |
| | αMSLN | QQKSGTSPKRWIYDTSKLASGVPGRFSGSGSGNSY | |
| | | SLTISSVEAEDDATYYCQQWSKHPLTFGSGTKVEI | |
| | | KRTVAAPSVFIFPPSDEQLKSGTASVVCLLNNFYP | |
| | | REAKVQWKVDNALQSGNSQESVTEQDSKDSTYSLS | |
| | | STLTLSKADYEKHKVYACEVTHQGLSSPVTKSFNR | |
| | | GEC | |
| BCA624 | HC (Knob) | QVQLQQSGPELEKPGASVKISCKASGYSFTGYTMN | 316 |
| (MOR) | αMSLN | WVKQSHGKSLEWIGLITPYNGASSYNQKFRGKATL | |
| (424 Format) | αCD28 scFv | TVDKSSSTAYMDLLSLTSEDSAVYFCARGGYDGRG | |
| | | FDYWGSGTPVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDGGGGSGGGGSQVQLVQSGAEVK | |
| | | KPGASVKVSCKASGYTFTSYYIHWVRQAPGQCLEW | |
| | | IGCIYPGNVNTNYNEKFKDRATLTVDTSISTAYME | |
| | | LSRLRSDDTAVYFCTRSHYGLDWNFDVWGQGTTVT | |
| | | VSSGGGGSGGGGSGGGGSDIQMTQSPSSLSASVGD | |
| | | RVTITCHASQNIYVWLNWYQQKPGKAPKLLIYKAS | |
| | | NLHTGVPSRFSGSGSGTDFTLTISSLQPEDFATYY | |
| | | CQQGQTYPYTFGCGTKVEIKEPKSSDKTHTCPPCP | |
| | | APEAAGGPSVFLFPPKPKDTLMISRTPEVTCVVVD | |
| | | VSHEDPEVKFNWYVDGVEVHNAKTKPREEQYNSTY | |
| | | RVVSVLTVLHQDWLNGKEYKCKVSNKALAAPIEKT | |
| | | ISKAKGQPREPQVYTLPPCRDELTKNQVSLWCLVK | |
| | | GFYPSDIAVEWESNGQPENNYKTTPPVLDSDGSFF | |
| | | LYSKLTVDKSRWQQGNVFSCSVMHEALHNHYTQKS | |
| | | LSLSPGK | |
| | HC (Hole) | QVQLQQSGPELEKPGASVKISCKASGYSFTGYTMN | 317 |
| | αMSLN | WVKQSHGKSLEWIGLITPYNGASSYNQKFRGKATL | |
| | αCD3 scFv | TVDKSSSTAYMDLLSLTSEDSAVYFCARGGYDGRG | |
| | | FDYWGSGTPVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDGGGGSGGGGSEVQLVESGGGLV | |
| | | QPGGSLKLSCAASGFTFNKYAMNWVRQAPGKCLEW | |
| | | VARIRSKYNNYATYYADSVKDRFTISRDDSKNTAY | |
| | | LQMNNLKTEDTAVYYCVRHGNFGNSYISYWAYWGQ | |
| | | GTLVTVSSGGGGSGGGGSGGGGSQTVVTQEPSLTV | |
| | | SPGGTVTLTCGSSTGAVTSGYYPNWVQQKPGQAPR | |
| | | GLIGGTKFLAPGTPARFSGSLLGGKAALTLSGVQP | |
| | | EDEAEYYCALWYSNRWVFGCGTKLTVLEPKSSDKT | |
| | | HTCPPCPAPEAAGGPSVFLFPPKPKDTLMISRTPE | |
| | | VTCVVVDVSHEDPEVKFNWYVDGVEVHNAKTKPRE | |
| | | EQYNSTYRVVSVLTVLHQDWLNGKEYKCKVSNKAL | |
| | | AAPIEKTISKAKGQPREPQVCTLPPSRDELTKNQV | |
| | | SLSCAVKGFYPSDIAVEWESNGQPENNYKTTPPVL | |
| | | DSDGSFFLVSKLTVDKSRWQQGNVFSCSVMHEALH | |
| | | NHYTQKSLSLSPGK | |
| | LC | DIELTQSPAIMSASPGEKVTMTCSASSSVSYMHWY | 313 |
| | αMSLN | QQKSGTSPKRWIYDTSKLASGVPGRFSGSGSGNSY | |
| | | SLTISSVEAEDDATYYCQQWSKHPLTFGSGTKVEI | |
| | | KRTVAAPSVFIFPPSDEQLKSGTASVVCLLNNFYP | |
| | | REAKVQWKVDNALQSGNSQESVTEQDSKDSTYSLS | |
| | | STLTLSKADYEKHKVYACEVTHQGLSSPVTKSFNR | |
| | | GEC | |
| BCA429 | HC | QVQLQQSGPELEKPGASVKISCKASGYSFTGYTMN | 417 |
| (MOR) | | WVKQSHGKSLEWIGLITPYNGASSYNQKFRGKATL | |
| | | TVDKSSSTAYMDLLSLTSEDSAVYFCARGGYDGRG | |
| | | FDYWGSGTPVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPEAAGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALAAPIEKTISKAKGQPREPQV | |
| | | YTLPPSRDELTKNQVSLTCLVKGFYPSDIAVEWES | |
| | | NGQPENNYKTTPPVLDSDGSFFLYSKLTVDKSRWQ | |
| | | QGNVFSCSVMHEALHNHYTQKSLSLSPGK | |
| BCA605 | HC (Knob) | EVQLQQSGPVLVKPGASVKISCKASGYSFTGYYMH | 320 |
| (FB6) | αMSLN | WVRQSLVKRLEWIGRINPYTGVPSYKHNFKDKASL | |
| (405 Format) | αCD3 scFv | TVDKSSSTAYMELHSLTSEDSAVYYCARELGGYWG | |
| | | QGTTLTVSSASTKGPSVFPLAPSSKSTSGGTAALG | |
| | | CLVKDYFPEPVTVSWNSGALTSGVHTFPAVLQSSG | |
| | | LYSLSSVVTVPSSSLGTQTYICNVNHKPSNTKVDK | |
| | | KVEPKSCDKTHTCPPCPAPEAAGGPSVFLFPPKPK | |
| | | DTLMISRTPEVTCVVVDVSHEDPEVKFNWYVDGVE | |
| | | VHNAKTKPREEQYNSTYRVVSVLTVLHQDWLNGKE | |
| | | YKCKVSNKALAAPIEKTISKAKGQPREPQVYTLPP | |
| | | CRDELTKNQVSLWCLVKGFYPSDIAVEWESNGQPE | |
| | | NNYKTTPPVLDSDGSFFLYSKLTVDKSRWQQGNVF | |
| | | SCSVMHEALHNHYTQKSLSLSPGKGGGGSGGGGSG | |
| | | GGGSEVQLVESGGGLVQPGGSLKLSCAASGFTFNK | |
| | | YAMNWVRQAPGKCLEWVARIRSKYNNYATYYADSV | |
| | | KDRFTISRDDSKNTAYLQMNNLKTEDTAVYYCVRH | |
| | | GNFGNSYISYWAYWGQGTLVTVSSGGGGSGGGGSG | |
| | | GGGSQTVVTQEPSLTVSPGGTVTLTCGSSTGAVTS | |
| | | GYYPNWVQQKPGQAPRGLIGGTKFLAPGTPARFSG | |
| | | SLLGGKAALTLSGVQPEDEAEYYCALWYSNRWVFG | |
| | | CGTKLTVL | |
| | HC (Hole) | EVQLQQSGPVLVKPGASVKISCKASGYSFTGYYMH | 321 |
| | αMSLN | WVRQSLVKRLEWIGRINPYTGVPSYKHNFKDKASL | |
| | αCD28 scFv | TVDKSSSTAYMELHSLTSEDSAVYYCARELGGYWG | |
| | | QGTTLTVSSASTKGPSVFPLAPSSKSTSGGTAALG | |
| | | CLVKDYFPEPVTVSWNSGALTSGVHTFPAVLQSSG | |
| | | LYSLSSVVTVPSSSLGTQTYICNVNHKPSNTKVDK | |
| | | KVEPKSCDKTHTCPPCPAPEAAGGPSVFLFPPKPK | |
| | | DTLMISRTPEVTCVVVDVSHEDPEVKFNWYVDGVE | |
| | | VHNAKTKPREEQYNSTYRVVSVLTVLHQDWLNGKE | |
| | | YKCKVSNKALAAPIEKTISKAKGQPREPQVCTLPP | |
| | | SRDELTKNQVSLSCAVKGFYPSDIAVEWESNGQPE | |
| | | NNYKTTPPVLDSDGSFFLVSKLTVDKSRWQQGNVF | |
| | | SCSVMHEALHNHYTQKSLSLSPGKGGGGSGGGGSG | |
| | | GGGSQVQLVQSGAEVKKPGASVKVSCKASGYTFTS | |
| | | YYIHWVRQAPGQCLEWIGCIYPGNVNTNYNEKFKD | |
| | | RATLTVDTSISTAYMELSRLRSDDTAVYFCTRSHY | |
| | | GLDWNFDVWGQGTTVTVSSGGGGSGGGGSGGGGSD | |
| | | IQMTQSPSSLSASVGDRVTITCHASQNIYVWLNWY | |
| | | QQKPGKAPKLLIYKASNLHTGVPSRFSGSGSGTDF | |
| | | TLTISSLQPEDFATYYCQQGQTYPYTFGCGTKVEI | |
| | | K | |
| | LC | QAVVTQESALTTSPGETVTLTCRSSTGAVTTGNYP | 322 |
| | αMSLN | NWVQEKPDHLFTGLIAGTNNRAPGVPARFSGSLIG | |
| | | DKAALTITGAQTEDEAIYFCALWFSSHWVFGGGTK | |
| | | LTVLRTVAAPSVFIFPPSDEQLKSGTASVVCLLNN | |
| | | FYPREAKVQWKVDNALQSGNSQESVTEQDSKDSTY | |
| | | SLSSTLTLSKADYEKHKVYACEVTHQGLSSPVTKS | |
| | | FNRGEC | |
| BCA606 | HC (Knob) | QTVVTQEPSLTVSPGGTVTLTCGSSTGAVTSGYYP | 323 |
| (FB6) | αCD3 scFv | NWVQQKPGQAPRGLIGGTKFLAPGTPARFSGSLLG | |
| (406 Format) | αMSLN | GKAALTLSGVQPEDEAEYYCALWYSNRWVFGCGTK | |
| | | LTVLGGGGSGGGGSGGGGSEVQLVESGGGLVQPGG | |
| | | SLKLSCAASGFTFNKYAMNWVRQAPGKCLEWVARI | |
| | | RSKYNNYATYYADSVKDRFTISRDDSKNTAYLQMN | |
| | | NLKTEDTAVYYCVRHGNFGNSYISYWAYWGQGTLV | |
| | | TVSSASTKGPEVQLQQSGPVLVKPGASVKISCKAS | |
| | | GYSFTGYYMHWVRQSLVKRLEWIGRINPYTGVPSY | |
| | | KHNFKDKASLTVDKSSSTAYMELHSLTSEDSAVYY | |
| | | CARELGGYWGQGTTLTVSSASTKGPSVFPLAPSSK | |
| | | STSGGTAALGCLVKDYFPEPVTVSWNSGALTSGVH | |
| | | TFPAVLQSSGLYSLSSVVTVPSSSLGTQTYICNVN | |
| | | HKPSNTKVDKKVEPKSCDKTHTCPPCPAPEAAGGP | |
| | | SVFLFPPKPKDTLMISRTPEVTCVVVDVSHEDPEV | |
| | | KFNWYVDGVEVHNAKTKPREEQYNSTYRVVSVLTV | |
| | | LHQDWLNGKEYKCKVSNKALAAPIEKTISKAKGQP | |
| | | REPQVYTLPPCRDELTKNQVSLWCLVKGFYPSDIA | |
| | | VEWESNGQPENNYKTTPPVLDSDGSFFLYSKLTVD | |
| | | KSRWQQGNVFSCSVMHEALHNHYTQKSLSLSPGK | |
| | HC (Hole) | DIQMTQSPSSLSASVGDRVTITCHASQNIYVWLNW | 324 |
| | αCD28 scFv | YQQKPGKAPKLLIYKASNLHTGVPSRFSGSGSGTD | |
| | αMSLN | FTLTISSLQPEDFATYYCQQGQTYPYTFGCGTKVE | |
| | | IKGGGGSGGGGSGGGGSQVQLVQSGAEVKKPGASV | |
| | | KVSCKASGYTFTSYYIHWVRQAPGQCLEWIGCIYP | |
| | | GNVNTNYNEKFKDRATLTVDTSISTAYMELSRLRS | |
| | | DDTAVYFCTRSHYGLDWNFDVWGQGTTVTVSSAST | |
| | | KGPEVQLQQSGPVLVKPGASVKISCKASGYSFTGY | |
| | | YMHWVRQSLVKRLEWIGRINPYTGVPSYKHNFKDK | |
| | | ASLTVDKSSSTAYMELHSLTSEDSAVYYCARELGG | |
| | | YWGQGTTLTVSSASTKGPSVFPLAPSSKSTSGGTA | |
| | | ALGCLVKDYFPEPVTVSWNSGALTSGVHTFPAVLQ | |
| | | SSGLYSLSSVVTVPSSSLGTQTYICNVNHKPSNTK | |
| | | VDKKVEPKSCDKTHTCPPCPAPEAAGGPSVFLFPP | |
| | | KPKDTLMISRTPEVTCVVVDVSHEDPEVKFNWYVD | |
| | | GVEVHNAKTKPREEQYNSTYRVVSVLTVLHQDWLN | |
| | | GKEYKCKVSNKALAAPIEKTISKAKGQPREPQVCT | |
| | | LPPSRDELTKNQVSLSCAVKGFYPSDIAVEWESNG | |
| | | QPENNYKTTPPVLDSDGSFFLVSKLTVDKSRWQQG | |
| | | NVFSCSVMHEALHNHYTQKSLSLSPGK | |
| | LC | QAVVTQESALTTSPGETVTLTCRSSTGAVTTGNYP | 322 |
| | αMSLN | NWVQEKPDHLFTGLIAGTNNRAPGVPARFSGSLIG | |
| | | DKAALTITGAQTEDEAIYFCALWFSSHWVFGGGTK | |
| | | LTVLRTVAAPSVFIFPPSDEQLKSGTASVVCLLNN | |
| | | FYPREAKVQWKVDNALQSGNSQESVTEQDSKDSTY | |
| | | SLSSTLTLSKADYEKHKVYACEVTHQGLSSPVTKS | |
| | | FNRGEC | |
| BCA624 | HC (Knob) | EVQLQQSGPVLVKPGASVKISCKASGYSFTGYYMH | 325 |
| (FB6) | αMSLN | WVRQSLVKRLEWIGRINPYTGVPSYKHNFKDKASL | |
| (424 Format) | αCD28 scFv | TVDKSSSTAYMELHSLTSEDSAVYYCARELGGYWG | |
| | | QGTTLTVSSASTKGPSVFPLAPSSKSTSGGTAALG | |
| | | CLVKDYFPEPVTVSWNSGALTSGVHTFPAVLQSSG | |
| | | LYSLSSVVTVPSSSLGTQTYICNVNHKPSNTKVDK | |
| | | KVEPKSCDGGGGSGGGGSQVQLVQSGAEVKKPGAS | |
| | | VKVSCKASGYTFTSYYIHWVRQAPGQCLEWIGCIY | |
| | | PGNVNTNYNEKFKDRATLTVDTSISTAYMELSRLR | |
| | | SDDTAVYFCTRSHYGLDWNFDVWGQGTTVTVSSGG | |
| | | GGSGGGGSGGGGSDIQMTQSPSSLSASVGDRVTIT | |
| | | CHASQNIYVWLNWYQQKPGKAPKLLIYKASNLHTG | |
| | | VPSRFSGSGSGTDFTLTISSLQPEDFATYYCQQGQ | |
| | | TYPYTFGCGTKVEIKEPKSSDKTHTCPPCPAPEAA | |
| | | GGPSVFLFPPKPKDTLMISRTPEVTCVVVDVSHED | |
| | | PEVKFNWYVDGVEVHNAKTKPREEQYNSTYRVVSV | |
| | | LTVLHQDWLNGKEYKCKVSNKALAAPIEKTISKAK | |
| | | GQPREPQVYTLPPCRDELTKNQVSLWCLVKGFYPS | |
| | | DIAVEWESNGQPENNYKTTPPVLDSDGSFFLYSKL | |
| | | TVDKSRWQQGNVFSCSVMHEALHNHYTQKSLSLSP | |
| | | GK | |
| | HC (Hole) | EVQLQQSGPVLVKPGASVKISCKASGYSFTGYYMH | 326 |
| | αMSLN | WVRQSLVKRLEWIGRINPYTGVPSYKHNFKDKASL | |
| | αCD3 scFv | TVDKSSSTAYMELHSLTSEDSAVYYCARELGGYWG | |
| | | QGTTLTVSSASTKGPSVFPLAPSSKSTSGGTAALG | |
| | | CLVKDYFPEPVTVSWNSGALTSGVHTFPAVLQSSG | |
| | | LYSLSSVVTVPSSSLGTQTYICNVNHKPSNTKVDK | |
| | | KVEPKSCDGGGGSGGGGSEVQLVESGGGLVQPGGS | |
| | | LKLSCAASGFTFNKYAMNWVRQAPGKCLEWVARIR | |
| | | SKYNNYATYYADSVKDRFTISRDDSKNTAYLQMNN | |
| | | LKTEDTAVYYCVRHGNFGNSYISYWAYWGQGTLVT | |
| | | VSSGGGGSGGGGSGGGGSQTVVTQEPSLTVSPGGT | |
| | | VTLTCGSSTGAVTSGYYPNWVQQKPGQAPRGLIGG | |
| | | TKFLAPGTPARFSGSLLGGKAALTLSGVQPEDEAE | |
| | | YYCALWYSNRWVFGCGTKLTVLEPKSSDKTHTCPP | |
| | | CPAPEAAGGPSVFLFPPKPKDTLMISRTPEVTCVV | |
| | | VDVSHEDPEVKFNWYVDGVEVHNAKTKPREEQYNS | |
| | | TYRVVSVLTVLHQDWLNGKEYKCKVSNKALAAPIE | |
| | | KTISKAKGQPREPQVCTLPPSRDELTKNQVSLSCA | |
| | | VKGFYPSDIAVEWESNGQPENNYKTTPPVLDSDGS | |
| | | FFLVSKLTVDKSRWQQGNVFSCSVMHEALHNHYTQ | |
| | | KSLSLSPGK | |
| | LC | QAVVTQESALTTSPGETVTLTCRSSTGAVTTGNYP | 322 |
| | αMSLN | NWVQEKPDHLFTGLIAGTNNRAPGVPARFSGSLIG | |
| | | DKAALTITGAQTEDEAIYFCALWFSSHWVFGGGTK | |
| | | LTVLRTVAAPSVFIFPPSDEQLKSGTASVVCLLNN | |
| | | FYPREAKVQWKVDNALQSGNSQESVTEQDSKDSTY | |
| | | SLSSTLTLSKADYEKHKVYACEVTHQGLSSPVTKS | |
| | | FNRGEC | |
| BCA401 | HC | EVQLVESGGGLVQPGGSLRLSCAASGFNIKDTYIH | 261 |
| (αHER2) | | WVRQAPGKGLEWVARIYPTNGYTRYADSVKGRFTI | |
| (Control) | | SADTSKNTAYLQMNSLRAEDTAVYYCSRWGGDGFY | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPELLGGPSVFL | |
| | | FPPKPKDTLMISRTPEVTCVVVDVSHEDPEVKFNW | |
| | | YVDGVEVHNAKTKPREEQYNSTYRVVSVLTVLHQD | |
| | | WLNGKEYKCKVSNKALPAPIEKTISKAKGQPREPQ | |
| | | VYTLPPSREEMTKNQVSLTCLVKGFYPSDIAVEWE | |
| | | SNGQPENNYKTTPPVLDSDGSFFLYSKLTVDKSRW | |
| | | QQGNVFSCSVMHEALHNHYTQKSLSLSPGK | |
| | LC | DIQMTQSPSSLSASVGDRVTITCRASQDVNTAVAW | 239 |
| | | YQQKPGKAPKLLIYSASFLYSGVPSRFSGSRSGTD | |
| | | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA402 | HC | QVQLVQSGAEVKKPGASVKVSCKASGYTFTSYYIH | 262 |
| (αCD28 IgG1) | | WVRQAPGQGLEWIGCIYPGNVNTNYNEKFKDRATL | |
| (Control) | | TVDTSISTAYMELSRLRSDDTAVYFCTRSHYGLDW | |
| | | NFDVWGQGTTVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPELLGGPSVFL | |
| | | FPPKPKDTLMISRTPEVTCVVVDVSHEDPEVKFNW | |
| | | YVDGVEVHNAKTKPREEQYNSTYRVVSVLTVLHQD | |
| | | WLNGKEYKCKVSNKALPAPIEKTISKAKGQPREPQ | |
| | | VYTLPPSREEMTKNQVSLTCLVKGFYPSDIAVEWE | |
| | | SNGQPENNYKTTPPVLDSDGSFFLYSKLTVDKSRW | |
| | | QQGNVFSCSVMHEALHNHYTQKSLSLSPGK | |
| | LC | DIQMTQSPSSLSASVGDRVTITCHASQNIYVWLNW | 263 |
| | | YQQKPGKAPKLLIYKASNLHTGVPSRFSGSGSGTD | |
| | | FTLTISSLQPEDFATYYCQQGQTYPYTFGGGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA402 | HC | QVQLVQSGAEVKKPGASVKVSCKASGYTFTSYYIH | 264 |
| (αCD28 IgG4) | | WVRQAPGQGLEWIGCIYPGNVNTNYNEKFKDRATL | |
| (Control) | | TVDTSISTAYMELSRLRSDDTAVYFCTRSHYGLDW | |
| | | NFDVWGQGTTVTVSSASTKGPSVFPLAPCSRSTSE | |
| | | STAALGCLVKDYFPEPVTVSWNSGALTSGVHTFPA | |
| | | VLQSSGLYSLSSVVTVPSSSLGTKTYTCNVDHKPS | |
| | | NTKVDKRVESKYGPPCPSCPAPEFLGGPSVFLFPP | |
| | | KPKDTLMISRTPEVTCVVVDVSQEDPEVQFNWYVD | |
| | | GVEVHNAKTKPREEQFNSTYRVVSVLTVLHQDWLN | |
| | | GKEYKCKVSNKGLPSSIEKTISKAKGQPREPQVYT | |
| | | LPPSQEEMTKNQVSLTCLVKGFYPSDIAVEWESNG | |
| | | QPENNYKTTPPVLDSDGSFFLYSRLTVDKSRWQEG | |
| | | NVFSCSVMHEALHNHYTQKSLSLSLG | |
| | LC | DIQMTQSPSSLSASVGDRVTITCHASQNIYVWLNW | 265 |
| | | YQQKPGKAPKLLIYKASNLHTGVPSRFSGSGSGTD | |
| | | FTLTISSLQPEDFATYYCQQGQTYPYTFGGGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA403 | HC | EVQLVESGGGLVQPGGSLKLSCAASGFTFNKYAMN | 266 |
| (αCD3ε) | | WVRQAPGKGLEWVARIRSKYNNYATYYADSVKDRF | |
| (Control) | | TISRDDSKNTAYLQMNNLKTEDTAVYYCVRHGNFG | |
| | | NSYISYWAYWGQGTLVTVSSASTKGPSVFPLAPSS | |
| | | KSTSGGTAALGCLVKDYFPEPVTVSWNSGALTSGV | |
| | | HTFPAVLQSSGLYSLSSVVTVPSSSLGTQTYICNV | |
| | | NHKPSNTKVDKKVEPKSCDKTHTCPPCPAPELLGG | |
| | | PSVFLFPPKPKDTLMISRTPEVTCVVVDVSHEDPE | |
| | | VKFNWYVDGVEVHNAKTKPREEQYNSTYRVVSVLT | |
| | | VLHQDWLNGKEYKCKVSNKALPAPIEKTISKAKGQ | |
| | | PREPQVYTLPPSREEMTKNQVSLTCLVKGFYPSDI | |
| | | AVEWESNGQPENNYKTTPPVLDSDGSFFLYSKLTV | |
| | | DKSRWQQGNVFSCSVMHEALHNHYTQKSLSLSPGK | |
| | LC | QTVVTQEPSLTVSPGGTVTLTCGSSTGAVTSGYYP | 267 |
| | | NWVQQKPGQAPRGLIGGTKFLAPGTPARFSGSLLG | |
| | | GKAALTLSGVQPEDEAEYYCALWYSNRWVFGGGTK | |
| | | LTVLRTVAAPSVFIFPPSDEQLKSGTASVVCLLNN | |
| | | FYPREAKVQWKVDNALQSGNSQESVTEQDSKDSTY | |
| | | SLSSTLTLSKADYEKHKVYACEVTHQGLSSPVTKS | |
| | | FNRGEC | |
| Cetuximab | HC | QVQLKQSGPGLVQPSQSLSITCTVSGFSLINYGVH | 268 |
| (αEGFR | | WVRQSPGKGLEWLGVIWSGGNTDYNTPFTSRLSIN | |
| Control) | | KDNSKSQVFFKMNSLQSNDTAIYYCARALTYYDYE | |
| | | FAYWGQGTLVTVSAASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSN | |
| | | TKVDKKVEPKSCDKTHTCPPCPAPELLGGPSVFLF | |
| | | PPKPKDTLMISRTPEVTCVVVDVSHEDPEVKFNWY | |
| | | VDGVEVHNAKTKPREEQYNSTYRVVSVLTVLHQDW | |
| | | LNGKEYKCKVSNKALPAPIEKTISKAKGQPREPQV | |
| | | YTLPPSREEMTKNQVSLTCLVKGFYPSDIAVEWES | |
| | | NGQPENNYKTTPPVLDSDGSFFLYSKLTVDKSRWQ | |
| | | QGNVFSCSVMHEALHNHYTQKSLSLSPGK | |
| | LC | DILLTQSPVILSVSPGERVSFSCRASQSIGTNIHW | 256 |
| | | YQQRINGSPRLLIKYASESISGIPSRFSGSGSGTD | |
| | | FTLSINSVESEDIADYYCQQNNNWPTTFGAGTKLE | |
| | | LKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| BCA429 | HC | QVQLQQSGPELEKPGASVKISCKASGYSFTGY | 417 |
| (α MSLN | | TMNWVKQSHGKSLEWIGLITPYNGASSYNQKF | |
| Control) | | RGKATLTVDKSSSTAYMDLLSLTSEDSAVYFC | |
| | | ARGGYDGRGFDYWGSGTPVTVSSASTKGPSVF | |
| | | PLAPSSKSTSGGTAALGCLVKDYFPEPVTVSW | |
| | | NSGALTSGVHTFPAVLQSSGLYSLSSVVTVPS | |
| | | SSLGTQTYICNVNHKPSNTKVDKKVEPKSCDK | |
| | | THTCPPCPAPEAAGGPSVFLFPPKPKDTLMIS | |
| | | RTPEVTCVVVDVSHEDPEVKFNWYVDGVEVHN | |
| | | AKTKPREEQYNSTYRVVSVLTVLHQDWLNGKE | |
| | | YKCKVSNKALAAPIEKTISKAKGQPREPQVYT | |
| | | LPPSRDELTKNQVSLTCLVKGFYPSDIAVEWE | |
| | | SNGQPENNYKTTPPVLDSDGSFFLYSKLTVDK | |
| | | SRWQQGNVFSCSVMHEALHNHYTQKSLSLSPG | |
| | | K | |
| | LC | DIELTQSPAIMSASPGEKVTMTCSASSSVSYMHWY | 418 |
| | | QQKSGTSPKRWIYDTSKLASGVPGRFSGSGSGNSY | |
| | | SLTISSVEAEDDATYYCQQWSKHPLTFGSGTKVEI | |
| | | KRTVAAPSVFIFPPSDEQLKSGTASVVCLLNNFYP | |
| | | REAKVQWKVDNALQSGNSQESVTEQDSKDSTYSLS | |
| | | STLTLSKADYEKHKVYACEVTHQGLSSPVTKSFNR | |
| | | GEC | |
| BCA410 | HC (Knob) | EVQLVESGGGLVQPGGSLRLSCAASGFNIKDTYIH | 269 |
| | αHER2 | WVRQAPGKGLEWVARIYPTNGYTRYADSVKGRFTI | |
| | | SADTSKNTAYLQMNSLRAEDTAVYYCSRWGGDGFY | |
| | | AMDYWGQGTLVTVSSASTKGPSVFPLAPCSRSTSE | |
| | | STAALGCLVKDYFPEPVTVSWNSGALTSGVHTFPA | |
| | | VLQSSGLYSLSSVVTVPSSSLGTKTYTCNVDHKPS | |
| | | NTKVDKRVESKYGPPCPPCPAPEFLGGPSVFLFPP | |
| | | KPKDTLMISRTPEVTCVVVDVSQEDPEVQFNWYVD | |
| | | GVEVHNAKTKPREEQFNSTYRVVSVLTVLHQDWLN | |
| | | GKEYKCKVSNKGLPSSIEKTISKAKGQPREPQVYT | |
| | | LPPCQEEMTKNQVSLWCLVKGFYPSDIAVEWESNG | |
| | | QPENNYKTTPPVLDSDGSFFLYSKLTVDKSRWQEG | |
| | | NVFSCSVMHEALHNHYTQKSLSLSLGK | |
| | HC (Hole) | QVQLQESGPGLVKPSQTLSLTCTVSGFSLSDYGVH | 270 |
| | αCD3 | WVRQPPGKGLEWLGVIWAGGGTNYNPSLKSRKTIS | |
| | αCD28 | KDTSKNQVSLKLSSVTAADTAVYYCARDKGYSYYY | |
| | | SMDYWGQGTTVTVSSSQVQLVESGGGVVQPGRSLR | |
| | | LSCAASGFTFTKAWMHWVRQAPGKQLEWVAQIKDK | |
| | | SNSYATYYADSVKGRFTISRDDSKNTLYLQMNSLR | |
| | | AEDTAVYYCRGVYYALSPFDYWGQGTLVTVSSRTA | |
| | | STKGPSVFPLAPCSRSTSESTAALGCLVKDYFPEP | |
| | | VTVSWNSGALTSGVHTFPAVLQSSGLYSLSSVVTV | |
| | | PSSSLGTKTYTCNVDHKPSNTKVDKRVESKYGPPC | |
| | | PPCPAPEFLGGPSVFLFPPKPKDTLMISRTPEVTC | |
| | | VVVDVSQEDPEVQFNWYVDGVEVHNAKTKPREEQF | |
| | | NSTYRVVSVLTVLHQDWLNGKEYKCKVSNKGLPSS | |
| | | IEKTISKAKGQPREPQVCTLPPSQEEMTKNQVSLS | |
| | | CAVKGFYPSDIAVEWESNGQPENNYKTTPPVLDSD | |
| | | GSFFLVSKLTVDKSRWQEGNVFSCSVMHEALHNHY | |
| | | TQKSLSLSLGK | |
| | LC | DIQMTQSPSSLSASVGDRVTITCRASQDVNTAVAW | 239 |
| | αHER2 | YQQKPGKAPKLLIYSASFLYSGVPSRFSGSRSGTD | |
| | | FTLTISSLQPEDFATYYCQQHYTTPPTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| | LC | DIVMTQTPLSLSVTPGQPASISCKSSQSLVHNNAN | 271 |
| | αCD28 | TYLSWYLQKPGQSPQSLIYKVSNRFSGVPDRFSGS | |
| | αCD3 | GSGTDFTLKISRVEAEDVGVYYCGQGTQYPFTFGS | |
| | | GTKVEIKGQPKAAPDIVLTQSPASLAVSPGQRATI | |
| | | TCRASESVEYYVTSLMQWYQQKPGQPPKLLIFAAS | |
| | | NVESGVPARFSGSGSGTDFTLTINPVEANDVANYY | |
| | | CQQSRKVPYTFGQGTKLEIKTKGPSRTVAAPSVFI | |
| | | FPPSDEQLKSGTASVVCLLNNFYPREAKVQWKVDN | |
| | | ALQSGNSQESVTEQDSKDSTYSLSSTLTLSKADYE | |
| | | KHKVYACEVTHQGLSSPVTKSFNRGEC | |

### 6.2 Example 2. TAA Binding of Multispecific Proteins

Binding of the multispecific proteins to HER2 was assessed by ELISA. Briefly, 96-well Maxi-Sorp plates (Nunc) were coated with HER2 extracellular domain (Acrobiosystem) at 4° C. overnight. The following day, the plates were washed with wash buffer (0.05% Tween-20 (Sigma-Aldrich) in filtered DPBS (Gibco) and blocked using SuperBlock™ blocking buffer (Thermo Fisher) for 1.5-2 hours. The plates were then incubated with serially diluted primary antibody [multispecific proteins and human isotype control antibody, hIgG (Bioxcell)] made in Assay diluent (wash Buffer containing 10% SuperBlock™) for 1 hour at room temperature (RT). This was followed by a wash with wash buffer and subsequent incubation with secondary antibody (Donkey anti-human IgG H+L peroxidase (Jackson ImmunoResearch) diluted 1:25000 in assay diluent) for 1 hour at RT. After incubation, the plates were washed with wash buffer. The colour was developed by adding TMB substrate solution (Sigma-Aldrich) and the reaction was stopped using Stop Solution (1N H₂SO₄). The optical density of each well was determined immediately, using a microplate reader at 450 nm with 630 nm as the reference wavelength.

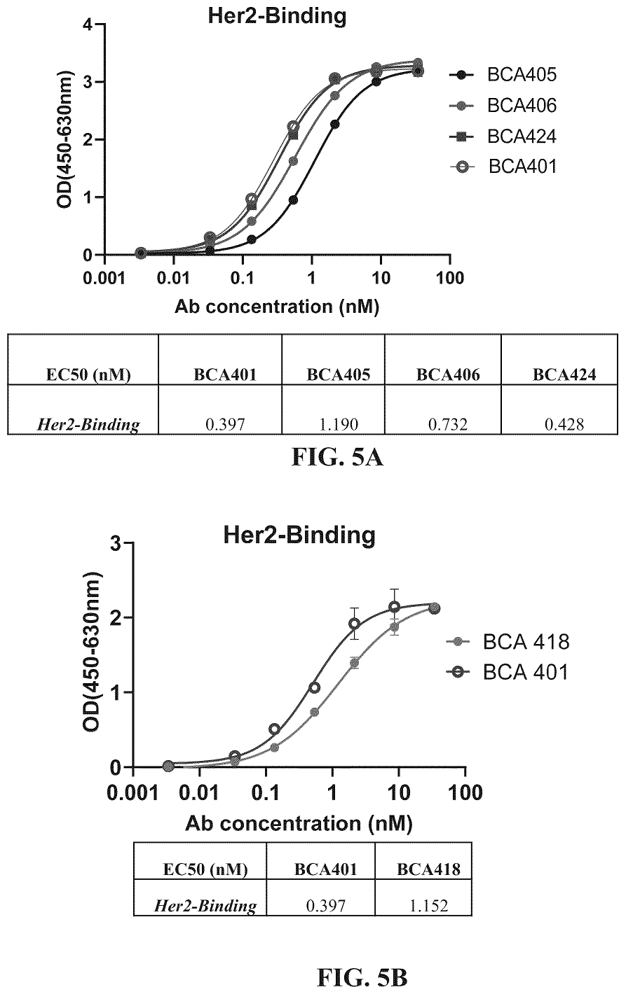

The multispecific proteins, BCA405, BCA406, BCA418, and BCA424, showed comparable EC50 values to the control antibody—BCA401 (FIG. 5A and FIG. 5B).

Binding of the multispecific proteins with EGFR as TAA, BCA405.EG, BCA406.EG, and BCA424.EG, to EGFR was assessed by a flow cytometry based binding assay using A431(EGFRhigh) cells. Briefly, A431(EGFRhigh) cells were incubated with serially diluted primary antibody (multispecific proteins and human isotype control antibody, hIgG (Bioxcell) in ice-cold 1×FACS staining buffer (1×PBS with 2% FBS and 0.5M EDTA) for 40 min at 4° C. This was followed by wash and subsequent incubation with secondary antibody (Goat anti-human IgG Fc specific-FITC (Sigma-Aldrich); 1:100 in ice-cold 1×FACS Staining buffer). This was followed by a wash and incubation with 7-AAD (BD-Biosciences) in PBS for 10 min at RT. Cells were then acquired on Cytoflex flow cytometer (Becton Dickinson). Data was analysed using FlowJo software.

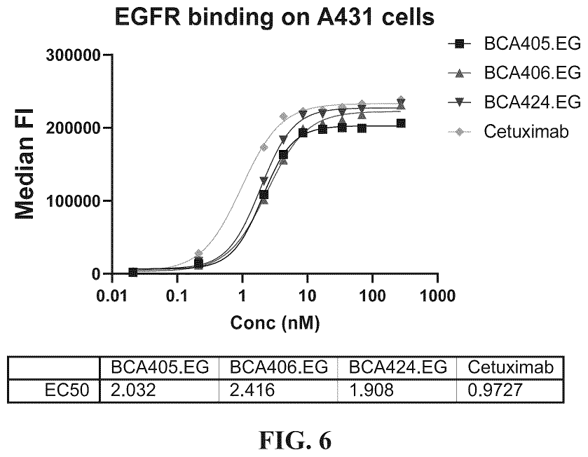

BCA405.EG, BCA406.EG, and BCA424.EG showed comparable binding to the control antibody-cetuximab (FIG. 6).

Overall, the data suggests that the binding of the multispecific proteins to the TAA (e.g., HER2 or EGFR) is very similar to control antibodies (i.e., trastuzumab (BCA401) or cetuximab, respectively).

### 6.3 Example 3. CD3 Binding of Multispecific Proteins

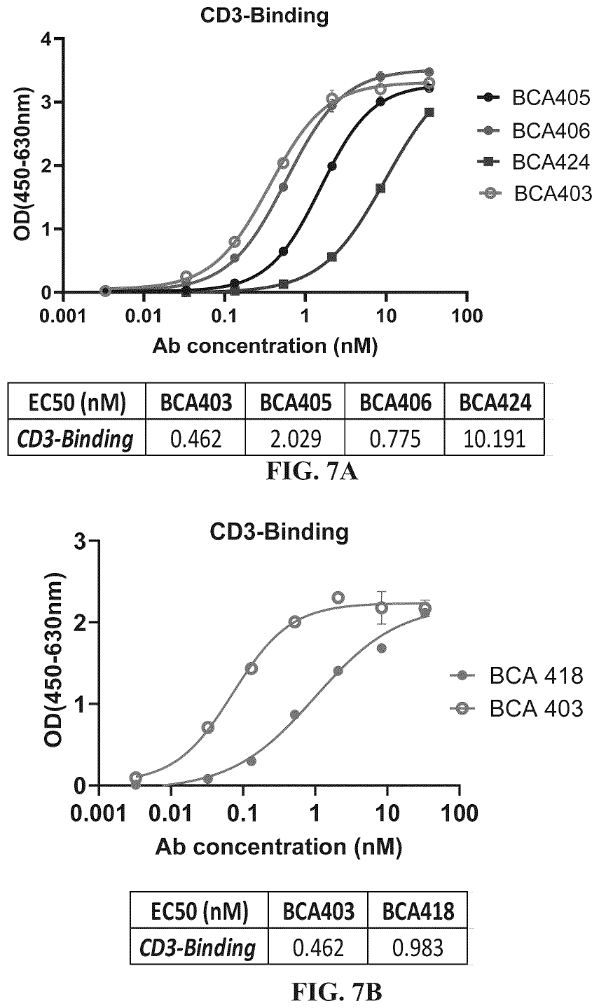

Binding of the multispecific proteins to CD3 was assessed by ELISA. Briefly, 96-well Maxi-Sorp plates (Nunc) were coated with CD3 extracellular domain (Acrobiosystem) at 4° C. overnight. The following day, the plates were washed with wash Buffer (0.05% Tween-20 (Sigma-Aldrich) in filtered DPBS (Gibco)) and blocked using SuperBlock™ blocking buffer (Thermo Fisher) for 1.5-2 hours. The plates were then incubated with serially diluted primary antibody [multispecific proteins and human isotype control antibody, hIgG (Bioxcell)] made in Assay diluent (wash buffer containing 10% SuperBlock™) for 1 hour at RT. This was followed by wash with wash buffer and subsequent incubation with secondary antibody (Donkey anti-human IgG H+L peroxidase (Jackson ImmunoResearch) diluted 1:25000 in assay diluent) for 1 hour at RT. After incubation, plates were washed with wash buffer. The colour was developed by adding TMB substrate solution (Sigma-Aldrich) and the reaction was stopped using Stop Solution (1N H₂SO₄). The optical density of each well was determined immediately, using a microplate reader at 450 nm with 630 nm as the reference wavelength. Each multispecific protein (i.e., BCA405, BCA406, BCA424 (FIG. 7A) and BCA418 (FIG. 7B)) showed binding to CD3.

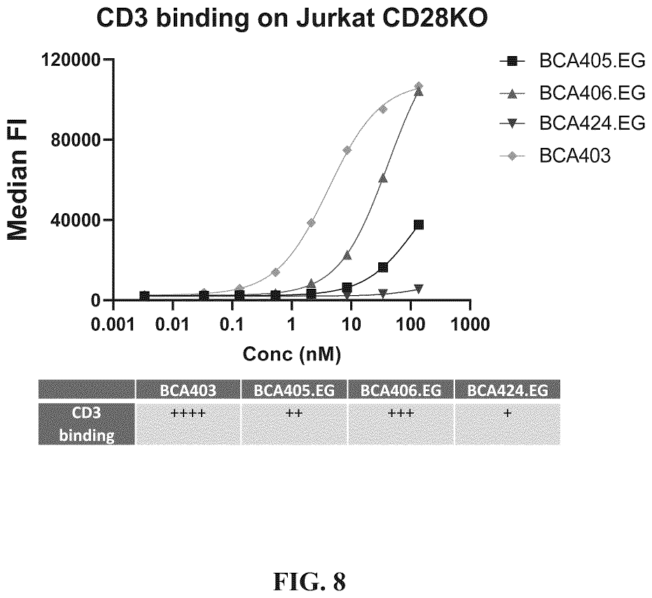

Binding of BCA405.EG, BCA406.EG, and BCA424.EG to CD3 was also assessed using a flow cytometry-based assay. Briefly, CD28 knock out Jurkat cells were incubated with serially diluted primary antibody (multispecific proteins and human isotype control antibody, hIgG (Bioxcell)) in ice-cold 1×FACS staining buffer (1×PBS with 2% FBS and 0.5M EDTA) for 40 min at 4° C. This was followed by wash and subsequent incubation with secondary antibody (Goat anti-human IgG Fc specific-FITC (Sigma-Aldrich); 1:100 in ice-cold 1×FACS Staining buffer). This was followed by a wash and incubation with 7-AAD (BD-Biosciences) in PBS for 10 min at RT. Cells were then acquired on Cytoflex flow cytometer (Becton Dickinson). Data was analysed using FlowJo software. BCA424.EG showed the maximum attenuation in CD3 binding followed by BCA405.EG and BCA406.EG (FIG. 8). The higher attenuation in the cell based binding assay compared to the ELISA could be due to better flexibility of the multispecific proteins while binding to cell surface receptors vs plate coated antigen and/or variation in antigen to multispecific protein ratios between the two methods.

### 6.4 Example 4. CD28 Binding of Multispecific Proteins

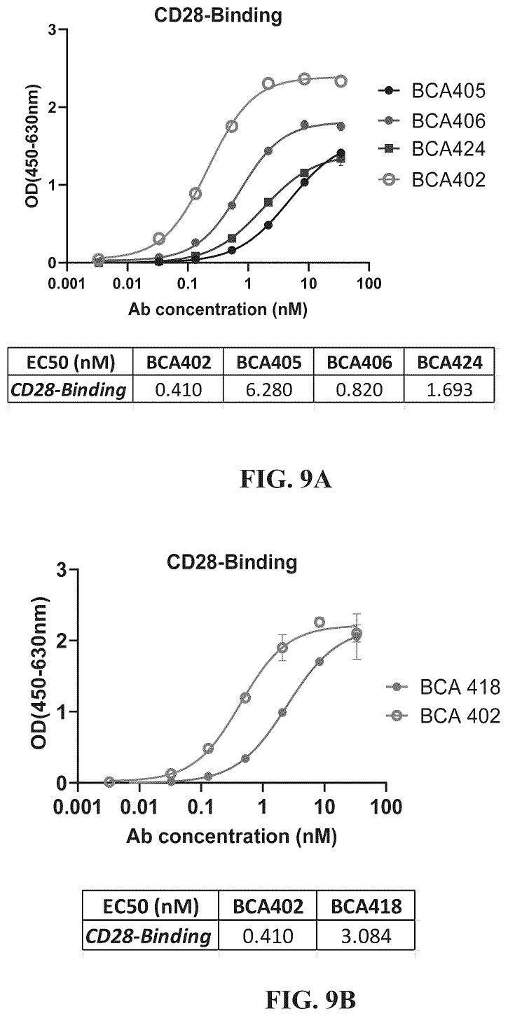

Binding of the multispecific proteins to CD28 was assessed by ELISA. Briefly, 96-well Maxi-Sorp plates (Nunc) were coated with CD28 extracellular domain (Acrobiosystem) at 4° C. overnight. The following day, the plates were washed with wash Buffer (0.05% Tween-20 (Sigma-Aldrich) in filtered DPBS (Gibco)) and blocked using SuperBlock™ blocking buffer (Thermo Fisher) for 1.5-2 hours. The plates were then incubated with serially diluted primary antibody [multispecific proteins and human isotype control antibody, hIgG (Bioxcell)] made in Assay diluent (wash buffer containing 10% SuperBlock™) for 1 hour at RT. This was followed by wash with wash buffer and subsequent incubation with secondary antibody (Donkey anti-human IgG H+L peroxidase (Jackson ImmunoResearch) diluted 1:25000 in assay diluent) for 1 hour at RT. After incubation, plates were washed with wash buffer. The colour was developed by adding TMB substrate solution (Sigma-Aldrich) and the reaction was stopped using Stop Solution (1N H₂SO₄). The optical density of each well was determined immediately, using a microplate reader at 450 nm with 630 nm as the reference wavelength. Each multispecific protein (i.e., BCA405, BCA406, BCA424 (FIG. 9A) and BCA418 (FIG. 9B)) showed binding to CD28.

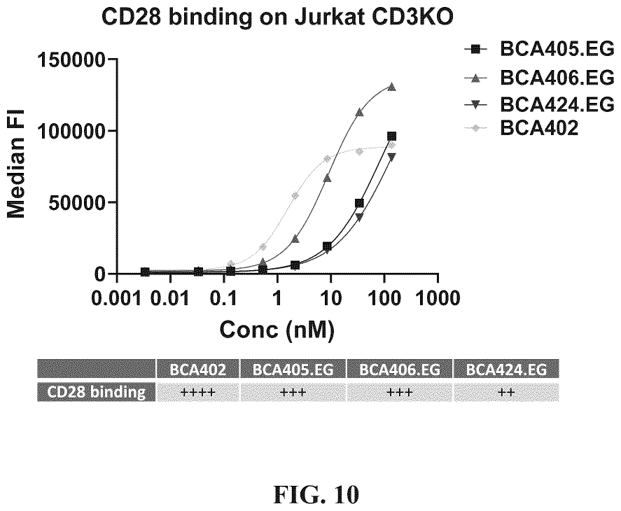

Binding of BCA405.EG, BCA406.EG, and BCA424.EG was assessed by flow cytometry based binding assay. Briefly, CD3 knock out Jurkat cells were incubated with serially diluted primary antibody (multispecific proteins and human isotype control antibody, hIgG (Bioxcell)) in ice-cold 1×FACS staining buffer (1×PBS with 2% FBS and 0.5M EDTA) for 40 min at 4° C. This was followed by wash and subsequent incubation with secondary antibody (Goat anti-human IgG Fc specific-FITC (Sigma-Aldrich); 1:100 in ice-cold 1×FACS Staining buffer). This was followed by a wash and incubation with 7-AAD (BD-Biosciences) in PBS for 10 min at RT. Cells were then acquired on Cytoflex flow cytometer (Becton Dickinson). Data was analysed using FlowJo software. Each multispecific protein (i.e., BCA405, BCA406, BCA424 (FIG. 9A) and BCA418 (FIG. 10)) showed binding to CD28.

### 6.5 Example 5. Multispecific Protein Mediated Tumor Cell Cytotoxicity

Anti-CD3 and anti-CD28 antibodies, respectively, provide antigen independent TCR signalling (Signal 1), and co-stimulation (Signal 2) required for T cell activation, differentiation, survival, and memory immune response (Hwang, Jeong-Ryul et al. “Recent insights of T cell receptor-mediated signaling pathways for T cell activation and development.” Experimental & molecular medicine vol. 52, 5 (2020): 750-761. doi:10.1038/s12276-020-0435-8; Esensten, Jonathan H et al. “CD28 Costimulation: From Mechanism to Therapy.” Immunity vol. 44, 5 (2016): 973-88. doi:10.1016/j.immuni.2016.04.020; the entire contents of each of which is incorporated herein by reference for all purposes). Activated T-cells secrete pro-inflammatory cytokines like IFN-γ, TNF-α and cytolytic molecules like Granzyme-B required for tumour cell killing (Raphael, Itay et al. “T cell subsets and their signature cytokines in autoimmune and inflammatory diseases.” Cytokine vol. 74, 1 (2015): 5-17. doi:10.1016/j.cyto.2014.09.011; Bevington, Sarah L et al. “T Cell Receptor and Cytokine Signaling Can Function at Different Stages to Establish and Maintain Transcriptional Memory and Enable T Helper Cell Differentiation.” Frontiers in immunology vol. 8 204. 3 Mar. 2017, doi:10.3389/fimmu.2017.00204; the entire contents of each of which is incorporated herein by reference for all purposes)

The cytolytic potential of the multispecific proteins was evaluated using co-culture assays. Briefly, HER2 (SKBR3) or EGFR (BxPC3 EGFR high) overexpressing target cells were co-cultured with human PBMCs at T:E ratio of 1:10 for 48 h. A total of 1×10⁴ tumor cells (HER2 expressing (SKBR3) or EGFR expressing (BxPC3 EGFR high) (target cells) per well were seeded in white-walled, clear bottom (flat) 96-well plates (Nunc) and incubated at 37° C. and 5% CO₂ overnight for cell attachment. On the same day PBMCs (effector) were revived in PBMC revival media (RPMI-1640 (2C) (Gibco)+10% HI-FBS+100 IU/mL (Gibco) rhIL-2 (R&D Systems), filtered) and incubated overnight at 37° C. and 5% CO₂. PBMCs were added to target cells at an effector to target cell ratio of 10:1 and incubated for 48 hours in the presence of the indicated concentrations of the multispecific proteins or a hIgG control. At the end of incubation, plates were spun, and a part of culture medium was transferred to a new 96-well plate. The extent of cell lysis in the target cells was determined using either BioGlo (Promega Corp.) (when using Luciferase expressing tumor cells) or CytotoxGlo (Promega Corp.). For BioGlo, 80 μL/well of BioGlo reagent was added, incubated at RT in dark for 5 mins and luminescence reading was taken on Cytation 5 (BioTek Instruments). For CytotoxGlo, digitonin was added to Target+digitonin well (complete cell lysis control) and incubated for 15 minutes at RT. 50 μL/well of CytotoxGlo reagent was added to all sample and control wells, incubated at RT in dark for 15 mins and luminescence reading was taken on Cytation 5 instrument. Percent cytotoxicity was evaluated by luminescence-based readouts using Bioglo or cytotox glo. Formula used for calculation of cytotoxicity: % Cytotoxicity=(Sample RLU−RLU of spontaneous death)/(RLU of complete cell killing with Digitonin−RLU of spontaneous death)×100. RLU=Relative Luminescence Units.

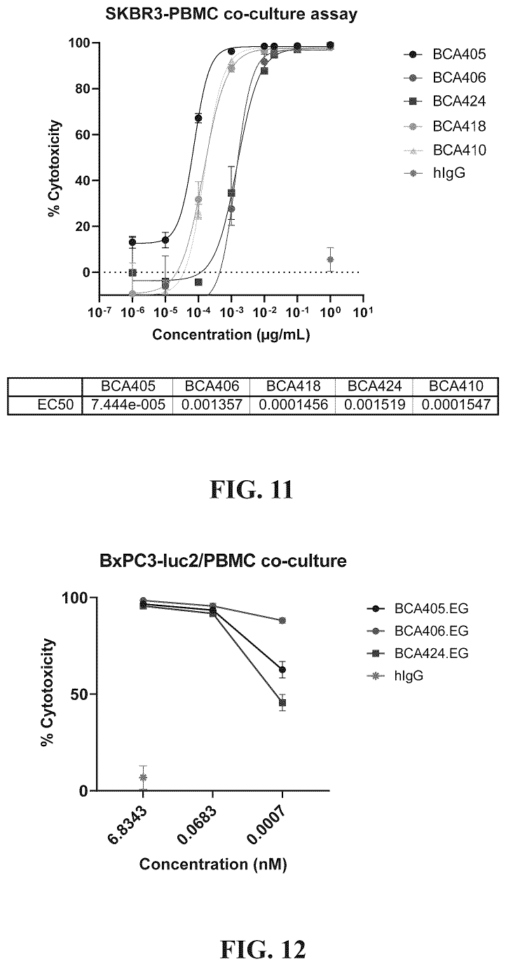

For the evaluation of dose dependent cytotoxicity, BCA418 and BCA410 were compared to the human IgG control (FIG. 11). The comparison of BCA405, BCA406, BCA424 EC50 values indicate that BCA405 shows the highest cytotoxicity of SKBR3 cells followed by BCA418 and BCA410 (control). Compared to the remainder of the multispecific proteins, BCA424 and BCA406 showed lower EC50 values, but still are highly potent as the proteins showed complete killing of target cells at the highest concentration (FIG. 11). Overall, all the multispecific proteins shows comparable cytotoxicity of SKBR3 cells in the in vitro assay.

Similarly, BCA405.EG, BCA424.EG and BCA406.EG showed higher killing of BxPC3 cells (EGFRhigh) in the human PBMC based co-culture assay (FIG. 12). The data suggest that the multispecific proteins targeting HER2 or EGFR are able to induce target cell killing at the concentrations tested.

### 6.6 Example 6. Immune Cell Synapse Formation & Cytokine Release Induced by Multispecific Proteins

To investigate the type of immune response induced by the multispecific proteins, the ability of the multispecific proteins to induce a Th1 response in PBMCs co-cultured with tumour cells was assessed. Th1 cytokines, e.g., IL-2, IFNγ, and TNF, are involved in activation of innate immune cells during an inflammatory response and can help in the development of cytotoxic lymphocytes (CD8+ T cells and natural killer (NK) cells) which would then produce Granzyme B and perforin for tumour cell killing (Rousalova, Ilona, and Evzen Krepela. “Granzyme B-induced apoptosis in cancer cells and its regulation (review).” International journal of oncology vol. 37, 6 (2010): 1361-78. doi:10.3892/ijo_00000788; Cullen, S P et al. “Granzymes in cancer and immunity.” Cell death and differentiation vol. 17, 4 (2010): 616-23. doi:10.1038/cdd.2009.206; the entire contents of each of which is incorporated herein by reference for all purposes). Th1 cytokines are indicators of good prognosis in cancer patients as well (Lee, Hae Lim et al. “Inflammatory cytokines and change of Th1/Th2 balance as prognostic indicators for hepatocellular carcinoma in patients treated with transarterial chemoembolization.” Scientific reports vol. 9, 1 3260. 1 Mar. 2019, doi:10.1038/s41598-019-40078-8; the entire contents of which is incorporated herein by reference for all purposes). A high IL-6 release in the presence of cancer cells would cause acute inflammation leading to tumour cell killing, activation of lymphocytes, and prevention of Treg differentiation (Gabay, Cem. “Interleukin-6 and chronic inflammation.” Arthritis research & therapy vol. 8 Suppl 2,Suppl 2 (2006): S3. doi:10.1186/ar1917; Tanaka, Toshio et al. “IL-6 in inflammation, immunity, and disease.” Cold Spring Harbor perspectives in biology vol. 6, 10 a016295. 4 Sep. 2014, doi:10.1101/cshperspect.a016295; the entire contents of each of which is incorporated herein by reference for all purposes).

Cytokine release was assessed using a cytokine release assay (CRA). Briefly, the supernatants from an in vitro cytolysis assay (see below), an in vitro T cell activation and memory cell differentiation assay (see below), and an in vitro Bcl-xL assay (see below) were used for evaluation of cytokine release. Cytokine level determination was performed using either Human Th1/Th2/Th17 CBA Kit (BD Bioscience) or Cytometric Bead Array (CBA) Human Flex Set (BD Bioscience) according to manufacturer's instructions. Data was analysed using FCAP Array software and graphs were plotted on GraphPad Prism.

The in vitro cytolysis assay was performed as follows. A total of 1×10⁴ tumor cells (target) per well were seeded in white-walled, clear bottom (flat) 96-well plates (Nunc) and incubated at 37° C. and 5% CO₂ overnight for cell attachment. On the same day PBMCs (effector) were revived in PBMC revival media (RPMI-1640 (2C) (Gibco)+10% HI-FBS+100 IU/mL (Gibco) rhIL-2 (R&D Systems), filtered) and incubated overnight at 37° C. and 5% CO₂. PBMCs were added to target cells at an effector to target cell ratio of 10:1. Additionally, PBMCs in the absence of target cells were also included in the assay. The cells were incubated for 48 hours in the presence of the indicated concentrations of the multispecific proteins or a hIgG control. At the end of incubation, plates were spun, and a part of culture medium was transferred to a new 96-well plate. The extent of cell lysis in the target cells was determined using either BioGlo (Promega Corp.) (when using Luciferase expressing tumor cells) or CytotoxGlo (Promega Corp.). For BioGlo, 80 μL/well of BioGlo reagent was added, incubated at RT in dark for 5 mins and luminescence reading was taken on Cytation 5 (BioTek Instruments). For CytotoxGlo, digitonin was added to Target+digitonin well (complete cell lysis control) and incubated for 15 minutes at RT. 50 μL/well of CytotoxGlo reagent was added to all sample and control wells, incubated at RT in dark for 15 mins and luminescence reading was taken on Cytation 5 instrument. Supernatant from the assay plates were collected and stored in −80° C. and evaluated for cytokine release using ELISA or CBA.

The in vitro T cell activation and memory cell differentiation assay was performed as follows. The target tumor cells were labeled with the Violet Proliferation Dye 450 (VPD-450) (BD Biosciences). A total of 1×10⁴ labelled tumor cells (target) per well were seeded in white-walled, clear bottom (flat) 96-well plates (Nunc) and incubated at 37° C. and 5% CO₂ overnight for cell attachment. On the same day PBMCs (effector) were revived in PBMC revival media (RPMI-1640 (2C)+10% HI-FBS+100 IU/mL rhIL-2, filtered) and incubated overnight at 37° C. and 5% CO₂. PBMCs were added to target cells at an effector to target cell ratio of 10:1 in total volume of 200 μL and incubated for 3 days or 7 days in the presence of the indicated concentrations of the multispecific proteins or a hIgG control. At the end of each incubation period, plates were spun, and a part of culture medium was transferred to a new 96-well plate. Cells were treated with pre-warmed Accutase (Sigma-Aldrich) for 15 mins at RT, harvested and incubated with Fc Block (Anti-Hu Fc receptor binding inhibitor antibodies, Thermo Scientific, diluted 1:5 in 1×PBS) for 20 minutes at 4° C. (in dark). Cells were then washed with 1×PBS and stained with Fixable viability dye-506 (FVD-506 (Thermo Scientific), 1:1000 diluted in 1×PBS) for 30 minutes at 4° C. (in dark). This is followed by wash with FACS staining buffer and staining with cocktail of anti-CD3 APC, anti-CD8 FITC, anti-CD25 APC H7, anti-CD45RO PE (BD Biosciences), anti-CD4 AF700 and anti-CCR7 PerCPCy5.5 (Biolegend). After incubation, cells were washed with FACS staining buffer and resuspended in 300 μL of 1×PBS. 50 μL of Countbright Absolute counting beads (1:5 diluted) (Invitrogen) were added to all the sample tubes and a fixed number of bead events were acquired in Cytoflex flow cytometer (Becton Dickinson). Data was analysed using FlowJo software.

The in vitro Bcl-xL assay was performed as follows. A total of 5×10³ tumor cells (target) per well were seeded in white-walled, clear bottom (flat) 96-well plates (Nunc) and incubated at 37° C. and 5% CO₂ overnight for cell attachment. On the same day PBMCs (effector) were revived in PBMC revival media (RPMI-1640 (2C)+10% HI-FBS) and incubated overnight at 37° C. and 5% CO₂. PBMCs were added to target cells at an effector to target cell ratio of 20:1 and incubated for 48 hours in the presence of the indicated concentrations of the multispecific proteins or a hIgG control. At the end of incubation period plates were spun, and a part of the culture medium was transferred to a new 96-well plate. Cells were treated with accutase for 15 mins at RT, harvested and incubated with Fc Block (Anti-Hu Fc receptor binding inhibitor antibodies), 1:5 diluted in Staining buffer-1 (SB-1, 1×PBS with 2% FBS and 0.5M EDTA) for 20 minutes at 4° C. (in dark), washed with 1×PBS and stained with Fixable viability dye-506 (FVD-506, 1:1000 diluted in 1×PBS) for 30 minutes at 4° C. (in dark). This is followed by wash with SB-1 and staining with cocktail of surface antibodies (CD3-BV421 (BD Biosciences), CD4-Alexa Fluor 647 (BD Biosciences), CD8-FITC (BD Pharmingen), CD25-APC-H7 (BD Pharmingen). After incubation, cells were washed with SB-1 and fixed using 1×BD Lyse/Fix buffer (BD Biosciences) for 10 min in 37° C. water bath. Cells were washed with SB-2 (1×PBS with 2% FBS), supernatant discarded and stored in remaining diluted fixative overnight at 4° C. On next day cells were once washed with SB-2 and permeabilized using ice-cold BD Perm III buffer (pre-chilled at −20° C. prior to addition to cells). The fixed/permeabilized cells were stained with anti-Bcl-xL-PE (1:50 dilution) in antibody dilution buffer for 1 h at RT or using anti-Bcl-xL primary antibody (1:100 dilution, 1 h at RT) followed by a wash with antibody dilution buffer and an anti-rabbit IgG (H+L)-PE secondary antibody (1:250 dilution), incubated for 30 min at RT, followed by wash and resuspension in 1×PBS. Both anti-Bcl-xL primary antibodies and anti-rabbit IgG (H+L)-PE secondary antibodies were from Cell Signaling Technologies. Stained cells were acquired in Cytoflex flow cytometer (Becton Dickinson). Data was analysed using FlowJo software.

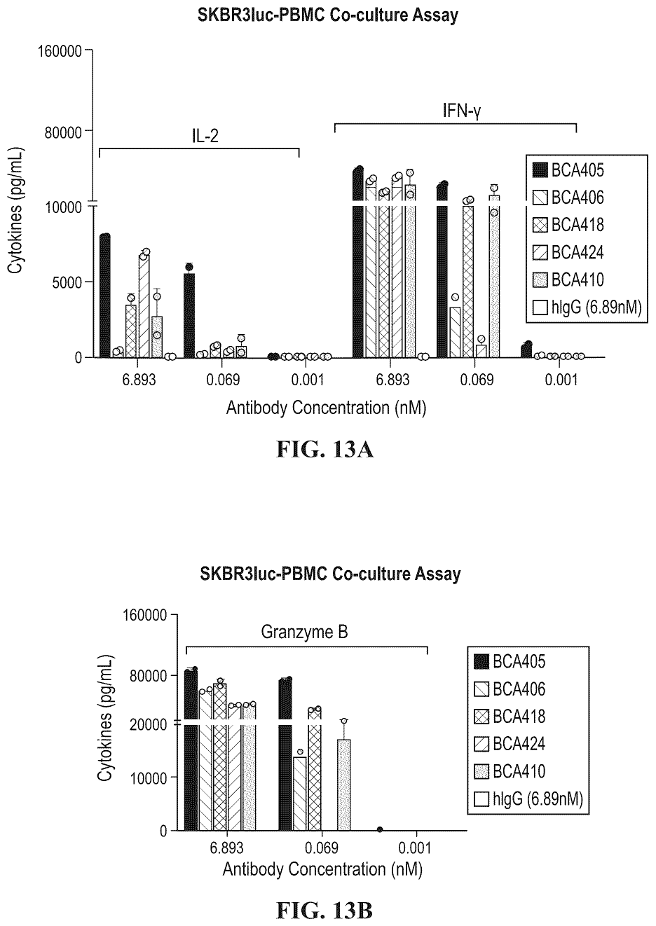

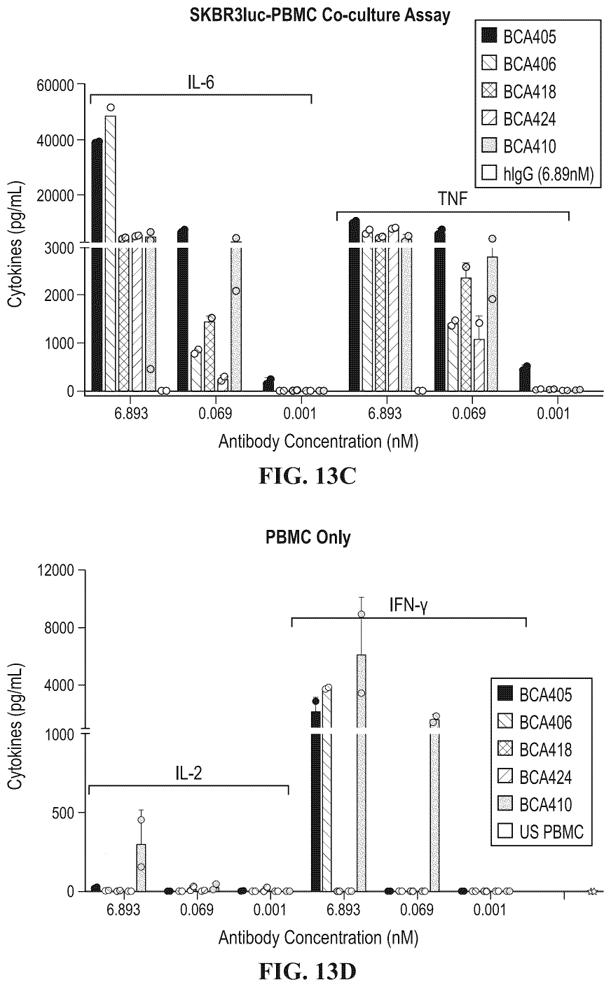

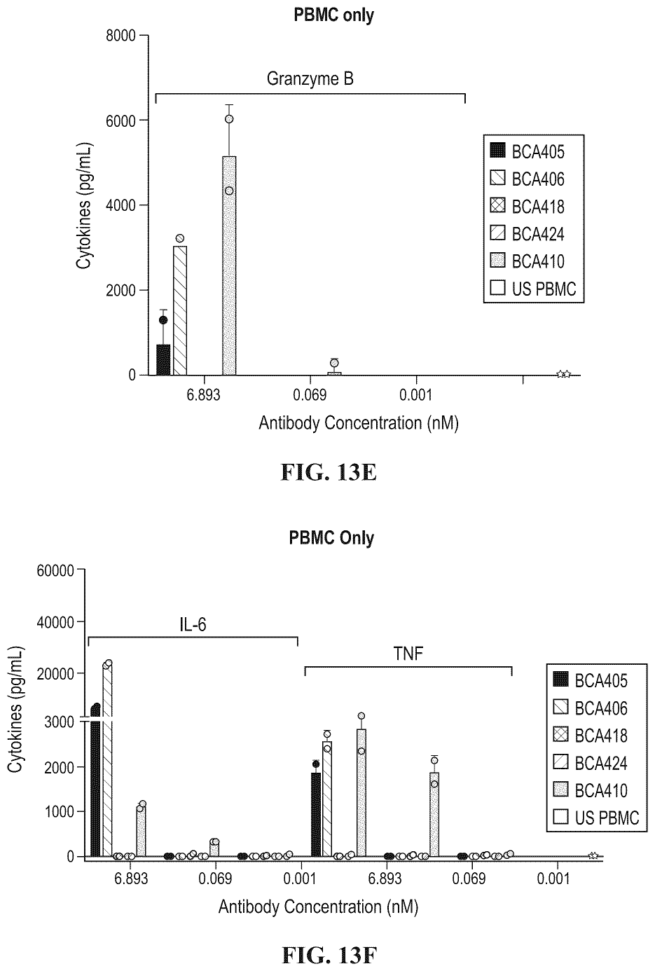

Upon forming a synapse with the cancer cell, BCA405 most potently engaged T cells to induce release of IL-2, IFNγ, and Granzyme B, indicating the proliferation and activation of T-cells by the multispecific proteins, comparable or better than the BCA410 control (FIG. 13A and FIG. 13B). Further, targeted induction of pro-inflammatory cytokines TNF and IL-6 was observed when T-cell engagement occurred in the presence of cancer cells (FIG. 13C). As mentioned previously, this is indicative of an acute inflammation that would be cytotoxic to tumour. Among the multispecific proteins BCA418 and BCA424 showed negligible IL-6 release but good IL-2 and IFN7 release after forming the synapse between tumour cell and PBMC. In the case of BCA424, this could be attributed to the less exposed positions of anti-CD3 and anti-CD28 arms compared to that in the BCA405 and BCA406 formats.

To evaluate whether the multispecific proteins exhibit systemic cytotoxicity, the cytokine release from direct stimulation of PBMCs with the multispecific proteins was assessed. Systemic toxicity, because of cytokine release syndrome (CRS) is the most severe mode of action-related side effect of immunotherapy with T-cell engagers (TCEs). CRS occurs due peripheral T-cell activation, subsequently leading to release of cytokines and sustained inflammatory response (Cosenza, Maria et al. “Cytokine Release Syndrome Associated with T-Cell-Based Therapies for Hematological Malignancies: Pathophysiology, Clinical Presentation, and Treatment.” International journal of molecular sciences vol. 22, 14 7652. 17 Jul. 2021, doi:10.3390/ijms22147652; the entire contents of which is incorporated herein by reference for all purposes). In the absence of the cancer cell synapse i.e., upon direct stimulation of PBMCs, there was minimal/no release of IL-2, IFN-7, and Granzyme B by these antibodies when compared to BCA410 (control) (FIG. 13D-13E). BCA406 showed higher IL-6 and TNF compared to BCA410 and other tri-specific antibodies. The TNFα level in BCA405 was comparable to BCA410. BCA418 and BCA424 did not show any release of these cytokines(FIG. 13F).

### 6.7 Example 7. T-Cell Activation and Bel-xL Expression Induced by Multispecific Proteins

T cell activation through TCR signalling causes proliferation, exhaustion, and ultimately death of T cells. CD28 co-stimulation increases the survival of T cells by enhancing IL-2 release and expression of anti-apoptotic protein, Bcl-xL on activated T cells (Boise, L H et al. “CD28 costimulation can promote T cell survival by enhancing the expression of Bcl-XL.” Immunity vol. 3, 1 (1995): 87-98. doi:10.1016/1074-7613(95)90161-2; Broome, H E et al. “Expression of Bcl-2, Bcl-x, and Bax after T cell activation and IL-2 withdrawal.” Journal of immunology (Baltimore, Md.: 1950) vol. 155, 5 (1995): 2311-7; the entire contents of each of which is incorporated herein by reference for all purposes).

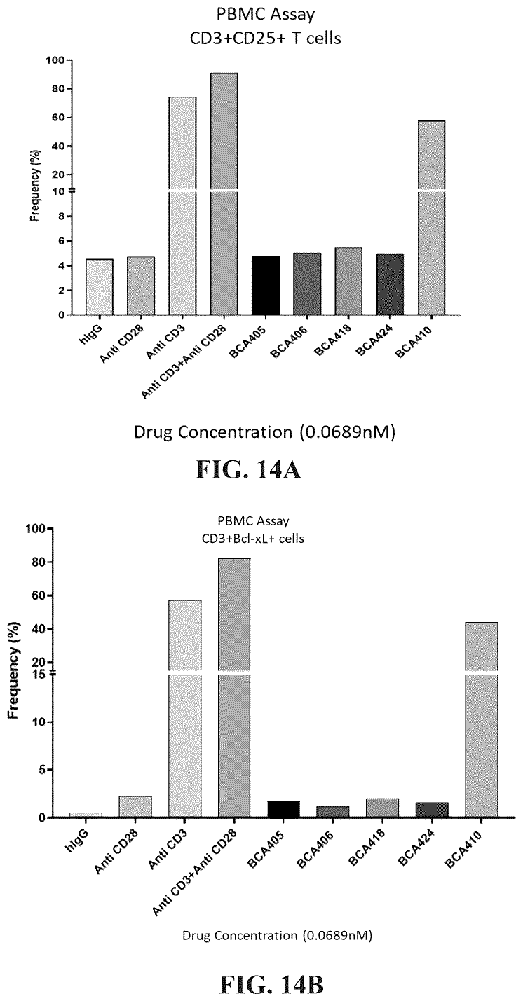

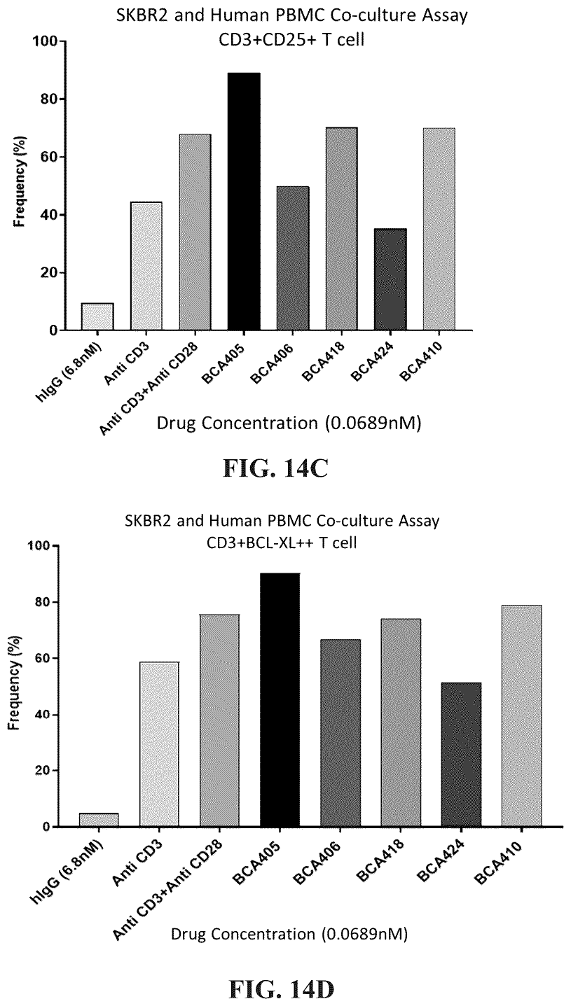

hPBMCs were activated with multispecific proteins (BCA405, BCA406, BCA418 and BCA424) in the presence or absence of SKBR3 cells. The target to effector ratio was maintained at 1:20. After 48 h of incubation, T cell activation and Bcl-xL expression was assessed using flow cytometry. In the absence of target cells, BCA405, BCA406, BCA418 and BCA424 did not induce T cell activation and Bcl-xL expression. Expression of CD3+CD25+ T cells and CD3+Bcl-xL+ T cells was equivalent to human IgG control. Anti-CD3, anti-CD3 with anti-CD28 combination and BCA410, used as control molecules, showed high T cell activation and Bcl-xL expression (FIG. 14A and FIG. 14B). In the presence of SKBR3 cells, due to the synapse formation, BCA405, BCA406, BCA418 and BCA424 showed an induction of T cell activation and Bcl-xL expression, similar to the control mAbs (FIG. 14C and FIG. 14D). The presence of anti-CD28 in the tri-specific formats helps in the induction of T-cell activation and overexpression of anti-apoptotic protein, Bcl-xL, only in the presence of target cells. This is not true in the absence of SKBR3 cells suggesting the importance of CD28 co-stimulation and target engagement by these formats.

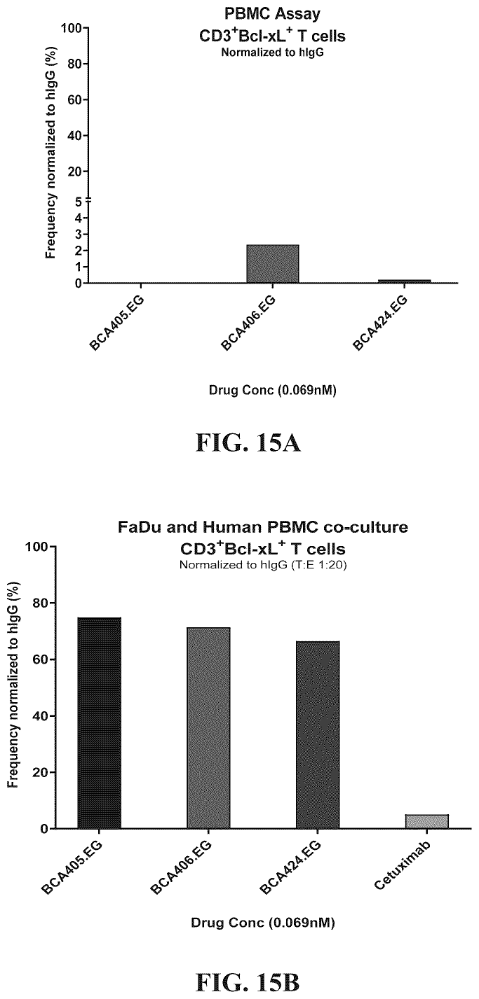

T cell activation and induction of Bcl-xL expression by the tri-specific antibodies is dependent on antibody format but independent of the TAA. In co-culture assay, using FaDu cells and human PBMCs, BCA405.EG, BCA406.EG, and BCA424.EG showed enhanced expression of CD3+CD25+ T cells (data not shown) and CD3+Bcl-xL+ T cells. This induction was not observed with Cetuximab in co-culture assays or by the tri-specific antibodies in the absence of FaDu cells (FIG. 15A and FIG. 15B).

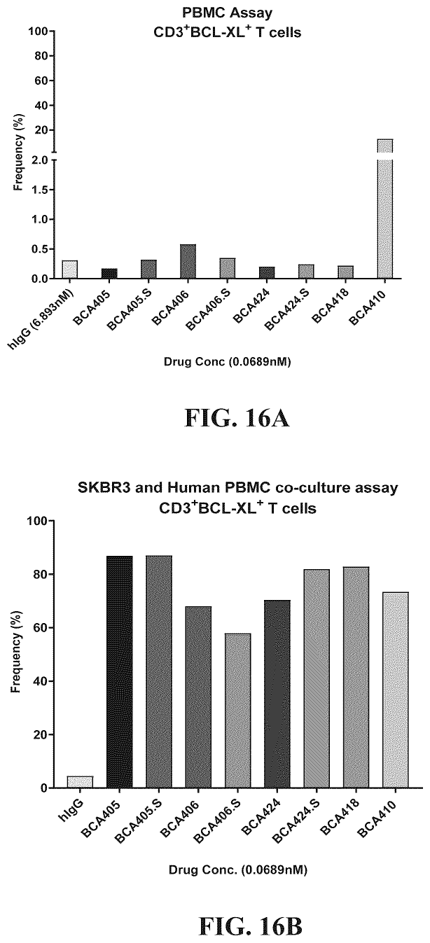

In this assay, induction of Bcl-xL expression by the tri-specific antibodies is also independent of CD3 or CD28 sequences. In co-culture assay, using SKBR3 cells and human PBMCs, BCA405, BCA406, and BCA424 showed similar frequency of T cell activation and Bcl-xL expression as BCA405.S, BCA406.S, and BCA424.S. The latter formats have different CD3 sequence. The data suggest that CD25 activation and Bcl-xL expression by T cells is dependent on antibody formats and not CD3 sequence in this assay (FIG. 16A and FIG. 16B).

### 6.8 Example 8. Dose Dependent and Anti-CD3 Sequence Dependent Cytotoxicity of Target Cells Induced by Multispecific Proteins

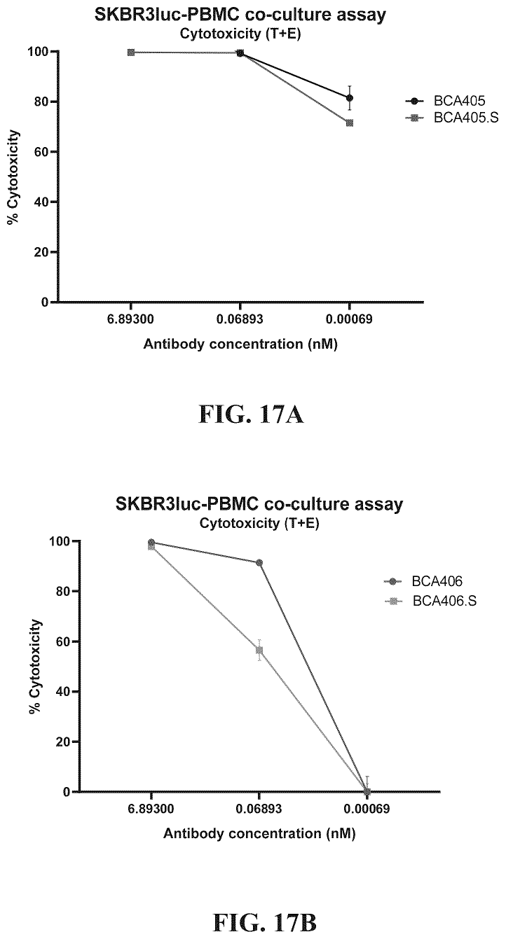

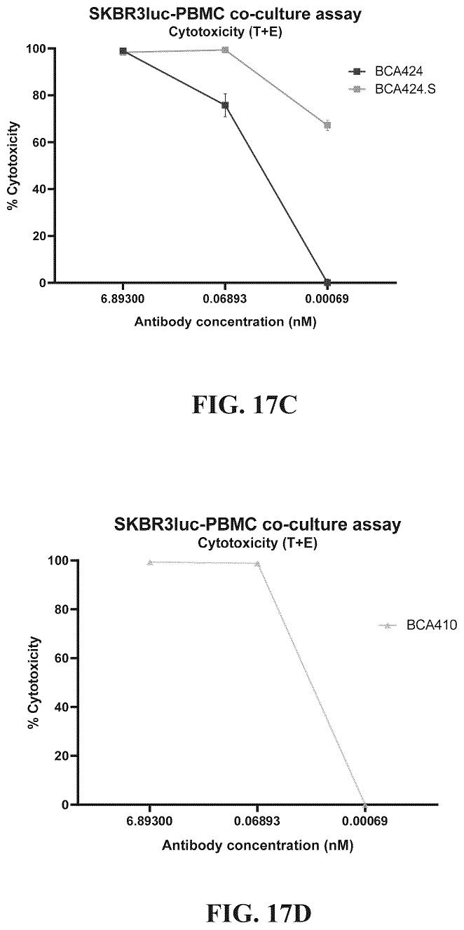

Cytotoxicity assays (as described above in Example 5) using SKBR3 cells as target and hPBMC as effectors were performed to assess whether functional activity of the multispecific proteins is dependent on CD3 sequence. BCA405 and BCA405.S showed comparable cytotoxicity of SKBR3 cells suggesting that T cell activity in this format is independent of CD3 sequence (FIG. 17A). BCA406 and BCA406.S showed comparable cytotoxicity at highest and lowest concentrations tested. But at mid concentration, BCA406 showed better killing of target cells compared to BCA406.S (FIG. 17B). BCA424.S showed enhanced cytotoxicity of SKBR3 cells than BCA424 (FIG. 17C). BCA405, BCA405.S and BCA424.S showed high tumour cell killing even at 0.00069 nM indicating that these formats are highly potent (FIG. 17A and FIG. 17C). As expected, BCA410 (control) showed dose dependent tumour killing activity (FIG. 17D).

### 6.9 Example 9. CD4+T and CD8+ T Cell Activation and Memory Phenotype Induced by Multispecific Proteins Only in the Presence of Target and Effector Cells

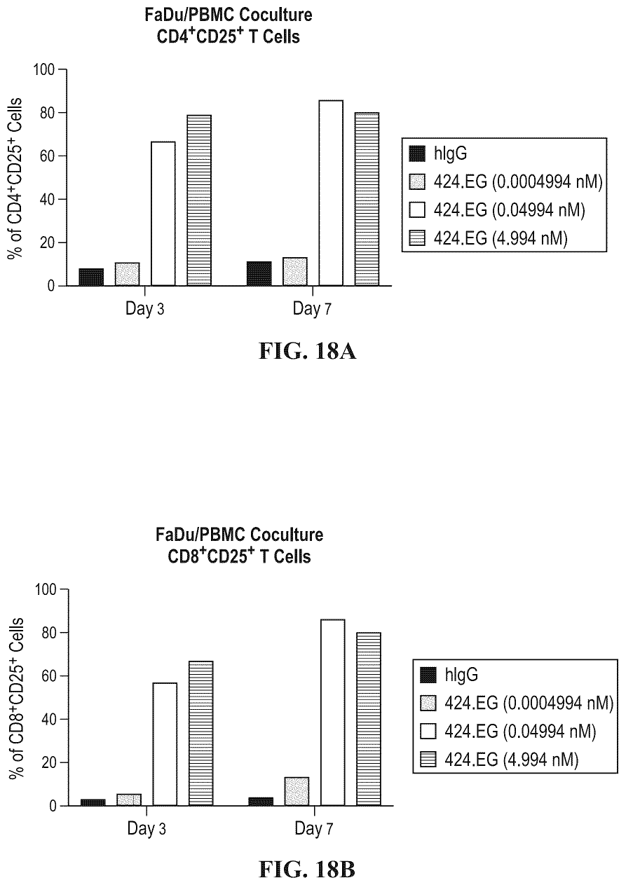

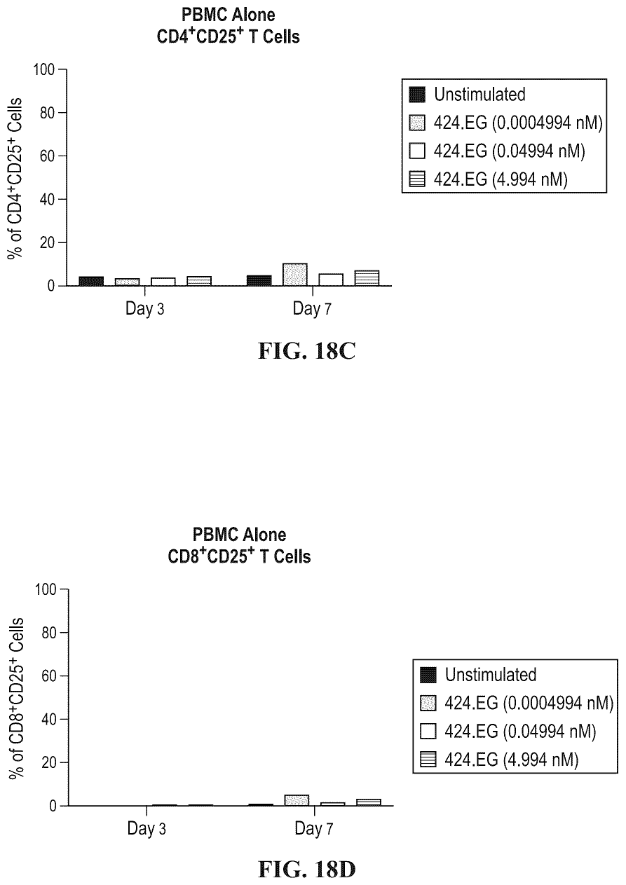

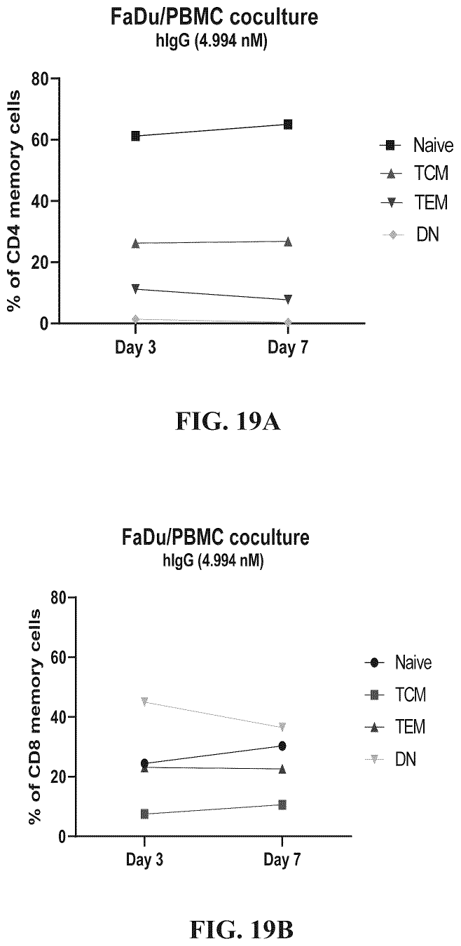

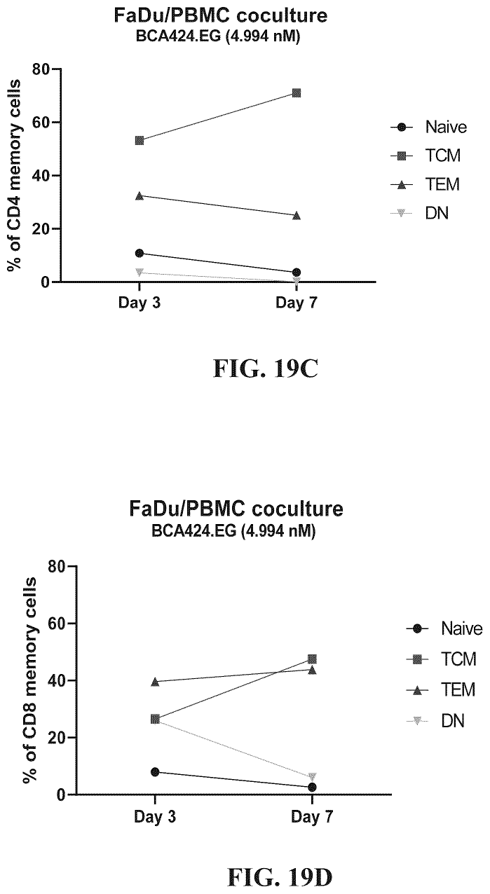

In the earlier assays, activation of T cells in the presence of multispecific proteins was evaluated until day 2 in co-culture assays using FaDu cells and human PBMCs. Next, we assessed the effect of BCA424.EG on T cell activation until day 7. In the presence of target cells, BCA424.EG showed dose dependent CD4+T and CD8+ T cell activation on day 3 and day 7 (FIG. 18A and FIG. 18B). T cell activation was not observed in the absence of target cells (FIG. 18C and FIG. 18D). An increase in memory T cells is often associated with better prognosis in cancer (Han, Jichang et al. “Memory CD8+ T cell responses to cancer.” Seminars in immunology vol. 49 (2020): 101435. doi:10.1016/j.smim.2020.101435; the entire contents of which is incorporated herein by reference). The effect of BCA424.EG in inducing effector memory T cell (TEM) and central memory T cell (TCM) phenotype was also evaluated. HIgG showed higher proportion of naïve cells compared to TEM and TCM suggesting minimal T cell activation and differentiation (FIG. 19A and FIG. 19B). Unlike the hIgG control, BCA424.EG (multispecific protein) enhanced frequency of CD4+ and CD8+TEM and TCM cells in the presence of target cells (FIG. 19C and FIG. 19D). Overall, the data suggest that BCA424.EG has the potential to enhance the differentiation of CD4+ and CD8+ to effector and central memory T cell phenotypes.

### 6.10 Example 10. Prolonged and Temporal Induction of Proinflammatory Cytokines Only in the Presence of Target and Effector Cells

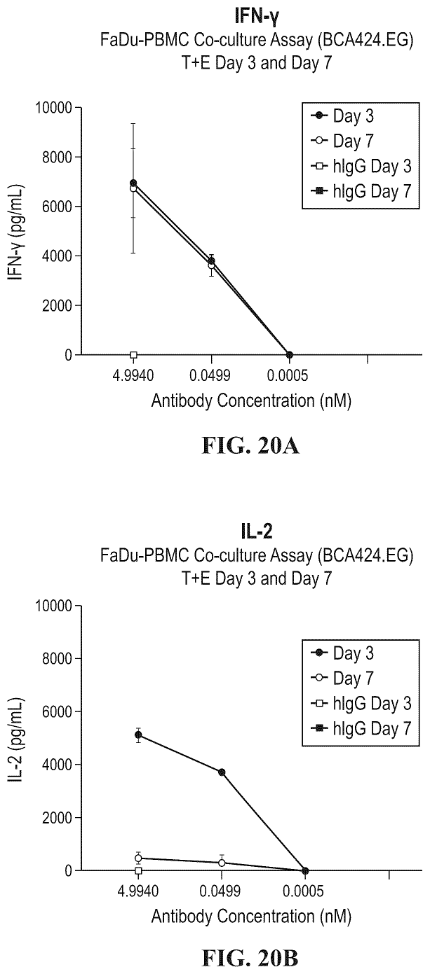

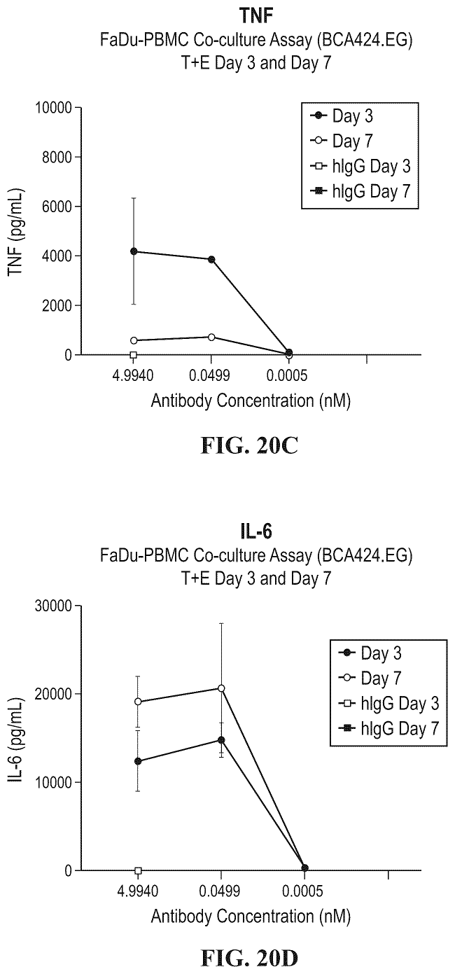

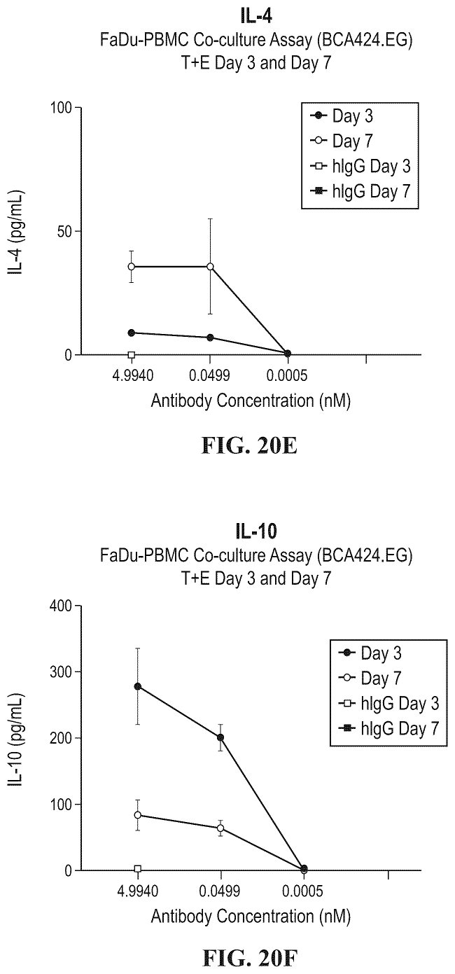

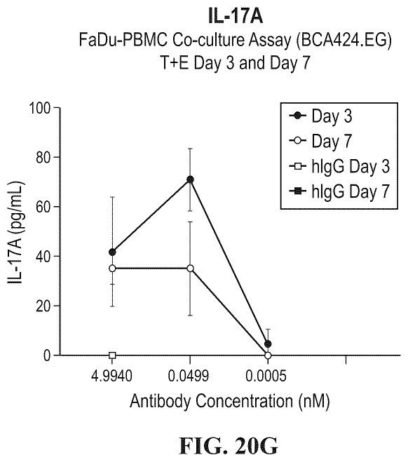

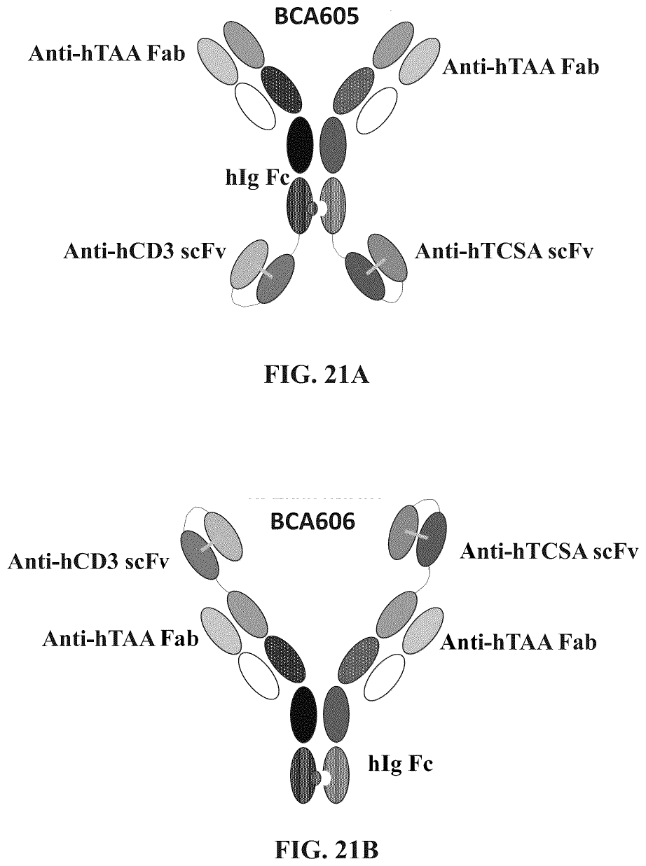

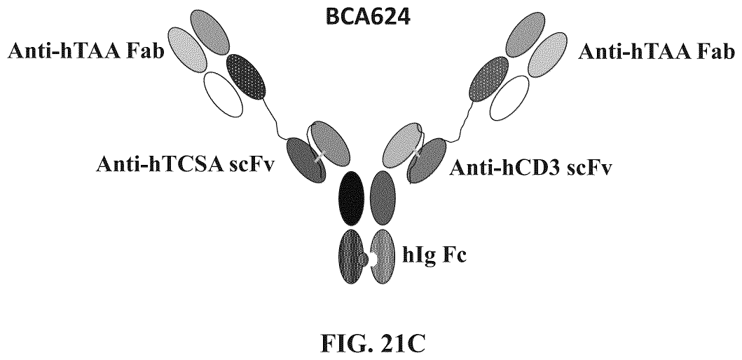

BCA424.EG showed minimal or no release of Th1, Th2 or Th17 cytokines by PBMCs in the absence of target cells (data not shown). In FaDu-PBMC co-cultures stimulated with BCA424.EG, cytokines released by activated T cells in the presence of BCA424.EG was also evaluated on day 3 and day 7. IFN-7 level was comparable between both the time points and showed a dose dependent release (FIG. 20A). IL-2 (FIG. 20B), TNF (FIG. 20C), and IL-10 (FIG. 20F) showed higher release on day 3 compared to day 7, whereas IL-6 (FIG. 20D) showed a reverse trend. Reduction in IL-2 level on day 7 could be due to rapid T cell proliferation and differentiation (Cheng, Laurence E et al. “Enhanced signaling through the IL-2 receptor in CD8+ T cells regulated by antigen recognition results in preferential proliferation and expansion of responding CD8+ T cells rather than promotion of cell death.” Proceedings of the National Academy of Sciences of the United States of America vol. 99, 5 (2002): 3001-6. doi:10.1073/pnas.052676899; Cho, Jae-Ho et al. “Unique features of naive CD8+ T cell activation by IL-2.” Journal of immunology (Baltimore, Md.: 1950) vol. 191, 11 (2013): 5559-73. doi:10.4049/jimmunol.1302293; the entire contents of both of which is incorporated herein by reference for all purposes). IL-6 has been shown to suppress TNF production induced by lipopolysaccharide (LPS) or phytohemagglutinins (PHA) (Schindler, R et al. “Correlations and interactions in the production of interleukin-6 (IL-6), IL-1, and tumor necrosis factor (TNF) in human blood mononuclear cells: IL-6 suppresses IL-1 and TNF.” Blood vol. 75, 1 (1990): 40-7; the entire contents of which is incorporated herein by reference for all purposes) while IL-10 inhibits the production of IL-6 by T cells (Hempel, L et al. “Interleukin-10 directly inhibits the interleukin-6 production in T-cells.” Scandinavian journal of immunology vol. 41, 5 (1995): 462-6. doi:10.1111/j.1365-3083.1995.tb03593.x; the entire contents of which is incorporated herein by reference for all purposes). Thus, lower level of IL-10 at day 7 could be reciprocally regulated by the increased level of IL-6 at day 7. Furthermore, the high level of IL-6 at day 7 might lead to reduction in TNF levels at day 7 compared to day 3 as shown by earlier research (Schindler, R et al. “Correlations and interactions in the production of interleukin-6 (IL-6), IL-1, and tumor necrosis factor (TNF) in human blood mononuclear cells: IL-6 suppresses IL-1 and TNF.” Blood vol. 75, 1 (1990): 40-7; the entire contents of which is incorporated herein by reference for all purposes). IL-4 (FIG. 20E) and IL-17 (FIG. 20G) secretion levels were low. The induction of cytokines by BCA424.EG is Th1-biased at day 3 and the cytokines are temporally regulated at day 7, and both the mechanisms influence anti-tumour immune response. Overall, the data suggest that in the presence of target cells, the tri-specific antibody increases the release of pro-inflammatory cytokines for anti-tumour activity.

### 6.11 Example 11. TAA Binding of Multispecific Proteins

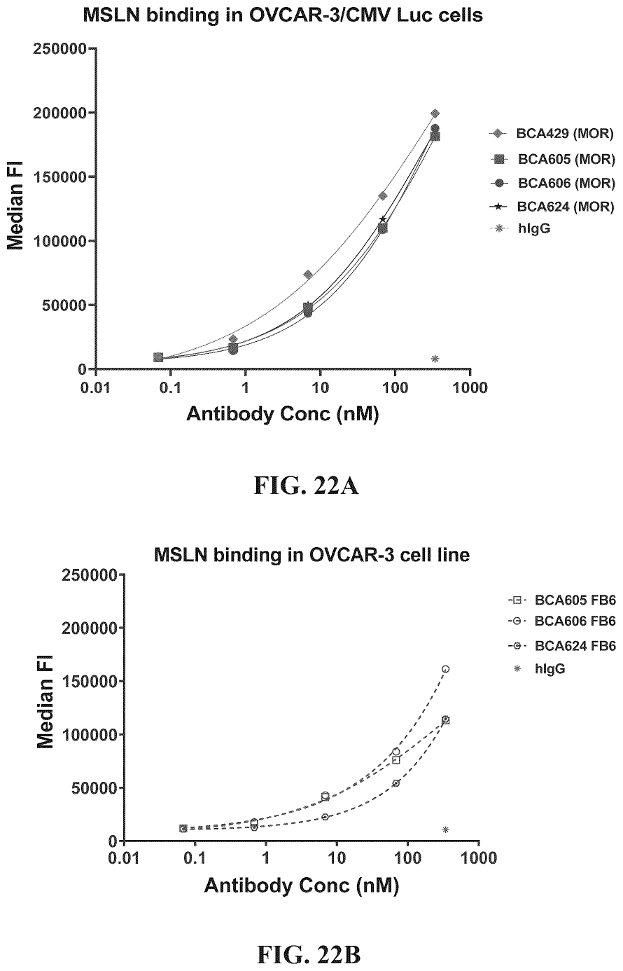

Binding of the multispecific proteins (BCA605 (MOR), BCA606 (MOR), BCA624 (MOR), BCA605 (FB6), BCA606 (FB6), BCA624 (FB6)) to mesothelin (MSLN) was assessed using a flow cytometry-based binding assay using OVCAR3/CMV Luc (MSLN high) cells. Briefly, OVCAR3/CMV Luc (MSLN high) cells were incubated with serially diluted primary antibody (multispecific protein or human isotype control antibody (hIgG (Bioxcell)) in ice-cold 1×FACS staining buffer (1×PBS with 2% FBS and 0.5M EDTA) for 40 min at 4° C. This was followed by a wash and subsequent incubation with secondary antibody (Goat anti-human IgG Fc specific-FITC (Sigma-Aldrich); 1:100 in ice-cold 1×FACS Staining buffer). This was followed by a wash and incubation with 7-AAD (BD-Biosciences) in PBS for 10 min at room temperature (RT). Cells were then acquired on Cytoflex flow cytometer (Becton Dickinson). Data was analyzed using FlowJo software. BCA605 (MOR), BCA606 (MOR) and BCA624 (MOR) multispecific antibodies showed binding to mesothelin and comparable binding to the control anti-MSLN antibody (BCA429) (FIG. 22A); and BCA605 (FB6), BCA606 (FB6) and BCA624 (FB6) multispecific antibodies also showed binding to mesothelin (FIG. 22B).

### 6.12 Example 12. CD28 Binding of Multispecific Proteins

Binding of the multispecific proteins, (BCA605 (MOR), BCA606 (MOR), BCA624 (MOR), BCA605 (FB6), BCA606 (FB6), BCA624 (FB6)) to CD28 was assessed by a flow cytometry-based binding assay using CD3e knockout Jurkat cells. Briefly, CD3e knockout Jurkat cells were incubated with serially diluted primary antibody (multispecific proteins and human isotype control antibody (hIgG (Bioxcell)), BCA402-anti-CD28 mAb, BCA403-anti-CD3 mAb) in ice-cold 1×FACS staining buffer (1×PBS with 2% FBS and 0.5M EDTA) for 40 min at 4° C. This was followed by a wash and subsequent incubation with secondary antibody (Goat anti-human IgG Fc specific-FITC (Sigma-Aldrich); 1:100 in ice-cold 1×FACS Staining buffer). This was followed by a wash and incubation with 7-AAD (BD-Biosciences) in PBS for 10 min at RT. Cells were then acquired on Cytoflex flow cytometer (Becton Dickinson). Data was analyzed using FlowJo software.

The EC50 value (nM) of CD28 binding of the indicated multispecific antibodies as measured by flow cytometry is set forth in Table 18.

TABLE 18
EC50 (nM) of CD28 Binding of the Indicated Multispecific Antibody

| | BCA605 | BCA606 | BCA624 | BCA605 | BCA606 | BCA624 |
|---|---|---|---|---|---|---|
| | (MOR) | (MOR) | (MOR) | (FB6) | (FB6) | (FB6) |
| EC50 | 74.77 | 23.91 | 108 | 38.06 | 58.83 | 6.738 |
| (nM) | | | | | | |

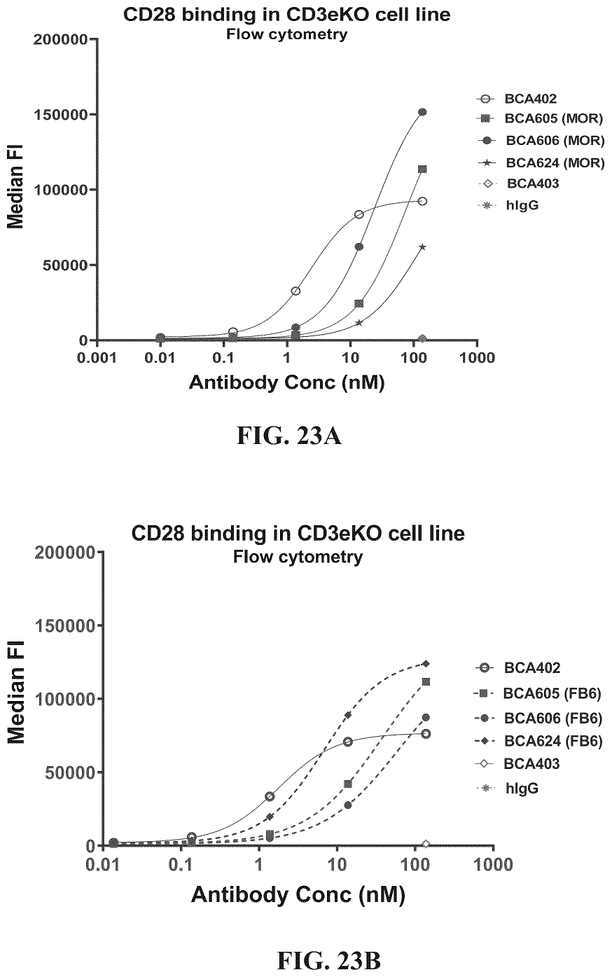

At higher antibody concentrations, the binding of BCA605 (MOR), BCA605(FB6) and BCA606 (MOR), BCA606 (FB6) are comparable to BCA402 (positive control anti-CD28 mAb) and slightly attenuated at lower concentrations (FIG. 23A and FIG. 23B). BCA624 (MOR) binding to CD28 is attenuated compared to BCA402, followed by BCA605(MOR) and BCA606(MOR) (FIG. 23A). The binding of BCA624 (FB6) is comparable to BCA402 binding, with BCA605(FB6) and BCA606(FB6) attenuated at lower concentrations (FIG. 23B).

### 6.13 Example 13. CD3 Binding of Multispecific Proteins

Binding of the multispecific proteins (BCA605 (MOR), BCA606 (MOR), BCA624 (MOR), BCA605 (FB6), BCA606 (FB6), BCA624 (FB6)) to CD3 was first assessed by ELISA. Briefly, 96-well Maxi-Sorp plates (Nunc) were coated with 1 μg/mL CD3 extracellular domain (Acrobiosystem) at 4° C. overnight. The following day, the plates were washed with wash Buffer (0.05% Tween-20 (Sigma-Aldrich) in filtered DPBS (Gibco)) and blocked using SuperBlock™ blocking buffer (Thermo Fisher) for 1.5-2 hours. The plates were then incubated with serially diluted primary antibody (multispecific proteins, human isotype control antibody (hIgG (Bioxcell)) made in Assay diluent (wash buffer containing 10% SuperBlock™) for 1 hour at RT. This was followed by wash with wash buffer and subsequent incubation with secondary antibody (Donkey anti-human IgG H+L peroxidase (Jackson ImmunoResearch) diluted 1:25000 in assay diluent) for 1 hour at RT. After incubation, plates were washed with wash buffer. The colour was developed by adding TMB substrate solution (Sigma-Aldrich) and the reaction was stopped using Stop Solution (1N H2SO4). The optical density of each well was determined immediately, using a microplate reader at 450 nm with 630 nm as the reference wavelength.

Binding of the multispecific proteins, (BCA605 (MOR), BCA606 (MOR), BCA624 (MOR), BCA605 (FB6), BCA606 (FB6), BCA624 (FB6) to CD3 was also assessed by a flow cytometry-based binding assay using CD28 knockout Jurkat cells. Briefly, CD28 knockout Jurkat cells were incubated with serially diluted primary antibody (multispecific proteins, human isotype control antibody (hIgG (Bioxcell), BCA402, anti-CD28 mAb, BCA403, anti-CD3 mAb) in ice-cold 1×FACS staining buffer (1×PBS with 2% FBS and 0.5M EDTA) for 40 min at 4° C. This was followed by a wash and subsequent incubation with secondary antibody (Goat anti-human IgG Fc specific-FITC (Sigma-Aldrich); 1:100 in ice-cold 1×FACS Staining buffer). This was followed by a wash and incubation with 7-AAD (BD-Biosciences) in PBS for 10 min at RT. Cells were then acquired on Cytoflex flow cytometer (Becton Dickinson). Data was analysed using FlowJo software.

The EC50 value (nM) of CD3 binding of the indicated multispecific antibodies as measured by ELISA is set forth in Table 19.

TABLE 19
EC50 (nM) of CD3 Binding of the Indicated Multispecific Antibody

| | BCA605 | BCA606 | BCA624 | BCA605 | BCA606 | BCA624 |
|---|---|---|---|---|---|---|
| | (MOR) | (MOR) | (MOR) | (FB6) | (FB6) | (FB6) |
| EC50 | No binding | ++ | No binding | No binding | No binding | + |
| (nM) | | | | | | |

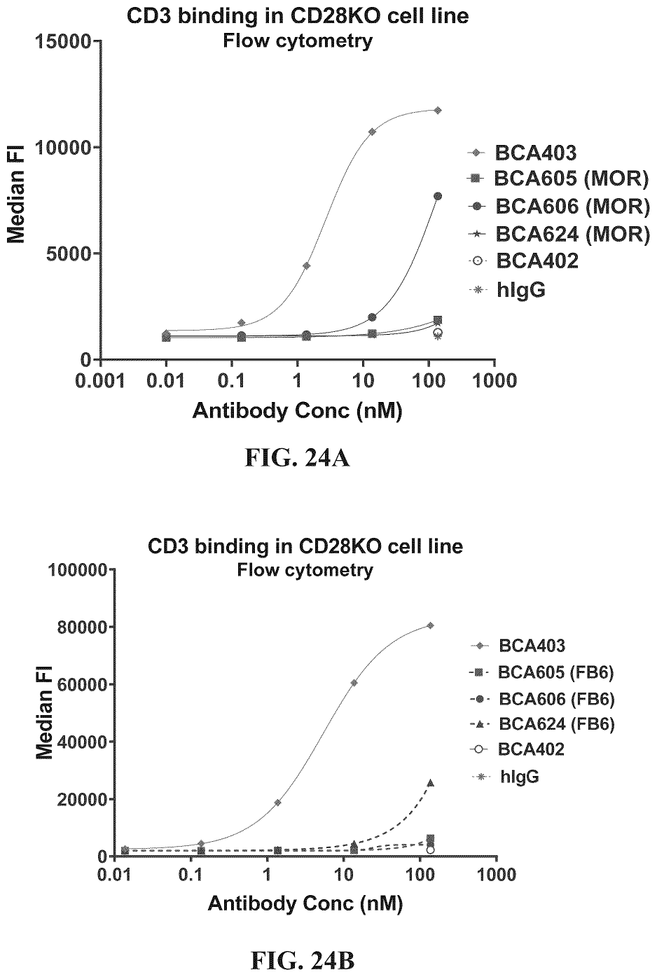

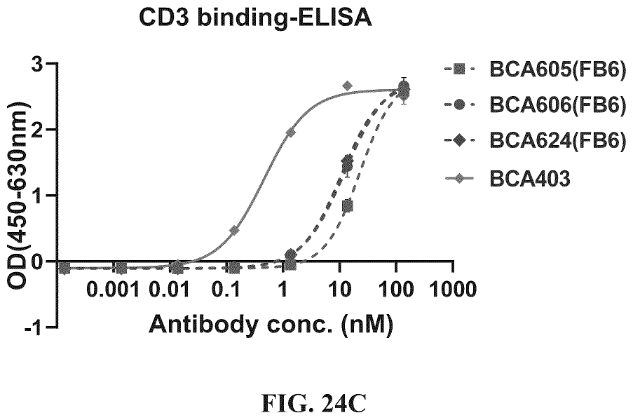

The three multispecific antibody formats show attenuated CD3 binding compared to BCA403 (anti-CD3 mAb, positive control). At the highest concentration, among the MOR antibodies, BCA606(MOR) shows highest binding (FIG. 24A) and BCA624(FB6) showing the highest binding among the FB6 antibodies (FIG. 24B). The FB6 clones of all three formats showed attenuated binding in flow cytometry assay (FIG. 24B) with improved binding was observed in ELISA assay (FIG. 24C). Although FB6 multispecific antibodies showed attenuated binding in the ELISA assay, dose-dependent binding was observed (FIG. 24C).

### 6.14 Example 14. Multispecific Protein Mediated Tumor Cell Cytotoxicity

The cytolytic potential of the multispecific proteins was evaluated using co-culture assays. Briefly, OVCAR3 CMV/luc (MSLN high cells) and SKOV3 CMV/Luc (MSLN low cells) target cells were co-cultured with human PBMCs at T:E (target: effector) ratio of 1:10 for 48 h. A total of 1×10⁴ tumor (target) cells per well were seeded in white-walled, clear bottom (flat) 96-well plates (Nunc) and incubated at 37° C. and 5% C02 overnight for cell attachment. On the same day PBMCs (effector) were revived in PBMC revival media (RPMI-1640 (2C) (Gibco)+10% HI-FBS+100 IU/mL (Gibco) rhIL-2 (R&D Systems), filtered) and incubated overnight at 37° C. and 5% CO₂. PBMCs were added to target cells at an effector to target cell ratio of 10:1 and incubated for 48 hours in the presence of the indicated concentrations of the multispecific proteins or a hIgG control. At the end of incubation, plates were spun, and a part of culture medium was transferred to a new 96-well plate. The extent of cell lysis in the target cells was determined using either BioGlo (Promega Corp.) 80 μL/well of BioGlo reagent was added, incubated at RT in dark for 5 mins and luminescence reading was taken on Cytation 5 (BioTek Instruments). Percent cytotoxicity was evaluated by luminescence-based readouts using Bioglo.

The EC50 value (nM) for OVACAR3-CMV Luc toxicity of the indicated multispecific antibodies is set forth in Table 20.

TABLE 20
EC50 (nM) for OVACAR3-CMV Luc Toxicity
of the Indicated Multispecific Antibody

| PBMC | BCA605 | BCA606 | BCA624 | BCA605 | BCA606 | BCA624 |
|---|---|---|---|---|---|---|
| Lot # | (MOR) | (MOR) | (MOR) | (FB6) | (FB6) | (FB6) |
| 020822D | 0.026 | 0.016 | 0.003 | 0.060 | 1.379 | 17.870 |
| 020822A | 0.022 | 0.005 | 0.000 | NA | NA | NA |
| 113368 | 0.236 | 0.005 | 0.005 | NA | NA | NA |
| 090120B | 0.101 | 0.008 | 0.002 | 0.025 | 0.185 | — |
| Average | 0.096 | 0.009 | 0.003 | 0.043 | 0.782 | 17.870 |

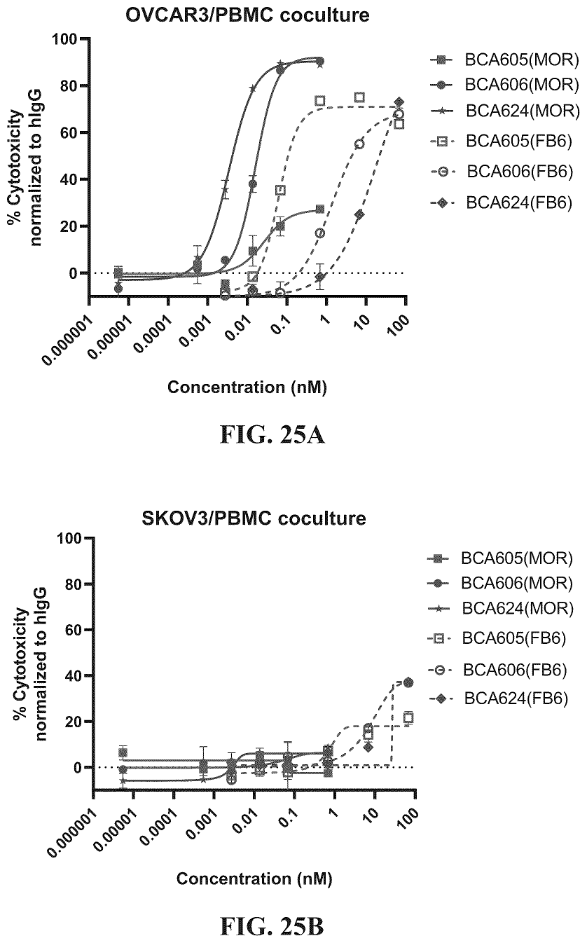

Low/no cytotoxicity was observed in SKOV3 cells that express low levels of MSLN (FIG. 25C), suggesting that the anti-TAA arm (e.g., anti-MSLN) arm is a cardinal driver of antibody binding to the target cells and simultaneous activation of T cells due to CD3 and CD28 engagement. Differences in cytotoxicity between the formats could potentially be attributed to differences in binding of the multispecific proteins to MSLN, CD3 and CD28 or structural differences (e.g., the membrane proximity of the FB6 binding epitope which may lead to steric hindrance of anti-CD3 and anti-CD28 binding in the BCA606 and BCA624 formats and not in BCA605).

### 6.15 Example 15. Cytokine Release

Cytokine release from the in vitro target cell-PBMC co-culture assay supernatant in Example 14 was assessed using a cytokine release assay (CRA). Cytokine level determination was performed using either Human Th1/Th2/Th17 CBA Kit (BD Bioscience) or Cytometric Bead Array (CBA) Human Flex Set (BD Bioscience) according to manufacturer's instructions. Data was analysed using FCAP Array software and graphs were plotted on GraphPad Prism.

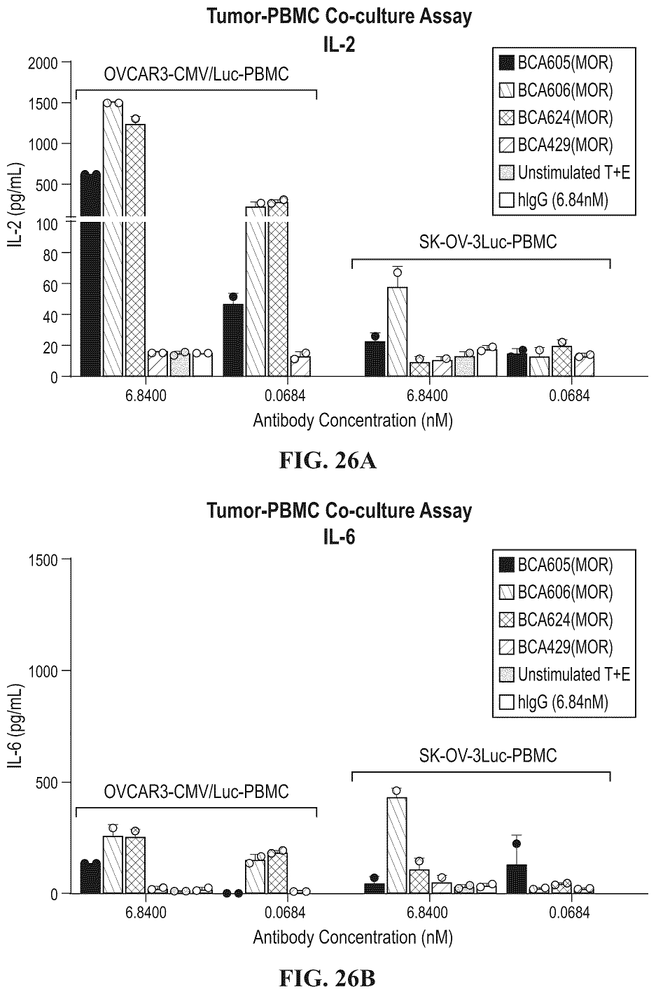

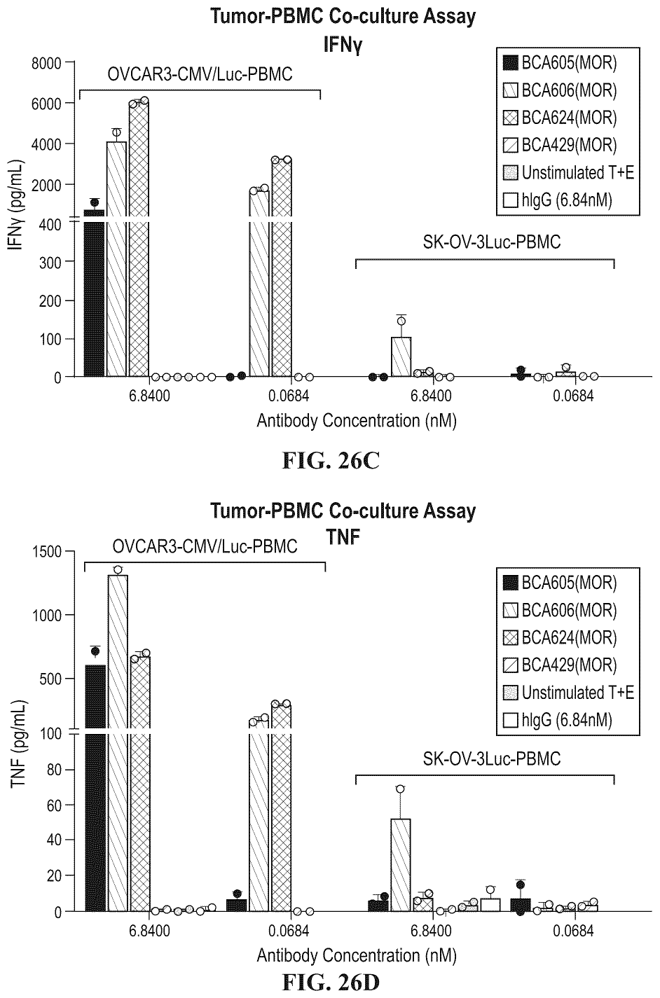

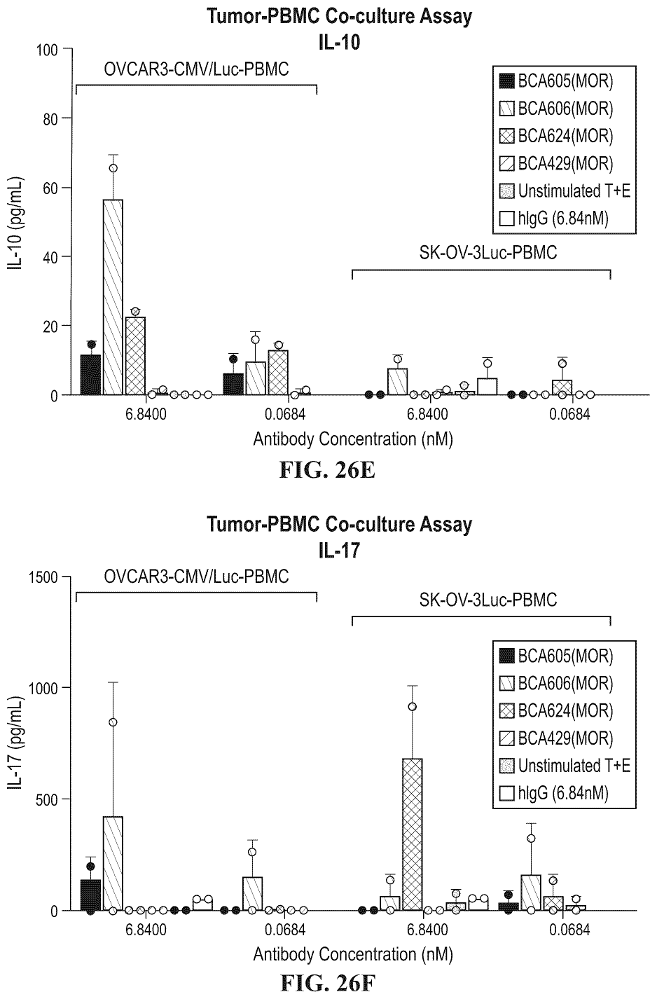

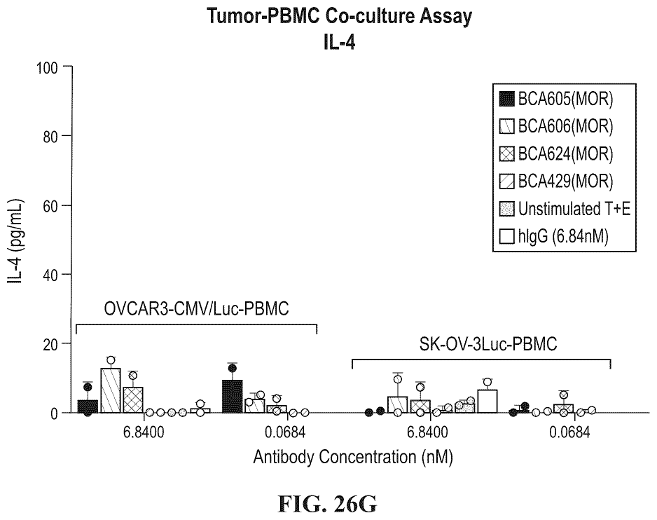

Higher cytokine release overall was observed from PBMC cocultured with OVCAR3 CMV/luc (express high levels of MSLN) while PBMC coculture with SKOV3 CMV/luc cells (express low levels of MSLN) produced negligible levels of most cytokines (FIGS. 26A-26G). Showing that tumor-immune cell synapse is critical for cytokine release to occur; thus, prospectively mitigating the risk of peripheral CD3 engagement and associated cytokine release syndrome (CRS). BCA606 (MOR) and BCA624(MOR) showed higher cytokine release compared to BCA605 (MOR) concurrent with the CD3 binding profiles. Dose-dependent cytokine release was observed (FIGS. 26A-26G). Higher levels of IL-2, IFN7 and TNFα and low levels of IL-10 and IL-4 are indicative of anti-tumor immune responses, predominantly Th1 responses, in this coculture model.

### 6.16 Example 16. IFNγ Release

ELISA was performed to analyse the IFN7 levels in the cell culture supernatant from PBMCs treated with soluble or tethered multispecific antibodies: data from two PBMC lots is shown. Human IFN7 Quantikine ELISA Kit (R&D Systems) was used as per manufacturer's instructions. The optical density of each well was determined immediately after completion of the ELISA protocol, using a microplate reader at 450 nm with 540 nm as the reference wavelength.

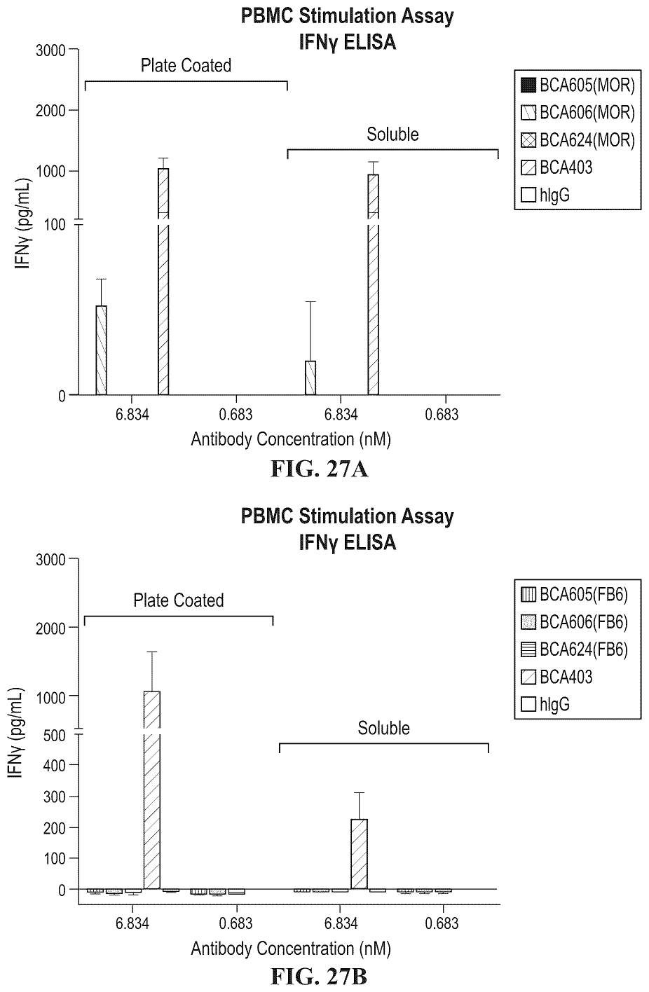

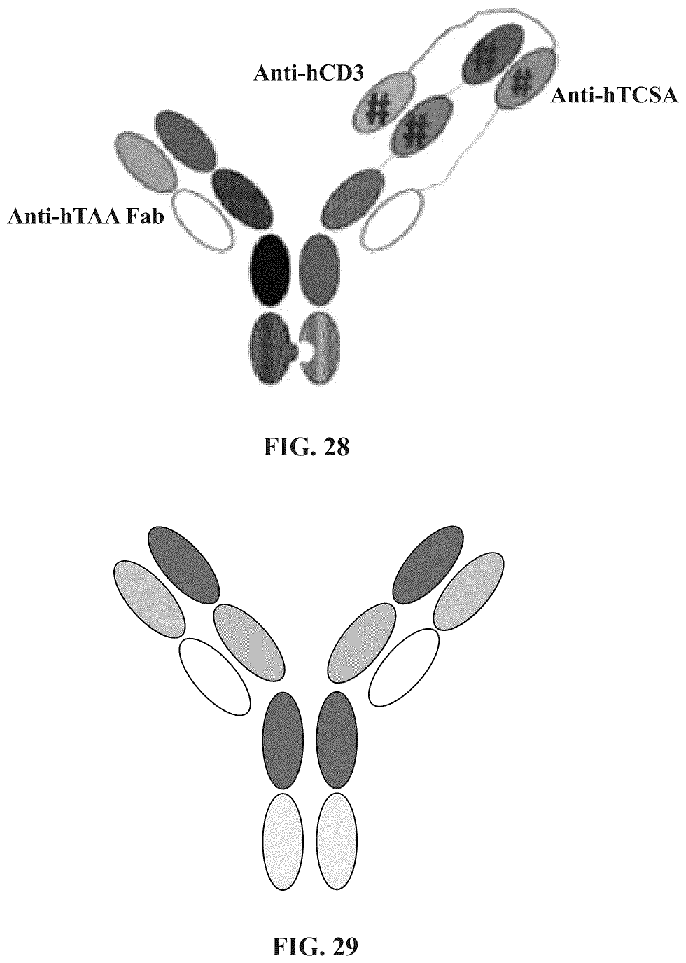

Immobilized/tethered (i.e., plate coated) anti-CD3 showed enhanced cross-linking to T cells relative to the soluble form of anti-CD3, hence providing a stronger activation signal. Higher IFNγ release was observed from tethered versus soluble BCA606 (MOR) mediated stimulation of PBMC which is indicative of the ability of this antibody to potentially directly stimulate PBMCs (FIG. 27A). IFNγ release is <100 pg/ml in the PBMC donor tested. None of the FB6 antibodies induced IFNγ release directly from PBMCs concurrent with the finding that CD3 binding of these antibodies was attenuated (FIG. 27B).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed is:

1. A multispecific protein comprising:
(a) a full-length antibody comprising:
(i) a first light chain comprising from N- to C-terminus a light chain variable region (VL) region and a light chain constant region (CL) region;
(ii) a first heavy chain comprising from N- to C-terminus a heavy chain variable region (VH) region, a CH1 region, a hinge region, a CH2 region, and a CH3 region;
(iii) a second heavy chain comprising from N- to C-terminus a VH region, a CH1 region, a hinge region, a CH2 region, and a CH3 region;
(iv) a second light chain comprising from N- to C-terminus a VL region and a CL region;
wherein said first light chain and said first heavy chain associate to form a first antigen binding domain;
wherein said second light chain and said second heavy chain associate to form a second antigen binding domain; and
wherein said first heavy chain and said second heavy chain associate to form a dimer;
(b) a first single chain variable fragment (scFv) operably connected to the C-terminus of said CH3 region of said first heavy chain of said full-length antibody, wherein said first scFv comprises from N- to C-terminus (i) a VH region, a peptide linker, and a VL region, or (ii) a VL region, a peptide linker, and a VH region; and
(c) a second scFv operably connected to the C-terminus of said CH3 region of said second heavy chain of said full-length antibody, wherein said second scFv comprises from N- to C-terminus (i) a VH region, a peptide linker, and a VL region, or (ii) a VL region, a peptide linker, and a VH region;
wherein said first antigen binding domain of said full-length antibody specifically binds to a first human tumor associated antigen (hTAA);
wherein said second antigen binding domain of said full-length antibody specifically binds to a second hTAA;
wherein said first scFv specifically binds to a human T-cell co-stimulatory antigen (hTCSA) (e.g., hCD28, hCD2);
wherein said second scFv specifically binds to human CD3 (hCD3); and
wherein said CH3 region of said first heavy chain of said full-length antibody comprises one or more amino acid modification (e.g., substitution) relative to the amino acid sequence of a reference CH3 region that does not contain said one or more amino acid modification (e.g., a reference CH3 region, e.g., a wild-type CH3 region, e.g., SEQ ID NO: 101),
wherein said CH3 region of said second heavy chain of said of the full-length antibody comprises one or more amino acid modification (e.g., substitution) relative to the amino acid sequence of a reference CH3 region that does not contain said one or more amino acid modification (e.g., a wild-type CH3 region, e.g., SEQ ID NO: 101); and
wherein said one or more amino acid modification in said CH3 region of said first heavy chain of said full-length antibody is different from said one or more amino acid modification in said CH3 region of said second heavy chain of said full-length antibody;
wherein said one or more amino acid modification in said CH3 region of said first heavy chain of said full-length antibody and said one or more amino acid modification in said CH3 region of said second heavy chain of said full-length antibody promote heterodimerization of said first and second heavy chain of said full-length antibody;
wherein said CH2 region of said first heavy chain of said full-length antibody comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said CH2 region of said second heavy chain of said full-length antibody comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said one or more amino acid modification in said CH2 region of said first heavy chain of said full-length antibody and said one or more amino acid modification in said CH2 region of said second heavy chain of said full-length antibody reduce or abolish one or more of the following heavy chain effector functions relative to a reference heavy chain that does not contain said one or more amino acid modification (e.g., a heavy chain comprising a wild type CH2 region, e.g., SEQ ID NO: 100): antibody dependent cellular cytotoxicity (ADCC), antibody dependent cellular phagocytosis (ADCP), complement dependent cytotoxicity (CDC), and/or binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

2. The multispecific protein of claim 1, wherein said first scFv is operably connected to the C-terminus of said CH3 region of said first heavy chain of said full-length antibody directly through a peptide bond.

3. The multispecific protein of claim 1, wherein said first scFv is operably connected to the C-terminus of said CH3 region of said first heavy chain of said full-length antibody indirectly through a first peptide linker.

4. The multispecific protein of claim 3, wherein the amino acid sequence of said first peptide linker comprises or consists of glycine or glycine and serine amino acid residues.

5. The multispecific protein of any one of claim 3 or 4, wherein the amino acid sequence of said first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

6. The multispecific protein of any one of the preceding claims, wherein said second scFv is operably connected to the C-terminus of said CH3 region of said second heavy chain of said full-length antibody directly through a peptide bond.

7. The multispecific protein of any one of claims 1-5, wherein said second scFv is operably connected to the C-terminus of said CH3 region of said second heavy chain of said full-length antibody through a second peptide linker.

8. The multispecific protein of claim 7, wherein the amino acid sequence of said second peptide linker comprises or consists of glycine or glycine and serine amino acid residues.

9. The multispecific protein of any one of claim 7 or 8, wherein the amino acid of said second peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

10. The multispecific protein of any one of the preceding claims, wherein said first scFv comprises from N- to C-terminus: a VL region, a peptide linker, and a VH region; and wherein the N-terminus of said VL region of said scFv is operably connected to the C-terminus of said CH3 region of said first heavy chain of said full-length antibody.

11. The multispecific protein of any one of the preceding claims, wherein said first scFv comprises from N- to C-terminus: a VH region, a peptide linker, and a VL region; and wherein the N-terminus of said VH region of said scFv is operably connected to the C-terminus of said CH3 region of said first heavy chain of said full-length antibody.

12. The multispecific protein of any one of the preceding claims, wherein said second scFv comprises from N- to C-terminus: a VL region, a peptide linker, and a VH region; and wherein the N-terminus of said VL region of said scFv is operably connected to the C-terminus of said CH3 region of said second heavy chain of said full-length antibody.

13. The multispecific protein of any one of the preceding claims, wherein said second scFv comprises from N- to C-terminus: a VH region, a peptide linker, and a VL region; and wherein the N-terminus of said VH region of said scFv is operably connected to the C-terminus of said CH3 region of said second heavy chain of said full-length antibody.

14. A multispecific protein comprising:
(a) a full-length antibody comprising:
(i) a first light chain comprising from N- to C-terminus a VL region and a CL region;
(ii) a first heavy chain comprising from N- to C-terminus a VH region, a CH1 region, a hinge region, a CH2 region, and a CH3 region;
(iii) a second heavy chain comprising from N- to C-terminus a VH region, a CH1 region, a hinge region, a CH2 region, and a CH3 region;
(iv) a second light chain comprising from N- to C-terminus a VL region and a CL region;
wherein the first light chain and first heavy chain associate to form a first antigen binding domain;
wherein the second light chain and the second heavy chain associate to form a second antigen binding domain; and
wherein the first heavy chain and second heavy chain associate to form a dimer;
(b) a first scFv operably connected to the N-terminus of said first heavy chain of said full-length antibody, wherein said first scFv comprises from N- to C-terminus (i) a VH region, a peptide linker, and a VL region, or (ii) a VL region, a peptide linker, and a VH region; and
(c) a second scFv operably connected to the N-terminus of said second heavy chain of said full-length antibody, wherein said second scFv comprises from N- to C-terminus (i) a VH region, a peptide linker, and a VL region, or (ii) a VL region, a peptide linker, and a VH region;
wherein said first antigen binding domain of said full-length antibody specifically binds to a first hTAA;
wherein said second antigen binding domain of said full-length antibody specifically binds to a second hTAA;
wherein said first scFv specifically binds to a hTCSA (e.g., hCD28, hCD2);
wherein said second scFv specifically binds to hCD3; and
wherein said CH3 region of said first heavy chain of said full-length antibody comprises one or more amino acid modification (e.g., substitution) relative to the amino acid sequence of a reference CH3 region that does not contain said one or more amino acid modification (e.g., a wild-type CH3 region, e.g., SEQ ID NO: 101),
wherein said CH3 region of said second heavy chain of said of the full-length antibody comprises one or more amino acid modification (e.g., substitution) relative to the amino acid sequence of a reference CH3 region that does not contain said one or more amino acid modification (e.g., a wild-type CH3 region, e.g., SEQ ID NO: 101); and
wherein said one or more amino acid modification in said CH3 region of said first heavy chain of said full-length antibody is different from said one or more amino acid modification in said CH3 region of said second heavy chain of said full-length antibody;
wherein said one or more amino acid modification in said CH3 region of said first heavy chain of said full-length antibody and said one or more amino acid modification in said CH3 region of said second heavy chain of said full-length antibody promote heterodimerization of said first and second heavy chain of said full-length antibody;
wherein said CH2 region of said first heavy chain of said full-length antibody comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said CH2 region of said second heavy chain of said full-length antibody comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said one or more amino acid modification in said CH2 region of said first heavy chain of said full-length antibody and said one or more amino acid modification in said CH2 region of said second heavy chain of said full-length antibody reduce or abolish one or more of the following heavy chain effector functions relative to a reference heavy chain that does not contain said one or more amino acid modification (e.g., a heavy chain comprising a wild type CH2 region, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

15. The multispecific protein of claim 14, wherein said first scFv is operably connected to the N-terminus of said VH region of said first heavy chain of said full-length antibody directly through a peptide bond.

16. The multispecific protein of claim 14, wherein said first scFv is operably connected to the N-terminus of said VH region of said first heavy chain of said full-length antibody through a first peptide linker.

17. The multispecific protein of claim 16, wherein the amino acid sequence of said first peptide linker comprises or consists of glycine or glycine and serine amino acid residues.

18. The multispecific protein of any one of claim 15 or 16, wherein the amino acid sequence of said first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

19. The multispecific protein of any one of claims 14-18, wherein said second scFv is operably connected to the N-terminus of said VH region of said second heavy chain of said full-length antibody directly through a peptide bond.

20. The multispecific protein of any one of claims 14-18, wherein said second scFv is operably connected to the N-terminus of said VH region of said second heavy chain of said full-length antibody through a second peptide linker.

21. The multispecific protein of claim 20, wherein the amino acid sequence of said second peptide linker comprises or consists of glycine or glycine and serine amino acid residues.

22. The multispecific protein of any one of claim 20 or 21, wherein the amino acid of said second peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

23. The multispecific protein of any one of claims 14-22, wherein said first scFv comprises from N- to C-terminus: a VL region, a peptide linker, and a VH region; and wherein the C-terminus of said VH region of said scFv is operably connected to the N-terminus of said VH region of said first heavy chain of said full-length antibody.

24. The multispecific protein of any one of claims 14-23, wherein said first scFv comprises from N- to C-terminus: a VH region, a peptide linker, and a VL region; and wherein the C-terminus of said VL region of said scFv is operably connected to the N-terminus of said VH region of said first heavy chain of said full-length antibody.

25. The multispecific protein of any one of claims 14-24, wherein said second scFv comprises from N- to C-terminus: a VL region, a peptide linker, and a VH region; and wherein the C-terminus of said VH region of said scFv is operably connected to the N-terminus of said VH region of said second heavy chain of said full-length antibody.

26. The multispecific protein of any one of claims 14-25, wherein said second scFv comprises from N- to C-terminus: a VH region, a peptide linker, and a VL region; and wherein the C-terminus of said VL region of said scFv is operably connected to the N-terminus of said VH region of said second heavy chain of said full-length antibody.

27. The multispecific protein of any one of the preceding claims, wherein said first hTAA and said second hTAA are expressed by (e.g., on the surface of) the same tumor cell.

28. The multispecific protein of any one of the preceding claims, wherein said first hTAA and said second hTAA are the same.

29. The multispecific protein of any one of the preceding claims, wherein said first scFv and said second scFv specifically bind the same epitope of the same hTAA.

30. The multispecific protein of any one of the preceding claims, wherein said first scFv and said second scFv specifically bind different epitopes of the same hTAA.

31. The multispecific protein of any one of the preceding claims, wherein said first tumor associated antigen is hEGFR, hMSLN, hPSMA, or hHER2; and said second tumor associated antigen is hEGFR, hMSLN, hPSMA, or hHER2.

32. The multispecific protein of any one of the preceding claims, wherein the amino acid sequence of said VH region of said first scFv and the amino acid sequence of said VL region of said first scFv each comprises a cysteine amino acid residue, wherein said cysteine amino acid residues are capable of forming a disulfide bond.

33. The multispecific protein of claim 32, wherein the amino acid sequence of said VH region of said first scFv comprises a cysteine at amino acid position 97, 98, 99, 100, 101, 102, 103, 104, 105, or 106, amino acid numbering according to Kabat; and the amino acid sequence of said VL region of said first scFv comprises a cysteine at amino acid position 40, 41, 42, 43, 44, 45, 46, or 47, amino acid numbering according to Kabat.

34. The multispecific protein of claim 32 or 33, wherein the amino acid sequence of said VH region of said second scFv comprises a cysteine at amino acid position 97, 98, 99, 100, 101, 102, 103, 104, 105, or 106, amino acid numbering according to Kabat; and the amino acid sequence of said VL region of said second scFv comprises a cysteine at amino acid position 40, 41, 42, 43, 44, 45, 46, or 47, amino acid numbering according to Kabat.

35. The multispecific protein of any one of the preceding claims, wherein said full-length antibody is a human IgG (hIgG) antibody.

36. The multispecific protein of any one of the preceding claims, wherein said full-length antibody is a hIgG1 or hIgG4 antibody.

37. The multispecific protein of any one of the preceding claims, wherein said full-length antibody is an IgG1 antibody, and wherein the amino acid sequence of said first heavy chain of said full-length antibody comprises an amino acid substitution at amino acid positions T366, L368, and Y407, numbering according to the EU index of Kabat.

38. The multispecific protein of any one of the preceding claims, wherein said full-length antibody is an IgG1 antibody, and wherein the amino acid sequence of said first heavy chain of said full-length antibody comprises a serine at amino acid position T366, an alanine at amino acid position L368, and a valine at amino acid position Y407, numbering according to the EU index of Kabat.

39. The multispecific protein of any one of the preceding claims, wherein said full-length antibody is an IgG1 antibody, and wherein the amino acid sequence of said first heavy chain of said full-length antibody comprises an amino acid substitution at amino acid position Y349, numbering according to the EU index of Kabat.

40. The multispecific protein of any one of the preceding claims, wherein the full-length antibody is an IgG1 antibody, and wherein the amino acid sequence of said first heavy chain of said full-length antibody comprises a cysteine at amino acid position Y349, numbering according to the EU index of Kabat.

41. The multispecific protein of any one of the preceding claims, wherein the full-length antibody is an IgG1 antibody, and wherein the amino acid sequence of said second heavy chain of said full-length antibody comprises an amino acid substitution at amino acid position T366, numbering according to the EU index of Kabat.

42. The multispecific protein of any one of the preceding claims, wherein the full-length antibody is an IgG1 antibody, and wherein the amino acid sequence of said second heavy chain of said full-length antibody comprises a tryptophan at amino acid position T366, numbering according to the EU index of Kabat.

43. The multispecific protein of any one of the preceding claims, wherein the full-length antibody is an IgG1 antibody, and wherein the amino acid sequence of said second heavy chain of said full-length antibody comprises an amino acid substitution at amino acid position S354, numbering according to the EU index of Kabat.

44. The multispecific protein of any one of the preceding claims, wherein the full-length antibody is an IgG1 antibody, and wherein the amino acid sequence of said second heavy chain of said full-length antibody comprises a cysteine at amino acid position S354, numbering according to the EU index of Kabat.

45. The multispecific protein of any one of claims 1-36, wherein the full-length antibody is an IgG1 antibody, and wherein the amino acid sequence of said second heavy chain of said full-length antibody comprises an amino acid substitution at amino acid positions T366, L368, and Y407, numbering according to the EU index of Kabat.

46. The multispecific protein of any one of claims 1-36 or 45, wherein the full-length antibody is an IgG1 antibody, and wherein the amino acid sequence of said second heavy chain of said full-length antibody comprises a serine at amino acid position T366, an alanine at amino acid position L368, and a valine at amino acid position Y407, numbering according to the EU index of Kabat.

47. The multispecific protein of any one of claims 1-36 or 45-46, wherein the full-length antibody is an IgG1 antibody, and wherein the amino acid sequence of said second heavy chain of said full-length antibody comprises an amino acid substitution at amino acid position Y349, numbering according to the EU index of Kabat.

48. The multispecific protein of any one of claims 1-36 or 45-47, wherein the full-length antibody is an IgG1 antibody, and wherein the amino acid sequence of said second heavy chain of said full-length antibody comprises a cysteine at amino acid position Y349, numbering according to the EU index of Kabat.

49. The multispecific protein of any one of claims 1-36 or 45-48, wherein the full-length antibody is an IgG1 antibody, and wherein the amino acid sequence of said first heavy chain of said full-length antibody comprises an amino acid substitution at amino acid position T366, numbering according to the EU index of Kabat.

50. The multispecific protein of any one of claims 1-36 or 45-49, wherein the full-length antibody is an IgG1 antibody, and wherein the amino acid sequence of said first heavy chain of said full-length antibody comprises a tryptophan at amino acid position T366, numbering according to the EU index of Kabat.

51. The multispecific protein of any one of claims 1-36 or 45-50, wherein the full-length antibody is an IgG1 antibody, and wherein the amino acid sequence of said first heavy chain of said full-length antibody comprises an amino acid substitution at amino acid position S354, numbering according to the EU index of Kabat.

52. The multispecific protein of any one of claims 1-36 or 45-51, wherein the full-length antibody is an IgG1 antibody, and wherein the amino acid sequence of said first heavy chain of said full-length antibody comprises a cysteine at amino acid position S354, numbering according to the EU index of Kabat.

53. The multispecific protein of any one of the preceding claims, wherein said multispecific protein does not substantially mediate ADCC, does not substantially mediate ADCP, does not substantially mediate CDC, and/or does not bind to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

54. The multispecific protein of any one of the preceding claims, wherein said full-length antibody is an IgG1 antibody, and wherein the amino acid sequence of said first heavy chain and said second heavy chain each comprises an amino acid substitution at amino acid position L234, and/or an amino acid substitution at amino acid position L235, numbering according to the EU index of Kabat.

55. The multispecific protein of any one of the preceding claims, wherein said full-length antibody is an IgG1 antibody, and wherein the amino acid sequence of said first heavy chain and said second heavy chain each comprises an alanine at amino acid position L234 and/or an alanine at amino acid position L235, numbering according to the EU index of Kabat.

56. The multispecific protein of any one of the preceding claims, wherein said full-length antibody is an IgG1 antibody, and wherein the amino acid sequence of said first heavy chain and said second heavy chain each comprises an amino acid substitution at amino acid position L234, an amino acid substitution at amino acid position L235, and/or an amino acid substitution at amino acid position P329, numbering according to the EU index of Kabat.

57. The multispecific protein of any one of the preceding claims, wherein said full-length antibody is an IgG1 antibody, and wherein the amino acid sequence of said first heavy chain and said second heavy chain each comprises an alanine at amino acid position L234, an alanine at amino acid position L235, and/or an alanine at amino acid position P329, numbering according to the EU index of Kabat.

58. The multispecific protein of any one of the preceding claims, wherein said full-length antibody is an IgG4 antibody, and wherein the amino acid sequence of said first heavy chain and said second heavy chain each comprises an alanine at amino acid position L234, an alanine at amino acid position L235, and/or a glycine at amino acid position P329, numbering according to the EU index of Kabat.

59. The multispecific protein of any one of the preceding claims, wherein said full-length antibody is an IgG4 antibody, and wherein the amino acid sequence of said first heavy chain and said second heavy chain each comprises an amino acid substitution at amino acid position S228, an amino acid substitution at amino acid position F234, and/or an amino acid substitution at amino acid position E235, numbering according to the EU index of Kabat.

60. The multispecific protein of any one of the preceding claims, wherein said full-length antibody is an IgG4 antibody, and wherein the amino acid sequence of said first heavy chain and said second heavy chain each comprises a proline at amino acid position S228, an alanine at amino acid position F234, and/or an alanine at amino acid position E235, numbering according to the EU index of Kabat.

61. A multispecific protein comprising:
(a) first Fab comprising (i) a first Fab heavy chain comprising from N- to C-terminus a first VH region and a first CH1 region and (ii) a first light chain comprising from N- to C-terminus a first VL region and a first CL region;
(b) a first scFv operably connected to the C-terminus of said first CH1 region of said first Fab, wherein the first scFv comprises from N- to C-terminus (i) a VH region, a peptide linker, and a VL region; or (ii) a VL region, a peptide linker, and a VH region;
(c) a first Fc region operably connected to the C-terminus of said first scFv, wherein said first Fc region comprises from N- to C-terminus a CH2 region and a CH3 region; and
(d) second Fab comprising (i) a second Fab heavy chain comprising from N- to C-terminus a second VH region and a second CH1 region and (ii) a second light chain comprising from N- to C-terminus a second VL region and a first CL region;
(b) a second scFv operably connected to the C-terminus of said second CH1 of said second Fab, wherein said second scFv comprises from N- to C-terminus (i) a VH region, a peptide linker, and a VL region; or (ii) a VL region, a peptide linker, and a VH region;
(c) a second Fc region operably connected to the C-terminus of said second scFv, wherein said second Fc region comprises from N- to C-terminus a CH2 region and a CH3 region; and
wherein said first Fab specifically binds to a first human hTAA;
wherein said second Fab specifically binds to a second hTAA;
wherein said first scFv specifically binds to a hTCSA (e.g., hCD28, hCD2);
wherein said second scFv specifically binds to hCD3; and
wherein said CH3 region of said first Fc region comprises one or more amino acid modification (e.g., substitution) relative to the amino acid sequence of a reference CH3 region that does not contain said one or more amino acid modification (e.g., a wild-type CH3 region, e.g., SEQ ID NO: 101),
wherein said CH3 region of said second Fc region comprises one or more amino acid modification (e.g., substitution) relative to the amino acid sequence of a reference CH3 region that does not contain said one or more amino acid modification (e.g., a wild-type CH3 region, e.g., SEQ ID NO: 101); and
wherein said one or more amino acid modification in said CH3 region of said first Fc region is different from said one or more amino acid modification in said CH3 region of said Fc region;
wherein said one or more amino acid modification in said CH3 region of said first Fc region and said one or more amino acid modification in said CH3 region of said second Fc region promote heterodimerization of said first and second Fc region;
wherein said CH2 region of said first Fc region comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said CH2 region of said second Fc region comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said one or more amino acid modification in said CH2 region of said Fc region and said one or more amino acid modification in said CH2 region of said second Fc region reduce or abolish one or more of the following Fc region effector functions relative to a reference Fc region that does not contain said one or more amino acid modification (e.g., a heavy chain comprising a wild type CH2 domain, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

62. The multispecific protein of claim 61, wherein said first Fab is operably connected to said first scFv directly through a peptide bond.

63. The multispecific protein of claim 61, wherein said first Fab is operably connected said first scFv through a first peptide linker.

64. The multispecific protein of claim 63, wherein the amino acid sequence of said first peptide linker comprises or consists of glycine or glycine and serine amino acid residues.

65. The multispecific protein of any one of claim 63 or 64, wherein the amino acid sequence of said first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

66. The multispecific protein of any one of claims 61-65, wherein said second Fab is operably connected to said second scFv directly through a peptide bond.

67. The multispecific protein of any one of claims 61-65, wherein said second Fab is operably connected to said second scFv through a second peptide linker.

68. The multispecific protein of claim 67, wherein the amino acid sequence of said second peptide linker comprises or consists of glycine or glycine and serine amino acid residues.

69. The multispecific protein of any one of claim 67 or 68, wherein the amino acid of said second peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

70. The multispecific protein of any one of claims 61-69, wherein said first scFv comprises from N- to C-terminus: a VL region, a peptide linker, and a VH region; and wherein the N-terminus of said VL region of said scFv is operably connected to the C-terminus of said CH1 region of said first Fab.

71. The multispecific protein of any one of claims 61-70, wherein said first scFv comprises from N- to C-terminus: a VH region, a peptide linker, and a VL region; and wherein the N-terminus of said VH region of said scFv is operably connected to the C-terminus of said CH1 region of said first Fab.

72. The multispecific protein of any one of claims 61-71, wherein said second scFv comprises from N- to C-terminus: a VL region, a peptide linker, and a VH region; and wherein the C-terminus of said VH region of said scFv is operably connected to the N-terminus of said CH1 region of said second Fab.

73. The multispecific protein of any one of claims 61-72, wherein said second scFv comprises from N- to C-terminus: a VH region, a peptide linker, and a VL region; and wherein the C-terminus of said VL region of said scFv is operably connected to the N-terminus of said CH1 region of said second Fab.

74. The multispecific protein of any one of claims 61-73, wherein said first hTAA and said second hTAA are expressed by (e.g., on the surface of) the same tumor cell.

75. The multispecific protein of any one of claims 61-74, wherein said first hTAA and said second hTAA are the same.

76. The multispecific protein of any one of claims 61-75, wherein said first scFv and said second scFv specifically bind the same epitope of the same hTAA.

77. The multispecific protein of any one of claims 61-76, wherein said first scFv and said second scFv specifically bind different epitopes of the same hTAA.

78. The multispecific protein of any one of claims 61-77, wherein said first tumor associated antigen is hEGFR, hMSLN, hPSMA, or hHER2; and said second tumor associated antigen is hEGFR, hMSLN, hPSMA, or hHER2.

79. The multispecific protein of any one of claims 61-78, wherein the amino acid sequence of said VH region of said first scFv and the amino acid sequence of said VL region of said first scFv each comprises a cysteine amino acid residue, wherein said cysteine amino acid residues are capable of forming a disulfide bond.

80. The multispecific protein of claim 79, wherein the amino acid sequence of said VH region of said first scFv comprises a cysteine at amino acid position 97, 98, 99, 100, 101, 102, 103, 104, 105, or 106, amino acid numbering according to Kabat; and the amino acid sequence of said VL region of said first scFv comprises a cysteine at amino acid position 40, 41, 42, 43, 44, 45, 46, or 47, amino acid numbering according to Kabat.

81. The multispecific protein of claim 79 or 80, wherein the amino acid sequence of said VH region of said second scFv comprises a cysteine at amino acid position 97, 98, 99, 100, 101, 102, 103, 104, 105, or 106, amino acid numbering according to Kabat; and the amino acid sequence of said VL region of said second scFv comprises a cysteine at amino acid position 40, 41, 42, 43, 44, 45, 46, or 47, amino acid numbering according to Kabat.

82. A multispecific protein comprising:
(a) a scFv comprising from N- to C-terminus (i) a VH region, a peptide linker, and a VL region, or (ii) a VL region, a peptide linker, and a VH region;
(b) a Fab comprising (i) a Fab heavy chain comprising from N- to C-terminus a VH region and a CH1 region and (ii) a light chain comprising from N- to C-terminus a VL region and a CL region;
(c) a first Fc region comprising from N- to C-terminus a CH2 region and a CH3 region;
(d) a second Fc region comprising from N- to C-terminus a CH2 region and a CH3 region;
(e) an IgM CH2 mFab comprising (i) an IgM CH2 mFab heavy chain comprising from N- to C-terminus a VH region and an IgM CH2 region and (ii) an IgM CH2 mFab light chain comprising from N- to C-terminus a VL region and an IgM CH2 region;
wherein the C-terminus of said scFv is operably connected to the N-terminus of said Fab heavy chain of said first Fab;
wherein the C-terminus of said Fab heavy chain is operably connected to the N-terminus of said first Fc region;
wherein the C-terminus of said IgM CH2 mFab heavy chain is operably connected to the N-terminus of said second Fc region;
wherein said scFv specifically binds a hTCSA (e.g., hCD28, hCD2);
wherein said Fab specifically binds hCD3;
wherein said IgM CH2 mFab specifically binds a human tumor associated antigen (hTAA);
wherein said CH3 region of said first Fc region comprises one or more amino acid modification (e.g., substitution) relative to the amino acid sequence of a reference CH3 region that does not contain said one or more amino acid modification (e.g., a wild-type CH3 region, e.g., SEQ ID NO: 101),
wherein said CH3 region of said second Fc region comprises one or more amino acid modification (e.g., substitution) relative to the amino acid sequence of a reference CH3 region that does not contain said one or more amino acid modification (e.g., a wild-type CH3 region, e.g., SEQ ID NO: 101); and
wherein said one or more amino acid modification in said CH3 region of said first Fc region is different from said one or more amino acid modification in said CH3 region of said Fc region;
wherein said one or more amino acid modification in said CH3 region of said first Fc region and said one or more amino acid modification in said CH3 region of said second Fc region promote heterodimerization of said first and second heavy chain;
wherein said CH2 region of said first Fc region comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said CH2 region of said second Fc region comprises one or more amino acid modification (e.g., substitution) relative to the amino acid sequence of a reference CH2 region that does not contain said one or more amino acid modification (e.g., a wild-type CH2 region, e.g., SEQ ID NO: 100);
wherein said one or more amino acid modification in said CH2 region of said Fc region and said one or more amino acid modification in said CH2 region of said second Fc region reduce or abolish one or more of the following Fc region effector functions relative to a reference Fc region that does not contain said one or more amino acid modification (e.g., a heavy chain comprising a wild type CH2 domain, e.g., SEQ ID NO: 100): ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

83. The multispecific protein of claim 82, wherein said scFv is operably connected to the N-terminus of said Fab heavy chain of said first Fab directly through a peptide bond.

84. The multispecific protein of claim 82, wherein said scFv is operably connected to the N-terminus of said Fab heavy chain of said first Fab through a first peptide linker.

85. The multispecific protein of claim 84, wherein the amino acid sequence of said first peptide linker comprises or consists of glycine or glycine and serine amino acid residues.

86. The multispecific protein of any one of claim 84 or 85, wherein the amino acid sequence of said first peptide linker comprises or consists of (a) the amino acid sequence of any one of SEQ ID NOS: 217-236 or 318-319; or (b) the amino acid sequence of any one of SEQ ID NOS: 217-236 or 318-319 comprising or consisting of 1, 2, or 3 amino acid substitutions.

87. The multispecific protein of any one of claims 82-86, wherein said scFv comprises from N- to C-terminus: a VL region, a peptide linker, and a VH region; and wherein the C-terminus of said VH region of said scFv is operably connected to the N-terminus of said Fab heavy chain of said first Fab.

88. The multispecific protein of any one of claims 82-87, wherein said scFv comprises from N- to C-terminus: a VH region, a peptide linker, and a VL region; and wherein the C-terminus of said VL region of said scFv is operably connected to the N-terminus of said Fab heavy chain of said first Fab.

89. The multispecific protein of any one of claims 82-88, wherein said tumor associated antigen is hEGFR, hMSLN, hPSMA, or hHER2; and said second tumor associated antigen is hEGFR, hMSLN, hPSMA, or hHER2.

90. The multispecific protein of any one of claims 82-89, wherein the amino acid sequence of said VH region of said first scFv and the amino acid sequence of said VL region of said first scFv each comprises a cysteine amino acid residue, wherein said cysteine amino acid residues are capable of forming a disulfide bond.

91. The multispecific protein of claim 90, wherein the amino acid sequence of said VH region of said first scFv comprises a cysteine at amino acid position 97, 98, 99, 100, 101, 102, 103, 104, 105, or 106, amino acid numbering according to Kabat; and the amino acid sequence of said VL region of said first scFv comprises a cysteine at amino acid position 40, 41, 42, 43, 44, 45, 46, or 47, amino acid numbering according to Kabat.

92. The multispecific protein of claim 90 or 91, wherein the amino acid sequence of said VH region of said second scFv comprises a cysteine at amino acid position 97, 98, 99, 100, 101, 102, 103, 104, 105, or 106, amino acid numbering according to Kabat; and the amino acid sequence of said VL region of said second scFv comprises a cysteine at amino acid position 40, 41, 42, 43, 44, 45, 46, or 47, amino acid numbering according to Kabat.

93. The multispecific protein of any one of claims 61-92, wherein said first Fc region and said second Fc region are a human IgG (hIgG) isotype.

94. The multispecific protein of any one of claims 61-93, wherein said first Fc region and said second Fc region are hIgG1 or hIgG4 isotype.

95. The multispecific protein of any one of claims 61-94, wherein said first Fc region and said second Fc region are IgG1 isotype, and wherein the amino acid sequence of said first Fc region comprises an amino acid substitution at amino acid positions T366, L368, and Y407, numbering according to the EU index of Kabat.

96. The multispecific protein of any one of claims 61-95, wherein said first Fc region and said second Fc region are IgG1 isotype, and wherein the amino acid sequence of said first Fc region comprises a serine at amino acid position T366, an alanine at amino acid position L368, and a valine at amino acid position Y407, numbering according to the EU index of Kabat.

97. The multispecific protein of any one of claims 61-96, wherein said first Fc region and said second Fc region are IgG1 isotype, and wherein the amino acid sequence of said first Fc region comprises an amino acid substitution at amino acid position Y349, numbering according to the EU index of Kabat.

98. The multispecific protein of any one of claims 61-97, wherein said first Fc region and said second Fc region are IgG1 isotype, and wherein the amino acid sequence of said first Fc region comprises a cysteine at amino acid position Y349, numbering according to the EU index of Kabat.

99. The multispecific protein of any one of claims 61-98, wherein said first Fc region and said second Fc region are IgG1 isotype, and wherein the amino acid sequence of said second Fc region comprises an amino acid substitution at amino acid position T366, numbering according to the EU index of Kabat.

100. The multispecific protein of any one of claims 61-99, wherein said first Fc region and said second Fc region are IgG1 isotype, and wherein the amino acid sequence of said second Fc region comprises a tryptophan at amino acid position T366, numbering according to the EU index of Kabat.

101. The multispecific protein of any one of claims 61-100, wherein said first Fc region and said second Fc region are IgG1 isotype, and wherein the amino acid sequence of said second Fc region comprises an amino acid substitution at amino acid position S354, numbering according to the EU index of Kabat.

102. The multispecific protein of any one of claims 61-101, wherein said first Fc region and said second Fc region are IgG1 isotype, and wherein the amino acid sequence of said second Fc region comprises a cysteine at amino acid position S354, numbering according to the EU index of Kabat.

103. The multispecific protein of any one of claims 61-102, wherein said first Fc region and said second Fc region are IgG1 isotype, and wherein the amino acid sequence of said second Fc region comprises an amino acid substitution at amino acid positions T366, L368, and Y407, numbering according to the EU index of Kabat.

104. The multispecific protein of any one of claims 61-94 or 103, wherein said first Fc region and said second Fc region are IgG1 isotype, and wherein the amino acid sequence of said second Fc region comprises a serine at amino acid position T366, an alanine at amino acid position L368, and a valine at amino acid position Y407, numbering according to the EU index of Kabat.

105. The multispecific protein of any one of claims 61-94 or 103-104, wherein said first Fc region and said second Fc region are IgG1 isotype, and wherein the amino acid sequence of said second Fc region comprises an amino acid substitution at amino acid position Y349, numbering according to the EU index of Kabat.

106. The multispecific protein of any one of claims 61-94 or 103-105, wherein said first Fc region and said second Fc region are IgG1 isotype, and wherein the amino acid sequence of said second Fc region comprises a cysteine at amino acid position Y349, numbering according to the EU index of Kabat.

107. The multispecific protein of any one of claims 61-94 or 103-106, wherein said first Fc region and said second Fc region are IgG1 isotype, and wherein the amino acid sequence of said first Fc region an amino acid substitution at amino acid position T366, numbering according to the EU index of Kabat.

108. The multispecific protein of any one of claims 61-94 or 103-107, wherein said first Fc region and said second Fc region are IgG1 isotype, and wherein the amino acid sequence of said first Fc region comprises a tryptophan at amino acid position T366, numbering according to the EU index of Kabat.

109. The multispecific protein of any one of claims 61-94 or 103-108, wherein said first Fc region and said second Fc region are IgG1 isotype, and wherein the amino acid sequence of said first Fc region comprises an amino acid substitution at amino acid position S354, numbering according to the EU index of Kabat.

110. The multispecific protein of any one of claims 61-94 or 103-109, wherein said first Fc region and said second Fc region are IgG1 isotype, and wherein the amino acid sequence of said first Fc region comprises a cysteine at amino acid position S354, numbering according to the EU index of Kabat.

111. The multispecific protein of any one of claims 61-110, wherein said multispecific protein does not substantially mediate ADCC, does not substantially mediate ADCP, does not substantially mediate CDC, and/or does not bind to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

112. The multispecific protein of any one of claims 61-111, wherein said first Fc region and said second Fc region are IgG1 isotype, and wherein the amino acid sequence of said first Fc region and said second Fc region each comprises an amino acid substitution at amino acid position L234, and/or an amino acid substitution at amino acid position L235, numbering according to the EU index of Kabat.

113. The multispecific protein of any one of claims 61-112, wherein said first Fc region and said second Fc region are IgG1 isotype, and wherein the amino acid sequence of said first Fc region and said second Fc region each comprises an alanine at amino acid position L234 and/or an alanine at amino acid position L235, numbering according to the EU index of Kabat.

114. The multispecific protein of any one of claims 61-113, wherein said first Fc region and said second Fc region are IgG1 isotype, and wherein the amino acid sequence of said first Fc region and said second Fc region each comprises an amino acid substitution at amino acid position L234, an amino acid substitution at amino acid position L235, and/or an amino acid substitution at amino acid position P329, numbering according to the EU index of Kabat.

115. The multispecific protein of any one of claims 61-114, wherein said first Fc region and said second Fc region are IgG1 isotype, and wherein the amino acid sequence of said first Fc region and said second Fc region each comprises an alanine at amino acid position L234, an alanine at amino acid position L235, and/or an alanine at amino acid position P329, numbering according to the EU index of Kabat.

116. The multispecific protein of any one of claims 61-115, wherein said first Fc region and said second Fc region are IgG1 isotype, and wherein the amino acid sequence of said first Fc region and said second Fc region each comprises an alanine at amino acid position L234, an alanine at amino acid position L235, and/or a glycine at amino acid position P329, numbering according to the EU index of Kabat.

117. The multispecific protein of any one of claims 61-116, wherein said first Fc region and said second Fc region are IgG4 isotype, and wherein the amino acid sequence of said first Fc region and said second Fc region each comprises an amino acid substitution at amino acid position S228, an amino acid substitution at amino acid position F234, and/or an amino acid substitution at amino acid position E235, numbering according to the EU index of Kabat.

118. The multispecific protein of any one of claims 61-117, wherein said first Fc region and said second Fc region are IgG4 isotype, and wherein the amino acid sequence of said first Fc region and said second Fc region each comprises a proline at amino acid position S228, an alanine at amino acid position F234, and/or an alanine at amino acid position E235, numbering according to the EU index of Kabat.

119. The multispecific protein of any one of claims 61-118, wherein said tumor associated antigen is hEGFR, hMSLN, hPSMA, or hHER2; and said second tumor associated antigen hEGFR, hMSLN, hPSMA, or hHER2.

120. The multispecific protein of any one of the preceding claims, wherein said hTCSA is hCD28, hCD2, hCD137, hCD27, hCD278, hCD134, or hCD40

121. The multispecific protein of any one of the preceding claims, wherein said hTCSA is hCD28.

122. The multispecific protein of any one of the preceding claims, wherein said hTCSA is hCD2.

123. A polynucleotide encoding the multispecific protein of any one of the preceding claims or one or more polypeptide thereof.

124. The polynucleotide of claim 123, wherein said polynucleotide is RNA (e.g., mRNA) or DNA.

125. The polynucleotide of claim 123 or 124, wherein the polynucleotide is codon optimized.

126. An expression vector comprising the polynucleotide of any one of claims 123-125.

127. The expression vector of claim 126, wherein said expression vector is a viral vector or a plasmid.

128. A host cell comprising the multispecific protein of any one of claims 1-122, the polynucleotide of any one of claims 123-125, or the expression vector of any one of claims 126-127.

129. A carrier comprising the multispecific protein of any one of claims 1-122, the polynucleotide of any one of claims 123-125, or the expression vector of any one of claims 126-127.

130. The carrier of claim 129, wherein said carrier is a lipid nanoparticle, liposome, lipoplex, or nanoliposome.

131. A pharmaceutical composition comprising the multispecific protein of any one of claims 1-122, the polynucleotide of any one of claims 123-125, the expression vector of any one of claims 126-127, the host cell of claim 128, or the carrier of any one of claims 129-130, and a pharmaceutically acceptable excipient.

132. A kit comprising the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131.

133. A method of making the multispecific protein of any one of claims 1-122, comprising:
introducing into a population of in vitro or ex vivo cells the polynucleotide of any one of claims 123-125 or the vector of any one of claims 126-127,
culturing said population of cells under conditions sufficient for said population of cells to express said multispecific protein; and
optionally isolating and/or purifying said multispecific protein.

134. A method of delivering a multispecific protein, polynucleotide, expression vector, host cell, carrier, or pharmaceutical composition to a subject, the method comprising administering the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 to the subject, to thereby deliver the multispecific protein, polynucleotide, expression vector, host cell, carrier, or pharmaceutical composition to the subject.

135. A method of inducing an immune response in a subject, the method comprising administering the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 to the subject, to thereby induce an immune response in the subject.

136. A method of activating a T cell or population of T cells in a subject, the method comprising administering the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 to the subject, to thereby activate a T cell or population of T cells in the subject.

137. A method of preventing or treating a cancer in a subject, the method comprising administering the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 to the subject in need thereof, to thereby prevent or treat the cancer in the subject.

138. The method of claim 137, wherein the cancer is a solid tumor.

139. The method of claim 137 or 138, wherein the cancer is breast cancer, ovarian cancer, endometrial cancer, uterine cancer, cervical cancer, anal cancer, prostate cancer, rectal cancer, kidney cancer, bladder cancer, colon cancer, liver cancer, pancreatic cancer, thyroid cancer, thymus cancer, lung cancer, bronchus cancer, skin cancer, brain cancer, spinal cord cancer, head cancer, neck cancer, lip cancer, or oral cavity cancer.

140. The multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 for use in a method of preventing or treating cancer in a subject, the method comprising administering to the subject the multispecific protein, the polynucleotide, the expression vector, the host cell, the carrier, or the pharmaceutical composition, to thereby prevent or treat the cancer in the subject.

141. Use of the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 for the manufacture of a medicament for preventing or treating cancer in a subject, comprising administering to the subject the multispecific protein, the polynucleotide, the expression vector, the host cell, the carrier, or the pharmaceutical composition, to thereby prevent or treat the cancer in the subject.

142. The multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 for use in a method of inducing an immune response in a subject, the method comprising administering to the subject the multispecific protein, the polynucleotide, the expression vector, the host cell, the carrier, or the pharmaceutical composition, to thereby induce an immune response in the subject.

143. Use of the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 for the manufacture of a medicament for inducing an immune response in a subject, comprising administering to the subject the multispecific protein, the polynucleotide, the expression vector, the host cell, the carrier, or the pharmaceutical composition, to thereby induce an immune response in the subject.

144. The multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 for use in a method of activating a T cell or population of T cells in a subject, the method comprising administering to the subject the multispecific protein, the polynucleotide, the expression vector, the host cell, the carrier, or the pharmaceutical composition, to thereby activate a T cell or population of T cells in the subject.

145. Use of the multispecific protein of any one of claims 1-122, the polynucleotide of any one of claims 123-125, the expression vector of any one of claims 126-127, the host cell of claim 128, the carrier of any one of claims 129-130, or the pharmaceutical composition of claim 131 for the manufacture of a medicament for activating a T cell or population of T cells in a subject, comprising administering to the subject the multispecific protein, the polynucleotide, the expression vector, the host cell, the carrier, or the pharmaceutical composition, to thereby activate a T cell or population of T cells in the subject.
